(19) United States
(12) Patent Application Publication
(10) Pub. No.: US 2024/0268396 A1
(43) Pub. Date: Aug. 15, 2024

(54) VESICLE FORMULATIONS FOR DELIVERY OF ANTIFUNGAL NUCLEIC ACIDS

(71) Applicant: The Regents of the University of California, Oakland, CA (US)

(72) Inventor: Hailing Jin, Oakland, CA (US)

(21) Appl. No.: 18/570,033

(22) PCT Filed: Jun. 16, 2022
(86) PCT No.: PCT/US2022/033879

§ 371 Date: Dec. 13, 2023

Related U.S. Application Data
(60) Provisional application No. 63/211,962, filed on Jun. 17, 2021.

(51) Int. Cl. A01N 63/60; A01N 25/04; A01P 3/00

(52) U.S. Cl. CPC A01N 63/60; A01N 25/04; A01P 3/00

(57) ABSTRACT

Compositions comprising an antifungal RNA and a lipid vesicle are provided, wherein the antifungal RNA comprises a double-stranded RNA, a small RNA, or a small RNA duplex. The lipid vesicle may be, for example, a plant-derived vesicle or an artificial vesicle containing a tertiary amine cationic lipid. For example, the RNA may target a dicer-like (DCL) gene or a long terminal repeat (LTR) region of a fungal pathogen such as Botrytis or Verticillium. Methods for increasing pathogen resistance in plants are also described.

### STATEMENT AS TO RIGHTS TO INVENTIONS MADE UNDER FEDERALLY SPONSORED RESEARCH AND DEVELOPMENT

This invention was made with government support under Grant No. IOS-2017314 awarded by the National Science Foundation. The government has certain rights in the invention.

### BACKGROUND OF THE INVENTION

Fungal pathogens are a threat to global food security and can cause crop yield losses of up to 20% along with additional postharvest product losses of up to 10%. Currently, resistant strains of fungi to every major fungicide used in agriculture have been identified. In order to continue to safeguard global food security, novel strategies for combatting fungal pathogens must be developed. Recent advances have included Spray-Induced Gene Silencing (SIGS), where antifungal RNAs are applied to plant material through spray application. SIGS techniques utilize RNAi technology which allows for the versatile design of antifungal RNAs that are species specific and target multiple genes simultaneously. SIGS has been successfully utilized to control a wide variety of fungal pathogens, insects, and viruses. A major drawback to SIGS approaches is the instability of RNA in the environment, which can be rapidly broken down by RNAses or when exposed to rainfall, high humidity, and UV light. Further, many fungal pathogens are soil-borne, and dsRNAs are rapidly broken down in the soil.

### BRIEF SUMMARY OF THE INVENTION

Provided herein are compositions comprising an antifungal RNA and a lipid vesicle. In some embodiments, the antifungal RNA comprises a double-stranded RNA, a small RNA, or a small RNA duplex. In some embodiments, the lipid vesicle is an artificial vesicle comprising a tertiary amine cationic lipid. In some embodiments, the lipid vesicle is a natural plant-derived vesicle. The vesicle may be, for example, a micelle, a small unilamellar vesicle, a large unilamellar vesicle, or a multilamellar vesicle. The cationic lipid may be an amine such as N-(1-(2,3-dioleoyloxy)propyl)-N,N,N-trimethylammonium chloride (DOTAP), N,N-dimethyl-2,3-dioleyloxy)propylamine (DODMA), or the like. In some embodiments, the vesicle further comprises a sterol. In some embodiments, the antifungal RNA targets the dicer-like (DCL) genes of a fungal pathogen such as Botrytis or Verticillium. In some embodiments, the antifungal RNA targets genes such as those involved in the pathogen trafficking/secretion pathways (e.g., vacuolar protein sorting 51 (VPS51), dynactin (DCTN1), and suppressor of actin (SAC1) of such pathogens. In some embodiments, the antifungal RNA targets a long terminal repeat (LTR) region of such pathogens.

Also provided herein are methods for increasing pathogen resistance in plants. The methods include contacting the plant with an antifungal RNA composition according to the present disclosure. For example, vesicles containing antifungal RNA may be sprayed onto crops or ornamental plants so as to protect pre-harvest crops and post-harvest products, including but not limited to, fruits, vegetables, and flowers.

### BRIEF DESCRIPTION OF THE DRAWINGS

FIGS. 1A-1D: dsRNA loaded into AVs is shielded from nuclease degradation and easily taken up by Botrytis cinerea. (FIG. 1A) AV-Bc-DCL1/2-dsRNA (the dsRNA contains the RNA fragments targeting Bc DCL1 and DCL2) lipoplexes were formed at a range of indicated charge ratios (N:P) and incubated for 2 h at room temperature before being loaded onto 2% agarose gel. Complete loading was achieved to an AVs:dsRNA mass ratio of 4:1. (FIG. 1B) The stability of naked- and AV-Bc-DCL1/2-dsRNA was tested after MNase treatment. Bc-DCL1/2-dsRNA was released from AVs using 1% Triton X-100 before gel electrophoresis. (FIG. 1C) Fluorescein-labeled naked-Bc-DCL1/2 dsRNA, AV-Bc-DCL1/2-dsRNA, and AV-Bc-DCL1/2-dsRNA+Triton and MNase. (FIG. 1D) Fluorescein-labeled naked- or AV-Bc-DCL1/2-dsRNA were added to B. cinerea spores and fluorescent signals were detected in B. cinerea cells after culturing on PDA medium for 10 h. MNase treatment was performed 30 min before image acquisition. Fluorescence signals remained visible in the B. cinerea cells treated with AV-Bc-DCL 1/2-dsRNA using Triton X-100 and MNase treatment before observation. Scale bars, 20 μm

FIG. 2A-2E: Alternative AV formulations protect dsRNA from nuclease degradation and are easily taken up by Botrytis cinerea (FIG. 2A) DOTAP AV-Bc-DCL1/2-dsRNA lipoplexes were formed at a range of indicated charge ratios (N:P) and incubated for 2 h at room temperature before being loaded onto 2% agarose gel. Complete loading was achieved to an AVs:dsRNA mass ratio of 1:1. (FIG. 2B) DODMA AV-Bc-DCL1/2-dsRNA lipoplexes were formed at a range of indicated charge ratios (N:P) and incubated for 2 h at room temperature before being loaded onto 2% agarose gel. Complete loading was achieved to an AVs:dsRNA mass ratio of 4:1. (FIG. 2C) The stability of naked-, DOTAP-, and DODMA-Bc-DCL 1/2-dsRNA was tested after MNase treatment. Bc-DCL1/2-dsRNA was released from AVs using 1% Triton X-100 before gel electrophoresis. (FIG. 2D) The size distributions of the dsRNA-loaded AV formulations were determined using dynamic light scattering. Data shown is the average of three individual measurements. (FIG. 2E) Analysis of B. cinerea uptake of fluorescein-labeled dsRNA encapsulated in three different AV formulations (DOTAP+PEG, DOTAP and DODMA) after 3 and 16 hours of incubation. Fluorescence signals are visible in the B. cinerea cells treated with the three AV-Bc-DCL1/2-dsRNA using Triton X-100 and MNase treatment before observation.

FIGS. 3A-3C: Treatment with all DOTAP+PEG, DOTAP and DODMA AV-dsRNA formulations provide prolonged protection against B. cinerea in tomato fruits. (FIG. 3A) Tomato fruits were pre-treated with naked- or AV(DOTAP+PEG)-Bc-VDS-dsRNA, AV(DOTAP)-Bc-VDS-dsRNA and AV(DODMA)-Bc-VDS-dsRNA, for 1, 5, and 10 days, then inoculated with B. cinerea. Pictures were taken at 5 dpi. (FIG. 3B) Relative lesion sizes were measured with the help of ImageJ software. Error bars indicate the SD. Statistical significance (Student's t-test): *, P<0.05. (FIG. 3C) Relative fungal biomass was quantified by qPCR. Fungal RNA relative to tomato RNA was measured by assaying the fungal actin gene and the tomato actin gene by qPCR using RNA extracted from the infected fruits at 5 dpi. Statistical significance (Student's t-test): *,P<0.05; ** P<0.01.

FIGS. 4A and 4B: Treatment with AV-dsRNA provides prolonged protection against B. cinerea in tomato fruits, grape berries and V vinfera leaves. (FIG. 4A) Tomato fruits and grape berries, as well as grape leaves were pre-treated with naked- or AV-Bc-VDS-dsRNA, for 1, 5, and 10 days; or 1, 7, 14, and 21 days respectively, then inoculated with B. cinerea. Pictures were taken at 5 dpi (fruits) or 5 dpi (grape leaves). (FIG. 4B) Relative lesion sizes were measured with the help of ImageJ software. Error bars indicate the SD. Statistical significance (Student's t-test): * P<0.05.

FIG. 5A shows fluorescently labeled dsRNA encapsulated in natural extracellular vesicles.

FIG. 5B shows that natural extracellular vesicle-encapsulated Bc-DCL1/2-dsRNA efficiently inhibited the fungal disease caused by B. cinerea.

FIGS. 6A-6C: Externally applied naked-dsRNAs or AVs-dsRNA inhibited pathogen virulence. (FIG. 6A) External application of naked- and AV-Bc-VDS-dsRNA (the dsRNA contains the RNA fragments targeting the following three Botrytis genes VPS51, DCTN1 and SAC1), as well as the application of naked- and AV-Bc-DCL1/2-dsRNA (20 μl at a concentration of 20 ng μl⁻¹ of synthetic RNAs), inhibited B. cinerea virulence on tomato fruits, grape berries, lettuce leaves and rose petals compared to the water, AVs empty, naked- or AV-YFP-dsRNA treatments. (FIG. 6B) Relative lesion sizes were measured at 5 dpi on tomato and grape fruits, and at 3 dpi on lettuce leaves and rose petals, and with the help of ImageJ software. Error bars indicate the SD of 10 samples, and three technical repeats were conducted for relative lesion sizes. Statistical significance (Student's t-test): *, P<0.05. (FIG. 6C) Relative expression of the target genes in the pathogen.

FIGS. 7A-7E: Adherence and stability of dsRNA loaded into AVs on Arabidopsis leaves. (FIG. 7A) CLSM analysis of Arabidopsis leaves 1 dpt before and after a water rinsing treatment shows the capability of AVs to protect dsRNA molecules from the mechanical action exerted by the water. Scale bars, 50 m. (FIG. 7B) Arabidopsis leaves were treated with Fluorescein-labeled naked- or AV-dsRNA for 1 and 10 days. The fluorescent signals on the surface of leaves were observed using CLSM. Scale bars, 50 m. (FIG. 7C) The AV-Bc-VDS-dsRNA is highly stable compared with Naked-Bc-VDS-dsRNA on Arabidopsis leaves at dpt, as detected by Northern Blot. (FIG. 7D) Lesions on Arabidopsis leaves inoculated with B. cinerea at 1, 3, and 14 dpt. (FIG. 7E) Relative lesion sizes were measured 3 dpi with the help of ImageJ software. Error bars indicate the SD. Statistical significance (Student's t-test): *, P<0.05.

FIGS. 8A and 8B: Natural EVs were isolated from the juice of different fruits and vegetables, including watermelon, carrots, lemon, orange, tomato and cucumber, etc. and characterization of PDEVs from fruit and vegetable juices. EVs were collected from various fruit and vegetable juices using differential ultracentrifugation and characterized using transmission electron microscopy (TEM) and nanoparticle tracking analysis (NTA). (FIG. 8A) Representative TEM image of lime EVs compared to TEM of plant EVs. (FIG. 8B). Representative size distribution of plant EVs and PDEVs as determined using NTA. The distributions shown are the average of three 60-second videos.

FIGS. 9A and 9B: PDEVs can be loaded with dsRNA and deliver dsRNA to B. cinerea. (FIG. 9A) Equal concentrations of PDEVs were loaded with either 40 or 80 ng of dsRNA (1^{st} and 2nd lane of each set respectively) after 2 hrs at room temperature. RNA loading differences were observed based on PDEV juice source. (FIG. 9B) B. cinerea was incubated with either naked fluorescein-labeled dsRNA or fluorescein-labeled dsRNA loaded into PDEVs for 3 hours. Pictures were taken using confocal laser scanning microscopy. Fluorescence signals are visible in B. cinerea cells treated with either naked dsRNA or PDEVs, indicating dsRNA uptake and delivery. Samples were treated with Triton X-100 and MNase 30 mins prior to imaging to disrupt EVs not taken up by the fungal cells and degrade free dsRNA, respectively.

FIG. 10: PDEVs loaded with dsRNA can provide protection to plant material against B. cinerea infection. PDEVs were loaded with 100 ng/μL of VDS dsRNA overnight and tomato fruits were then treated with 20 μL of water, naked VDS dsRNA, or the PDEVs+VDS dsRNA. The next day, tomatoes were inoculated with B. cinerea spores and lesions were measured 5 days post inoculation. ** denotes p<0.01 compared to water.

### DETAILED DESCRIPTION OF THE INVENTION

Provided herein are vesicles for stabilization and delivery of antifungal RNAs to fungal pathogens. These artificial vesicles can be used in Spray-Induced Gene Silencing (SIGS) approaches to protect crops and post-harvest plant material from fungal pathogens and other pests. Once loaded with pathogen or pest targeting RNAs, the Artificial Vesicles can be sprayed onto plant tissues to confer protection against the pathogen or pest.

### I. Definitions

The term “pathogen resistance” refers to an increase in the ability of a plant to prevent or resist pathogen infection or pathogen-induced symptoms. Pathogen resistance can be increased resistance relative to a particular pathogen species or genus (e.g., Botrytis), increased resistance to multiple pathogens, or increased resistance to all pathogens (e.g., systemic acquired resistance). In some embodiments, resistance of a plant to a pathogen is “increased” when one or more symptoms of pathogen infection are reduced relative to a control (e.g., a plant in which a polynucleotide that inhibits expression of a fungal pathogen DCL gene is not expressed).

“Pathogens” include, but are not limited to, viruses, bacteria, nematodes, fungi or insects (see, e.g., Agrios, Plant Pathology (Academic Press, San Diego, CA (1988)). In some embodiments, the pathogen is a fungal pathogen.

The terms “nucleic acid” and “polynucleotide” refer to a single or double-stranded polymer of deoxyribonucleotide or ribonucleotide bases read from the 5′ end to the 3′ end. Nucleic acids may also include modified nucleotides that permit correct read through by a polymerase and do not significantly alter expression of a polypeptide encoded by that nucleic acid.

Two nucleic acid sequences or polypeptides are said to be “identical” if the sequence of nucleotides or amino acid residues, respectively, in the two sequences is the same when aligned for maximum correspondence as described below. “Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. When percentage of sequence identity is used in reference to proteins or peptides, it is recognized that residue positions that are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, California, USA).

The terms “substantial identity” and “substantially identical,” as used in the context of polynucleotide or polypeptide sequences, refer to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, WI), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

The term “complementary to” is used herein to mean that a polynucleotide sequence is complementary to all or a portion of a reference polynucleotide sequence. In some embodiments, a polynucleotide sequence is complementary to at least 15, at least 20, at least 25, at least 30, at least 40, at least 50, at least 75, at least 100, at least 125, at least 150, at least 175, at least 200, or more contiguous nucleotides of a reference polynucleotide sequence. In some embodiments, a polynucleotide sequence is “substantially complementary” to a reference polynucleotide sequence if at least 70%, at least 75%, at least 80%, at least 85%, at least 90%, or at least 95% of the polynucleotide sequence is complementary to the reference polynucleotide sequence.

The term “promoter,” as used herein, refers to a polynucleotide sequence capable of driving transcription of a coding sequence in a cell. Promoters may include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “plant promoter” is a promoter capable of initiating transcription in plant cells. A “constitutive promoter” is one that is capable of initiating transcription in nearly all tissue types, whereas a “tissue-specific promoter” initiates transcription only in one or a few particular tissue types. An “inducible promoter” is one that initiates transcription only under particular environmental conditions or developmental conditions.

The term “plant” includes whole plants, shoot vegetative organs and/or structures (e.g., leaves, stems and tubers), roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, plant tissue (e.g., vascular tissue, ground tissue, and the like), cells (e.g., guard cells, egg cells, trichomes and the like), and progeny of same. A particular plant may be, for example, an angiosperm (a monocotyledonous or dicotyledonous plant), a gymnosperm, a fern, or a multicellular alga. Plants may be of a variety of ploidy levels, including aneuploid, polyploid, diploid, haploid, and hemizygous.

As used herein, the term “vesicle” encompasses any compartment enclosed by a lipid structure such as a lipid monolayer or a lipid bilayer. The vesicles may be, for example, liposomes, lipid micelles, and non-micellar lipid particles. The vesicle may be an artificial vesicle prepared in vitro, or a natural vesicle prepared from a plant or other organism. Vesicles include unilamellar vesicles containing a single lipid bilayer and generally having diameter in the range of about 20 nm to 10 μm. “Small unilamellar vesicles,” or SUVs typically range from about 20 nm to about 200 nm in size. Vesicles can also be multilamellar, which generally have a diameter in the range of 1 to 10 m. Vesicles may also be below 20 nm in size.

As used herein, the term “vesicle size” refers to the outer diameter of the vesicle. Average particle size can be determined by a number of techniques including dynamic light scattering (DLS), quasi-elastic light scattering (QELS), and electron microscopy.

As used herein, the term “polydispersity index” refers to the size distribution of a population of vesicles. Polydispersity index can be determined by a number of techniques including dynamic light scattering (DLS), quasi-elastic light scattering (QELS), and electron microscopy. Polydispersity index (PDI) is usually calculated as:

$$P D I = {( \frac{σ}{d} )}^{2}$$

i.e., the square of (standard deviation/mean diameter).

As used herein, the term “lipid” refers to lipid molecules that can include fats, waxes, steroids, cholesterol, fat-soluble vitamins, monoglycerides, diglycerides, phospholipids, sphingolipids, glycolipids, cationic or anionic lipids, derivatized lipids, and the like. Lipids can form micelles, monolayers, and bilayer membranes. The lipids can self-assemble into vesicles as described herein.

As used herein, the term “cationic lipid” refers to a positively charged amphiphile, which generally contains a hydrophilic headgroup which is positively charged (e.g., via the protonation of one or several amino groups and a hydrophobic portion (e.g., containing a steroid or one or more alkyl chains).

As used herein, the term “sterol” refers to a steroid containing at least one hydroxyl group. A steroid is characterized by the presence of a fused, tetracyclic gonane ring system. Sterols include, but are not limited to, cholesterol (i.e., 2,15-dimethyl-14-(1,5-dimethylhexyl)-tetracyclo[8.7.0.0^{2,7}.0^{11,15}]heptacos-7-en-5-ol; Chemical Abstracts Services Registry No. 57-88-5).

As used herein, the term “about” indicates a close range around a numerical value when used to modify that specific value. If “X” were the value, for example, “about X” would indicate a value from 0.9X to 1.1X, e.g., a value from 0.95X to 1.05X, or a value from 0.98X to 1.02X, or a value from 0.99X to 1.01X. Any reference to “about X” specifically indicates at least the values X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.1X, and values within this range.

### II. Antifungal RNA Vesicle Compositions

Provided herein are compositions comprising an antifungal RNA and a lipid vesicle for delivery of the RNA to fungal pathogens on plants. The antifungal RNA comprises a double-stranded RNA, a small RNA, or a small RNA duplex. In some embodiments, the lipid vesicle comprises a cationic lipid that complexes with the RNA (e.g., a tertiary amine cationic lipid). In some embodiments, the lipid vesicle is a natural, plant-derived lipid vesicle (e.g., an extracellular vesicle, a plant-derived extracellular vesicle (PDEV)). Vesicles according to the present disclosure may contain a variety of cationic lipids and other lipids, including fats, waxes, steroids, sterols, cholesterol, fat-soluble vitamins, monoglycerides, diglycerides, phospholipids, sphingolipids, glycolipids, amphiphilic or anionic lipids, and the like.

In some embodiments, the cationic lipid comprises a tertiary amine cationic lipid. Examples of such lipids include, but are not limited to, N,N-dimethyl-2,3-dioleyloxy)propylamine (DODMA), N,N-dioleoyl-N,N-dimethylammonium chloride (DODAC), N,N-distearyl-N,N-dimethylammonium bromide (DDAB).

The vesicles may further contain a primary amine, a secondary amine, a quaternary amine, or a combination thereof. The vesicles may contain, for example, N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), and. The ratio of amine in the cationic lipid to phosphate in the RNA may vary, e.g., from about 1:1 to about 10:1. In some embodiments, the ratio of amine in the cationic lipid to phosphate in the RNA is about 4:1. In some embodiments, the vesicles are substantially free or entirely free of quaternary amines such as DOTAP.

In some embodiments, the vesicles contain at least one sterol. The sterol may be, for example, cholesterol or a cholesterol derivative, such as 2,15-dimethyl-14-(1,5-dimethylhexyl)tetracyclo[8.7.0.0^{2,7}.0^{11,15}]heptacos-7-en-5-ol). The vesicles can contain other steroids, characterized by the presence of a fused, tetracyclic gonane ring system. Examples of steroids include, but are not limited to, cholic acid, progesterone, cortisone, aldosterone, testosterone, dehydroepiandrosterone, and estradiol. Synthetic steroids and derivatives thereof are also contemplated for use in the vesicles. In some embodiments, the vesicles contain cationic lipid and cholesterol in a molar ratio ranging from about 1:1 to about 10:1. The vesicles may contain, for example, DODMA:Chol in a ratio of about 2:1.

In some embodiments, the vesicles also contain a (polyethylene glycol)-lipid, also referred to as a PEG-lipid. The term “PEG-lipid” refers to a poly(ethylene glycol) polymer covalently bonded to a hydrophobic or amphiphilic lipid moiety. The lipid moiety can include fats, waxes, steroids, fat-soluble vitamins, monoglycerides, diglycerides, phospholipids, and sphingolipids. For example, the PEG-lipid may be a diacyl-phosphatidylethanolamine-N-[methoxy(polyethylene glycol)] or an N-acyl-sphingosine-1-{succinyl[methoxy(polyethylene glycol)]}. The molecular weight of the PEG in the PEG-lipid is generally from about 500 to about 5000 Daltons (Da; g/mol). The PEG in the PEG-lipid can have a linear or branched structure. In some embodiments, the (polyethylene glycol)-lipid is a (polyethylene glycol)-phosphatidylethanolamine. The vesicles may include any suitable poly(ethylene glycol)-lipid derivative (PEG-lipid). In some embodiments, the PEG-lipid is a diacyl-phosphatidylethanolamine-N-[methoxy(polyethylene glycol)]. The molecular weight of the poly(ethylene glycol) in the PEG-lipid is generally in the range of from about 500 Daltons (Da) to about 5000 Da. The poly(ethylene glycol) can have a molecular weight of, for example, about 750 Da, about 1000 Da, about 2500 Da, or about 5000 Da, or about 10,000 Da, or any molecular weight within this range. In some embodiments, the PEG-lipid is selected from distearoyl-phosphatidylethanolamine-N-[methoxy(polyethylene glycol)-2000] (DSPE-PEG-2000) and distearoyl-phosphatidylethanolamine-N-[methoxy(polyethylene glycol)-5000] (DSPE-PEG-5000). The molar ratio of the cationic lipid to the DSPE-PEG ranges from about 1:0.05 to about 1:1. In some embodiments, the vesicles contain DOTAP:Chol:DSPE-PEG-2000 in a ratio of about 2:1:0.1. In some embodiments, the vesicles are substantially free or entirely free of PEG-lipids.

In some embodiments, the vesicle comprises an amphiphilic lipid such as a phosphatidylcholine lipid. Suitable phosphatidylcholine lipids include saturated PCs and unsaturated PCs. Examples of saturated PCs include 1,2-dilauroyl-sn-glycero-3-phosphocholine (DLPC), 1,2-dimyristoyl-sn-glycero-3-phosphocholine (dimyristoylphosphatidylcholine; DMPC), 1,2-distearoyl-sn-glycero-3-phosphocholine (distearoylphosphatidylcholine; DSPC), 1,2-dioleoyl-sn-glycero-3-phosphocholine (DOPC), 1,2-dipalmitoyl-sn-glycero-3-phosphocholine (dipalmitoylphosphatidylcholine; DPPC), 1-myristoyl-2-palmitoyl-sn-glycero-3-phosphocholine (MPPC), 1-palmitoyl-2-myristoyl-sn-glycero-3-phosphocholine (PMPC), 1-myristoyl-2-stearoyl-sn-glycero-3-phosphocholine (MSPC), 1-palmitoyl-2-stearoyl-sn-glycero-3-phosphocholine (PSPC), 1-stearoyl-2-palmitoyl-sn-glycero-3-phosphocholine (SPPC), and 1-stearoyl-2-myristoyl-sn-glycero-3-phosphocholine (SMPC).

Examples of unsaturated PCs include, but are not limited to, 1,2-dimyristoleoyl-sn-glycero-3-phosphocholine, 1,2-dimyristelaidoyl-sn-glycero-3-phosphocholine, 1,2-dipamiltoleoyl-sn-glycero-3-phosphocholine, 1,2-dipalmitelaidoyl-sn-glycero-3-phosphocholine, 1,2-dioleoyl-sn-glycero-3-phosphocholine (DOPC), 1,2-dielaidoyl-sn-glycero-3-phosphocholine, 1,2-dipetroselenoyl-sn-glycero-3-phosphocholine, 1,2-dilinoleoyl-sn-glycero-3-phosphocholine, 1-palmitoyl-2-oleoyl-sn-glycero-3-phosphocholine (palmitoyloleoylphosphatidylcholine; POPC), 1-palmitoyl-2-linoleoyl-sn-glycero-3-phosphocholine, 1-stearoyl-2-oleoyl-sn-glycero-3-phosphocholine (SOPC), 1-stearoyl-2-linoleoyl-sn-glycero-3-phosphocholine, 1-oleoyl-2-myristoyl-sn-glycero-3-phosphocholine (OMPC), 1-oleoyl-2-palmitoyl-sn-glycero-3-phosphocholine (OPPC), and 1-oleoyl-2-stearoyl-sn-glycero-3-phosphocholine (OSPC). Lipid extracts, such as egg PC, heart extract, brain extract, liver extract, soy PC, and hydrogenated soy PC (HSPC) may also be employed.

Other suitable phospholipids include phosphatidic acids (PAs), phosphatidylethanolamines (PEs), phosphatidylglycerols (PGs), phosphatidylserine (PSs), and phosphatidylinositol (PIs). Examples of such phospholipids include, but are not limited to, dimyristoylphosphatidylglycerol (DMPG), distearoylphosphatidylglycerol (DSPG), dioleoylphosphatidylglycerol (DOPG), dipalmitoylphosphatidylglycerol (DPPG), dimyristoylphosphatidylserine (DMPS), distearoylphosphatidylserine (DSPS), dioleoylphosphatidylserine (DOPS), dipalmitoylphosphatidylserine (DPPS), dioleoylphosphatidylethanolamine (DOPE), palmitoyloleoylphosphatidylcholine (POPC), palmitoyloleoylphosphatidylethanolamine (POPE), dipalmitoylphosphatidylethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearoyl-2-oleoyl-phosphatidyethanolamine (SOPE), dielaidoylphosphoethanolamine (transDOPE), and cardiolipin.

The vesicles may be unilamellar, containing a single lipid bilayer and generally having a diameter in the range of about 20 to about 400 nm. The vesicles can also be multilamellar, which generally have a diameter in the range of 1 to 10 m. In some embodiments, vesicles can include multilamellar vesicles (MLVs; e.g., from about 1 m to about 10 m in size), large unilamellar vesicles (LUVs; e.g., from a few hundred nanometers to about 10 m in size), and small unilamellar vesicles (SUVs; e.g., from about 20 nm to about 200 nm in size). In some embodiments, the vesicles are lipid micelles (e.g., below about 20 nm in size).

Populations of vesicles described herein may be polydisperse, may have low polydispersities, or may be monodisperse. In some embodiments, the vesicles have a polydispersity index that is less than 0.3, less than 0.2, less than 0.15, or less than 0.10, as measured by DLS.

Lipid vesicles can be prepared by hydrating a dried lipid film (prepared via evaporation of a mixture of the lipid and an organic solvent in a suitable vessel) with water or an aqueous solution (e.g., 5% dextrose in RNase-free deionized water). Hydration of lipid films typically results in a suspension of multilamellar vesicles (MLVs). Alternatively, MLVs can be formed by diluting a solution of a lipid in a suitable solvent, such as a C₁₋₄ alkanol, with water or an aqueous solution. Unilamellar vesicles can be formed from MLVs via sonication or extrusion through membranes with defined pore sizes. Encapsulation of RNAs can be conducted by including the RNAs in the aqueous solution used for film hydration or lipid dilution during MLV formation. RNAs can also be encapsulated in pre-formed vesicles.

Natural lipid vesicles can also be produced by various plants, and may be obtained from leaves, fruits, or other plant tissue. Vegetables for use in preparation of the plant-derived vesicles include, but are not limited to, species of Abutilon, Acacia, Acmella, Althaea, Amaranthus, Apium, Atriplex, Barbarea, Barringtonia, Basella, Beta, Borago, Brassica, Calamus, Campanula, Capparis, Celosia, Centella, Chenopodium, Chrysanthemum, Cichorium, Cirsium, Claytonia, Cleome, Cnidoscolus, Coccinia, Colocasia, Corchorus, Coriandrum, Crambe, Crassocephalum, Cratoxylum, Crithmum, Crotalaria, Cryptotaenia, Cucumis, Cucurbita, Cyclanthera, Cynara, Diplazium, Diplotaxis, Erythrina, Eruca, Emex, Eryngium, Foeniculum, Galactites, Galinsoga, Glechoma, Glinus, Gnetum, Gynura, Halimione, Hibiscus, Hirschfeldia, Honckenya, Houttuynia, Hydrophyllum, Hyoseris, Hypochaeris, Inula, Ipomoea, Kleinhovia, Lablab, Lactuca, Lagenaria, Lallemantia, Lamium, Lapsana, Launaea, Leichhardtia, Leontodon, Lepidium, Leucaena, Levisticum, Limnocharis, Limnophila, Lysimachia, Malva, Manihot, Marsilea, Matteuccia, Megacarpaea, Melanthera, Mentha, Mertensia, Mesembryanthemum, Mimulus, Mirabilis, Morinda, Moringa, Mycelis, Myrianthus, Myriophyllum, Myrrhis, Nasturtium, Neptunia, Nymphaea, Nymphoides, Ocimum, O., Oenanthe, Oenothera, Onoclea, Oroxylum, Oryza, Osmorhiza, Osmunda, Oxalis, Oxyria, Pachira, Paederia, Parkia, Parkinsonia, Pastinaca, Patrinia, Paulownia, Pedalium, Peperomia, Pereskia, Pergularia, Perilla, Persicaria, Petasites, Petroselinum, Peucedanum, Phaseolus, Phragmites, Phyla, Phyllanthus, Phyteuma, Phytolacca, Pimpinella, Pinus, Piper, Pipturus, Pisonia, Pistacia, Pistia, Pisum, Plantago, Pluchea, Podophyllum, Poliomintha, Polygonum, Poncirus, Pontederia, Portulaca, Portulacaria, Primula, Pringlea, Prosopis, Prunella, Psoralea, Pteris, Ptychosperma, Pulicaria, Pulmonaria, Puya, Pyrus, Ranunculus, Raphanus, Raphia, Reichardia, Rhamnus, Rheum, Rhexia, Rhodiola, Rhododendron, Rhopalostylis, Ribes, Rorippa, Rosa, Roystonea, Rubus, Rumex, Salicornia, Salix, Salsola, Salvadora, Sambucus, Sanguisorba, Sassafras, Sauropus, Saxifraga, Schleichera, Scolymus, Scorzonera, Scutellaria, Sechium, Sedum, Senna, Sesamum, Sesbania, Sesuvium, Setaria, Sicyos, Sida, Sidalcea, Silaum, Silene, Silybum, Sinapis, Sisymbrium, Sium, Smyrnium, Solenostemon, Solidago, Sonchus, Sophora, Spathiphyllum, Sphenoclea, Sphenostylis, Spilanthes, Spinacia, Spirodela, Spondias, Stanleya, Stellaria, Stenochlaena, Sterculia, Strychnos, Suaeda, Symphytum, Synedrella, Syzygium, Talinum, Tanacetum, Taraxacum, “Telfairia, Telosma, Tetracarpidium, Tetragonia, Thalia, Thespesia, Thlaspi, Thymus, Tiliacora, Toddalia, Toona, Tordylium, Trachycarpus, Tradescantia, Tragopogon, Trianthema, Trichodesma, Trifolium, Trigonella, Trillium, Tropaeolum, Tulbaghia, Tussilago, Typha, Ullucus, Ulmus, Urena, Urtica, Valerianella, Vallaris, Verbena, Vernonia, Veronica, Veronicastrum, Viola, Vitex, Vitis, Wasabia, Wisteria, Wolffia, Xanthoceras, Xanthosoma, Ximenia, Zanthoxylum, and/or Zingiber. For example, plant-derived vesicles may be prepared from various varieties of lettuce, cabbage, chard, collard, beet, chicory, cress, spinach, endives, kale, parsley, or the like. One of skill in the art will appreciate that a designation as “fruit” or “vegetable” will not materially affect the use of any particular plant as a source for plant-derived vesicles. Squashes such as calabash (Lagenaria siceraria) or tomatoes (Solanum lycopersicum), for example, may be termed as fruits and/or vegetables in common usage.

Fruits for use in preparation of the plant-derived vesicles include, but are not limited to, species of Acronychia, Acrotriche, Actinidia, Aegle, Aglaia, Amelanchier, Ananas, Annona, Antidesma, Arbutus, Archirhodomyrtus, Arctostaphylos, Ardisia, Aristotelia, Aronia, Artocarpus, Asimina, Austromyrtus, Averrhoa, Azadirachta, Baccaurea, Berberis, Billardiera, Blighia, Boquila, Borassus, Bouea, Buchanania, Bunchosia, Butia, Byrsonima, Calamus, Calligonum, Canarium, Capparis, Carica, Carissa, Carnegiea, Carpobrotus, Caryocar, Casimiroa, Cassytha, Celtis, Cereus, Choerospondias, Chrysobalanus, Chrysophyllum, Citropsis, Citrullus, Citrus, Clausena, Coccoloba, Cocos, Coffea, Cola, Cornus, Crataegus, Crescentia, Cucumis, Cydonia, Dacryodes, Davidsonia, Decaisnea, Dialium, Dillenia, Dimocarpus, Diospyros, Diploglottis, Dovyalis, Duguetia, Durio, Elaeagnus, Elaeis, Eleiodoxa, Empetrum, Eriobotrya, Euclea, Eugenia, Eupomatia, Euterpe, Feijoa, Ficus, Flacourtia, Fragaria, Fuchsia, Garcinia, Gaultheria, Genipa, Glenniea, Gomortega, Grewia, Hancornia, Heteromeles, Hippophae, Hydnora, Hylocereus, Hymenaea, Inga, Irvingia, Kunzea, Lansium, Lardizabala, Licania, Limonia, Litchi, Litsea, Lodoicea, Lonicera, Lycium, Maclura, Mahonia, Malpighia, Malus, Mammea, Mangifera, Manilkara, Mauritia, Melastoma, Melicoccus, Melodorum, Mespilus, Mimusops, Momordica, Monstera, Morinda, Morus, Muntingia, Murraya, Musa, Myrciaria, Myrica, Myristica, Myrtillocactus, Nephelium, Opuntia, Owenia, Pachycereus, Pandanus, Pangium, Parajubaea, Parkia, Passiflora, Pentadiplandra, Phoenix, Phyllanthus, Physalis, Pithecellobium, Planchonia, Platonia, Pleiogynium, Plinia, Podophyllum, Pourouma, Pouteria, Prunus, Pseudocydonia, Psidium, Punica, Pyracantha, Pyrus, Quararibea, Ribes, Rollinia, Rosa, Rubus, Sageretia, Salacca, Sambucus, Sandoricum, Santalum, Sclerocarya, Serenoa, Shepherdia, Sicana, Siraitia, Solanum, Sorbus, Spondias, Stelechocarpus, Strychnos, Synsepalum, Syzygium, Tamarindus, Terminalia, Theobroma, Trichosanthes, Triphasia, Ugni, Vaccinium, Vangueria, Vanilla, Viburnum, Vitis, Ximenia, or Ziziphus. For example, plant-derived vesicles may be prepared from various varieties of orange, lemon, lime, grapefruit, tangerine, cherry, peach, plum, pear, apple, apricot, pluot, nectarine, banana, plantain, watermelon, cantaloupe, casaba, cucumber, pineapple, passionfruit, mango, kiwi, starfruit, blueberry, raspberry, strawberry, durian, gooseberry, currant, grape, cranberry, fig, or the like.

In some embodiments, natural lipid vesicles are obtained from Nicotiana benthamiana leaves, ginger plants, melon, tomato, lemon, cherry, or grape. Such vesicles can be isolated by techniques including, but not limited to, sequential centrifugation and sequential filtration, or by using commercially available purification kits, e.g., exoEasy Maxi Kit (Qiagen).

As a non-limiting example, leaf extracellular fluid or extracted fruit juice can be sequentially centrifuged at 1000×g for 10 min, and 10 000×g for 40 min to remove large particles. The supernatant can then be centrifuged at 100-150,000×g for 90 min to collect extracellular vesicles (e.g., plant-derived extracellular vesicles (PDEVs)). Leaf extracellular fluid or extracted fruit juice can also be subjected to sequential filtration for lipid vesicle purification. First, floating cells and cell debris can be depleted by using a 0.1 μm Millipore Express (PES) membrane Stericup Filter Unit. The filtrate can then be further filtered through a 500-kDa MWCO mPES hollow fiber MidiKros filter module to remove free proteins, with vesicles retained as retentate. Optional further separation of exosomes can be achieved by filtering using 100-nm Track Etch filter (Millipore, Billerica, MA, USA). Natural lipid vesicles can be also isolated by exoEasy Maxi Kit (Qiagen). The exoEasy Maxi Kit uses a membrane-based affinity binding step to isolate exosomes and other vesicles from serum and plasma or cell culture supernatant.

### II. Antifungal RNAs

RNAi is the phenomenon in which when a double-stranded RNA having a sequence identical or similar to that of the target gene is introduced into a cell, the expressions of both the inserted exogenous gene and target endogenous gene are suppressed. The double-stranded RNA may be formed from two separate complementary RNAs or may be a single RNA molecule that comprises internally complementary sequences that form a double-stranded RNA region. RNAi is also known to be effective in plants in reducing levels of RNA of expressed by target gene of interest (see, e.g., Chuang, C. F. & Meyerowitz, E. M., Proc. Natl. Acad. Sci. USA 97: 4985 (2000); Waterhouse et al., Proc. Natl. Acad. Sci. USA 95:13959-13964 (1998); Tabara et al. Science 282:430-431 (1998); Matthew, Comp Funct. Genom. 5: 240-244 (2004); Lu, et al., Nucleic Acids Research 32(21):e171 (2004)).

RNA in the vesicles can target any gene of interest, e.g., a gene from a pathogen of interest. In some embodiments, the RNA targets a fungal pathogen. Examples of plant fungal pathogens include, but are not limited to, Botyritis, Verticillium, Rhizoctonia, Aspergillus, Sclerotinia, Magnaporthe, Puccinia, Fusarium, Mycosphaerella, Blumeria, and Melampsora. See, e.g., Dean et al. (Mol Plant Pathol 13:804 (2012)); Wang and Jin, et al. Nature Plants, 2, 16151 (2016); Qiao and Jin, et al. Plant Biotechnology Journal, 2021, doi: 10.1111/pbi.13589; WO 2016/176324; and WO 2019/079044, which are incorporated herein by reference in their entirety. Although the sequences used for RNAi need not be completely identical to the target gene sequences, they may be at least 70%, 80%, 90%, 95% or more identical to the target gene sequence. The RNA can comprise modifications, e.g., to sugar or purine or pyrimidine residues, to enhance stability. For example, branched nucleotide analogs can be incorporated into RNA. Suitable ribonucleotide modifications include, but are not limited to, replacement of the 2′-hydroxyl group of one or more than one ribonucleotide e.g., with a 2′-amino or 2′-methyl group; and the replacement of one or more than one ribonucleotide by the same number of corresponding locked nucleotides, wherein the sugar ring is chemically modified, preferably by a 2′-O 4′-C methylene bridge.

The RNAi polynucleotides can encompass the full-length target RNA or may correspond to a fragment of the target RNA. In some cases, the fragment will have fewer than 100, 200, 300, 400, 500 600, 700, 800, 900 or 1,000 nucleotides corresponding to the target sequence. In addition, in some embodiments, these fragments are at least, e.g., 10, 15, 20, 50, 100, 150, 200, or more nucleotides in length. Short dsRNAs (e.g., between 18-30 base pairs in length) may contain varying degrees of complementarity to their target mRNA in the antisense strand. In some embodiments, an RNA molecule may include hairpin RNAs comprising a single-stranded loop region and a base-paired stem of an inversely repeated sequence. In some embodiments, such an RNA may have overhanging bases on the 5′ or 3′ end of the sense strand and/or the antisense strand. In some cases, fragments for use in RNAi will be at least substantially similar to regions of a target gene that do not occur in other genes in the organism or may be selected to have as little similarity to other organism transcripts as possible, e.g., selected by comparison to sequences in analyzing publicly-available sequence databases.

In some embodiments, the pathogen DCL gene or DCL promoter to be targeted or silenced is from a viral, bacterial, fungal, nematode, oomycete, or insect pathogen. In some embodiments, the DCL gene is from a fungal pathogen. In some embodiments, the pathogen is Botyritis. In some embodiments, the pathogen is Botyritis cinerea. In some embodiments, the pathogen is Verticillium. In some embodiments, the pathogen is V. dahilae. In some embodiments, the pathogen is Aspergillus, Sclerotinia, or Rhizoctonia.

In some embodiments, one or more pathogen DCL genes is targeted, silenced, or inhibited in order to increase resistance to the pathogen in a plant by expressing in the plant, or contacting to the plant, a polynucleotide that inhibits expression of the pathogen DCL gene or that is complementary to the DCL gene or a fragment thereof. In some embodiments, the polynucleotide comprises an antisense nucleic acid that is complementary to the DCL gene or a fragment thereof. In some embodiments, the polynucleotide comprises a double stranded nucleic acid that targets the DCL gene, or its promoter, or a fragment thereof. In some embodiments, the polynucleotide comprises a double-stranded nucleic acid having a sequence that is identical or substantially similar (at least 60%, 65%, 70%, 75%, 80%, 85%, 90%, 91%, 92%, 93%, 94%, 95%, 96%, 97%, 98%, or 99% identical) to the DCL gene or a fragment thereof. In some embodiments, a “fragment” of a DCL gene or promoter comprises a sequence of at least 15, 16, 17, 18, 19, 20, 21, 22, 23, 24, 25, 26, 27, 28, 29, 30, 20, 30, 40, 50, 60, 70, 80, 90, 100, 150, 200, 250, 300, 350, 400, 450, 500 or more contiguous nucleotides of the DCL gene or promoter (e.g., comprises at least (e.g., at least 15, 16, 17, 18, 19, 20, 21, 22, 23, 24, 25, 26, 27, 28, 29, 30, 40, 50, 60, 70, 80, 90, 100, 150, 200, 250, 300, 350, 400, 450, 500 or more contiguous nucleotides of any of SEQ ID NO:1, SEQ ID NO:3, SEQ ID NO:5, SEQ ID NO:7, SEQ ID NO:9, SEQ ID NO:10, SEQ ID NO:11, SEQ ID NO:12, SEQ ID NO:28, SEQ ID NO:29, SEQ ID NO:30, or SEQ ID NO:31). In some embodiments, the double stranded nucleic acid is a small RNA duplex or a double stranded RNA.

In some embodiments, the polynucleotide inhibits expression of a fungal pathogen DCL gene that encodes a Botrytis or Verticillium DCL protein. In some embodiments, the polynucleotide inhibits expression of a fungal DCL gene that encodes a Botrytis DCL protein that is identical or substantially identical (e.g., at least 60%, 65%, 70%, 75%, 80%, 85%, 90%, 91%, 92%, 93%, 94%, 95%, 96%, 97%, 98%, or 99% identical) to SEQ ID NO:2 or SEQ ID NO:4, or a fragment thereof. In some embodiments, the polynucleotide inhibits expression of a fungal DCL gene that encodes a Verticillium DCL protein that is identical or substantially identical (e.g., at least 60%, 65%, 70%, 75%, 80%, 85%, 90%, 91%, 92%, 93%, 94%, 95%, 96%, 97%, 98%, or 99% identical) to SEQ ID NO:6 or SEQ ID NO:8, or a fragment thereof.

In some embodiments, the polynucleotide comprises a sequence that is identical or substantially identical (e.g., at least 60%, 65%, 70%, 75%, 80%, 85%, 90%, 91%, 92%, 93%, 94%, 95%, 96%, 97%, 98%, or 99% identical) to SEQ ID NO:1 or SEQ ID NO:3 or a fragment thereof, or a complement thereof. In some embodiments, the polynucleotide comprises a sequence that is identical or substantially identical (e.g., at least 60%, 65%, 70%, 75%, 80%, 85%, 90%, 91%, 92%, 93%, 94%, 95%, 96%, 97%, 98%, or 99% identical) to SEQ ID NO:5 or SEQ ID NO:7 or a fragment thereof, or a complement thereof. In some embodiments, the polynucleotide comprises a sequence that is identical or substantially identical (e.g., at least 60%, 65%, 70%, 75%, 80%, 85%, 90%, 91%, 92%, 93%, 94%, 95%, 96%, 97%, 98%, or 99% identical) to SEQ ID NO:9, SEQ ID NO:10, SEQ ID NO:11, or SEQ ID NO:12, or a fragment thereof, or a complement thereof.

In some embodiments, the polynucleotide comprises an inverted repeat of a sequence that is identical or substantially identical (e.g., at least 60%, 65%, 70%, 75%, 80%, 85%, 90%, 91%, 92%, 93%, 94%, 95%, 96%, 97%, 98%, or 99% identical) to any of SEQ ID NO:1, SEQ ID NO:3, SEQ ID NO:5, SEQ ID NO:7, SEQ ID NO:9, SEQ ID NO:10, SEQ ID NO: 11, or SEQ ID NO:12, or a fragment thereof, or a complement thereof. In some embodiments, the polynucleotide comprises a spacer in between the inverted repeat sequences.

In some embodiments, the polynucleotide targets a promoter region of a fungal pathogen DCL gene. For example, in some embodiments, the polynucleotide targets a promoter region within the sequence of any of SEQ ID NO:28, SEQ ID NO:29, SEQ ID NO:30, or SEQ ID NO:31.

In some embodiments, two or more fungal pathogen DCL genes or promoters are targeted (e.g., two, three, four or more DCL genes or promoters from the same fungal pathogen or from two or more fungal pathogens). In some embodiments, two or more Botrytis DCL genes or promoters are targeted. For example, in some embodiments, two or more of SEQ ID NO:1, SEQ ID NO:3, SEQ ID NO:28, and SEQ ID NO:29, or a fragment of any thereof, are targeted for inhibition of expression. In some embodiments, two or more Verticillium DCL genes or promoters are targeted. For example, in some embodiments, two or more of SEQ ID NO:5, SEQ ID NO:7, SEQ ID NO:30, or SEQ ID NO:31, or a fragment of any thereof, are targeted for inhibition of expression.

In some embodiments, the antifungal RNA targets a gene that is involved in vesicle trafficking, or a pathogen gene that is targeted by host sRNAs. Examples of such targets are include, but are not limited to, those set forth in Table 1 and Table 2 below.

TABLE 1
Botrytis cinerea target genes that are involved in vesicle trafficking

| Gene | Target gene | | | Aligned | Homolog in |
|---|---|---|---|---|---|
| name | ID | Gene description | Targeted by At_siRNA | score | Sclerotinia |
| DTCN | BC1G_10508 | Dynactin protein | TAS1c-siR483 (tasiRNA) | 4.25 | SS1G_04144 |
| VPS51 | BC1G_10728 | VPS51 family | TAS1c-siR483 (tasiRNA) | 3.5 | SS1G_09028 |
| | | protein | | | |
| SAC1 | BC1G_08464 | Polyphosphoinositide | TAS2-siR453 (tasiRNA) | 3.5 | SS1G_10257 |
| | | phosphatase | | | |
| VPS52 | BC1G_09781 | Vps52/Sac2 family | MIR159A (MicroRNA) | 4.5 | SS1G_01875 |
| | | protein | | | |
| Rgd1p | BC1G_15133 | GTPase activating | MIR396A (MicroRNA) | 4 | SS1G_03990 |
| | | protein | | | |
| UFD1 | BC1G_10526 | Endoplasmic | S10018 (IGN) | 4.5 | SS1G_04151 |
| | | reticulum-associated | | | |
| | | Ubiquitin fusion | | | |
| | | degradation protein | | | |
| | | UFD1 | | | |
| Integral | BC1G_03606 | Hypothetical protein | S10140 (IGN) | 4.5 | None |
| | | similar to integral | | | |
| | | membrane protein | | | |
| Sec31p | BC1G_03372 | WH2 motif protein | S1353733 (ORF) | 3 | SS1G_06679 |
| Gyp5p | BC1G_04258 | GTPase-activating | S1353733 (ORF) | 4 | SS1G_10712 |
| | | protein | | | |
| Pan1p | BC1G_09414 | Cytoskeleton | S1353733 (ORF) | 3 | SS1G_05987 |
| | | regulatory protein | | | |
| Srv2p | BC1G_14507 | Adenylyl cyclase- | S1353733 (ORF) | 3 | SS1G_13327 |
| | | associated protein | | | |

TABLE 2
Botrytis cinerea genes targeted by host sRNAs

| | | | | | | Target gene |
|---|---|---|---|---|---|---|
| Target | Putative function of | GO_biological | Targeted | sRNA | Aligned | alignment |
| gene ID | target gene | process | by sRNA | type | score | sRNA 3'-5' |
| BC1G_10728 | Conserved | vesicle transport | TAS1c- | tasiRNA | 3.5 | :\|\|x\|x\|x\|\|\|\|\|\|\|\|\|\|\|\|\|x |
| | hypothetical VPS51 | | siR483 | | | |
| | protein | | | | | |
| BC1G_10508 | Predicted dynactin | vesicle transport | TAS1c- | tasiRNA | 4.25 | \|\|\|\|\|\|x:\|\|\|\|\|\|\|\|:\|\|\|xx |
| | protein | | siR483 | | | |
| BC1G_08464 | Polyphosphoinositide | vesicle transport | TAS2- | tasiRNA | 3.5 | :\|\|\|\|\|\|\|x\|\|\|\|\|\|x\|\|\|\|\| |
| | phosphatase | | siR453 | | | |
| BC1G_15133 | Hypothetical protein | vesicle transport | MIR396A | miRNA | 4 | \|:\|\|\|:\|\|x\|\|\|\|\|\|\|\|\|x\|\| |
| | similar to GTPase | | | | | |
| | activating protein | | | | | |
| BC1G_09781 | Hypothetical protein | vesicle transport | MIR159A | miRNA | 4.5 | \|\|\|\|x\|\|\|\|:\|\|\|\|\|x\|\|\|\|: |
| | similar to | | | | | |
| | Vps52/Sac2 family | | | | | |
| | protein | | | | | |
| BC1G_05327 | Pyruvate | metabolic | IGN-siR1 | IGN | 4.5 | x\|x\|x\|\|\|\|\|\|\|\|\|\|\|\|x\|\|\|: |
| | carboxylase | process | | | | |
| BC1G_15423 | Predicted FAD | metabolic | TAS1c- | tasiRNA | 3.75 | \|\|\|x:\|\|\|\|\|\|\|\|\|\|\|\|:\|\|: |
| | binding protein | process | siR602 | | | |
| BC1G_09454 | Retinol | metabolic | MIR157A | miRNA | 2.5 | x\|\|\|\|\|\|\|x\|\|\|\|\|\|\|\|\|\|\|: |
| | dehydrogenase 12 | process | | | | |
| BC1G_15945 | Hypothetical protein | regulation of | MIR396A | miRNA | 4 | \|:\|x\|:\|\|\|\|\|\|\|\|\|\|\|\|x\|\| |
| | similar to GAL4-like | transcription | | | | |
| | transcription factor | | | | | |
| BC1G_14887 | Histone-lysine N- | regulation of | MIR396A | miRNA | 3 | :\|x\|\|:\|\|\|\|\|:\|\|\|\|\|\|\|\|\| |
| | methyltransferase | transcription | | | | |
| BC1G_07589 | Histone-lysine N- | regulation of | MIR396A | miRNA | 4.5 | x\|\|\|\|\|\|:\|\|\|x\|\|\|\|\|\|\|:\| |
| | methyltransferase | transcription | | | | |
| BC1G_05475 | Hypothetical protein | biosynthetic | MIR159B | miRNA | 4.5 | \|\|x\|\|\|\|:\|\|\|\|\|\|x\|\|\|\|:\| |
| | similar to | process | | | | |
| | microcystin | | | | | |
| | synthetase | | | | | |
| BC1T_07401 | Botrytis cinerea | biosynthetic | S10044 | TE | 4.5 | \|\|x\|:\|\|\|\|\|\|\|:\|\|\|\|\|\|x\| |
| | (B05.10) | process | | | | |
| | glutaminyl-tRNA | | | | | |
| | synthetase | | | | | |
| BC1G_09015 | Dual specificity | signal | MIR158A | miRNA | 3.5 | \|x\|\|\|\|x\|:\|\|\|\|\|\|\|\|\|\|: |
| | protein kinase | transduction | | | | |
| | POM1 | | | | | |
| BC1G_03832 | R3H domain of | cell cycle | MIR159A | miRNA | 4 | \|\|\|\|xx\|x\|\|\|\|\|\|\|\|\|\|\|\|\| |
| | encore-like and | | | | | |
| | DIP1-like protein | | | | | |
| BC1G_09907 | Predicted membrane | cell wall | MIR168 | miRNA | 4.5 | x\|\|x\|x\|\|:\|\|\|\|\|\|\|\|\|\|\|x |
| | protein involved in | biogenesis | | | | |
| | the export of O- | | | | | |
| | antigen and teichoic | | | | | |
| | acid | | | | | |
| BC1G_02544 | Hypothetical protein | unknown | MIR166A | miRNA | 4.5 | \|\|\|x\|\|x\|\|\|\|\|\|\|\|\|\|\|x\|: |
| | similar to | | | | | |
| | B230380D07Rik | | | | | |
| | protein | | | | | |
| BC1G_11528 | Predicted protein | unknown | MIR159B | miRNA | 3.5 | \|\|x\|\|\|\|::\|\|\|\|\|\|\|:\|\|\|\| |
| BC1G_11528 | Predicted protein | unknown | MIR159A | miRNA | 4.5 | x\|x\|\|\|\|::\|\|\|\|\|\|\|:\|\|\|\| |
| BC1G_04218 | Predicted protein | unknown | MIR396A | miRNA | 4.25 | \|\|\|\|x:\|\|\|\|\|\|\|\|\|x\|\|\|\|\| |
| BC1G_00860 | Domain of unknown | unknown | MIR158A | miRNA | 4.5 | \|\|\|x\|\|\|x\|\|\|\|\|\|\|\|\|x\|: |
| | function (DUF4211) | | | | | |
| | protein | | | | | |
| BC1G_04811 | redicted protein | unknown | S10086 | IGN | 3 | \|\|\|\|x\|\|\|\|\|\|\|\|\|:\|:\|\|\| |
| BC1G_05162 | Predicted protein | unknown | S10131 | ORF | 4.5 | x\|x\|\|\|x\|:\|\|\|\|\|\|:\|\|\|\|\| |
| BC1G_06835 | Predicted protein | unknown | S10131 | ORF | 3 | \|:\|x\|\|\|x\|\|\|\|\|\|\|\|\|\|\|\|: |
| BC1G_10526 | Endoplasmic | vesicle transport | S10018 | IGN | 4.5 | x\|:\|\|\|\|x\|\|\|\|\|\|x\|\|\|\|\| |
| | reticulum- | | | | | |
| | associatedUbiquitin | | | | | |
| | fusion degradation | | | | | |
| | protein UFD1 | | | | | |
| BC1G_03606 | Hypothetical protein | vesicle transport | S10140 | IGN | 4.5 | \|x\|:\|\|\|\|\|\|\|\|\|:\|:\|\|\|x |
| | similar to integral | | | | | |
| | membrane protein | | | | | |
| BC1G_04443 | Ketol-acid | metabolic | S10052 | IGN | 4 | x\|x\|\|x\|\|\|\|\|:\|\|\|\|\|\|\|\| |
| | reductoisomerase | process | | | | |
| BC1G_12479 | Isopenicillin N | metabolic | S10117 | IGN | 4 | \|\|\|xx\|\|x\|\|\|\|\|\|\|\|\|\|\|\| |
| | synthase and related | process | | | | |
| | dioxygenases | | | | | |
| BC1G_06676 | Fatty-acid amide | metabolic | MIR8167 | miRNA | 4.5 | \|:\|\|\|:\|:\|\|\|:\|x\|\|\|\|\|\|\| |
| | hydrolase 1 | process | | | | |
| BC1G_12472 | Serine threonine- | regulation of | S10131 | ORF | 4.5 | \|\|\|\|:\|x\|\|\|\|\|\|x\|:\|\|\|\|\| |
| | protein phosphatase | transcription | | | | |
| | dullard protein | | | | | |
| BC1G_02471 | RNA polymerase III | regulation of | S10071 | IGN | 4 | x\|\|\|\|\|\|\|\|\|\|\|\|\|\|\|\|\|x\|\|x |
| | | transcription | | | | |
| BC1G_03511 | Hypothetical protein | biosynthetic | S10083 | Anti-ORF | 3.5 | x\|:\|\|x\|\|x\|\|\|\|\|\|\|\|\|\|\|\|\|\| |
| | similar to peptide | process | | | | |
| | synthetase | | | | | |
| BC1G_03981 | Hypothetical protein | regulation of | MIR8167 | miRNA | 4.5 | \|\|\|:\|\|x\|\|\|\|\|\|\|\|\|x\|\|:\| |
| | similar to | transport | | | | |
| | sulfate/anion | | | | | |
| | exchanger | | | | | |
| BC1G_14507 | 70-kDa adenylyl | vesicle transport | S1353733 | ORF | 3 | x\|\|x\|\|x\|\|\|\|\|\|\|\|\|\|\|\|\|\| |
| | cyclase-associated | | | | | |
| | protein | | | | | |
| BC1G_09414 | Protein similar to | vesicle transport | S1353733 | ORF | 3 | x\|\|x\|\|x\|\|\|\|\|\|\|\|\|\|\|\|\|\| |
| | actin cytoskeleton- | | | | | |
| | regulatory complex | | | | | |
| | protein PAN1 | | | | | |
| BC1G_04258 | GTPase-activating | vesicle transport | S1353733 | ORF | 4 | x\|\|\|\|\|x\|\|\|\|\|\|\|\|\|\|\|x\|\| |
| | protein GYP5 | | | | | |
| BC1G_03372 | Hypothetical WH2 | vesicle transport | S1353733 | ORF | 3 | x\|\|x\|\|\|\|\|\|\|\|\|\|\|\|\|\|\|:\| |
| | motif protein | | | | | |
| BC1G_14667 | Predicted protein | unknown | MIR396B | miRNA | 4.5 | ::\|x\|\|\|x\|\|\|\|\|\|\|\|\|\|\|\|x |
| BC1G_14204 | Predicted protein | unknown | S1353733 | ORF | 3.5 | \|:\|x\|\|x\|\|\|\|\|\|\|\|\|\|:\|\|\| |
| BC1G_10316 | Predicted protein | unknown | S1353733 | ORF | 4.5 | x\|:\|\|\|\|:\|\|\|\|x\|\|\|\|\|\|\|: |
| BC1G_05030 | Predicted protein | unknown | S1353733 | ORF | 4.25 | x:\|\|\|\|\|\|\|\|\|\|\|\|x\|\|\|\|\|\| |
| BC1G_00624 | Predicted protein | unknown | S1353733 | ORF | 4 | x\|\|x\|\|\|\|\|\|\|\|\|\|\|\|\|\|:\|x |
| BC1G_15490 | Bifunctional P- | metabolic | MIR396A* | miRNA | 4.5 | \|x\|:\|\|:\|:\|\|\|\|\|\|\|\|x\|\|\| |
| | 450/NADPH-P450 | process | | | | |
| | reductase | | | | | |
| BC1G_14979 | Hypothetical protein | metabolic | S1353733 | ORF | 3 | x\|\|x\|\|x\|\|\|\|\|\|\|\|\|\|\|\|\|\| |
| | similar to | process | | | | |
| | mitochondrial ATP | | | | | |
| | synthase B | | | | | |
| BC1G_14979 | Hypothetical protein | metabolic | MIR396B | miRNA | 4 | \|\|\|\|\|\|\|\|\|:\|x\|\|\|\|\|\|\|:\| |
| | similar to | process | | | | |
| | mitochondrial ATP | | | | | |
| | synthase B | | | | | |
| BC1G_12936 | 2-deoxy-D- | metabolic | MIR396A* | miRNA | 4 | \|\|\|x\|\|\|x\|\|\|\|\|\|\|\|x\|\|\|\| |
| | gluconate 3- | process | | | | |
| | dehydrogenase | | | | | |
| BC1G_04424 | Hypothetical protein | regulation of | S1353733 | ORF | 3 | x\|\|x\|\|\|x\|\|\|\|\|\|\|\|\|\|\|\|\| |
| | similar to ITC1 | transcription | | | | |
| BC1G_14463 | Hypothetical protein | mitotic cell | S1353733 | ORF | 4 | x\|\|x\|\|x\|\|\|\|:\|\|\|\|\|\|\|\|\| |
| | similar to Uso1p | cycle | | | | |
| BC1G_10235 | Hypothetical protein | mitotic cell | S1353733 | ORF | 4 | \|\|\|x\|\|x\|\|\|\|\|\|\|\|\|\|\|x\|\| |
| | similar to Smc4p | cycle | | | | |
| BC1G_12627 | Hypothetical protein | cell wall | S1353733 | ORF | 4.25 | \|\|:\|\|:x\|:\|\|\|\|\|\|\|\|\|:\|\| |
| | similar to cell wall | biogenesis | | | | |
| | synthesis protein | | | | | |
| BC1G_09656 | Hypothetical protein | cell wall | S1353733 | ORF | 4.5 | x\|\|x\|\|\|:\|\|\|\|\|\|\|\|\|\|:\|x |
| | similar to HKR1 | biogenesis | | | | |
| BC1G_07658 | Hypothetical protein | RNA catabolic | S1353733 | ORF | 4.5 | \|::\|:\|\|\|\|\|\|:\|\|\|\|\|\|:\|: |
| | similar to | process | | | | |
| | endoglucanase IV | | | | | |
| BC1G_02429 | Ribonuclease HI | RNA catabolic | S1353733 | ORF | 4 | x\|\|\|:\|\|\|:\|\|:\|\|\|\|\|:\|\|\| |
| | large subunit | process | | | | |
| BC1T_09103 | Botrytis cinerea | cell cycle | S1092315 | TE | 4.5 | \|\|x\|\|\|\|\|\|:\|\|:\|\|\|\|\|\|\|x\| |
| | (B05.10) | | | | | |
| | hypothetical protein | | | | | |
| | similar to cell | | | | | |
| | division cycle | | | | | |
| | mutant | | | | | |
| BC1G_02638 | Cell cycle | cell cycle | S1353733 | ORF | 4.5 | x\|\|x\|\|x\|\|\|\|\|\|\|:\|\|\|\|\|: |
| | checkpoint protein | | | | | |
| | RAD17 | | | | | |
| BC1G_02869 | Guanine nucleotide- | cell proliferation | S1353733 | ORF | 4 | \|\|\|\|:\|x\|\|x\|\|\|\|\|\|\|\|\|\|: |
| | binding protein | | | | | |
| | G(I)/G(S)/G(T) | | | | | |
| | subunit beta-1 | | | | | |
| BC1G_09169 | Hypothetical protein | cell proliferation | S1353733 | ORF | 4 | x\|\|x\|\|x\|\|\|\|\|\|\|\|\|\|\|:\|\| |
| | similar to calpain 2 | | | | | |
| | catalytic subunit | | | | | |
| BC1G_07037 | Hypothetical protein | tRNA | S519888 | ORF | 4.5 | :\|x\|\|\|\|\|\|\|\|\|:\|\|\|\|\|x\|\| |
| | similar to Msf1p | processing | | | | |
| BC1G_10614 | Hypothetical protein | cell surface | MIR396A* | miRNA | 4.5 | :\|\|x\|x\|x\|\|\|\|\|\|\|\|\|\|\|\|x |
| | similar to GAMM1 | receptor | | | | |
| | protein | signaling | | | | |
| | | pathway | | | | |

In some embodiments, the RNA targets sequence in a vacuolar protein sorting 51 (VPS51) gene (e.g., SEQ ID NO: 34 or SEQ TD NO:35), a dynactin (DCTN1) gene (e.g., SEQ ID NO:32 or SEQ ID NO:33), or a suppressor of actin (SAC1) gene of a fungal pathogen (e.g., SEQ ID NO:36 or SEQ ID NO:37). In some embodiments, the antifungal RNA may include a sequence targeting two or more such genes (e.g., Bc-VPS5I+DCTN1+SAC1-dsRNAs according to SEQ ID NO: 38). Other such targets are described in WO 2019/079044, which is incorporated herein by reference in its entirety.

In some embodiments, the antifungal RNA targets other virulence factor genes, such as polygalacturonase gene (e.g., R. solani-PG as set forth in SEQ ID NO:40) or an exo-polygalacturonase gene (e.g., A. niger pgxB as set forth in SEQ ID NO:42) of a fungal pathogen. The antifungal sRNA may have, for example, a sequence as set forth in SEQ ID NO:41 or SEQ ID NO:43.

The LTR regions that generate most small RNA effectors can be targeted for silencing. In some embodiments, such as for B. cinerea, sRNA effectors are derived from LTR retrotransposon regions. Additionally, the promoter regions of LTRs can also be targeted for silencing. Targeting of LTR promoter regions can trigger transcriptional gene silencing, which would avoid random silencing of host genes by LTR small RNAs.

In some embodiments, the polynucleotide targets or inhibits expression of a pathogen LTR region or of a promoter region of a pathogen LTR, wherein the pathogen is a fungal pathogen. In some embodiments, the pathogen is Botyritis. In some embodiments, the pathogen is Botyritis cinerea. In some embodiments, the pathogen is Verticillium. In some embodiments, the pathogen is V. dahilae.

In some embodiments, the polynucleotide targets a sequence of any of SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, or SEQ ID NO:27 or a fragment thereof, or a complement thereof. In some embodiments, a “fragment” of a LTR region or LTR promoter comprises a sequence of at least 15, 20, 30, 40, 50, 60, 70, 80, 90, 100, 150, 200, 250, 300, 350, 400, 450, 500 or more contiguous nucleotides of the LTR region or LTR promoter (e.g., comprises at least 15, 20, 30, 40, 50, 60, 70, 80, 90, 100, 150, 200, 250, 300, 350, 400, 450, 500 or more contiguous nucleotides of any of SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, or SEQ ID NO:27).

In some embodiments, the polynucleotide comprises an antisense nucleic acid that is complementary to any of SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 or a fragment thereof. In some embodiments, the polynucleotide comprises a double-stranded nucleic acid having a sequence that is identical or substantially similar (at least 60%, 65%, 70%, 75%, 80%, 85%, 90%, 91%, 92%, 93%, 94%, 95%, 96%, 97%, 98%, or 99% identical) to any of SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, or SEQ ID NO:27 or a fragment thereof. In some embodiments, the polynucleotide comprises an inverted repeat of a fragment of any of SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, or SEQ ID NO:27, and further comprises a spacer region separating the inverted repeat nucleotide sequences.

In some embodiments, the polynucleotide targets a promoter region of a fungal LTR. For example, in some embodiments, the polynucleotide targets a promoter region within the sequence of SEQ ID NO:27.

### IV. Methods for Increasing Pathogen Resistance in Plants

Also provided herein are methods for increasing pathogen resistance in plants. The methods include contacting the plant with an antifungal RNA composition according to the present disclosure. In some embodiments, the double-stranded RNA, small RNA, or small RNA duplex is sprayed onto the plant or the part of the plant.

In some embodiments, the plant is an ornamental plant. In some embodiments, the plant is a fruit- or vegetable-producing plant. In some embodiments, the part of the plant is a fruit, a vegetable, or a flower. The plant may be a species from the genera Allium, Asparagus, Atropa, Avena, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pisum, Pyrus, Prunus, Raphanus, Rosa, Secale, Senecio, Sinapis, Solanum, Solanaceae, Sorghum, Trigonella, Triticum, Vitis, Vigna, and Zea. In some embodiments, the plant is a vining plant, e.g., a species from the genus Vitis. In some embodiments, the plant is an ornamental plant, e.g., a species from the genus Rosa. In some embodiments, the plant is a monocot. In some embodiments, the plant is a dicot.

Antifungal RNA compositions may be applied to plants manually or in automated fashion. A crop sprayer or other such agricultural application machine may be used. A crop spray may contain a tank carried on a chassis, for trailing behind a tractor or for use as a self-propelled unit having an integral cab and engine. The machine may further include an extending boom which provides a transverse line of uniformly spaced spray nozzles connected by pipes to the tank. During operation the application machine may be moved across fields of crops to the RNA vesicle composition in a controlled manner. In addition, transgenic plants engineered to generate extracellular vesicles containing the antifungal RNA may be employed.

### V. EXAMPLES

### Example 1—Artificial Vesicles (AVs)

Artificial vesicles (AVs) for stabilization and delivery of antifungal RNAs to fungal pathogens were made and tested. The artificial vesicles contained various formulations of lipids, including:
(1) 1,2-dioleoyl-3-trimethylammonium-propane (DOTAP), 1,2-distearoyl-sn-glycero-3-phosphoethanolamine-N-[amino(polyethylene glycol)-2000] (PEG), and cholesterol.
(2) DOTAP and cholesterol; and
(3) 1,2-dioleyloxy-3-dimethylaminopropane (DODMA) and cholesterol.

Artificial vesicles for encapsulation of dsRNA or small RNA using the lipid film hydration method. DOTAP, cholesterol, and the optional reagent DSPE-PEG2000 (2:1:0.1) were dissolved in chloroform: methanol (4:1, v/v). After mixing the lipids, the organic solvent was evaporated under a fumehood for 120 min. The lipid film was hydrated using a solution of dsRNA or sRNA duplex in RNase-free dH2O. The amount of RNA used to hydrate the film was calculated from the charge ratio (N:P). After hydration at 4° C. overnight, the crude vesicles were subjected to extrusion by Mini-Extruder. A similar protocol was followed to generate DODMA (1,2-dioleyloxy-3-dimethylaminopropane) vesicles using 2:1 DODMA:cholesterol. Extrusion of vesicles was performed using a Mini-Extruder (Avanti Polar Lipids, Alabaster, USA). Lipid vesicles were extruded 11 times through a 0.4 m polycarbonate membrane.

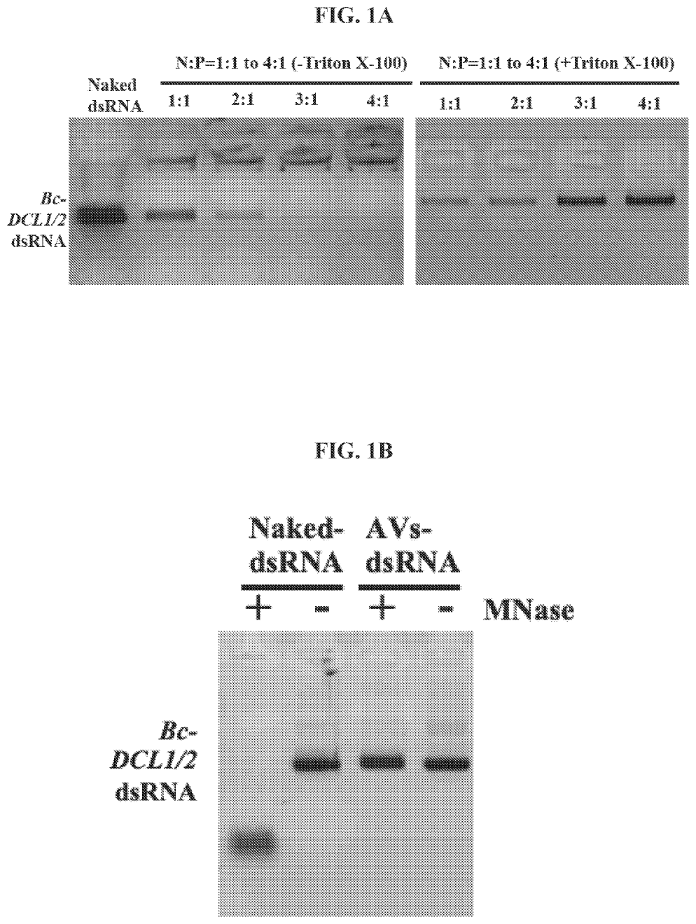

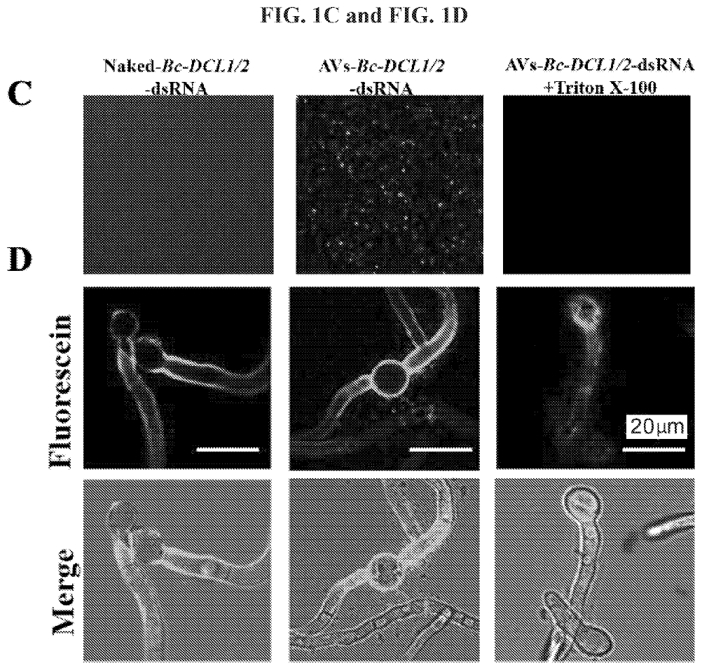

The fungal-gene targeting dsRNAs are easily loaded into the AVs, which protect the dsRNA from nuclease degradation (FIGS. 1A-1D). AV-Bc-DCL1/2-dsRNA lipoplexes were formed at a range of charge ratios (N:P), as indicated in FIG. 1A, and incubated for 2 h at room temperature before being loaded onto a 2% agarose gel. On the right, Bc-DCL1/2-dsRNA released from AV-Bc-DCL1/2-dsRNA after treatment with 1% Triton X-100 shows that complete loading was achieved to an AVs:dsRNA mass ratio of 4:1. The stability of naked- and AV-Bc-DCL1/2-dsRNA was tested after MNase treatment, as shown in FIG. 1B. Bc-DCL1/2-dsRNA was released from AVs using 1% Triton X-100 before gel electrophoresis. The mixture of AVs and dsRNA without incubation period (AVs/dsRNA), which do not form a lipoplex, was used as a control for excluding AVs interference to MNase activity.

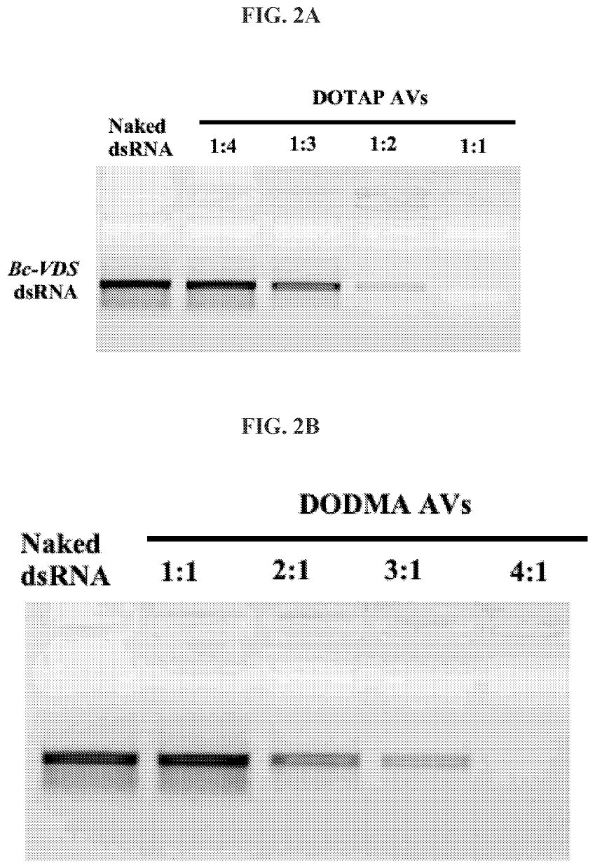

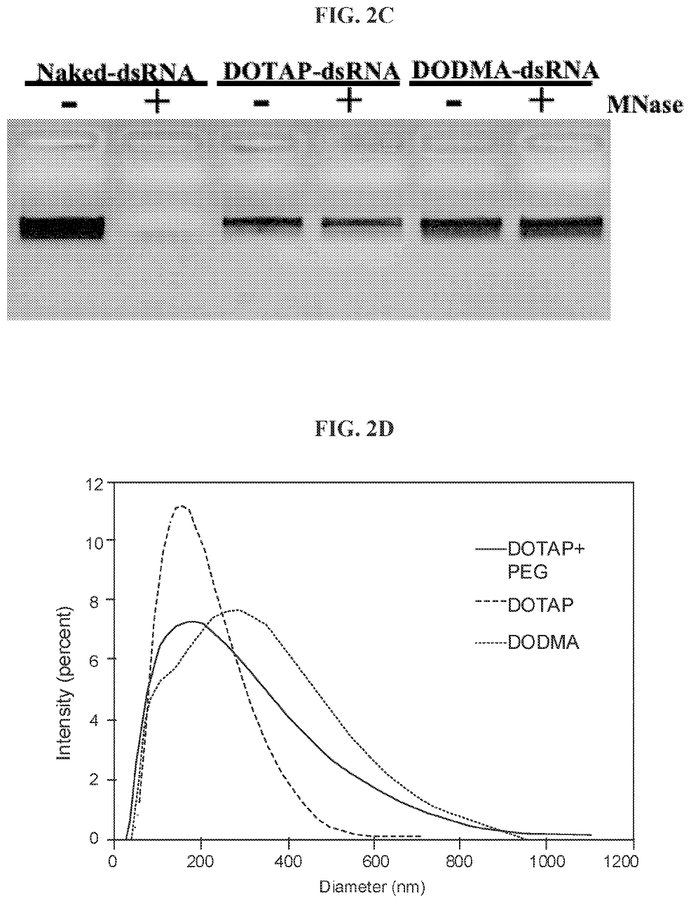

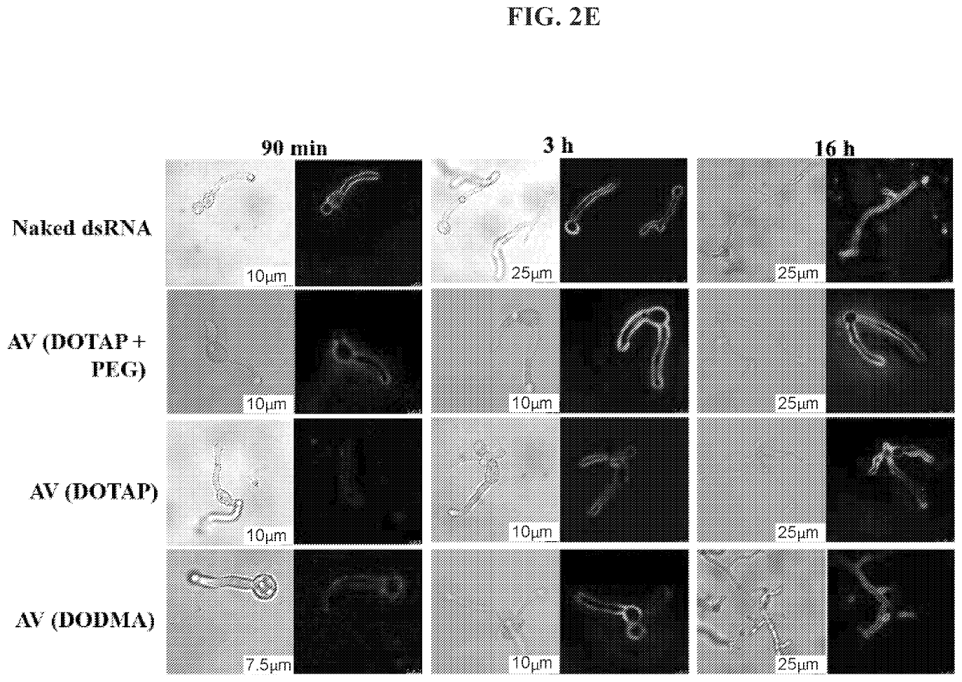

The vesicles are readily taken up by the target fungal pathogen, Botrytis cinerea. As shown in FIG. 1D, fluorescein-labeled naked- or AV-Bc-DCL1/2-dsRNA (SEQ ID NO:39) were added to B. cinerea spores and fluorescent signals were detected in B. cinerea cells after culturing on PDA medium for 10 h. MNase treatment was performed 30 min before image acquisition. Fluorescence signals remained visible in the B. cinerea cells treated with AV-Bc-DCL 1/2-dsRNA using Triton X-100 and MNase treatment before observation. Scale bars, 20 m. As shown in FIG. 2E, B. cinerea uptake of fluorescein-labeled dsRNA encapsulated in three different AV formulations (DOTAP+PEG, DOTAP and DODMA) was assessed after 3 and 16 hours of incubation. Fluorescence signals are visible in the B. cinerea cells treated with the three AV-Bc-DCL1/2-dsRNA using Triton X-100 and MNase treatment before observation.

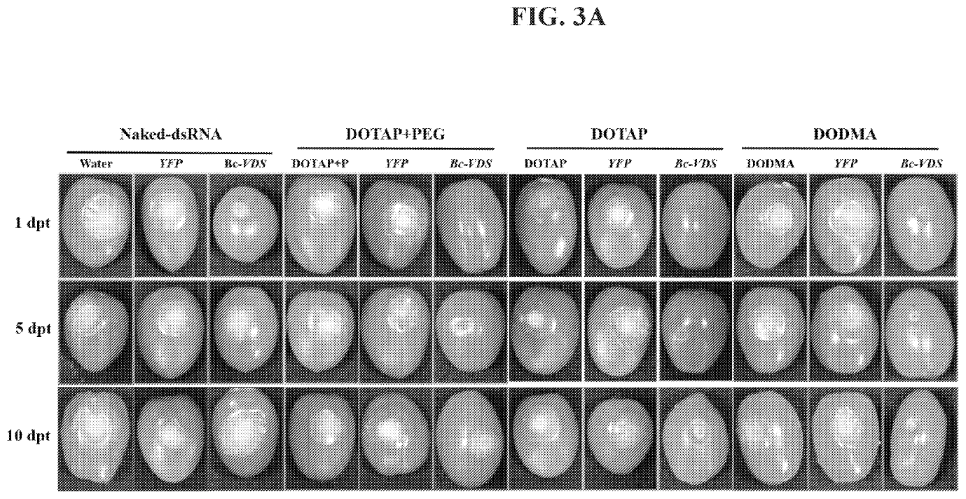

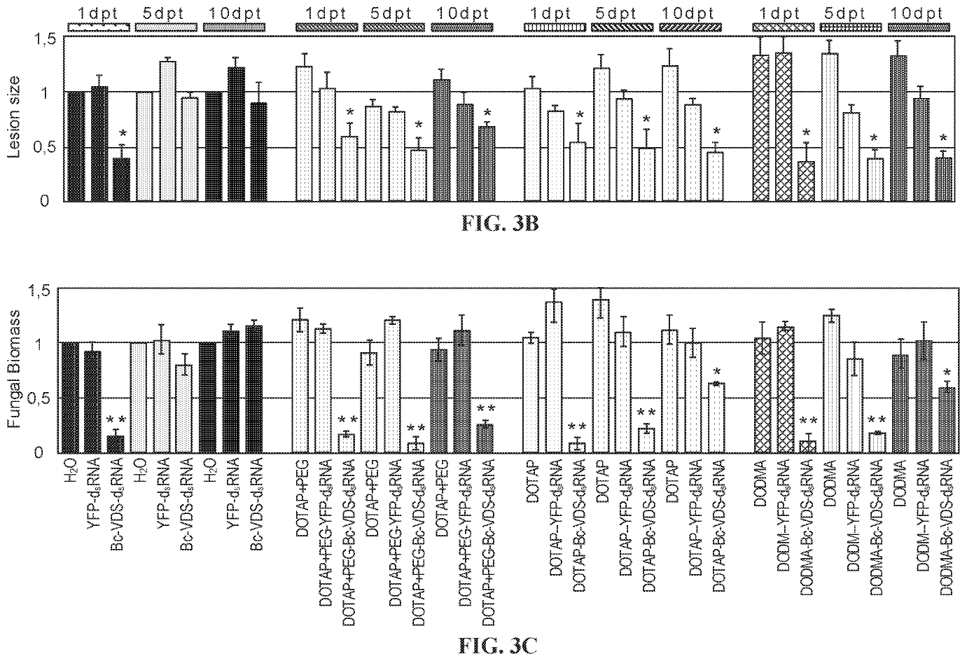

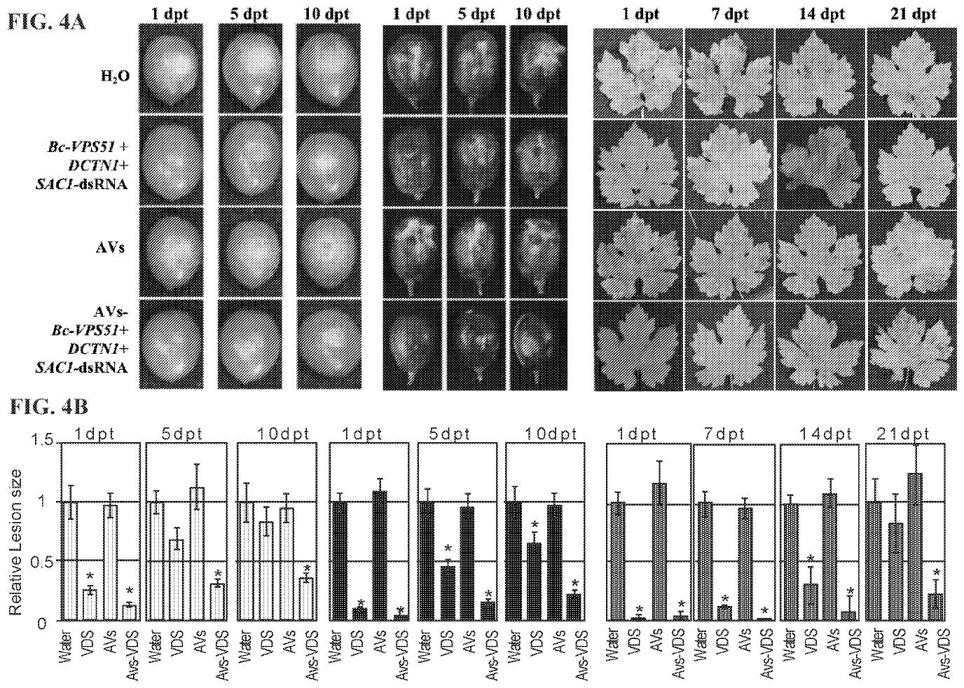

The vesicles can be utilized to protect both pre- and post-harvest plant materials (FIGS. 3A-3C, 4A, and 4B). For example, treatment with DOTAP+PEG, DOTAP, and DODMA AV-dsRNA formulations provide prolonged protection against B. cinerea in tomato fruits. FIG. 3A shows tomato fruits that were pre-treated with naked- or AV(DOTAP+PEG)-Bc-VDS-dsRNA, AV(DOTAP)-Bc-VDS-dsRNA, and AV(DODMA)-Bc-VDS-dsRNA, for 1, 5, and 10 days, then inoculated with B. cinerea. Pictures were taken at 5 dpi. Relative lesion sizes were measured with the help of ImageJ software, as shown in FIG. 3B. Error bars indicate the SD. Statistical significance (Student's t-test): *, P<0.05. Relative fungal biomass was quantified by qPCR, as shown in FIG. 3C. Fungal RNA relative to tomato RNA was measured by assaying the fungal actin gene and the tomato actin gene by qPCR using RNA extracted from the infected fruits at 5 dpi. Statistical significance (Student's t-test): *, P<0.05; **, P<0.01.

Treatment with AV-dsRNA also provides prolonged protection against B. cinerea in grape berries and V. virifera leaves. FIG. 4A shows grape leaves that were pre-treated with naked- or AV-Bc-VDS-dsRNA, for 1, 7, 14, and 21 days then inoculated with B. cinerea. Pictures were taken at 5 dpi. FIG. 4B shows elative lesion sizes were measured with the help of ImageJ software. Error bars indicate the SD. Statistical significance (Student's t-test): *, P<0.05.

RNA-fungicides developed for use in SIGS applications are an eco-friendly alternative to traditional pesticides, and offer a way to target specific pathogen genes without the need for generating a GMO crop. However, commercial adoption of RNA-based fungicides is currently hindered by the relative instability of RNA in the environment. When packaged into artificial vesicles as described herein, these pathogen-targeting RNAs maintain their antifungal effect for up to 10 days in tomato fruits (FIGS. 3A-3C) and 21 days in grape leaves (FIGS. 4A and 4B). In comparison, naked RNA largely lost its antifungal effect after 5 days on tomato fruits and 14 days on grape leaves (FIGS. 3A-3C, 4A, and 4B) clearly demonstrating that the packaging of RNAs in artificial vesicles extends the antifungal effect of the RNA.

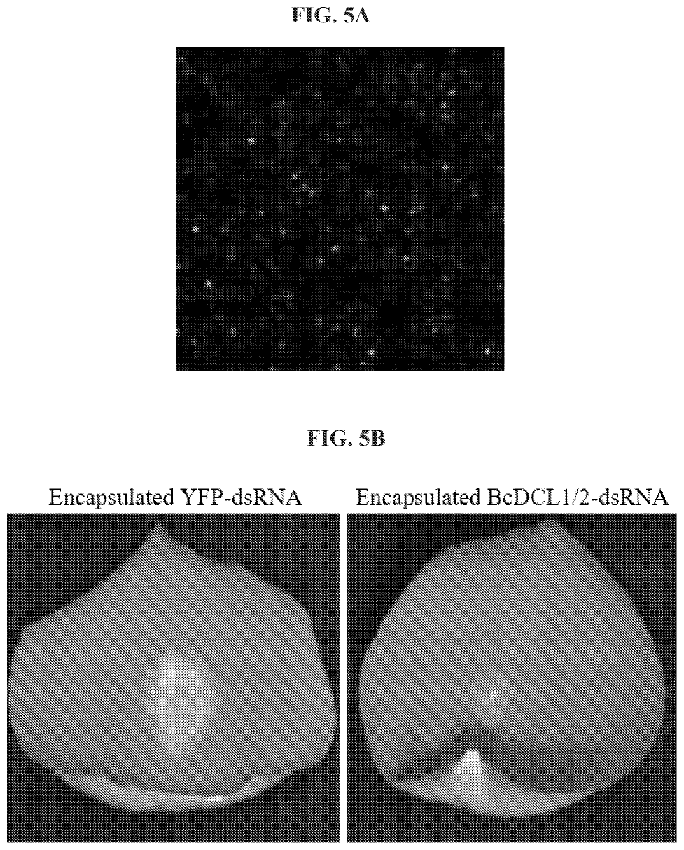

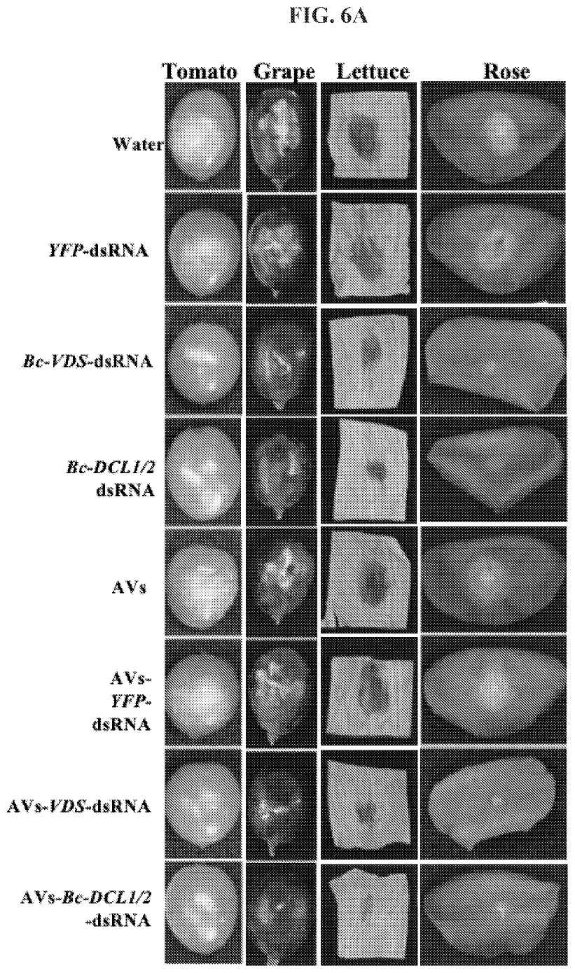

Extracellular vesicles were isolated from N. benthamiana as described above. Florescence-labeled dsRNAs were sufficiently encapsulated in the isolated natural extracellular vesicles, as shown in FIG. 5A. Bc-DCL1/2-dsRNA encapsulated by the extracellular vesicles efficiently inhibited the fungal disease caused by B. cinerea, as shown in FIG. 5B.

### Example 2—Artificial Nanovesicles for dsRNA Delivery in Spray Induced Gene Silencing for Crop Protection

### INTRODUCTION

Plant pathogens and pests are a major threat to global food security, causing crop yield losses of up to 20%, and postharvest product losses of up to 10% worldwide. Of these biotic threats, fungi represent some of the most aggressive and pervasive pathogens. For example, the causal agent of gray mold disease in over 1000 plant species, Botrytis cinerea, alone causes billions of dollars in annual crop yield losses. Alarmingly, this threat is projected to increase as rising temperatures associated with global climate change favor fungal pathogen growth. Currently, the most widely used plant pathogen control practices require routine application of fungicides which threaten the environment and can lead to the development of fungicide resistant pathogens. To safeguard global food security, an alternative, environmentally friendly fungal control method must be developed. Recent studies have shown that many aggressive fungal pathogens can take up RNAs from the environment. The RNAs, mostly double-stranded RNAs (dsRNAs) or small RNAs (sRNAs), can be designed to target fungal virulence-related genes for silencing. This discovery led to the development of Spray-Induced Gene Silencing (SIGS), where fungal virulence gene-targeting RNAs are topically applied to plant material to control fungal pathogens. SIGS can provide safe and powerful plant protection on both pre-harvest crops and post-harvest products against fungal pathogens that have high RNA uptake efficiency. SIGS RNAs can be versatilely designed to be species-specific, minimizing the risk of off-target effects on other organisms, and to target multiple genes and pathogens at once. Furthermore, because RNAi can tolerate multiple mismatches between sRNAs and target RNAs, fungal pathogens are less likely to develop resistance to SIGS RNAs than to traditional fungicides. Unlike host-induced gene silencing (HIGS), SIGS does not require the generation of transgenic plants, which still remains technically challenging in many crops and necessitates overcoming expensive and complicated regulatory hurdles.

One major drawback of SIGS is the relative instability of RNA in the environment, particularly when subjected to rainfall, high humidity, or UV light. Thus, improving environmental RNA stability is critical for successful SIGS applications. Described herein are fungal gene-targeting RNAs packaged in liposomes, termed artificial nanovesicles (AVs), for use in SIGS applications. As demonstrated herein, dsRNA-packaged in AVs can be successfully utilized in crop protection strategies. Three types of AVs were synthesized and found to confer protection to loaded dsRNA, which remained detectable in large amounts on plant surfaces over a long period of time. When applied to plants, AV-dsRNA can extend the length of fungal protection conferred by fungicidal dsRNA to crops by over 10-fold. Overall, this work demonstrates how organic nanoparticles can be utilized to strengthen SIGS-based crop protection strategies.

### Results

Artificial Nanovesicles Protect and Efficiently Deliver dsRNA to the Fungal Pathogen Botrytis cinerea

PEGylated AVs were synthesized using the lipid film hydration method for cationic liposomes https://paperpile.com/e/aFrwRa/KQkzx. Specifically, AVs were generated using a mixture of the cationic lipid 1,2-dioleoyl-3-trimethylammonium-propane (DOTAP), cholesterol and 1,2-distearoyl-sn-glycero-3-phosphoethanolamine-N-[methoxy(polyethyleneglycol)-2000](DSPE-PEG2000). We then established the loading ratio necessary for the AVs to completely encapsulate dsRNAs of interest. Exogenous treatment of Bc-DCL1/2-dsRNA, a dsRNA integrating fragments of the Dicer-like 1 (252 bp) and Dicer-like 2 (238 pb) sequences from Botrytis cinerea, on the plant leaf surface can efficiently inhibit fungal disease. Thus, several charge ratios (N:P where N=# of positively-charged polymer nitrogen groups and P=# of negatively-charged nucleic acid phosphate groups) between AVs and the Bc-DCL1/2-dsRNA, from 1:1 to 4:1, were examined to identify the minimum amount of AVs required to bind all the dsRNA present in the solution. We concluded that a 4:1 (AV:dsRNA) ratio was the minimum ratio needed for dsRNA loading as Bc-DCL1/2-dsRNA loaded into AVs at this ratio could not migrate from the loading well due to complete association with the AVs (FIG. 1A).

The ability of the AVs to prevent nuclease degradation was then validated under different enzymatic hydrolysis conditions. Naked and AV-loaded Bc-DCL1/2-dsRNA were both treated with Micrococcal Nuclease (MNase). As seen in FIG. 1B, the naked-Bc-DCL1/2-dsRNA exhibited greater degradation after MNase treatment as compared to the Bc-DCL1/2-dsRNA released from the AV-Bc-DCL1/2-dsRNA using 1% Triton X-100. Thus, the AVs provide protection for dsRNA against nuclease degradation. To further confirm that the dsRNA is encapsulated and protected by the AVs, we used Fluorescein-12-UTP to label both naked-Bc-DCL 1/2-dsRNA and AV-Bc-DCL1/2-dsRNA. The Fluorescein-labeled naked-Bc-DCL1/2-dsRNA showed a diffused fluorescent signal when examined by confocal laser scanning microscopy (CLSM), while the Fluorescein-labeled AV-Bc-DCL1/2-dsRNA showed a punctuated fluorescent signal after MNase treatment, indicating encapsulation in the AVs (FIG. 1C). However, no fluorescent signal was observed when MNase was applied after rupturing the AVs by application of 1% Triton X-100 (FIG. 1C). Therefore, these results demonstrate that dsRNA can be efficiently encapsulated inside AVs, conferring nuclease protection.

Finally, we assessed the ability of the AVs as an efficient vehicle for dsRNA delivery to B. cinerea fungal cells. We compared fungal uptake of naked and AV-encapsulated Fluorescein-labeled dsRNA using CLSM. Fluorescent dsRNA was detected inside the fungal cells after application of either naked- or AV-Bc-DCL1/2-dsRNA to B. cinerea spores cultured on PDA plates (FIG. 1D). To eliminate any fluorescent signals coming from dsRNA or AV-dsRNA not inside the fungal hyphae, the CLSM analysis was carried out after Triton X-100 and MNase treatment. Under these conditions, fluorescent signals were still observed in the hyphae, supporting that the AV-dsRNA were taken up by the fungal cells (FIG. 1D).

External AV-dsRNA Application Triggers RNAi in B. cinerea

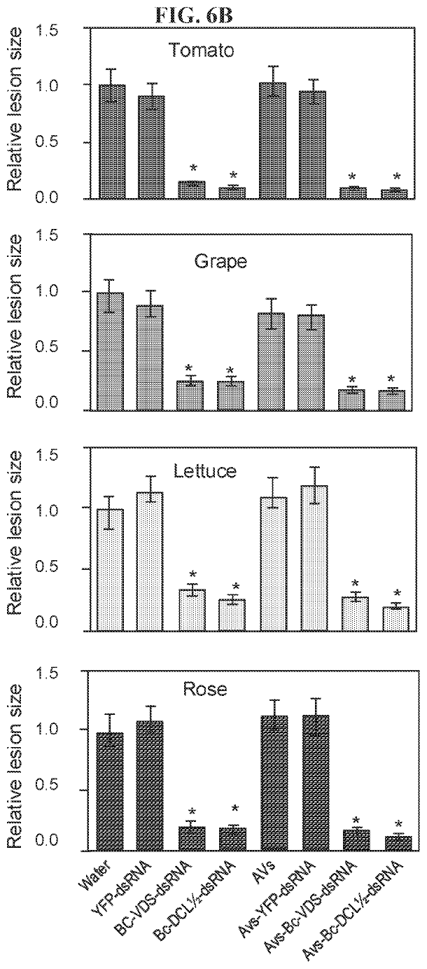

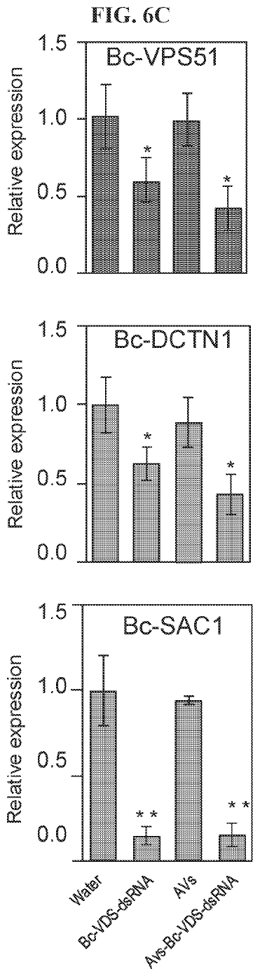

After demonstrating that the AVs could be loaded with dsRNA and taken up by fungal cells, we next examined if external AV-dsRNA application triggered RNAi in B. cinerea. Naked- and AV-dsRNA were externally applied to a variety of agriculturally relevant plant materials, including tomato and table grape fruits, lettuce leaves and rose petals, and a reduction of B. cinerea virulence was observed (FIG. 6A). Two fungal-gene targeting dsRNA sequences were used. One was the above-mentioned BcDCL1/2 sequence. The other was a sequence of 516 bp containing three fragments of B. cinerea genes involved in the vesicle-trafficking pathway: VPS51 (BCIG_10728), DCTN1 (BCIG_10508), and SAC1 (BCIG_08464).

Consequently, three dsRNAs were generated by in vitro transcription for loading into AVs: two of them specifically targeting B. cinerea virulence-related genes (Bc-DCL1/2 and Bc-VPS51+DCTN1+SAC1 (Bc-VDS)), while the third one was a non-specific target sequence (YFP) used as a negative control. All plant materials treated with naked- or AV-fungal gene targeting-dsRNA (Bc-DCL1/2 or -VDS) had reduced disease symptoms in comparison to the water treatment and YFP-dsRNA controls (FIGS. 6B and 6C). Further, both naked- and AV-Bc-VDS treatments decreased expression of the three targeted fungal virulence genes (FIG. 6C). Taken together, these results demonstrate how externally applied AV-dsRNA can inhibit pathogen virulence by suppression of dsRNA target genes and improve RNAi activity as compared to naked dsRNA.

AV-dsRNA Extends RNAi-Mediated Protection Against Gray Mold Disease Due to Enhanced dsRNA Stability and Durability

The instability of naked dsRNA currently limits the practical applications of SIGS. Though we demonstrated that AVs can protect dsRNA from nuclease degradation, environmental variables can also influence RNA stability, including leaf washing caused by rainfall events. Thus, in addition to enhancing RNAi efficiency in comparison to naked dsRNA, we were interested in evaluating if using the AV-dsRNA would prolong and improve the durability of the RNAi effect on B. cinerea.

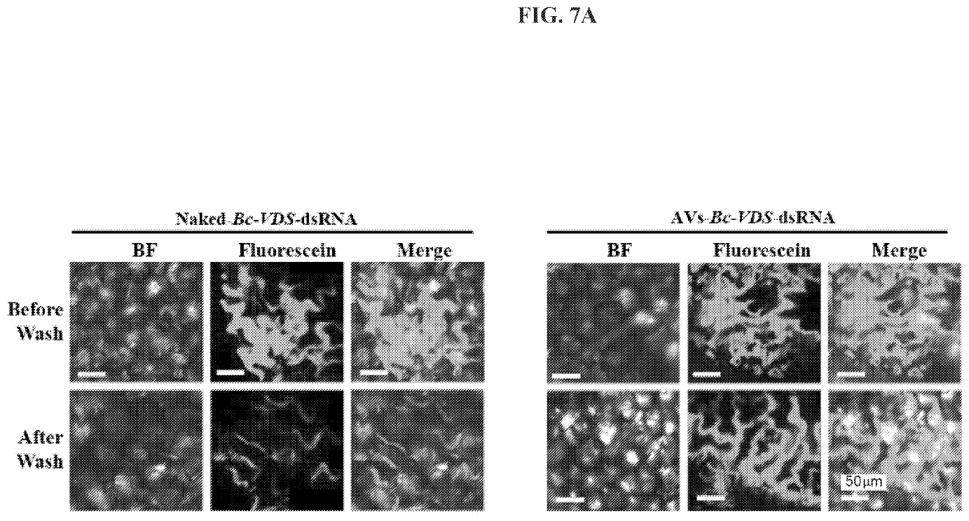

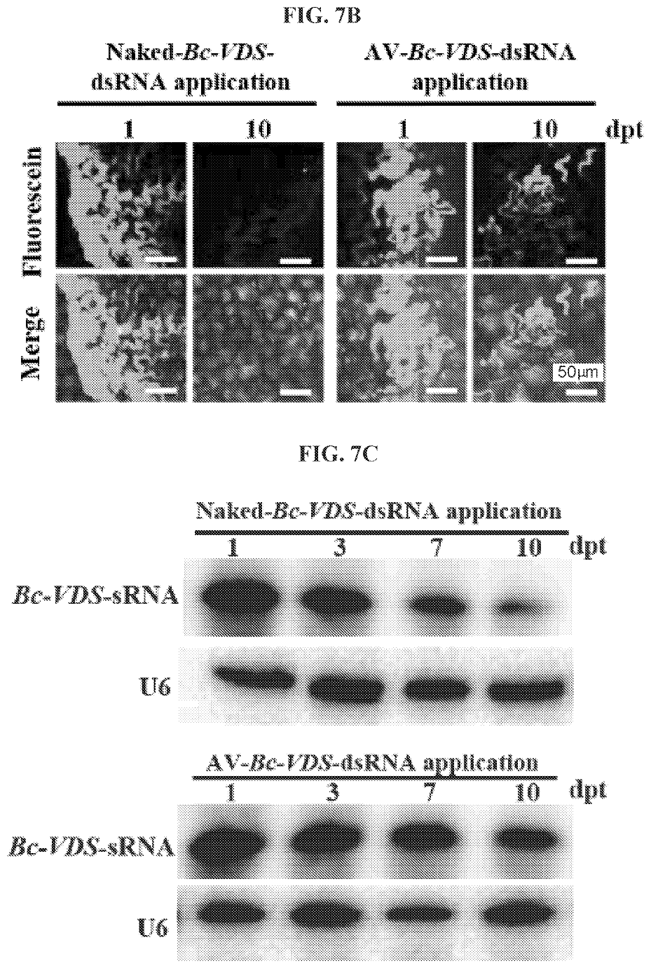

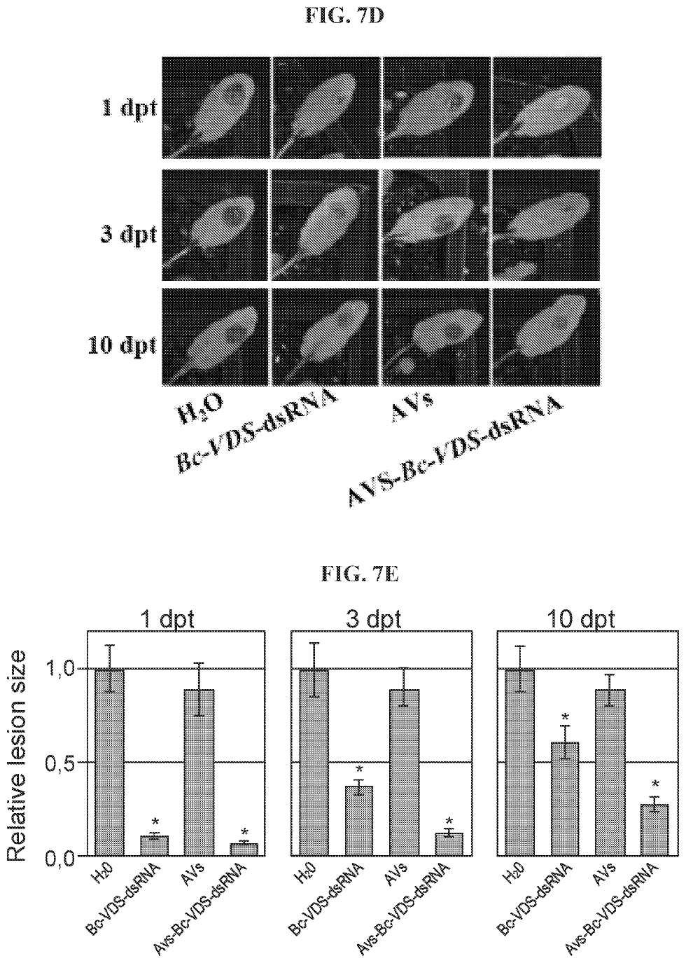

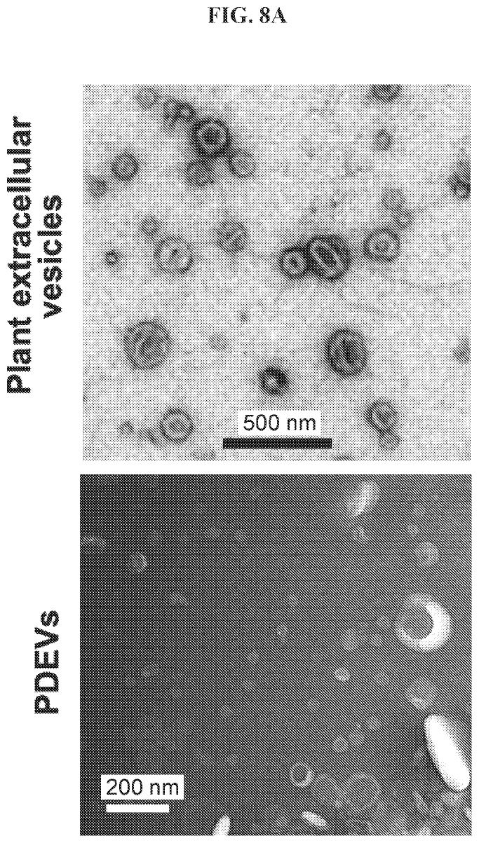

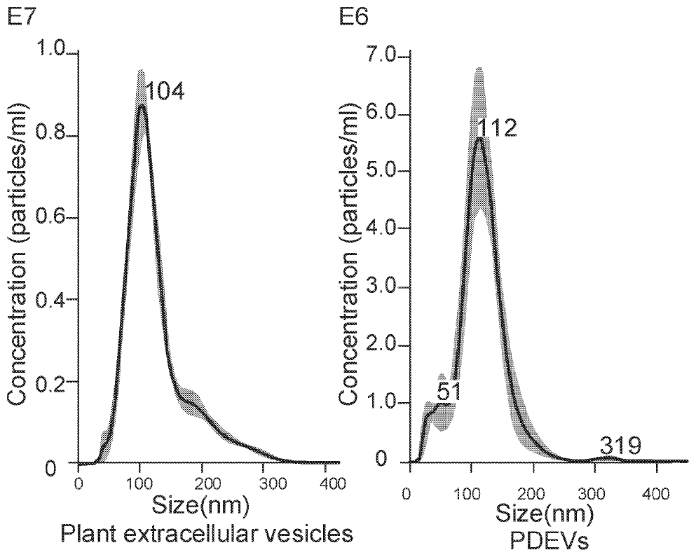

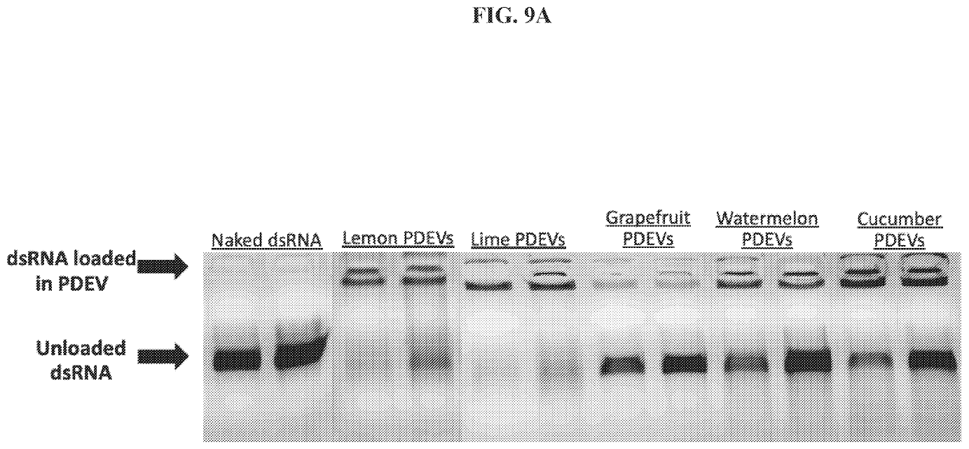

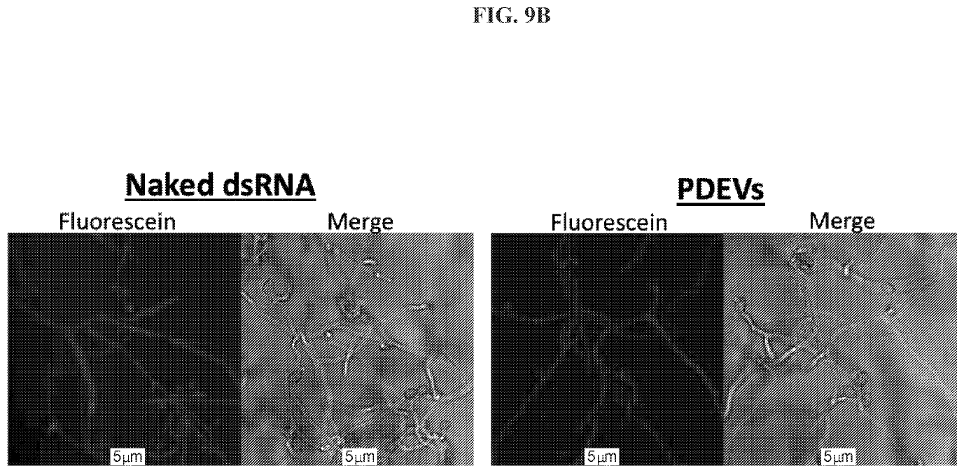

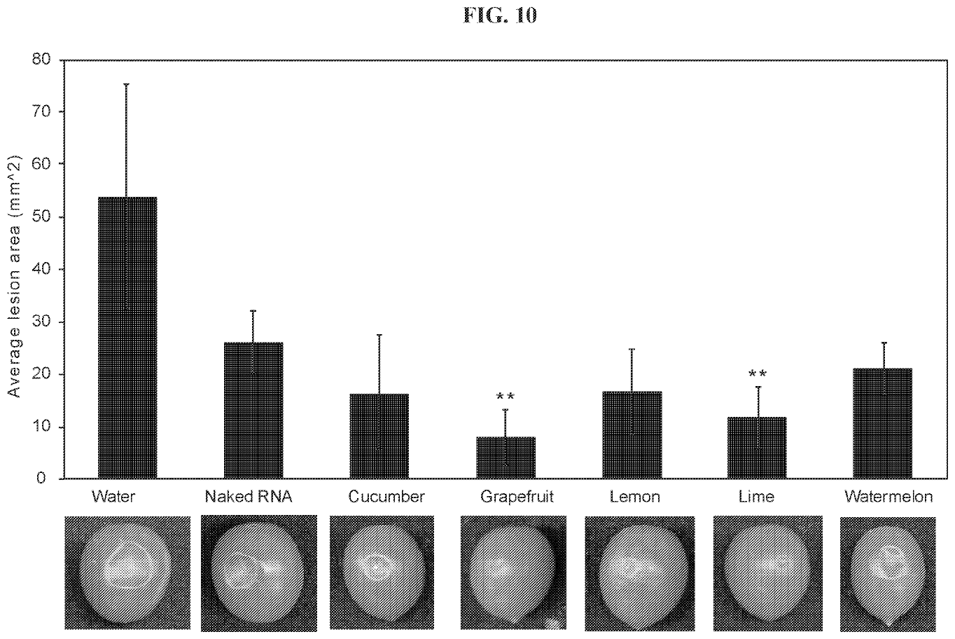

To assess the influence of washing on the stability and adherence of the AV-dsRNA to plant leaves, we analyzed the intact dsRNA content on the leaf surface using Fluorescein-labeled Bc-VDS-dsRNA and Northern blot analysis after water rinsing. The same concentration of Fluorescein-labeled naked- or AV-Bc-VDS-dsRNA (20 ng/μl) was applied to the surface of Arabidopsis leaves. After 24 h of incubation, the treated leaves were rinsed twice with water by vigorous pipetting. Immediately after, we found that the naked-dsRNA treated leaves showed a drastic decrease in fluorescence compared with AV-dsRNA treated leaves (FIG. 7A). These results suggest that most of the naked-dsRNA was washed off, whereas the AV-dsRNA largely remained on the leaves after rinsing (FIG. 7B). The effect of the AVs on dsRNA stability over time was also assessed. We observed a strong fluorescence signal after 10 days on Arabidopsis leaves that were treated with Fluorescein-labeled AV-dsRNA, indicating that AVs confer stability to dsRNA (FIG. 7C). By contrast, the naked-dsRNA application showed an undetectable fluorescent signal (FIG. 7B) and a weak hybridization signal on the Northern blot analysis, compared to AV-Bc-VDS-dsRNA treated leaves, which retained Bc-VDS-dsRNA (FIG. 7C). We further examined whether the AV-dsRNA remained biologically active over time and prolonged protection against B. cinerea compared to naked dsRNA. To this end, Arabidopsis leaves were inoculated with B. cinerea 1, 3, and 10 days post RNA treatment (dpt). Both naked- and AV-Bc-VDS-dsRNA treatments led to a clear reduction in lesion size over the time points assessed (FIG. 7D). However, the efficacy of the naked-VDS-dsRNA was reduced at a much faster rate than that of the AV-VDS-dsRNA, demonstrating that AVs can enhance the longevity of the RNAi effect of the loaded dsRNAs (FIG. 7E).

To examine if AV-dsRNAs could be similarly effective on economically important crops, we repeated these experiments using tomato fruits, grape fruits (V. lambusca var. Concord) and grape (V. vinfera) leaves. We applied naked- or AV-Bc-VDS-dsRNA on the surface of tomato and grape fruits and on the surface of grape leaves. Both the naked and AV-Bc-VDS-dsRNA applications led to weaker disease symptoms on tomato and grape fruits at 1, 5 and 10 dpt, as well as on detached grape leaves at 1, 7, 14 and 21 dpt, compared to the water or empty AV treatments (FIG. 4A). As we had observed in the Arabidopsis interactions, the AV-Bc-VDS-dsRNA applications greatly prolonged and improved the RNAi activity as compared to the naked-dsRNA over time for all plant materials (FIG. 4B). While the naked treatment lost the majority of its efficacy at 5-dpt in tomato fruits, 10-dpt in grape fruits, and 21-dpt in grape leaves, the AV-dsRNA treatments significantly reduced lesion sizes across all time points and plant material tested (FIG. 4B). These trends were also reflected in experiments on rose petals after the naked- and AV-Bc-VDS-dsRNA treatments. The enhanced reduction in lesion size observed specifically at the longer time points (i.e., 5, 10, 14, and 21 dpt) after AV-Bc-VDS-dsRNA application clearly demonstrates how AVs protect loaded dsRNA from degradation to extend the duration of plant protection against B. cinerea. Together, these results strongly support the ability of AVs to confer higher RNAi activity over time, effectively enhancing dsRNA stability for SIGS applications.

### Cost-Effective AV Formulations Also Provide Strong RNAi Activity

Our discovery that AVs can lengthen dsRNA mediated plant protection opens the door for its practical use in agricultural applications. Cost is a critical consideration for any crop protection strategy, so we next tested if more cost-effective AV formulations could be used for dsRNA delivery and RNAi activity. First, we removed the PEG, an expensive reagent in the formula, from our original DOTAP+PEG formulation, resulting in DOTAP AVs composed only of DOTAP and cholesterol in a 2:1 ratio. Additionally, we used a cheaper cationic lipid, 1,2-dioleyloxy-3-dimethylaminopropane (DODMA), in a 2:1 ratio with cholesterol to form DODMA AVs. DODMA has previously been utilized in drug delivery formulations, but has a tertiary amine and is an ionizable lipid compared to DOTAP, which could result in changes in RNA loading and activity. The DOTAP AVs were fully loaded with Bc-VDS dsRNA at a 1:1 N:P ratio (FIG. 2A), requiring the use of 4× fewer lipids than the DOTAP+PEG AVs, or the DODMA AVs, which were completely loaded at a 4:1 N:P ratio (FIG. 2B). Both DOTAP and DODMA formulations could effectively protect Bc-VDS dsRNA from nuclease degradation (FIG. 5C). The size distribution data for each AV formulation can be found in FIG. 2D. As expected, the z-average sizes of the DOTAP-derived AVs are similar, while the use of DODMA increases the z-average size.

Next, we examined if the different AV formulations influenced fungal dsRNA uptake or RNAi activity. After application of the different AV formulations, the fungal dsRNA uptake was tracked over 16 hours using CLSM. After 16 hours, all three AV formulations showed a similar amount of fungal RNA uptake, however, the uptake of DOTAP AVs was slower than that of DOTAP+PEG, or DODMA AVs, as evidenced by the weaker signal at the 90 minute and 3 hour time points (FIG. 2E). This could be due to differences in the AV chemistry. To confirm that the lower cost AV formulations have similar RNAi activity on B. cinerea over time as our original AV formulation, we performed treatments on tomato fruits. Both the DOTAP and DODMA formulations in complex with Bc-VDS-dsRNA trigger a steady RNAi effect on B. cinerea over time (FIGS. 3A-3C), significantly reducing lesion sizes at all time points (1, 5 and dpt). In addition, fungal biomass quantification indicated that the treatments with Bc-VDS-dsRNA encapsulated in DOTAP and DODMA AV formulations resulted in a statistically significant reduction of the fungal biomass at all time points. All AV-VDS-dsRNA treatments were also able to reduce expression of the targeted B. cinerea genes at all time points. Overall, these experiments demonstrate how new AV formulations that are more economical, but equally as effective, can be developed.

### DISCUSSION

These fungal gene-targeting RNAs developed for SIGS, are a new generation of environmentally-friendly “RNA fungicides” that offer a promising solution to mitigate the devastating impact of fungal plant diseases. However, commercial adoption of SIGS is still limited by the relative instability of naked dsRNA in the environment. Here, we demonstrate that packaging dsRNA in artificial nanovesicles stabilizes the dsRNA and extends the RNAi effect against the pathogen B. cinerea on different plant products.

The primary advantage that AV-dsRNA offers for SIGS over naked dsRNA is increased dsRNA stability. Here, we found that AVs protect loaded dsRNA against nucleases (FIGS. 1A-1D). This is crucial for extending the shelf-life of dsRNA products, since extracellular RNases and other ribonucleases have been identified on the fruits and the leaves of important economic crops such as tomato or tobacco. In addition, we have shown that the AV-dsRNA remains on the leaf surface for a longer period of time than naked dsRNA. Further, encapsulation of dsRNA by AVs also increases RNA adherence to the leaf after rinsing the leaf surface with water (FIGS. 7A-7E). Thus, use of the AVs for dsRNA delivery will greatly reduce the frequency and amount of spraying required for SIGS approaches in the field.

The key point of this work is that all of the described features of AV-dsRNA help to provide prolonged RNAi-mediated protection against B. cinerea on a wide range of plant products, especially for post-harvest products, compared to naked dsRNA applications. For example, protection was extended to 3 weeks (21 days) on V vinifera leaves (FIGS. 4A and 4B). This is similar to the extended protection provided by inorganic dsRNA complex formulations against viruses on Nicotiana tabacum cv. Xanthi leaves. This lengthened timespan of protection makes SIGS a much more agriculturally feasible crop protection strategy, changing the time needed between RNA applications from just a few days to up to a few weeks, enabling benefits in reducing the environmental and economic impact of such applications.

With agricultural applications in mind, we tested two more cost effective AV formulations. By removing the PEG from DOTAP-AVs, and DODMA-AVs, we can reduce the cost of AV synthesis. PEG is used in liposome preparations in clinical contexts to protect liposomes from immune cell recognition and prolong circulation time, however, this is not a concern in agricultural applications. Regardless, of the tested formulations, DOTAP+PEG was most effective in reducing fungal biomass at ten days post treatment, suggesting that PEG may play a role in enhancing fungal uptake efficiency of AVs. Meanwhile, DODMA and DOTAP AVs had comparable performance, and are both more cost-effective than the DOTAP+PEG, potentially making these formulations more suitable for agricultural use. Additionally, these efforts demonstrate how unique and effective AVs can be easily formulated and applied for SIGS applications.

In summary, we have provided strong evidence that an AV organic formulation confers protection to dsRNA that results in an effective and more durable RNAi effect against the fungal pathogen B. cinerea in a wide range of plant products, overcoming the main limitation of SIGS to date. This is one key step forward in the development of RNAi-based fungicides which will help reduce the volume of chemical fungicides sprayed on fields and offer a sustainable option to limit the impact of fungal pathogens on crop production and food security.

### Example 3—Isolation of Plant-Derived Extracellular Vesicles from Fruits and Vegetables

Step 1: Wash fruits and vegetables with soap and water. Remove any stickers.
Step 2: For citrus (lemons, lime, grapefruits, etc.), slice in half or quarters and collect juice using a juicer. For watermelon and cucumber, remove skin/rind and then slice into large chunks. Place chunks in blender and pulse on low for about 30 seconds or until chunks are homogenized. Do not blend for too long or seeds will be broken.
Step 3: Strain juice/homogenized chunks through a 4× folded Miracloth into a clean beaker to remove large chunks and pulp.
Step 4: Centrifuge juice at 1,500×g for 15 mins at 4° C. to pellet pulp and large debris.
Step 5: Transfer supernatant to another tube and centrifuge at 10,000×g for 30 mins at 4° C. to remove large particles. It may be necessary to repeat this step to ensure greater removal of the large particles and make filtration easier.
Step 6: Filter supernatant through a 0.45 um filter to remove large vesicles.
Step 7: Place filtered supernatant in ultracentrifuge tubes and centrifuge at 100,000×g for 1 hr.
Step 8: Resuspend vesicles in 1× PBS or vesicle isolation buffer.

### Example 4—Methods

### Plant Materials

Lettuce (iceberg lettuce, Lactuca sativa), rose petals (Rosa hybrida L.), tomato fruits (Solanum lycopersicum cv. Roma), and grape berries (Vitis labrusca cv. Concord) were purchased from a local supermarket. Host plants, including Arabidopsis thaliana, tomato (money maker), and grape plants were grown in the greenhouse in a 16/8 photoperiod regime at 24±1° C. before use in SIGS experiments.

Botrytis cinerea Culture and Infection Conditions

B. cinerea strain B05.10 was cultured on Malt Extract Agar (MEA) medium (malt extract 20 g, bacto protease peptone 10 g, agar 15 g per liter). Fungal mycelia used for genomic DNA and total RNA extraction were harvested from cultures grown on MEA medium covered by a sterile cellophane membrane. For B. cinerea infection, the B. cinerea spores were diluted in 1% Sabouraud Maltose Broth infection buffer to a final concentration of 10⁴ spores ml⁻¹ on tomato leaves and 10⁵ spores ml⁻¹ for drop inoculation on the other plant materials, 10 μl of spore suspension was used for drop inoculation of all plant materials used, except tomato fruits, in which 20 μl was used. Infected leaf tissues were cultured in a light incubator at 25° C. for 72 h and fruits for 120 h preserving constant and high humidity. Fungal biomass quantification was performed following the methods described by Gachon and Saindrenan. The p-values were calculated using Student's t-test for the comparison of two samples and using one-way ANOVA for the comparison of multiple samples.

### Synthesis and Characterization of Artificial Vesicles

PEGylated artificial vesicles were prepared following previously established protocols. In brief, PEGylated artificial vesicles were prepared by mixing 260 μl of 5% dextrose-RNase free dH2O with the lipid mix and re-hydrating overnight on a rocker at 4° C. The re-hydrated lipid mix was then diluted 4-fold and extruded 11 times using a Mini-Extruder with a 0.4 m membrane. PEGylated artificial vesicles-dsRNA (20 ng μl⁻¹) were prepared in the same manner by adding the appropriate amount of dsRNA to the 5% dextrose-RNase free dH2O before combining with the lipid mix. The average particle size of the artificial vesicles was determined using dynamic light scattering. All measurements were conducted at 25° C. using a Zetasizer Nano ZS instrument (Malvern Instruments Ltd, Malvern, Worcestershire, UK) and the samples were measured after 10-fold dilution in water. Data reported is the average of three independent measurements.

### In Vitro Synthesis of dsRNA

In vitro synthesis of dsRNA was based on established protocols. Following the MEGAscript® RNAi Kit instructions (Life Technologies, Carlsbad, CA), the T7 promoter sequence was introduced into both 5′ and 3′ ends of the RNAi fragments by PCR, respectively. After purification, the DNA fragments containing T7 promoters at both ends were used for in vitro transcription.

### In Vitro Naked- and AV-dsRNA Fluorescence Labeling for Confocal Microscopy

In vitro synthesis of dsRNA and labeling was performed based on established protocols. Briefly, Bc-DCL1/2-dsRNA was labeled using the Fluorescein RNA Labeling Mix Kit following the manufacturer's instructions (MilliporeSigma, St. Louis, MO). For confocal microscopy examination of fluorescent dsRNA trafficking into B. cinerea cells, 20 μl of 20 ng μl¹ fluorescent RNAs, either naked or loaded into AVs were applied onto 5 μl of 10⁵ spores ml-1. Germinating spores were grown on PDA medium and placed on microscope slides. The mycelium was treated by KCl buffer or 75 U Micrococcal Nuclease enzyme (Thermo Scientific, Waltham, MA) at 37° C. for 30 minutes, The fluorescent signal was analyzed using a Leica SP5 confocal microscope.

### External Application of RNAs on the Surface of Plant Materials

All RNAs were adjusted to a final concentration of 20 ng μl⁻¹ with RNase-free water before use. 20 μl of RNA (20 ng μl⁻¹) were used for drop treatment onto the surface of plant materials, or, approximately 1 mL was sprayed onto grape leaves before inoculation with B. cinerea.

Stability of dsRNAs Bound to AVs

The potential environmental degradation of dsRNA was investigated by exposure of naked-Bc-VPS51+DCTN+SAC1-dsRNA (200 ng) and AV-Bc-VDS-dsRNA (200 ng/2.5 μg) to Micrococcal nuclease enzyme (MNase) (Thermo Fisher) treatment in four replicate experiments. Samples were treated with 0.2 U μL⁻¹ MNase for 10 min at 37° C., and dsRNAs were released using 1% Triton X-100. All samples were visualized on a 2% agarose gel. The persistence of sprayed naked-Bc-VDS-dsRNAs and AV-Bc-VDS-dsRNAs (4:1) on leaves was assessed in two replicate experiments by total RNA extraction followed by northern blot analysis. 4-week old Arabidopsis plants were treated at day 0 with either a 201 drop of Bc-VPS51+DCTN1+SAC1-dsRNAs (20 ng μl⁻¹) or AV-Bc-VDS-dsRNAs (400:100 ng μl⁻¹) and maintained under greenhouse conditions. Single leaf samples were collected at 1, 3, 7, and 10 dpt. Total RNA was extracted using TRIzol and subjected to northern blot analysis as described above.

### VI. Exemplary Embodiments

Exemplary embodiments provided in accordance with the presently disclosed subject matter include, but are not limited to, the claims and the following embodiments:
1. A composition comprising an antifungal RNA and a lipid vesicle, wherein the antifungal RNA comprises a double-stranded RNA, a small RNA, or a small RNA duplex, and wherein the lipid vesicle is an artificial vesicle comprising a tertiary amine cationic lipid or a plant-derived vesicle.
2. The composition of embodiment 1, wherein the antifungal RNA targets a dicer-like (DCL) gene of a fungal pathogen.
3. The composition of embodiment 1, wherein the antifungal RNA targets the vacuolar protein sorting 51 (VPS51) gene, the dynactin (DCTN1) gene, or the suppressor of actin (SAC1) gene of a fungal pathogen, or a combination thereof.
4. The composition of embodiment 1, wherein the antifungal RNA targets a polygalacturonase gene or an exo-polygalacturonase gene of a fungal pathogen, or a combination thereof.
5. The composition of embodiment 1, wherein the antifungal RNA targets the long terminal repeat (LTR) region of a fungal pathogen, or a combination thereof.
6. The composition of any one of embodiments 2-5, wherein the pathogen is Botrytis, Sclerotinia, or Verticillium.
7. The composition of any one of embodiments 1-6, wherein the lipid vesicle is the plant-derived vesicle.
8. The composition of embodiment 7, wherein the antifungal RNA is not expressed by the plant from which the plant-derived vesicle is derived.
9. The composition of embodiment 7 or embodiment 8, wherein the plant-derived vesicle is obtained from N. benthamiana leaves, a fruit, a vegetable, or a combination thereof.
10. The composition of any one of embodiments 1-6, wherein the lipid vesicle is the artificial vesicle comprising the tertiary amine cationic lipid.
11. The composition of embodiment 10, wherein the cationic lipid is N,N-dimethyl-2,3-dioleyloxy)propylamine (DODMA) or a salt thereof.
12. The composition of embodiment 10 or embodiment 11, wherein the ratio of the secondary amine in the cationic lipid to phosphate in the RNA ranges from about 1:1 to about 10:1.
13. The composition of embodiment 13, wherein the ratio of secondary amine in the cationic lipid to phosphate in the RNA is about 4:1.
14. The composition of any one of embodiments 10-13, wherein the vesicle further comprises a sterol.
15. The composition of embodiment 14, comprising the cationic lipid and cholesterol in a molar ratio ranging from about 1:1 to about 10:1.
16. The composition of any one of embodiments 10-14, wherein the vesicle is a micelle, a small unilamellar vesicle, a large unilamellar vesicle, or a multilamellar vesicle.
17. A method of increasing pathogen resistance in a plant or a part of a plant, the method comprising contacting the plant or the part of the plan with a composition according to any one of embodiments 1-16.
18. The method of embodiment 17, wherein the double-stranded RNA, small RNA, or small RNA duplex is sprayed onto the plant or the part of the plant.
19. The method of embodiment 17 or embodiment 18, wherein the plant is a fruit- or vegetable-producing plant.
20. The method of any one of embodiments 17-19 wherein the part of the plant is a fruit, a vegetable, or a flower.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

INFORMAL SEQUENCE LISTING

SEQ ID NO: 1-Botrytis cinerea DCL1 genomic DNA sequence (selected RNAi
fragment marked by bolded text)
ATGACGAGAGACGCAGCAGCAGCAAAAAGTCTCTACCATTGGCGAAGAAAAGGCGTCACTC
CTTCAGCCGAAGAGGATCTTCTATCGTTTGATGATATTGTTACTGCCGTTCCACCTACAATCT
TGTCTTCGTCTGTCGCTCCATATACTTCTCGAGATAAGATACCTTCTGCATCTGGCAACGGAG
ATGCTATAGCAGATGTTAGCAGTGGTTACCTCAAACAGGCTACCGTATCTTCTCATTCTGCTC
AAGTCCGATCATCTTCAAACGGCAATCAAGGTGATGCCAAAAGTTCTCCCTCTCTTTCACCT
GATAGTAAACTGGAATTCATCTTTGGGCCTCCTTTAAGGGAGCCAGAGAAGCCATTCTTTAA
TAAATCTTCTTATTCGTTTCGAGATTCGAGAGGGTTGAGCAGAAATCGGGCTTCTTCTTCTAT
GGAAAATTCGAGAACTCTCGATCCAAAGATACTCAAACCAGTTATCATCAATAATCACCAGG
GCGAATGCTTCCAAGAGGCTTCCAGAACAGGTATACCTCAGGCTGATACTTTTGATAAATCT
TCCCTTGCTAAGACTGCGGATATGGATTTGTCACCAGTTTCTCACCATGCGGATGTGCTTGCG
ACGACGGTCACTGCACAGCATTCTGCAATAGCCGCCCAGAACGCAGCTCAAAGCTCTAAGA
TGCCAGGTCCTGAAGCTTTTTTACTTGCCGAAAAGGACGAGGCAGGTTCTCCCGTTGTTATAT
CACTGGGTTCTGCAAACCAAATTCCTTCTGGAAACATTTCTTTGCAGCTTGATTCACCATCTC
TGGAAAACCATTCTCCAAATGTGACCCCAATCAACAAAGTCCCTACACCATTCGCACTTTCT
ACAAGGACAACCGATGACGTTTTCGCAGAACTTAGGCGGCCTTTGCATCCCCAAGCTATTCA
GAGCCAGATTGATATCAAGACTTCCTCTTGTGTTGATAGTTATAACACGAATGATGAGATTC
TAGACAACAATCAAGGTTCCAATCAAAAAGATCTGCATGTTGTTGAAAAGGATAAGGAAGA
GGAAGAGGAAGAGGATATGAACCAAGCCATACCCGATATCAAACGTATCTCAGCACGAAAA
CAAAAGAACGCTGCCATATTTGACGTTTTTCTTAAGGAAGCTACCAAACTACCAAAGACAGA
AAAGACTTCACATGCGAATGATGAAGCAATTCAGTCTACTAGGTGGTTGATTGACCAAGCAG
AAAAACAGCATATTATAGAAAGTCCCAGGGACTATCAACTTGAATTGTTTGAGAAGGCAAA
GAAACAGAACATTATAGCTGTACTTGATACAGGATCTGGCAAGACATTCATTGCAGTTCTCT
TACTTCGGTGGATCATAGACCAAGAGCTTGAAGATAGAGCTATTGGCAAGCCTCATCGTGTT
TCATTCTTCCTGTGGAAGAAACGACTGGATACGAATATGGTCATTGTCTGCACTGCAGAAAT
TTTGCGCCAATGCCTGCACCATTCGTTTGTTACAATGGCTCAAATAAATCTGCTAATTTTCGA
TGAAGCCCACCATGCAAAGAAGGATCATCCTTATGCTAGGATTATTAAAGATTTTTATCGCA
ATGACACGGAAAAGGATATCGCTCTGCCTAAAATATTTGGGATGACAGCATCACCGGTAGA
TGCTAGAGATAATGTCAAGAAAGCTGCGGAAGAACTTGAAGGTTTGCTACACAGTCAAA
TATGTACTGCAGAAGATCCCAGCTTGCTGCAGTACTCAATCAAAGGTAAACCTGAGACT
CTTGCCTACTATGATCCCTTGGGCCCGAAATTCAATACTCCTCTTTATCTTCAAATGCT
CCCGCTTCTAAAAGACAATCCTATCTTTCGGAAGCCATTTGTATTTGGGACAGAAGCCA
GTAGAACTCTAGGATCTTGGTGTGTTGACCAGATCTGGACTTTCTGTCTTCAAGAAGAA
GAGTCTAAGAAACTACAAGCAAGGACGGAGCAGGCGCATCATAAGAAGAGAGTCCCGGAG
CCACTTGAAGTGCTAGAGAAACGCAAGGAACAACTTGAACAAGCCAAATCCATTGTCGAAA
ATCACACTTTCGAGCCACCACACTTTGCATCAAGATTATTGGATGATTTCACAACAAAAGTT
CACTATTCGAATAATTTATCTACTAAAGTCGTTGCTCTCTTGAGTATTCTCAAAGATCGTTTC
CAACGACCCACCAATGACAAGTGTATTGTATTTGTCAAAGAAAGATACACCGCACGCCTTCT
AGCCTCACTTCTCTCCACACC
TGAAGCTGGGACACCATTCTTGAAGGCTGCACCGCTGGTTGGTACTACGTCTGCTTCAGCCG
GGGAAATGCATATCACATTTAGATCACAAACTCTTACTATGCACAACTTTCGCAATGGTAAA
ATCAACTGCCTTATCGCAACATCAGTTGCTGAAGAAGGTCTTGACATTCCTGACTGTAACCT
CGTTGTCAGATTCGATTTGTACAATACAGTCATTCAGTACATTCAATCTAGAGGTCGTGCTAG
GCATATCAATTCAAGGTACTACCATATGGTAGAGAGCCACAACGAGGAACAGATTCGTACA
ATCAAAGAGGTTTTGAAGCATGAGAAAATGCTAAAGCTTTTTGCTTCTGCTCTTCCAGAAGA
TCGAAAATTGACCGGAAACAACTTCAATATGGATTACTTCCTCAGAAAAGAACGAGGCCAC
AGAATTTACCCTGTCCCGAATAGTGACGCAAAACTTACTTACAGAATGAGCTTAACGGTCCT
ATCTGCCTTCGTTGACTCACTTCCTCGAGCCCCAGAGTCGGTTCTTCGAGTGGATTATGTCGT
CACAACTGTCGATAAGCAGTTTATCTGTGAGGCCATTTTGCCAGAAGAAGCACCCATACGCG
GAGCAATTGGTCGGCCAGCAACAACTAAACAAGTGGCCAAATGCTCAGCAGCCTTTGAAAC
TTGTGTGATTCTGCACCAGAAAGGATACATCAACGACTACCTACTTTCTACATTTAAAAGAT
CAGCACACATGATGAGAAATGCACTTTTGGCTGTGGATGGAAAGAAGCAAGAAGCTTATGA
TATGCAGACTAAACCAACTTTATGGTCTTCGAAAGGGAAACAAGGCATATTTTATATGACTG
TCTTGTCTCTCAAATCTCCAGATAATCTTGACAGAGCATCTCAGCCATTGGGCTTACTGACAA
GATCACCCTTGCCTGATTTGCCAGAATTTGTTCTTCATTTCGGAGCAGGGCGAAACTCTCCAA
CCTCGTGCGTACCTCTCGCTTCCTCAATTACGCTCGAAAAAAACAAGCTTGACCAAGTTAAT
ATGTTCACCCTATGTTTATTCCAAGATGTGTTCAGTAAAGCATACAAATCAGATCCGGATAG
TATGCCATACTTTCTGGTTCCTATCAACTGCCTGAATGCTATTGTCGACTGGAAATCACAAAA
CCCAATGTCAATAATCGATTGGGAGACAGTTGAATATGTCCAAGACTTCGAGAATAAGCAA
GCTGATAAGCCATGGGAGCACAAGCCATGGTTAGGAAAGCCTGACGATTATTTCAAAGACA
AATTCATAACTGATCCCTTTGACGGGTCTCGAAAATTGTGGTCCGTTGGAATCACAAAAGAA
TACAGACCATTGGATCCAGTCCCACCAAACACGGCGCCCAGGAAGGGAGCTAGAAAGAACA
ATAGTAATATCATGGAGTATAGTTGTAGTCTCTGGGCAAAGGCTAGAGCAAAACGAACTTTT
GATGAAGAACAGCCTGTTATTGAAGCAACCTACATTTCACTTCGGAGAAATTTGCTTGATGA
ATTTGATGGAGGTGAGCTCGAGACTTCAAAGAAGAGTTTTATTATTTTAGAACCATTGAAGG
TATCACCTCTTCCAACTACCGTGGGTGCAATGGCCTATCTTTTACCTGCAATTATTCATCGAG
TTGAGTCATATCTCATTGCTCTTGAAGCAACAGACTTGTTACATCTTGATATCCGTCCTGATC
TTGCGCTAGAGGCTGTTACCAAGGATTCCGACAATTCTGGAGAGCATGGTGAGGAACAGAC
AAACTTTCAACGTGGAATGGGCAATAATTATGAACGATTGGAATTTCTTGGGGACTGCTTCT
TGAAGATGGGAACGTCAATATCTCTATACGGTCTAAATCCTGATAGTGATGAATTCCGCTAC
CATGTTGATCGTATGTGTCTGATTTGCAACAAAAATCTGTTCAATACGGCTTTGAAATTAGA
GCTTTACAAATACATTCGGTCGGCAGCCTTCAACCGACGAGCTTGGTATCCCGAAGGCCCCG
AATTATTAAGAGGAAAGACAGCCACGGCACCAAATACCCACAAGCTCGGCGATAAGTCAGT
TGCAGATGTTTGTGAAGCAATGATTGGAGCTGCTTTACTAAGCCACCACGAAAGCAAGTCCA
TGGATAATGCGGTTCGCGCCGTTACTGAAGTTGTCAATAGTGACAACCACAATGCTGTTGTA
TGGTCTGATTATTACAAATTGTATGAGAAACCAAAATGGCAAACTGCTACAGCTACAGCTGC
ACAAATAG
ATATGGCAAGACAAGTTGAAATGAAACATCCATATCATTTCAAACACCCACGCCTGTTAAGA
TCAGCTTTCATCCATCCGGCATACTTGTTCATCTATGAACAAATTCCTTGTTATCAACGTCTC
GAATTTTTGGGTGATTCGCTACTCGATATGGCATGTGTCAACTTCCTTTTTCACAACCACCCA
ACAAAAGATCCTCAGTGGCTCACTGAGCACAAGATGGCTATAGTATCCAATCAGTTTCTTGG
AGCTCTTTGTGTCAAATTAGGCTTCCACAAACATCTACTGACACTCGATTCTCAAGTTCAAAA
AATGATTGCAGATTACTCCTCAGATATCAATGAAGCTCTCATTCAAGCCAAAACGGACGCAA
AGAGAGTCGGCAAAGTAGAAGATGATTACGCTCGTGATTATTGGATTGCCGTCCGTCAACCT
CCTAAATGTCTTCCCGATATTGTAGAAGCATTCATTGGTGCCATTTTTGTCGACTCTGAGTAT
GACTACGGTGAAGTTGAGAAGTTCTTTGAAATGCATATCAGATGGTACTTTGAGGATATGGG
CATCTACGATACCTATGCTAACAAGCACCCAACCACTTTCCTTACTAATTTCTTGCAAAAGA
ACATGGGATGTGAGGACTGGGCACCAGTTAGTAAGGAAGTACCTGGAGAGGATGGTAGAAA
GAATGTTGTAGTTTGCGGGGTCATCATACACAATAAGGTGGTATCAACTGCCACTGCCGAAA
GTATGAGATATGCTAGGGTCGGAGCAGCGAGGAATGCCTTGAGAAAATTGGAGGGAATGAG
TGTCCGAGAATTCAGGGATGAATACGGGTGCTCATGTGAAGGTGATGTTGTTGATGAAGAG
GGCAATATTGAATTTGTTGAACGTGAAGACGGGATGGAGGGGATCGGTATGGGATATTGA
SEQ ID NO: 2-Botrytis cinerea DCL1 protein sequence
MTRDAAAAKSLYHWRRKGVTPSAEEDLLSFDDIVTAVPPTILSSSVAPYTSRDKIPSASGNGDAI
ADVSSGYLKQATVSSHSAQVRSSSNGNQGDAKSSPSLSPDSKLEFIFGPPLREPEKPFFNKSSYSFR
DSRGLSRNRASSSMENSRTLDPKILKPVIINNHQGECFQEASRTGIPQADTFDKSSLAKTADMDLS
PVSHHADVLATTVTAQHSAIAAQNAAQSSKMPGPEAFLLAEKDEAGSPVVISLGSANQIPSGNIS
LQLDSPSLENHSPNVTPINKVPTPFALSTRTTDDVFAELRRPLHPQAIQSQIDIKTSSCVDSYNTND
EILDNNQGSNQKDLHVVEKDKEEEEEEDMNQAIPDIKRISARKQKNAAIFDVFLKEATKLPKTEK
TSHANDEAIQSTRWLIDQAEKQHIIESPRDYQLELFEKAKKQNIIAVLDTGSGKTFIAVLLLRWIID
QELEDRAIGKPHRVSFFLWKKRLDTNMVIVCTAEILRQCLHHSFVTMAQINLLIFDEAHHAKKDH
PYARIIKDFYRNDTEKDIALPKIFGMTASPVDARDNVKKAAEELEGLLHSQICTAEDPSLLQYSIK
GKPETLAYYDPLGPKFNTPLYLQMLPLLKDNPIFRKPFVFGTEASRTLGSWCVDQIWTFCLQEEE
SKKLQARTEQAHHKKRVPEPLEVLEKRKEQLEQAKSIVENHTFEPPHFASRLLDDFTTKVHYSNN
LSTKVVALLSILKDRFQRPTNDKCIVFVKERYTARLLASLLSTPEAGTPFLKAAPLVGTTSASAGE
MHITFRSQTLTMHNFRNGKINCLIATSVAEEGLDIPDCNLVVRFDLYNTVIQYIQSRGRARHINSR
YYHMVESHNEEQIRTIKEVLKHEKMLKLFASALPEDRKLTGNNFNMDYFLRKERGHRIYPVPNS
DAKLTYRMSLTVLSAFVDSLPRAPESVLRVDYVVTTVDKQFICEAILPEEAPIRGAIGRPATTKQV
AKCSAAFETCVILHQKGYINDYLLSTFKRSAHMMRNALLAVDGKKQEAYDMQTKPTLWSSKG
KQGIFYMTVLSLKSPDNLDRASQPLGLLTRSPLPDLPEFVLHFGAGRNSPTSCVPLASSITLEKNK
LDQVNMFTLCLFQDVFSKAYKSDPDSMPYFLVPINCLNAIVDWKSQNPMSIIDWETVEYVQDFE
NKQADKPWEHKPWLGKPDDYFKDKFITDPFDGSRKLWSVGITKEYRPLDPVPPNTAPRKGARK
NNSNIMEYSCSLWAKARAKRTFDEEQPVIEATYISLRRNLLDEFDGGELETSKKSFIILEPLKVSPL
PTTVGAMAYLLPAIIHRVESYLIALEATDLLHLDIRPDLALEAVTKDSDNSGEHGEEQTNFQRGM
GNNYERLEFLGDCFLKMGTSISLYGLNPDSDEFRYHVDRMCLICNKNLFNTALKLELYKYIRSAA
FNRRAWYPEGPELLRGKTATAPNTHKLGDKSVADVCEAMIGAALLSHHESKSMDNAVRAVTEV
VNSDNHNAVVWSDYYKLYEKPKWQTATATAAQIDMARQVEMKHPYHFKHPRLLRSAFIHPAY
LFIYEQIPCYQRLEFLGDSLLDMACVNFLFHNHPTKDPQWLTEHKMAIVSNQFLGALCVKLGFH
KHLLTLDSQVQKMIADYSSDINEALIQAKTDAKRVGKVEDDYARDYWIAVRQPPKCLPDIVEAFI
GAIFVDSEYDYGEVEKFFEMHIRWYFEDMGIYDTYANKHPTTFLTNFLQKNMGCEDWAPVSKE
VPGEDGRKNVVVCGVIIHNKVVSTATAESMRYARVGAARNALRKLEGMSVREFRDEYGCSCEG
DVVDEEGNIEFVEREDGMEGIGMGY*
SEQ ID NO: 3-Botrytis cinerea DCL2 genomic DNA sequence (selected RNAi
fragment marked by bolded text)
ATGGAATACACTTCGGAACCTGACACTGACCCGGATACACGCGGTAGCCTTATCGATGGTCG
AGATGGGATTGAAGGGGATCTTATTGCTTTGACGTCTGGGGAACGACTTAATGAGACTGTAG
AGGATTTATGTAGTGACTCATCAGGATTGATTGTTGAGAATGAAGATGATGATAACAGCGCA
GGGGAGAAGGGAGAGATTGTGATAGTAACACCAAGAACATACCAACTGGAAATGTTGGAA
GAGAGTTTGAAAAGGAATGTCATCGTTGCGATGGATACAGGAAGTGGCAAGACACATGTGG
CCGTTCTCCGAATACTAGCGGAACTTGAGCGGATGAAGCCTGGCAAGATAATATGGTTTCTT
GCGCCTACCGTTGCGCTCTGTGCTCAGCATCACGAATATCTCCAGCTGAATATTCCCTCTGTT
TTGATCAAAATGCTTATTGGTGCTGATGGTGTGGATCGATGGACAGAGCAGAGACAGTGGG
ATACGGTCTTGAAGGATGTCAAGGTAGTCGTATCTTCCTATCAAGTTCTTCTAGATGCCCTTA
CACACGGATTCGTACGCATGGGGCGTCTGTCCTTGATCATTTTTGATGAAGCACATAATTGT
GTAAATAAAGCGCCAGGGGCTAAAATTATGAAATCTTTCTATCATCCGTATAAATCGATATT
CCCACTTCCCCACATTCTGGGCCTCTCGGCCAGCCCTGTCATGAGATCCAGTCCACAATCTT
TAAGTGATATCGAGGAGACTTTGGATGCCATTTGCTGCACGCCAAAAATACATCGAGC
AGATCTTCGCCTTCGAGTAAAGCTACCACTTCTATCTATTATCTACTATACCCCAGAGT
CAAATATCATCGTGACGAAAACTGTGGCGAGCCTGAGAAAGATTGTGCAAAGTCTCAA
CATTTTCGAAGACCCCTACGTTTTGACACTAAAAAGGAGTGATAGCGAAAAAAGTCAAC
GTGAGCTGGCGAAAGTACTCAAGAGTTTTAAGACATATAGTCAAACCCAATTAAAGTCA
ATCGACAAAACTAGCAACGAGATTATTCTTGTAGAGCTAGGCCCATGGGCTGCAGATTACTA
TATCTCAACAGTGGTGACGAGATACTTGAAGGCAATGTCGGCAAAGGACACTTTCATTGTTG
AAGATTCACCAGCTGCCGAGAAGCTATATATTGCCAAGGCTCTCAGACAAGTCGAAATCTCT
CCTTCAACTCTCTCAGATACAGGCAAAATTTCTAACAAGGTTGAAAAGCTACTGGGGATAAT
TGCGCAACAGAAGCCTCCCTTTTCCGCTATTATATTTGTCCAAGAAAGAGCCACGGTGTCTG
TGCTAGCCCATCTATTATCGCATCATCCATTGACAAAGGATCGTTTTAAGATTGGAACCATG
GTTGGCACATCCTTAAATGGCAAGCGTACAGACCAAATAGGAGAGCTTGTCGATGTTAATCA
ACAAAAAGACACTTTGTCAAGTTTCAAGCGTGGAAAAATTGATATCCTTATAGCTACAAATG
TATTGGAAGAGGGAATTGATGTTCCTGCCTGTAATCTAGTGATCTGCTTTAGTAAACCAGCA
AACCTCAAATCTTTCGTACAAAGACGAGGGCGAGCAAGACAGCAAGATTCTAAGCTGATTC
TTCTTGATGCTTCAGGTGATAAAGCGACAAATTGGCATGAGCTTGAAAGAAAAATGCGAGA
GGAGTACGGAAAGGAAATGCGAGAATTGCAACACATCTACGAAATTGAGACAGCTGATGAA
CAGTCGGAAGATGATAGGGTCTTGCGAATAGAAAGCACTGGGGCTCAATTAGACCTTGACA
GTGCTTTACCACATCTCTATCATTTCTGTTCAGTCTTAACAACAAAAGATTTTGTTGACCTCA
GGCCAGACTTCGTCTACTCCTCCGAACTGGGATCGGAATATGTTCGAGCAAAGGTCATCCTG
CCTGGATCGGTTTCTAAACCCCTGCGAGTCCATGAAAGCCGCGGATCGTGGTTGAGCGAGAG
GTCGGCTGCAAAAGATGCAGCGTTTGAGGCGTATTCCGCATTATACAGGGGGGGCTTAGTGA
ATGATAACCTACTGCCCCTGATGGTGCACGACAAAGTCATCGATGAGTTGACTTCAAAGCCC
GTGGATACTCGCGCGTCTCTTCTGGAGGTGAAGGAAAGATTAAATCCATGGATTGACATTGC
TAGAGCATGGAAAGAGGCAGAACACCATGCTGGAATTGTTCGCACATCGGTAATGATCTTC
AATGGGATGAAGCTGGAACTCTGTCTTCC
AATTGATCCACCGGCAATACCCCCATTAAAGCTTTATTGGGATGCTGACACCGAGTTCTTTGT
TGACTTTACAAACGATATCGAGATCGGCACCAGCGAGAATATGTTGGCACAGGCGTTGAAC
GATACCAATCTACTATTATCAGATCGTGGTCGTAAAGTTCACATCCAGTCACGTCGAACAGT
TGTGCAATTTATCTTGCTTCAAGATTCGGGCTCGCTCAGTTCAGATTGTTTTCCGGTTGACCC
CAACGGTAATATTAAAAGTACAGGTTTTATCAGAGAAGTCGGTAAACTAGAATCGCCCTACA
TCTTTGAAAAATGGTTGCCCAATGCACCAGAAGACGTCCCATATCTAGCTGTGGTTAAAGTA
AGTCGCCGTGCAGACTTTTTGCACAAGGTACAGAACGAAAAACCCTCGTCATTCACTAAACA
ATTCTCGTCTGTTCTACCTGCCTCGACATGTGTACAGGATGTAATGCCCGCACAGTTGTCTCG
GTTCGGCATGATGATTCCTTCCATCACACACCACATTGAGGTGCAACTCGTTGTAGACCGAC
TATCCAGGACCATCCTCAAGGATCTCGAAATTAGTGACCAGAGTCTTATTCAGACCGCCATC
ACACATGCCAGTTATTCGTTAGACTCGAATTATCAGCGTCTCGAATTTCTGGGCGACTCAATT
CTCAAATTGTGTACATCGGTACAATTGGTGGCAGAGCATCTAGATTGGCACGAAGGATATTT
GTCGGCTATGAAGGATCGTATCGTGTCCAATTCACGGTCATCAAGAGCGGCGGCTGAAGTCG
GTTTGGATGAGTATATAATGACCAAGAAATTCACAGGTGCAAAATGGCGACCAATGTACGT
GGATGATCTGGTCGTCACAGAACAAAAAACAAGAGAAATGTCCTCCAAAATTCTTTCCGAC
GTTGTGGAAGCACTCATCGGCGCATCTCTCCGGCCCGTCGAGCAAATCCTCGCATATACCTT
CACCAAAAAATCTCTCCTCGTCGAAGCCATGACGCACCCCTCTTACACCAGCGGCACGCAAT
CCCTCGAGCGACTCGAGTTCCTCGGCGATTCCATTCTCGACAACATCATCGTCACAGCCATG
TGGTCGCACTCGACGCCGCTCTCCCACTTCCACATGCATCTCCTGCGCTCTGCGCTCGTCAAC
GCCGATTTCCTCGCCTTTCTCTGCATGGAAATGAGCATCGACCAAAACGTCACCAATCTGAC
CGAAGGAAAAAACCATCGCATCCACGAAACCCACTCGCGACGCCGCGTTTCCCTCGTCAGTT
TTCTCCGTCACTCAAGCGTTCGTCTCTCTATCTATCAAAAAGAAGCGCTTTCTCGCCATGCAG
AATTGCGCGATCAGATCCTCGAGGCAATATACACCGGTGATACATTCCCCTGGGCTCTATTA
TCCCGATTGGACGCGCGGAAATTTTTCTCCGATATGATTGAGAGTTTGCTGGGCGCGGTATG
GATTGATAGCGGCTCGATGGAAGTGTGCACGCAGCTGATCGAAAGAATGGGCGTCCTGAGA
TACATGCGACGGATTTTGAAAGATGGCGTGCGCATCATGCATCCGAAGGAGGAACTGGGCA
TCGTGGCCGATTCTGAAAACGTCAGGTACGTTTTGCGGCGGGAGAAGATGGGTGGGGATGC
TACCGAGGTAAATGCGGACGCGGATGAAGAGGTACGCACGGAGTACCGGTGCACAGTATTT
GTGGGGGGGAGGAAATTGTAGAGGTGAGGGGTGGAGCGAGGAAAGAGGAGATTCAGGCA
AGGGCTGCGGAGCAGGCGGTGCGGATTTTGAAGGCGAGGGGTCATGAGAAGAGGAATGGG
GGTGCGGGGGAGGGGAAAAAGAGAAAATCGCTGGATGAATAG
SEQ ID NO: 4-Botrytis cinerea DCL2 protein sequence
MEYTSEPDTDPDTRGSLIDGRDGIEGDLIALTSGERLNETVEDLCSDSSGLIVENEDDDNSAGEKG
EIVIVTPRTYQLEMLEESLKRNVIVAMDTGSGKTHVAVLRILAELERMKPGKIIWFLAPTVALCA
QHHEYLQLNIPSVLIKMLIGADGVDRWTEQRQWDTVLKDVKVVVSSYQVLLDALTHGFVRMG
RLSLIIFDEAHNCVNKAPGAKIMKSFYHPYKSIFPLPHILGLSASPVMRSSPQSLSDIEETLDAICCT
PKIHRADLRLRVKLPLLSIIYYTPESNIIVTKTVASLRKIVQSLNIFEDPYVLTLKRSDSEKSQRELA
KVLKSFKTYSQTQLKSIDKTSNEIILVELGPWAADYYISTVVTRYLKAMSAKDTFIVEDSPAAEKL
YIAKALRQVEISPSTLSDTGKISNKVEKLLGIIAQQKPPFSAIIFVQERATVSVLAHLLSHHPLTKDR
FKIGTMVGTSLNGKRTDQIGELVDVNQQKDTLSSFKRGKIDILIATNVLEEGIDVPACNLVICFSK
PANLKSFVQRRGRARQQDSKLILLDASGDKATNWHELERKMREEYGKEMRELQHIYEIETADEQ
SEDDRVLRIESTGAQLDLDSALPHLYHFCSVLTTKDFVDLRPDFVYSSELGSEYVRAKVILPGSVS
KPLRVHESRGSWLSERSAAKDAAFEAYSALYRGGLVNDNLLPLMVHDKVIDELTSKPVDTRASL
LEVKERLNPWIDIARAWKEAEHHAGIVRTSVMIFNGMKLELCLPIDPPAIPPLKLYWDADTEFFV
DFTNDIEIGTSENMLAQALNDTNLLLSDRGRKVHIQSRRTVVQFILLQDSGSLSSDCFPVDPNGNI
KSTGFIREVGKLESPYIFEKWLPNAPEDVPYLAVVKVSRRADFLHKVQNEKPSSFTKQFSSVLPAS
TCVQDVMPAQLSRFGMMIPSITHHIEVQLVVDRLSRTILKDLEISDQSLIQTAITHASYSLDSNYQR
LEFLGDSILKLCTSVQLVAEHLDWHEGYLSAMKDRIVSNSRSSRAAAEVGLDEYIMTKKFTGAK
WRPMYVDDLVVTEQKTREMSSKILSDVVEALIGASLRPVEQILAYTFTKKSLLVEAMTHPSYTSG
TQSLERLEFLGDSILDNIIVTAMWSHSTPLSHFHMHLLRSALVNADFLAFLCMEMSIDQNVTNLT
EGKNHRIHETHSRRRVSLVSFLRHSSVRLSIYQKEALSRHAELRDQILEAIYTGDTFPWALLSRLD
ARKFFSDMIESLLGAVWIDSGSMEVCTQLIERMGVLRYMRRILKDGVRIMHPKEELGIVADSENV
RYVLRREKMGGDATEVNADADEEVRTEYRCTVFVGGEEIVEVRGGARKEEIQARAAEQAVRIL
KARGHEKRNGGAGEGKKRKSLDE*
SEQ ID NO: 5-Verticillium dahilae DCL (VAD_00471.1) genomic DNA sequence
(selected RNAi fragment marked by bolded text)
ATGACGACTGACGAGCTCTCTGTTGGTCTGGACGCCACCGGCATCTCAATCCTCGCAGATGG
ACCGGAAAACATATCGTCCAGCACATCAACATCTACGACTGGAAAGGAAGATGGATACCTC
TGTATCAACAGATTCACTCAGAATACCGCCACGACCCAGGACAACCAGAGCCGAGATTCTG
ACGACGATGAGGATGACTGCGGCAGCCACGATGAAGCTGACGAAGATTCAGACGAAAGAC
AGTACAGCATGACCCCAGAAAGGCCTCATAAAATTACCGAGAAGAAGCGCGCAGATCATGC
TGCCTTTCACGACTGGCTTCAGAGCAACTCCAGCGAGATTGCTCAGTCAACCCCTCAGCCGG
CTCAAAACCTCAACCACACCTCCACGGCCCTGATGGTACGCGAGAGTGAGAATCGTAAGAT
CATCGAAAATCCTCGGGAGTATCAGATTGAGCTCTTCGAGCGGGCGAAGCGAAAGAACATC
ATTGCCGTGTTACCCACTGGATCAGGAAAGACCTTAATCGCAGCCCTTCTTCTGCGACACAC
CCTCGAACAAGAAACCGCGGATCGACGCGCGGGCAAGCCCAAGAGAATCGCCTTTTTCCT
CGTGGAAAAGGTTGCTCTTGCCCTCCAACAGCACGCGGTTCTGGAGTGCAATCTGGAA
TTTCCCATTGACCGGGTATGCGGTGACATGGTACGGTCGGACTGGATCAAGGAGTCAT
GGATGAAAAGATGGGATGACAACATGGTCATGGTCTGCACCGCCGCCATCCTTCAGCA
ATGCCTTGCCAGATCATTCATCCGCATGGATCAGATCAACCTGCTTGTCTTCGATGAAG
CACATCACGCCAAGGGAAATCATCCGTACGCCCGGATCATCAAGGACTACTACATTACGG
AACCTGACAAAGAAAGGCGCCCCAAGATCTTCGGCATGACTGCCTCTCCGGTGGATGCCCTC
ACCGACGTCAAGATTGCTGCCGCTCAACTCGAAGGTTTGTTGCATAGTGAGATTGCGACAAT
CGAGGAGGACTCTGTATCATTCAAACAAATCCAGAAAGAGGTCGTCGAACAAGACTGCAAG
TACCCTGCCCTCGAACCACCCTTCACCACCAATCTTCATAAGAAGATCCAAGAACAGGTGCG
CTACAACAAGAACTTCGCAAAGGCGCTGAGCAATTCTTTAGAAATGTCGAGCTCCCTTGGCA
GCTGGTGTGTCGATCGCTTCTGGCAGATATTTCTGACCGAAGAAACCCTCGCGAGATTGGCA
GCGCAAACTGCACAAGACAACATTTTTGCCGATCGCGCCGAAAAGGAGCGCGTTGCCATTG
AGGAGGTCCGCAACATCATCAAGCAACATCAGTTCCTCCCAATCACCAAAACCCTGCAAGA
CTTGTCGTCCAAAGTGCTGTGCCTCCTCGGCCAACTGGAATTGCGCTTCAGTGCCCCTACCGA
TCACAAGTGCATCATCTTCGTGGAGAAACGAAACACAGCCATGATTCTGGCTCACCTCCTCT
CCTTGCCTGGTATTGGACCTCTATATCTGAAACCGGCTGCGCTTGTCGGGAACCCATCTGAC
AACAGCCCTCTTGCCATGTCGTACAAAGAGCAAGTGATGACAATAACAAAGTTCAGACGTG
GTGAATACAACTGTCTTCTCGCCACTTCTGTGGCCGAGGAGGGCATTGACATCGCAGACTGC
AACATTGTCATTCGATTCGATCTTTTCAACTCGGTGATTCAGTACATACAATCCAAAGGCCGC
GCTCGGCACTTGAACTCGGAGTATATTTGCATGGCCGAGCTAGGCAACGGCAAGCATACAA
GGGCGAAGATACAAGCAAATTATGACCTCTCCCTCATCCGCCAATTCTGCAGCACACTGCCA
GAAGACCGCAAGATCGTGGGCTGGGACCCCGAGGCAGCTCTTCACCATGGCGAGCGCGACC
ATAAGTTCCACATCGTTCCATCCACCGGGGCCAAACTCACCTGGAC
CGGCAGCCTCGTGGTTCTGTCAAATTTTGCCTCTTCTCTACAGGTGAACGACGAAACACTAA
GTCCTTCCTATATGGTCTCTCTCATCGGTAGCGAGTACATCTGCGAGGTCCAGCTTCCGAGCA
AGTCTCCCATTTTGAGCGTGTCAGGCACGCTCCAAAAGAACAAAGCAGAGGCCAGGTGCTC
CGCAGCGTTTGAGATGTGCATGAAGCTCATCAAAGGTGGGTTCATCAGCAGTCACCTTCAGC
CGACGTTTACCAGGAAGCTCCCGGCCATGCGAAACGCACGCCTAGCCATCAGCTCCAAGAA
GCGTGAACGGTACAATATGAGGGTCAAGCCAGAGGTATGGTCACGGCGTGGACCGGCATCC
TCTCTGTTCCTCACAGTCCTGAAGCTTCGTACACCTGGTGCATTGAACAGACCATCACAGCC
ACTCGCCCTCCTCACACGAGAGGCACTGCCAGAGCTTCCAGGAGTTCCGCTATTTTTCGGTA
ACTGTGGTCGGTCCATAGCGGAGGTAGTATCTGTGGCGAAACCCATGCACTTGGATGAAGTA
CGTCTAGACAGCCTCAGAGTATTCACCCTGCGCATTTTCAAAGATGTCTTCAGCAAGGTATA
CGATTCTCAAGTCGCAGACCTTCCATACTTCCTGGCACCTGCTGCTCATGACCACAGTCATGA
GTTCTCACCGAATGAAGACCCAGGGTCACTGATCGACTGGAGCCATCTGCTGTCGACCAAAG
AGGTTGAGTACTTGCCTTGGGATGAAGATCACAGTCCCAGCTTCTATCAAAGCAAGTTTGTG
ATTGATCCATACACGGGATCGCGCAAGCTGTTTCTCAGAGGTATTCGGACAGATCTCAAGCC
GACCGACTTGGTTCCAGATGGAGTTCCCGAACCCACATTCAGGCTCTGGAAGGACGTTGAGC
ATACCATAAAGGAATACAGCATCAGCCTCTGGGCAAAGAGTCGAGCCCGGAGAGCTGGCGA
ATGGTTGGACACTCAACCCGTGGTAGAAGCCGAGTTGGTCTCGCTGCGCCGGAATCTTCTCG
ACGAATTTGCCGATTCCAAGCATGAAGGGTCTAGGGTCTGTTATGTGATTCTCCAGCCGCTA
CAGATCTCAACACTCCCTGTCGAGGTCGTCGCTATGGCCTACAACTTTCCCGCCATCATCCAT
CGGATTGAATCGAATATGATCGCCCTTGACGCCTGCCGTATGTTGAACCTTCGAGTTCGTCCC
GACCTGGCTCTCGAGGCGATGACCAAAGATTCAAGCAACAGTGAAGAGCACGATCAGGAAA
AGATTGATTTCCAGGCCGGCATGGGCAATAATTATGAGCGACTCGAGTTTCTCGGAGACTGC
TTTCTCAAAATGGCAACCACCATCGCACTTTTTACTCGGATCCCTGACAGCAACGAGTTTGA
GTGTCACGTCGAGCGAATGCTTCTTATTTGCAACCAGAATCTGTTCAATGTCGCATTAAAGA
AGAACTTGCAAGAGTACATTCGATCAAAGCAATTCGATCGACGCAGTTGGTACCCCCAGGGT
CTGAAGCAGAAGGCGGGCAAAGCCCAAGGAGCACAAAACTCACACTCATTGGCCGACAAGT
CTATTGCTGATGTATGCGAGGCCATCATTGGCGCCTCATATTTGTCGTACACTGACGAGGGC
AACTTTGACATGGCCGTACGCGCTGTGACGGCCGTCGTGAGGAACAAAAATCACGACATGA
AATCATACGAGGACTATTACAAAGCATTTAAGATGCCGATCTGGCAAGCGGCGGAGCCAAG
TGCTGTGCAGATGGAAGCGTCTTTACAGATTAAAGAGCAGATGGGATATGAGTTCAAGTCTC
CTGCCCTGCTGCGGAGTGCCTTCAAGCACCCGTCCTACCCCCGTCAGTTTGAGAGCGTGCCC
AATTATCAGCGCCTCGAGTTCCTCGGTGACGCGCTTCTAGACATGGTCTGCGTAGACTTTCTC
TTCAGGAAGTTTCCCGACG
CCGATCCTCAATGGCTCACTGAACACAAGATGGCCATGGTTTCGAACCACTTCCTCGGAAGT
CTGAGTGTAGAGTTGGGCTTCTACCGGCGTGTCCTTCACTTTAACAGCATCATGGCCAATCA
AATCAAGGACTACGTCGACGCACTTACTCATGCACGCCAAGAAGCCGAAGCGGTGGCCCAG
ATCTCTGGCACAGTCTCGCGAGATTACTGGCTCAACGTGAAGCACCCCCCCAAATTCCTCTC
AGACGTGGTCGAGGCATACATCGGTGCTATTTTCGTTGATTCAGGATACGATTATGGCCAGG
TACAGGCGTTCTTCGAGAAGCATATCCGGCCTTTCTTCGCAGACATGGCGCTATATGATTCCT
TTGCCAGCAGCCACCCTGTCACAACGCTGGCGCGTATGATGCAGCAGGACTTTGGCTGCCAG
GACTGGCGGCTTCTTGTAAGTGAACTGCCGCCGAGCTGCGAAGACGGCGGGGCAGCTGCGA
TCACTGAGACGGAAGTGATTTGTGGGTTCATGGTCCACGGAAGAATCCTGCTACATGCCAAG
TCGTCGAGTGGACGGTACGCCAAAGTGGGTGCTGCAAAGAGAGCGGTCGAGAAGCTCATGG
GTCTCGGCAACGACAAAGAGGTCTTTCGGACGGACTTCGGCTGTGACTGTGACTGTGAAGGT
CAAGCAATCTAG
SEQ ID NO: 6-Verticillium dahilae DCL (VAD_00471.1) protein sequence
MTTDELSVGLDATGISILADGPENISSSTSTSTTGKEDGYLCINRFTQNTATTQDNQSRDSDDDED
DCGSHDEADEDSDERQYSMTPERPHKITEKKRADHAAFHDWLQSNSSEIAQSTPQPAQNLNHTS
TALMVRESENRKIIENPREYQIELFERAKRKNIIAVLPTGSGKTLIAALLLRHTLEQETADRRAGKP
KRIAFFLVEKVALALQQHAVLECNLEFPIDRVCGDMVRSDWIKESWMKRWDDNMVMVCTAAI
LQQCLARSFIRMDQINLLVFDEAHHAKGNHPYARIIKDYYITEPDKERRPKIFGMTASPVDALTD
VKIAAAQLEGLLHSEIATIEEDSVSFKQIQKEVVEQDCKYPALEPPFTTNLHKKIQEQVRYNKNFA
KALSNSLEMSSSLGSWCVDRFWQIFLTEETLARLAAQTAQDNIFADRAEKERVAIEEVRNIIKQH
QFLPITKTLQDLSSKVLCLLGQLELRFSAPTDHKCIIFVEKRNTAMILAHLLSLPGIGPLYLKPAAL
VGNPSDNSPLAMSYKEQVMTITKFRRGEYNCLLATSVAEEGIDIADCNIVIRFDLFNSVIQYIQSK
GRARHLNSEYICMAELGNGKHTRAKIQANYDLSLIRQFCSTLPEDRKIVGWDPEAALHHGERDH
KFHIVPSTGAKLTWTGSLVVLSNFASSLQVNDETLSPSYMVSLIGSEYICEVQLPSKSPILSVSGTL
QKNKAEARCSAAFEMCMKLIKGGFISSHLQPTFTRKLPAMRNARLAISSKKRERYNMRVKPEVW
SRRGPASSLFLTVLKLRTPGALNRPSQPLALLTREALPELPGVPLFFGNCGRSIAEVVSVAKPMHL
DEVRLDSLRVFTLRIFKDVFSKVYDSQVADLPYFLAPAAHDHSHEFSPNEDPGSLIDWSHLLSTK
EVEYLPWDEDHSPSFYQSKFVIDPYTGSRKLFLRGIRTDLKPTDLVPDGVPEPTFRLWKDVEHTIK
EYSISLWAKSRARRAGEWLDTQPVVEAELVSLRRNLLDEFADSKHEGSRVCYVILQPLQISTLPV
EVVAMAYNFPAIIHRIESNMIALDACRMLNLRVRPDLALEAMTKDSSNSEEHDQEKIDFQAGMG
NNYERLEFLGDCFLKMATTIALFTRIPDSNEFECHVERMLLICNQNLFNVALKKNLQEYIRSKQF
DRRSWYPQGLKQKAGKAQGAQNSHSLADKSIADVCEAIIGASYLSYTDEGNFDMAVRAVTAVV
RNKNHDMKSYEDYYKAFKMPIWQAAEPSAVQMEASLQIKEQMGYEFKSPALLRSAFKHPSYPR
QFESVPNYQRLEFLGDALLDMVCVDFLFRKFPDADPQWLTEHKMAMVSNHFLGSLSVELGFYR
RVLHFNSIMANQIKDYVDALTHARQEAEAVAQISGTVSRDYWLNVKHPPKFLSDVVEAYIGAIF
VDSGYDYGQVQAFFEKHIRPFFADMALYDSFASSHPVTTLARMMQQDFGCQDWRLLVSELPPS
CEDGGAAAITETEVICGFMVHGRILLHAKSSSGRYAKVGAAKRAVEKLMGLGNDKEVFRTDFG
CDCDCEGQAI*
SEQ ID NO: 7-Verticillium dahilae DCL (VAD_06945.1) genomic DNA sequence
(selected RNAi fragment marked by bolded text)
ATCACTCTACGGGTAAAAGCGCTGAGAGAATGATCATGATGAATTTCTATCATCCACGCAAA
CAATCGGCACTATCTGTTCCCCACGTCCTGGGACTGACCGCAAGCCCCATAATGCGATCTAG
GCTCGAAGGCCTTGAGGCACTGGAACAGACACTGGACTCGGTTTGCGTTACGCCCAGATTGC
ACCGAGATGACTTAATGACCCATGTCAAAAGGCCCACCGTCTGTTATGTCCATTACGAAACG
ACAGATGCTAAGGATGAGCCCAAGCCGGTCAGCATTTCAAGTCTTCGCGAAGCATGCAGAA
ATATGGACATCAGGCAAGATCCATACGTTATCTGTCTAAGAGACAAAGGCACTGATCGAGC
ACGACGTGAGCTCATCAAGGTCCTTACAAGCCATAAAACAGATTCGCAACAGCAAATGAAG
TCTTTCTTCAATCAAAGCTTGCGAGTCCTGCGAGATCTCGGGCCCTGGGCGGCCGAGTACTA
CATTTGGAAGGTTGTTACAGATTTTCTGGCAATCATTGAAGCAAGAGATCACCGCATGAATC
AACGGAATACCGAAGAAAAGCAGTATCTGGCCAACATCCTTCGACAAATCAGTATCAGCGA
GCCGCCAGTCAGCATGTTGAGTGCTCATAACACGTCGAACAAAGTAATGGTGCTCATGGAAT
ACTTGTCATCTAAAGCTACCGATGGTACTGTCGGGATCATATTTGTCAAAGAGCGATCAACT
GCGGCGATGCTTGCACACGTGATTGAGTCGCATCCACTGACACAGAATAGGCACTCGAGCGT
TGGGGTTGTTGTTGGTGCTTCCACTCATCTGGTAAGGAAGAAAGACATGTGGGATCTGTCTC
GAGCAGCCCACGAGACAGAGCCCCTTCTTCAGTTCAGATCTGGCCACCTCAATTTGCTCATC
GCCACGAGTGTGCTTGAAGAGGGCATCGACGTTCCTGCCTGCAACCTCGTGATCTGTTTTGA
TGAGCCCGAGAATCTCAAAGCCTTTGTCCAGCGGCGCGGCCGAGCCCGGAAGAAGGATTCT
AGCCTCGTGGTTCTTCTCCCCGGGACAGACCACGTGCCTCAGGACTGGGAAAGCATGGAAGC
GACAATGAGGACACACTACGAGAGAGAACAGCGCGAAATACAAATCATGGAGCAGATCGA
AGCATCCGAGTCTGCAAAGTACGAAGAGTACGTTGTCGAGAGTACTAATGCCAGACTCGAC
TTCGAGAACGCCAAAGCGCATCTCAGCAACTTTTGTGGGCAGCTCTCTCCCGGGGAGTTTAT
AGACAAGAGGCCCGAATACATACCCCGTGTGGTAGACAACGGAGTACCTCCATCTCTGAGG
GTCACGGTACTGTTGCCAAGCTATGTTCCAGCTGCCGTCCGCCATGCTGAGAGTCGTCGAAG
CTGGAAGTCGGAGCATCAGGCCTCAAAGGATGCCGCTTTTCAGGCATACGTGGCTCTTTACA
AAGCGGGACTGGTCAATGAACACATGCTTCCACTCACGGTAAAAGATATCGTACCCGCAAA
CGAACCTCGAGTAGCAACCTTGCAGGTCAATGGCCTCTTGAATGTCTGGCTTGGTATTGCCC
AGGCCTGGATCACGAGCACTGAAACCTGGTTAACTCCAGTGCACCTCCGAGACGCGACGGG
ATTGACGCGAGGAACGTATATCATGAGAATGCCGGTAGCATTGCCGGCACTGCCTTCCACGC
CGGTGTACTTCGATCGCGAAGGACCATGGCTTCTGGATTTTGGCCCACAAGAACGAAAGGA
GAATCTTGAAATGCCTGATCATACTTCAGTGCTGCTTGCACTCCACTTTGGCCATCACTGGTC
TATTGCTCATGGTCAGCAGCAGGTTATCAGCTTCGCTTCACAAGATGGCGA
ACTGAATATCAGGCAATTAAGTGCACGGGGTTTCACAACCGCAGATGCCGACCGAGAGGAA
ATGCTGTACCTGGTACGGGACGAGTCAGGATGCCCGTATGTGTACGACCACTTTCTAAATGG
CAAGCCGTCACTTGAACTTGTTCAACGACCTTTCCGGCGCATCGGGGACTCTCCAGGCTTTC
AAGACGCACCCAGTAACATCCCCTACTTGGCTCTCAGAAAGTGGCCGCGGTACCTGGCCCTC
TTGCACCAACAGAAGGTCAACGATCTACTGCCACAGGCGACAAACAAGAAGCCATATGCTA
GGGTTTATCCGGCACCGTGGGCGAAAGTCGACACGATTCCATTAGATCATGCTTACTTTGGG
GCGTTGATCCCTTTCATTTCACACATTGTCGAGGTTCGACTGGTTGCAGAACAGCTTTCCTCG
AGCCTACTTCGTGACCTCAATTTCTCAGATCCCTCTCTTGTCCTGGCGGCCATTAGCACTAAG
GGTTCCTTGGAAGCCACAAACTACGAGCGCCTTGAGCTTTTGGGTGACTCTATCCTCAAGCT
TTGCACCACGGCCAATGCCGCCGCTCTGCATGGCTTAGTGTCGAACTCGAGATTGTGTAGGG
CTGCACTGGATGCTGGCCTTGACAAATTTGTTCTAACTGAAAACTTCACTTGTCGCACGTGGC
GCCCTATCTACGTCAACGACATGATGGAAAAGGGTGCTCGCGACTCAGGACCCCGTATCATG
TCGACGAAGACGCTCGCCGATATTGTGGAAGCACTCATAGGGGCCGCATACATTGACGGTG
GCCTCCCAAAGGCACTTGGGTGCATTTCGATCTTCCTGAGGGAGCTCGATTGGAAACCGTTG
CCAGCTTGCCAGGAGATCCTTTACAGTTTGGCGTCCCCTGATGTGCCTTTGCCGCCAATGCTT
GTTCCGCTGGAGGACCTGATCGGCTACACGATGCATCTCCTCAAGACTGCTTCGGTCAACGG
CGATCTTCTAGGCTTCCTTGCACTCGAGTGCCATGCCGAGGAAGACGAGGTGATCATTGATA
TCGATTTTTCTCCTTCCGATACGGACTTCAATCCTCAAAATTCCGCCGGGGTGGAACAGAAG
CTCAAACAGACACGCCGGAAAATCCCCCTTTGGAAGTTTATGCGCCACTCCTCAATAGA
GGTTGTGCAGCAGCAGACCAAAGCTGCCAGCGTTCATGCCGATCTCCGAGGACAGATC
ATGCACGCTCTGGAACATGGGTCAAGCTACCCCTGGTCTCTTCTCGCCCGTTTACATCC
CGCAAAGTTCTTCTCCGACATGGTCGAAGCTGTACTGGGTGCCGTCTGGGTCGATTCG
GGCGACATGGGCGCGTGCATTCGTGTGGCGGAACGACTGGGCATTCTGCCTGTGCTCT
CCCGACTGGCAAAGGAGGACGTTCATGTGCTGCATCCGAAGCAAGAGCTGGGAGAGATC
GCTGGTCCCCGGACAGTCAAATATCTCCTCACTTTGCCCGAGGACGCAGCCGGCCTGCAAAG
TGCAACAAGAAAATATGCCTGCAAGGTCATGGTCGGGGATCGCTGTGTTGCAGAGGTGGAT
GACGGGGTCGCTCGAGATGAGGTTGAGACAAAGGCTGCAGAGGTTGCGGTACAGACCTTGA
AGAATGAACAGGCTGACGCGAAACAAGTAGCAGAACACTAA
SEQ ID NO: 8-Verticillium dahilae DCL (VAD_06945.1) protein sequence
MIMMNFYHPRKQSALSVPHVLGLTASPIMRSRLEGLEALEQTLDSVCVTPRLHRDDLMTHVKRP
TVCYVHYETTDAKDEPKPVSISSLREACRNMDIRQDPYVICLRDKGTDRARRELIKVLTSHKTDS
QQQMKSFFNQSLRVLRDLGPWAAEYYIWKVVTDFLAIIEARDHRMNQRNTEEKQYLANILRQIS
ISEPPVSMLSAHNTSNKVMVLMEYLSSKATDGTVGIIFVKERSTAAMLAHVIESHPLTQNRHSSV
GVVVGASTHLVRKKDMWDLSRAAHETEPLLQFRSGHLNLLIATSVLEEGIDVPACNLVICFDEPE
NLKAFVQRRGRARKKDSSLVVLLPGTDHVPQDWESMEATMRTHYEREQREIQIMEQIEASESAK
YEEYVVESTNARLDFENAKAHLSNFCGQLSPGEFIDKRPEYIPRVVDNGVPPSLRVTVLLPSYVP
AAVRHAESRRSWKSEHQASKDAAFQAYVALYKAGLVNEHMLPLTVKDIVPANEPRVATLQVN
GLLNVWLGIAQAWITSTETWLTPVHLRDATGLTRGTYIMRMPVALPALPSTPVYFDREGPWLLD
FGPQERKENLEMPDHTSVLLALHFGHHWSIAHGQQQVISFASQDGELNIRQLSARGFTTADADR
EEMLYLVRDESGCPYVYDHFLNGKPSLELVQRPFRRIGDSPGFQDAPSNIPYLALRKWPRYLALL
HQQKVNDLLPQATNKKPYARVYPAPWAKVDTIPLDHAYFGALIPFISHIVEVRLVAEQLSSSLLR
DLNFSDPSLVLAAISTKGSLEATNYERLELLGDSILKLCTTANAAALHGLVSNSRLCRAALDAGL
DKFVLTENFTCRTWRPIYVNDMMEKGARDSGPRIMSTKTLADIVEALIGAAYIDGGLPKALGCIS
IFLRELDWKPLPACQEILYSLASPDVPLPPMLVPLEDLIGYTMHLLKTASVNGDLLGFLALECHAE
EDEVIIDIDFSPSDTDFNPQNSAGVEQKLKQTRRKIPLWKFMRHSSIEVVQQQTKAASVHADLRG
QIMHALEHGSSYPWSLLARLHPAKFFSDMVEAVLGAVWVDSGDMGACIRVAERLGILPVLSRL
AKEDVHVLHPKQELGEIAGPRTVKYLLTLPEDAAGLQSATRKYACKVMVGDRCVAEVDDGVA
RDEVETKAAEVAVQTLKNEQADAKQVAEH*
SEQ ID NO: 9-RNAi fragment from B. cinerea DCL1 cDNA
TGCGGAAGAACTTGAAGGTTTGCTACACAGTCAAATATGTACTGCAGAAGATCCCAGCTTGC
TGCAGTACTCAATCAAAGGTAAACCTGAGACTCTTGCCTACTATGATCCCTTGGGCCCGAAA
TTCAATACTCCTCTTTATCTTCAAATGCTCCCGCTTCTAAAAGACAATCCTATCTTTCGGAAG
CCATTTGTATTTGGGACAGAAGCCAGTAGAACTCTAGGATCTTGGTGTGTTGACCAGATCTG
GACTTTCTGTC
SEQ ID NO: 10-RNAi fragment from B. cinerea DCL2 cDNA
TCTTTAAGTGATATCGAGGAGACTTTGGATGCCATTTGCTGCACGCCAAAAATACATCGAGC
AGATCTTCGCCTTCGAGTAAAGCTACCACTTCTATCTATTATCTACTATACCCCAGAGTCAAA
TATCATCGTGACGAAAACTGTGGCGAGCCTGAGAAAGATTGTGCAAAGTCTCAACATTTTCG
AAGACCCCTACGTTTTGACACTAAAAAGGAGTGATAGCGAAAAAAGTCAACGTGAGCTGGC
GAAAGTACTCAAGAGTTTTAAGACATATAGTCAAACCCAATTAAAGTC
SEQ ID NO: 11-RNAi fragment from V. dahliae DCL (VDAG_00471) cDNA
GGCAAGCCCAAGAGAATCGCCTTTTTCCTCGTGGAAAAGGTTGCTCTTGCCCTCCAACAGCA
CGCGGTTCTGGAGTGCAATCTGGAATTTCCCATTGACCGGGTATGCGGTGACATGGTACGGT
CGGACTGGATCAAGGAGTCATGGATGAAAAGATGGGATGACAACATGGTCATGGTCTGCAC
CGCCGCCATCCTTCAGCAATGCCTTGCCAGATCATTCATCCGCATGGATCAGATCAACCTGC
TTGTCTTCGATGAAGCACATCACGCCAAGGGAAATCATCCGTACGC
SEQ ID NO: 12-RNAi fragment from V. dahliae DCL (VDAG 06945.1) cDNA
ACAGACACGCCGGAAAATCCCCCTTTGGAAGTTTATGCGCCACTCCTCAATAGAGGTTGTGC
AGCAGCAGACCAAAGCTGCCAGCGTTCATGCCGATCTCCGAGGACAGATCATGCACGCTCT
GGAACATGGGTCAAGCTACCCCTGGTCTCTTCTCGCCCGTTTACATCCCGCAAAGTTCTTCTC
CGACATGGTCGAAGCTGTACTGGGTGCCGTCTGGGTCGATTCGGGCGACATGGGCGCGTGCA
TTCGTGTGGCGGAACGACTGGGCATTCTGCCTGTGCTCTCCCGACTGGCAAAGGAGGACGTT
CATGTGCTG
SEQ ID NO: 13-LTR for siR3
>B. cinerea (B05.10) Botrytis cinerea supercontig 1.56 [DNA] 218751-
219771-
CTCCTGGATCAGGCAGATGAATTAGGGAACTGATTTCGACCTTCCAGAGTTCTCTTTGCG
TGATGGGTCACTTGGGTTTGGTTGTCGGTATGCTGTGGGTTCGGAGGAGTTGTCCTTTCT
GGTTTCTTTGTTGGATAGTCCTTTTTGGGTAGCTTGGTGTGATGCATGCGTTCTGGGTGT
GGGTCTCGTGAGGTCTTTTTGTATCAAGTATTTTTAAGCTTTTTTCTTGTTCTCTTCTTT
TTCTGTATTGGTAATGCTTCTTCTTTATGATATTCTCCCATCGCTGCTTTCGCATTTTCT
AGGTTGTAGGGTGCTTCCCACGTGTCTTCCGCCGGTTGGTAGCCCTTCCATCGCACCAGG
TATTGCACACCTCTGCCTCTTTTTCTATGTGCTAAAATCCTTTCCACCTCATGCTCTATG
TGATCGTCAATTTCTTCTGGCGGCGGCGGGTCGGCGGTACCCTGTCGTTCGTGCCATGGT
TCGAGTAAAGAGACGTGGAATACATTGTGGATCTTGTAGGTGGGCGGTAATCTAAGTTCG
TATGCTTGCCCGCTGGTTTTTATACCCGTCACGACAAAGGGGCCTATAAATCGATCGGAG
AATTTTTTCTTAGGTCGCAGTTGTTTAATGTTCTTTGTGCTTAGCATCACCTTGTCCCCG
ATGCTATATCGCTGTGGTGACCCCTTCGTAGTGTTCTTGTTTTTCTGATTGGTTGCCGAT
TTCCAGAATTCTTTCAGCTTTTCTCTTTCTTTCTCTAAAGCGTCGATGCGCTCGCGTGCT
GCCGGCGCCCTTCCCTCTAAATCGGCGTCCTCGCCGATATAATGGAATGTGGGTTGGAAC
CCATACATAGCCTGGAATGGGCTTGTATTGGTTGTACTGTGCCATGTCGCGTTATATGTG
AATTCAGCAAGGGGCAATAGCGATGCCCAGTCGTCTTGCCTATAGTTGGTGTAGCAACTT
ATATAGTGAATCAAATTTTGGTTTTGTCGTTCGGTTTGACCGTCGGTCTGCGGGTGGAAC
G
SEQ ID NO: 14-LTR for siR5
>BC1G_08572.1 retrotransposable element Tf2 1 protein type 1
(Transcript: BCIT_08572)
ATGGCATCCAGAGCTACCGCCACAGGTCAATCTGCCGGAGACACCAACGACATCGAGATGA
CCGACGCCCCAAAGGAGATCACTATCAACGAAACCCTTAAGATCGCCTTACCAGACAAGTA
CCAAGGTAGTCGACAAGAGCTCGATACTTTCCTCTTACAACTTGAGATCTACTTTCGATTCAA
TGAGGACAAGTTCACTACCAAGGAATCCAAGAGTATATGGGCCGCATCATACCTTCGAGGT
GAAGCAACCAAATGGATCCAACCATATTTGCGCGACTATTTCGAGCATGACGATAAGGATC
GTATGCAACCCACCCGAACAATCTTCAATAGTTTTGAAGGATTTAAGACAGAGATTCGTAGA
ATCTTCGGAAATTCCAACGAGTTAGAGGTAGCGGAAGATAAGATCTTCAACCTCAAGCAGA
CAGGATCAGCATTGAAATATGCTACGGAATTTCGAAGATATGCTGGAACAACCAAGTGGGA
CGAAATCGCTATCATGAGTCACTACCGCAAGGGACTCAAACCAGAAGTCAGACTGGAATTA
GAAAGATCTGCCGAGAGTACAGATCTGAACGATCTAATTCAGGACTCCATCGAATCAGATG
ATCGTCTCTACAGATATCGACAAAGCCAGAGATCATACAAACCCCAAGGAAATCAGAAGCA
AGGGCGTTACCGCAAAAATGAGGGTAGACCACGTTACAATCCACAGAGATACGGAGACCCC
ATGGAACTAGACGCCACGCACTACACAAACGGGAACGATGACTCGGAAAAGAGACGAAGA
CGAGAAAACAACTTATGCTTTGAATGTGGAAAAGCAGGGCACCGAGCAGCAGACTGCCGAA
GCAAGAAGACAGGAGGAAAAAGGGGCAACTTCAAACCTAAGTTCGGCAAAGGCCAACTTA
ACGCTACCTTTACAATCCCAGAAAATCCAACTAAATCCGAAAATACTGAGACTTTCACCGTT
GAGGAATTCCAGCAATTACTAAAGGAATTACCACGAAATCAAGAGGGCATGAATGCAATAG
ACTTATGGGAGCAAGAGTATTACAGAACCCCAACACCCTCTGTGACAGAAGAAAGTCATCA
GGACGAGGCAGAAGCAGACCACGCCACGATGAGCTGGACAGCTTGCTATGATGAATTCTGC
GGAATCCATCGATCAGATAAAGAAGCAACCGGATGGTCCCCTAAGAAGAGAAAGACGAAG
AACCATCAGAATAATGTAACATGCGAGGATTTAACTCCCAATATAACTTCGCAAGAAGTTCG
CAAAGTTACCCAGCAGTTAAATGCTACGGGACAGGCAGGACAGATATACTGCAAGGTTCAG
ATAAATGGACACATACAATCAGCCATGATAGATTCAGGGGCTACAGGAAATTTTATTGCACC
GGAAGCTGAGACAATCCCAATACGAATGGGCATAACCCAACATACAGAGGTTATACAGCTT
GACGTTGTGCCATTGGGCCAACAACAGATCATCTTAGGAATGCCATGGTTAAAGGCACATAA
TCCGAAAATAGATTGGGCACAAGGAATTGTGACATTTGATCAGTGCAAAAGCGGTCACAGG
GACACGCTAGAGGCGTTCGCGAGACGTAACACGCGCCAAGGAGAGTTGAACGCGAACAACA
CCGGCGACGTAGGACACCCAGTCCAGGGTCCTCCATTAAGAGCGAAGGCCAGTACACCTCC
TCTACAAATGCAGAAGCCAACGACACGGCACGAAATCGCAATCGAGGCAAAAGAAAAGCCT
ACGATACCAGAACAGTACAAGAATTATGAACATGTTTTCAAAGAACCAGGGATCCATGAGG
CTTTACCGGAACACAAGCCATGGGATCATGAGATAATATTGGAGGAAGGCAAGATGCCTGT
GCACACCCCAATTTATTCAATGTCAGCCGATGAGTTAAAGAGGCTCAGAGAGTACATCGACG
ACAATTTAGCCAAGGGATGGATCAGGGAATCCGCGTCCCAAGTGGCCAGTCCAACTATGTG
GAAGGATCGATATCCACTTCCATTAGCTACGGAATTAAGAGATCGATTAGGCGGAGCTACG
ATATTTACCAAGATGGACCTACGTAATGGTTACCACTTGATCAGAATGAAGGAAGGCGAAG
AATGGAAAACCGCTTCAAAACAAGATACGGGCTATACGACTACTTTCATGAGGCTTATGAAC
AATGTGTTGTCACAATATTTGGATACTTGCTGTATATGCTACTTGGACGACATCCTAGTATAT
TCAAACAACAAGGTTCAACACATTAAGGACGTTAG
CAACATCCTCGAAAGCCTATCCAAGGCAGACTTGCTGTGCAAACCAAGCAAATGCGAATTCC
ATGTCACAGAGACAGAATTCTTGGGATTCACCGTATCAAGCCAAGGGCTCAAGATGAGCAA
AGGCAAGGTTAAGGCAGTGCTCGAATGGAAGCAGCCGACCACAATCAAGGAAGTACAATCC
TTTCTAGGGTTCGTCAACTTCTACAGAAGATTCATCAAGGGTTATTCAGGGATTACTACACCC
TTGACCACGTTAACCAGAAAAGATCAAGGAAGCTTCGAATGGACTGCCAAAGCACAGGAGT
CATTCGATACGCTCAAACAAGCAGTGGCAGAAGAGCCAATACTGTTGACTTTTGACCCAGAG
AAAGAAATCATAGTGGAGACGGACTCCTCGGATTTCGCTATAGGAGCAGTTCTGAGCCAAC
CGGGCCAGAATGGAAAATACCAGCCAATCGCATTCTACTCCCGAAAACTATCACCAGCCGA
ATTGAATTACGAGATATATGACAAAGAATTACTGGCGATAGTCGATGCATTTAGAGAATGGC
GAGTGTATTTGGAAGGATCGAAATACACGGTACAGGTGTATACAGATCATAAGAACTTGGTT
TACTTCACCACAACGAAGCAGTTAAACAGACGACAGGTCAGATGGTCGGAGACCATGGCCA
ACTACAATTTCAGAATTTCATATGTCAAAGGATCAGAAAACGCTAGAGCCGACGCTCTTAGC
CGAAAACCAGAATATCAAGAAAACAAAACGTACGAGTCATACGCTATATTCAAGAAAGACG
GCGAATCACTGGTTTACAATGCACCACAGCTTGCAGCAACACACCTGTTGGAAGACAACCAC
CTCAGGAAACAGATCCAATCACACTACGACAAGGATGCTACTGCCACACGCATACGCAAGA
CAATAGAACCAGGATTCACTATAGAAAATGATACCATATACTTTCATGGAAAAGTATACAT
TCCGAGTCAAATGACCAAGGAATTTGTGACGGAACAACACGGGTTGCCGGCACATGGACAC
CAAGGAATTGCAAGGACATTTGCAAGAATACGGGAAATCAGTTACTTCCCACGAATGAGAA
CGATAGTTGAAGAAGTTGTTGGAAATTGTGACACCTGCATACGAAACAAGTCATCACGACAT
GCTCCGTATGGTCAGCTCCAGACCCCAGACATGCCTTCTCAGCCATGGAAGTCCATCACATG
GGACTTTGTGGTCAAACTACCACTCTCAAAAGATCCTACTACAGGAATTGAGTACGACGCGA
TACTCAATATAGTAGACAGGCTAACGAAATTTGCATATATGATACCATTCAAGGAAACATGG
GATGCTGAGCAACTAGCATATGTGTTCCTAAGGATCATAGTAAGCATACACGGAGTACCAG
ATGAGATAATCTCGGATCGAGACAAGCTCTTTACCTCGAAATTCTGGACTACCTTATTAGCA
CTTATGGGTATCAAGAGAAAGCTATCGACATCTTTCCACCCACAAACAGATGGTCAAACAGA
GAGGACCAATCAGACAATGGAAGCATATCTTAGATGCTATCGTATAAAATCCCGATACCAC
AAGAAGTTAATGCCGAATCAGCGATAG
SEQ ID NO: 15-LTR for siR5
>BC1G_15284.1-enzymatic polyprotein
ATGGCATCCAGAGATATCGCCACAGGTCAATCTGCCGGAGACACCAACGACATCGAGATGA
CCGATGCCCCAAAAGAGATCACTATCAACGAAACACTTAAGATCGCCTTACCAGACAAGTA
CCAAGGTAGTCGACAAGAACTCGATACTTTCCTCTTACAACTTGAGATCTACTTCCGATTCA
ATGAGGACAAGTTCACTACCAAGGAATCCAAGAGTATATGGGCCGCATCATACCTTCGAGG
TGAAGCAACCAAATGGATCCAACCATATTTGCGCGACTATTTCGAGCATGACGATAAGGATC
GCATGCAACCCACCCGAACAATCTTCAATAGTTTTGAAGGATTTAAGACAGAGATTCGTAGA
ATCTTCGGAAATTCCAACGAGTTAGAGGTAGCGGAAGATAAGATCTTCAACCTCAAGCAGA
CAGGATCAGCATTGAAATATGCTACGGAATTTCGAAGATATGCTGGAACAACCAAGTGGGA
CGAAATCGCTATCATGAGTCACTACCGTAAGGGACTCAAACCAGAAGTCAGACTGGAATTA
GAAAGATCTGCCGAGAGTACAGATCTGAACGATCTAATTCAGGACTCCATCGAATCAGATG
ATCGTCTCTACAGATATCGACAAAGCCAGAGATCATACAAACCCCAAGGAAATCAGAAGCA
AGGGCGTTACCGCAAGAATGAGGGTAGACCACGTTACAACCCACAGAGATACGGAGACCCC
ATGGAACTAGACGCTACGCACTACACAAACGGGAACGATGACTCAGAAAAGAGACGAAGA
CGAGAAAACAACTTATGCTTTGAATGTGGAAAAGCAGGGCACCGAGCAGCAGAATGCCGAA
GCAAGAAGACAGGAGGAAAAAGGGGCAACTTCAAACCTAAGTTCGGCAAGGGCCAACTTA
ATGCCACCTTTGCAATCTCAGAAAACTCAACTAAACCCGAAAATACTGAGACTTTCACCGTT
GAGGAATTCCAGCAATTATTAGAGGAATTACCACGAAACCAAGAGGGCATGAATGCAATAG
ACTTATGGGAACAAGAGTATTACAGAACTCCAACACCCTCTGTGACAGAAGAAAGTCACCA
GGACGAGGCAGAAGCGGACCACGCCACGATAAGCTGGACAGCTTGCTATGACGAATTCTGC
GGAATCCATCGATCAGATAAAGAAGCAACCGGATGGTTCCCTAAGAAGAGAAAGACGAAG
AACCATCAGAATAATGTAACATGCGAGGATTTAACTCCCAATATAACTTCGCAAGAAGTTCG
CAAAGTTACCCAGCAGTTAAATGCTACGGGACAGGCAGGACAGATATACTGCAAGGTTCAG
ATAAATGGACACATACAATCAGCCATGATAGATTCAGGGGCTACAGGAAATTTTATTGCACC
AGAAGCGGCAAAGTACTTGGAAATACCACTTCAGAGGAAACAATACCCCTATCGATTGCAG
TTAGTTGACGGACAGCTAGCAGGGTCTGACGGAAAGATTTCGCAGGAGACAATCCCAGTAC
GAATGAGCATAACCCAACATACAGAGGTTATACAGCTTGATGTTGTGCCATTGGGCCAACAA
CAGATCATCTTAGGAATGCCATGGTTAAAGGCACATAATCCGAAAATAGATTGGGCACAAG
GAGTTGTGACATTTGATCAGTGCAAAAGCGGTCACAGGGACACGATAGAGGCGTCCGCGAG
ACGTAACACGCGCCAAGGAGAGTTGAACGCGAACAACACCGGCGACGTAGGACACCCAGTC
CAGGGTCCTCCATTAAGAGCGAAGGCCAGTACACCTCCTCTACAAATGCAGAAGCCAACGA
CACGGCACGAAATCGCAATCGAGGCAAAAGAAAGGCCTACGATACCAGAACAGTACAAGA
AATATGAACATGTTTTCAAAGAACCAGGGATCCATGAGGCTTTACCGGAACACAAGCCATG
GGATCATGAGATAATATTGGAGGAAGGCAAGATGCCTGTGCACACCCCAATTTATTCAATGT
CAGCCGATGAGTTAAAGAGGCTCAGAGAGTACATCGACGACAATTTAGCCAAGGGATGGAT
CAGGGAATCCGCGTCCCAAGTGGCCAGTCCAACTATGTGGGTACCCAAGAAGGATGGACCC
GATAGACTAGTTGTAGACTATAGAAAGCTTAACGCACTCACTAAGAAGGATCGATATCCACT
TCCATTAGCTACGGAATTAAGAGATCGATTAGGCGGAGCTACGATATTCACCAAGATGGACC
TACGTAATGGTTACCACTTGATCAGAATGAAGGAAGG
CGAAGAATGGAAAACCGCTTTCAAAACAAGATACGGGCTATACGAGTACCAAGTTATGCCA
TTCGGGCTAACCAACGCACCAGCTACTTTCATGAGGCTTATGAACAATGTGTTGTCACAATA
TTTGGATACTTGCTGTATATGCTACTTGGACGACATCCTAGTATATTCAAACAACAAGGTTCA
ACACATTAAGGACGTTAGCAACATCCTCGAAAGCCTATCCAAGGCAGACTTGCTGTGCAAAC
CAAGCAAATGCGAATTCCATGTCACAGAGACAGAATTCTTGGGATTCACCGTATCAAGCCAA
GGGCTCAAGATGAGCAAAGACAAGGTTAAGGCAGTGCTCGAATGGAAGCAGCCGACCACA
ATCAAGGAAGTACAATCCTTTCTAGGGTTCGTCAACTTCTACAGAAGATTTATCAAGGGTTA
TTCAGGGATTACTACACCCTTGACCACGTTAACCAGAAAAGATCAAGGAAGCTTCGAATGG
ACTGCCAAAGCACAGGAGTCATTCGATACGCTCAAACAAGCAGTGGCAGAAGAACCAATAC
TGTTGACTTTTGACCCAGAGAAAGAAATCATAGTGGAGACGGACTCCTCGGATTTCGCTATA
GGAGCAGTTCTGAGCCAACCGGGCCAGAATGGAAAATACCAGCCAATCGCATTCTACTCCC
GAAAACTATCACCAGCCGAATTGAATTACGAGATATATGACAAAGAATTACTGGCGATAGT
CGATGCATTTAGAGAATGGCGAGTGTATTTGGAAGGATCGAAATACACGGTACAGGTGTAT
ACAGATCATAAGAACTTGGTTTACTTCACCACAACGAAGCAGTTAAACAGACGACAGGTCA
GATGGTCGGAGACCATGGCCAACTACAATTTCAGAATTTCATATGTCAAAGGATCAGAAAA
CGCTAGAGCCGACGCTCTTAGCCGAAAACCAGAATATCAAGAAAACAAAACGTACGAGTCA
TACGCTATATTCAAGAAAGACGGCGAATCACTGGTTTACAATGCACCACAGCTTGCAGCAAC
ACACCTGTTGGAAGACAACCACCTCAGGAAACAGATCCAATCACACTACGACAAGGATGCT
ACTGCCACACGCATACGCAAGACAATAGAACCAGGATTCACTATAGAAAATGATACCATAT
ACTTTCATGGAAAAGTATACATTCCGAGTCAAATGACCAAGGAATTTGTGACGGAACAAC
ACGGGTTGCCGGCACATGGACACCAAGGAATTGCAAGGACATTTGCAAGAATACGGGAAAT
CAGTTACTTCCCACGAATGAGAACGATAGTTGAAGAAGTTGTTGGAAATTGTGACACCTGCA
TACGAAACAAGTCATCACGACATGCTCCGTATGGTCAGCTCCAGACCCCAGACATGCCTTCT
CAGCCATGGAAGTCCATCACATGGGACTTTGTGGTCAAACTACCACTCTCAAAGGATCCTAC
TACAGGAATTGAGTACGACGCGATACTCAATATAGTAGACAGGCTAACGAAATTTGCATAT
ATGATACCATTCAAGGAAACATGGGATGCTGAGCAACTAGCATATGTGTTCCTAAGGATCAT
AGTAAGCATACACGGAGTACCAGATGAGATAATCTCGGATCGAGACAAGCTCTTTACCTCA
AAATTCTGGACTACCTTATTAGCACTTATGGGTATCAAGAGAAAGCTATCGACATCTTTCCA
CCCACAAACAGATGGTCAAACAGAGAGGACCAATCAGACAATGGAAGCATATCTTAGATGC
TATGTAAATTATCGACAAGACAATTGGGTAGAGCTATTACCCATGGCACAGTTCGCATACAA
TACATCAGAAACGGAAACCACGAAAATCACACCAGCACGAGCTAATTTTGGGTTTAATCCA
CAAGCGTATAAAATCCCGATACCACAAGAAGTTAATGCCGAATCAGCGATAGTACAAATCG
AACAGCTGAAAGATCTCCAAGAGCAACTGGCTCTTGATCTAAGATTCATATCTTCCAGAACA
GCAGCGTACTACAATACGAAACGTAGTATGGAACCTACGCTTAAAGAGGGGGATAAAGTTT
ATTTGCTACGACGAAACATCGAAACCAAGAGACCAAGCAATAAACTCGACCACAGGAAACT
AGGACCATTCAAGATTGATAAGGTAATAGGAACGGTTAATTATCGATTGAAATTACCAGAC
ACAATGAATATCCACCCAGTATTCCACATATCCTTGCTCGAACCAGCACCACCAGGAGCGCC
AAATGCGCCATTTACAGAAATTGAACCAG
TCAACCCAAACGCCATATACGATGTCGAAACAATACTAGACTGCAAATACGTCAGAAACAA
GGTCAAGTATTTGATCAAATGGTTAGACTACCCACATTCAGAAAACACATGGGAACTCAAG
GAAGATCTCAGCTGCCCTGAGAAGCTACGGGCATTCCACCTGAAGTACCCACACCTGCCAAT
AAAGCCTCAAGATCCGCTTCGGACAACTCAGGCAAAGAAGGATCGAAGAAATCGAAGGAA
GAAGAATCAATAG
SEQ ID NO: 16-LTR for siR5
>BC1G_04408.1 retrotransposable element Tf2 1 protein type 1
ATGGCATCCAGAGCTACCGCCACAGGTCAGTCTACCGGAGATACCAACGACATCGAGATGA
CCGATGCCCCAAAGGAGATCACTATCAACGAAACACTTAAGATCGCCTTACCAGACAAGTA
CCAAGGTAGTCGACAAGAGCTCGATACTTTCCTCTTACAACTTGAGATCTACTTCCGATTCA
ATGAGGACAAGTTCACTACCAAGGAATCCAAGAGCATATGGGCTGCATCATACCTCCGAGG
TGAAGCAACCAAATGGATTCAACCATATTTGCGCGACTATTTCGAGCATGACGATAAGGATC
GCATGCAACCCACCCGAACAATCTTCAATAGTTTTGAAGGATTTAAGACAGAGATTCGTAGA
ATCTTCGGAAATTCCAACGAGTTAGAGGTAGCGGAAGATAAGATCTTCAACCTCAAGCAGA
CAGGATCAGCATTGAAATATGCTACGGAATTTCGAAGATATGCTGGAACAACCAAGTGGGA
CGAAATCGCTATCATGAGTCACTATCGTAAGGGACTCAAACCAGAAGTCAGACTGGAATTA
GAAAGATCTGCCGAGAGTACAGATCTGAACGATCTAATTCAGGACTCCATCGAATCAGATG
ATCGTCTCTACAGATATCGACAAAGCCAGAGATCATACAAACCCCAAGGAAATCAGAAGCA
AGGGCGTTACCGCAAAAATGAGGGTAGACCACGTTACAATCCACAGAGATACGGAGACCCC
ATGGAACTAGACGCCACGCACTACACAAACGGGAACGATGACTCGGAAAAGAGACGAAGA
CGAGAAAACAACTTATGCTTTGAATGTGGAAAAGCAGGGCACCGAGCAGCAGACTGCCGAA
GCAAGAAGACAGGAGGAAAAAGGGGCAACTTCAAACCTAAGTTCGGCAAAGGCCAACTTA
ACGCTACCTTTACAATCCCAGAAAATCCAACTAAATCCGAAAATACTGAGACTTTCACCGTT
GAGGAATTCCAGCAATTACTAAAGGAATTACCACGAAATCAAGAGGGCATGAATGCAATAG
ACTTATGGGAGCAAGAGTATTACAGAACCCCAACACCCTCTGTGACAGAAGAAAGTCATCA
GGACGAGGCAGAAGCAGACCACGCCACGATGAGCTGGACAGCTTGCTATGATGAATTCTGC
GGAATCCATCGATCAGATAAAGAAGCAACCGGATGGTTCCCTAAGAAGAGAAAGACGAAG
AACCATCAGAATAATGTAACATGCGAGGATTTAACTCCCAATATAACTTCGCAAGAAGTTCG
CAAAGTTACCCAGCAGTTAAATGCTACGGGACAGGCAGGACAGATATACTGCAAGGTTCAG
ATAAATGGACACATACAATCAGCCATGATAGATTCAGGGGCTACAGGAAATTTTATTGCACC
GGAAGCTGTAAAGTACTTGGGAATACCACTTCAAACGAAACAACACCCCTATCGATTGCAG
GACACGCTAGAGGCGTCCGCGAGACGTAACACGCGCCAAGGAGAGTTGAACGCGAACAAC
ACCGGCGACGTAGGACACCCAGTCCAGGGTCCTCCATTAAGAGCGAAGGCCAGTACACCTC
CTCTACAAATGCAGAAGCCAACGACACGGCACGAAATCGCAATCGAGGCAAAAGAAAGGC
CTACGATACCAGAACAGTACAAGAAATATGAACATGTTTTCAAAGAACCAGGGATCCATGA
GGCTTTACCAGAACACAAGCCATGGGATCATGAGATAATATTGGAGGAAGGCAAGATGCCT
GTGCACACCCCAATTTATTCAATGTCAGCCGATGAGTTAAAAAGGCTCAGAGAATACATCGA
CGACAATTTAGCCAAGGGATGGATCAGGGAATCCGCGTCCCAAGTGGCCAGTCCAACTATG
TGGGTACCCAAGAAGGATGGACCCGATAGACTAGTTGTAGACTATAGAAAGCTTAACGCAC
TCACTAAGAAGGATCGATATCCACTTCCATTAGCTACGGAATTAAGAGATCGATTAGGCGGA
GCTACGATATTTACCAAGATGGACCTACGTAATGGTTACCACTTGATCAGAATGAAGGAAGG
CGAAGAATGGAAGACCGCTTTCAAAACAAGATACGGGCTATACGAGTACCAAGTTATGCCG
TTCGGGCTAACCAACGCACCAGCTACTTTCATGAGGCTTATGAACAATGTGTTGTCACAATA
CTTGGATACTTGCTGTATATGCTACTTGGACGACATCCTAGTATATTCAAACAACAAGGTTC
AACACATTAAGGACGTTAGCAACATCCTCGAAAGCCT
ATCCAAGGCAGACTTGCTGTGCAAACCAAGCAAATGCGAATTCCATGTCACAGAGACAGAC
TTCTTGGGATTCACCGTATCAAGCCAAGGGCTCAAGATGAGCAAAGACAAGGTTAAGGCAG
TGCTCGAATGGAAACAGCCAACCACAATCAAGGAGGTACAATCCTTTCTAGGGTTCGTCAAC
TTCTACAGAAGATTTATCAAGGGTTATTCAGGGATTACTACACCCTTGACCACGTTAACCAG
AAAAGATCAAGGAAGCTTCGAATGGACTGCCAAAGCACAGGAGTCATTCGATACGCTCAAA
CAAGCAGTGGCAGAAGAGCCAATACTATTGACTTTTGACCCAGAGAAAGAAATCATAGTGG
AGACGGACTCCTCGGATTTCGCTATAGGAGCAGTTCTGAGCCAACCGGGCCAGAATGGAAA
ATACCAGCCAATCGCATTCTATTCCCGAAAACTATCACCAGCTGAGTTGAATTACGAGATAT
ATGACAAAGAATTGCTGGCGATAGTCGATGCATTTAGAGAATGGCGAGTATATTTGGAAGG
ATCGAAATACACAGTACAGGTGTATACAGATCATAAGAACTTGGTTTACTTCACCACAACGA
AGCAGTTAAACAGACGACAGGTCAGATGGTCGGAGACCATGGCCAACTACAATTTCAGAAT
TTCATATGTCAAAGGATCAGAAAACGCTAGAGCCGACGCTCTTAGCCGAAAACCAGAATAT
CAAGAAAACAAAACGTACGAGTCATACGCTATATTCAAGAAAGACGGCGAATCACTGGTTT
ACAATGCACCACAGCTTGCAGCAACACACCTGTTGGAAGACAACTACCTTAGGAAACAGAT
CCAATCACACTACGACAAGGATGCTACTGCCACACGCATACGTAAGACAATAGAACCAGGA
TTCACTATAGAAAATGATACCATATACTTTCATGGAAAAGTATACATTCCGAGTCAAATGA
CCAAGGAATTTGTGACGGAACAACACGGATTGCCGGCACATGGACACCAAGGAATTGCAAG
GACATTTGCAAGAATACGGGAAATCAGTTACTTCCCACGAATGAGAACGATAGTTGAAGAA
GTTGTTGGAAATTGTGACACCTGCATACGAAACAAGTCATCACGACATGCTCCGTATGGTCA
GCTCCAGACCCCAGACATGCCTTCTCAGCCATGGAAGTCCATCACATGGGACTTTGTGGTCA
AACTACCACTCTCCAAGGATCCTACTACAGGAATTGAGTACGACGCGATACTCAATATAGTA
GACAGGCTAACGAAATTTGCATATATGATACCATTCAAGGAAACATGGGATGCTGAGCAAC
TAGCATATGTGTTCCTTAGGATCATAGTAAGCATACACGGAGTACCAGATGAGATAATCTCG
GATCGAGACAAGCTCTTTACCTCGAAATTCTGGACTACCTTATTAGCACTTATGGGTATCAA
GAGAAAGCTATCGACATCTTTCCACCCACAAACAGATGGTCAAACAGAGAGGACCAATCAG
ACAATGGAAGCATATCTTAGATGCTATCGTATAAAATCCCGATACCACAAGAAGTTAATGCC
GAAT CAGCGATAG
SEQ ID NO: 17-LTR for siR5
>BC1G 12842.1 retrotransposable element Tf2 1 protein type 1
(Transcript: BCIT_12842)
ATGGCATCCAGAGCTACCGCCACAGGTCAGCCTACCGGAGATACCAACGACATCGAGATGA
CCGATGCCCCAAAGGAGATCACTATCAACGAAACCCTTAAGATCGCCTTACCAGACAAGTA
CCAAGGTAGTCGACAAGAGCTCGATACTTTCCTCTTACAACTTGAGATCTACTTTCGATTCAA
TGAGGACAAGTTCACTACCAAGGAATCCAAGAGTATATGGGCCGCATCATACCTTCGAGGT
GAAGCAACCAAATGGATCCAACCATATTTGCGCGACTATTTCGAGCATGACGATAAGGATC
GCATGCAACCCACCCGAACAATCTTCAATAGCTTTGAAGGATTTAAGACAGAGATTCGTAGA
ATCTTCGGAAATTCCAACGAGCTAGAGGTAGCGGAAGATAAGATCTTCAACCTCAAGCAGA
CAGGATCAGCATTGAAATATGCTACGGAATTTCGAAGATACGCTGGAACAACCAAGTGGGA
CGAAATCGCTATCATGAGTCACTACCGCAAGGGACTCAAACCAGAAGTCAGACTGGAATTA
GAAAGATCTGCCGAGAGTACAGATCTGAACGATCTAATTCAGGACTCCATCGAATCAGATG
ATCGTCTCTACAGATATCGACAAAGCCAGAGATCATACAAACCCCAAGGAAATCAGAAGCA
AGGGCGTTACCGCAAGAATGAGGGTAGACCACGTTACAATCCACAAAGATACGGAGACCCC
ATGGAACTAGACGCTACGCACTACACAAACGGGAACGATGACTCAGAAAAGAGACGAAGA
CGAGAAAACAACTTATGCTTTGAATGTGGAAAAGCAGGGCACCGAGCAGCAGACTGCCGAA
GCAAGAAGACAGGAGGAAAAAGGGGCAACTTCAAACCTAAGTTCGGCAAAGGCCAACTTA
ACGCTACCTTTACAATCCCAGAAAACCCAACTAAATCCGAAAATACTGAGACTTTCACCGTT
GAGGAATTCCAGCAATTACTAAAGGAATTACCACGAAATAAAGAGGGCATGAATGCAATAG
ACTTATGGGAACAAGAGTATTACAGAACCCCAACACCCTCTGTGACAGAAGAAAGTCACCA
GGACGAGGCAGAAGCGGACCACGCCACGATGAGCTGGACAGCTTGCTATGATGAATTCTGC
GGAATCCATCGATCAGATAAAGAAGCAACCGGATGGTTCCCCAAGAAAAGGAAGACGAAG
AACCATCAGAATAATGTAACATGCACGGATTTAACTTCAAATATAACTTCGCGAAAAGTTCG
CAAAGTTACCCAGCAGTTGAATGCTACGGGACAAGCAGGACAGATATACTGCAAGGTTCAG
ATAAATGGACACATACAATCAGCCATGATAGATTCAGGGGCTACAGGAAATTTTATTGCACC
AGAAGCTGCAAAGTACTTGGAAATACCACTTCAGACGAAACAATACCCCTATCGATTGCAGT
TAGTTGACGGACAGCTAGCAGGGTCTGACGGAAAGATTTCGCAGGAGACAATCCCAGTACG
AATGGGCATAACCCAACATACAGAGGTTATACAGCTTGACGTTGTGCCATTGGGCCAACAAC
AGATCATCTTAGGAATGCCATGGTTGAAGGCACATAATCCGAAAATAGATTGGGCACAAGG
AATTGTGACATTTGATCAGTGCAAAAGCGGTCACAGGGACACGCTAGAGGCGTCCGCGAGA
CGTAACACGCGCCAAGGAGAGTTGAACGCGAACAACACCGGCGACGTAGGACACCCAGTCC
AGGGTCCTCCATTAAGAGCGAAGGCCAGTACACCTCCTCTACAAATGCAGAAGCCAACGAC
ACGGCACGAAATCGCAATCGAGGCAAAAGAAAGGCCTACGATACCAGAACAGTACAAGAA
ATATGAACATGTTTTCAAAGAACCAGGGATCCATGAGGCTTTACCGGAACACAAGCCATGG
GATCATGAGATAATATTGGAGGAAGGCAAGATGCCTGTGCACACCCCAATTTATTCAATGTC
AGCCGATGAGTTAAAAAGGCTCAGAGAATACATCGACGACAATTTAGCCAAGGGATGGATC
AGGGAATCCGCGTCCCAAGTGGCCAGTCCAACTATGTGGGTACCCAAGAAGGATGGACCCG
ATAGACTAGTTGTAGACTATAGAAAGCTTAACGCACTCACTAAGAAGGATCGATATCCACTT
CCATTAGCTACGGAATTAAGAGATCGATTAGGCGGAGCTACGATATTCACCAAGATGGACCT
ACGTAATGGTTACCACTTGATCAGAATGAAGGAAGG
CGAAGAATGGAAAACCGCTTTCAAAACAAGATACGGGCTATACGAGTACCAAGTTATGCCG
TTCGGGCTAACCAACGCACCAGCTACTTTCATGAGGCTTATGAACAATGTGTTGTCACAATA
TTTGGATACTTGCTGTATATGCTACTTGGACGACATCCTAGTATATTCAAACAACAAGGTTCA
ACACATTAAGGACGTTAGCAGCATCCTCGAAAGTCTATCCAAAGCAGACTTGCTGTGCAAAC
CAAGCAAATGCGAATTCCATGTCACAGAAACAGAATTCTTGGGATTCACCGTATCAAGCCAA
GGGCTCAAGATGAGCAAAGACAAGGTTAAGGCAGTGCTCGAATGGAAGCAGCCGACCACA
ATCAAGGAAGTACAATCCTTTCTAGGATTTGTCAACTTCTATAGAAGATTTATCAAGGGTTA
TTCAGGGATTACTACACCCTTGACCACGTTAACCAGAAAAGATCAAGGAAGCTTCGAATGG
ACTGCCAAAGCACAGGAGTCATTCGATACACTCAAACAAGCAGTGGCAGAAGAACCAATAC
TGTTGACTTTTGACCCAGAGAAAGAAATCATAGTGGAAACGGATTCCTCAGATTTCGCTATA
GGAGCAGTTCTGAGCCAACCGGGCCAGAATGGAAAATACCAGCCAATCGCATTCTACTCCC
GAAAACTATCACCAGCCGAGTTGAATTACGAGATATATGACAAAGAATTACTGGCGATAGT
CGATGCATTTAGAGAATGGCGAGTATATTTGGAAGGATCGAAATACACAGTACAGGTGTAT
ACAGATCATAAGAACTTGGTTTACTTCACCACAACGAAGCAGTTAAACAGACGACAGGTCA
GATGGTCGGAGACCATGGCCAACTACAATTTCAGAATTTCATATGTCAAAGGATCAGAAAA
CGCTAGAGCCGACGCTCTTAGCCGAAAACCAGAATATCAAGAAAACAAAACGTACGAGTCA
TACGCTATATTCAAGAAAGACGGCGAATCACTGGTCTACAATGCACCACAGCTTGCAGCAAC
ACACCTGTTGGAAGACAACCACCTCAGAAAACAGATTCAATCACACTACGACAAGGATGCT
ACTGCCACACGCATACGCAAGACAATAGAACCAGGATTCACTATAGAAAATGATACCATAT
ACTTTCATGGAAAAGTATACATTCCGAGTCAAATGACCAAGGAATTTGTGACGGAACAAC
ATGGGTTGCCGGCACATGGACACCAAGGAATTGCAAGGACATTTGCAAGAATACGGGAAAT
CAGTTACTTCCCACGAATGAGAACGATAGTTGAAGAAGTTGTTGGAAATTGTGACACCTGCA
TACGAAACAAGTCATCACGACATGCTCCGTATGGTCAGCTCCAGACCCCAGACATGCCTTCT
CAGCCATGGAAGTCCATCACATGGGACTTTGTGGTCAAACTACCACTCTCAAAGGATCCTAC
TACAGGAATTGAGTACGACGCGATACTCAATATAGTAGACAGGCTAACGAAATTTGCATAT
ATGATACCATTCAAGGAAACATGGGATGCTGAACAACTAGCATATGTGTTCCTAAGGATCAT
AGTAAGCATACACGGAGTACCAGATGAGATAATCTCGGATCGAGACAAGCTCTTTACCTCA
AAATTCTGGACTACCTTATTAGCACTTATGGGTATCAAGAGAAAGCTATCGACATCTTTCCA
CCCACAAACAGATGGTCAAACAGAGAGGACCAATCAGACAATGGAAGCATATCTTAGATGC
TATGTAAATTATCGACAAGACAATTGGGTAGAACTATTACCTATGGCACAATTCGCATATAA
TACATCGGAAACGGAAACCACGAAAATCACACCAGCACGAGCTAATTTTGGGTTTAATCCA
CAAGCGTATAAAATCCCGATACCACAAGAAGTTAATGCCGAATCAGCAATAGTACAAGTCG
AACAGCTGAAAAATCTCCAAGAGCAACTGGCTCTTGATCTAAGATTCATATCTTCCAGAACA
GCAGCGTACTACAATACGAAACGTAGTATGGAACCTACGCTTAAAGAGGGGGATAAAGTTT
ATTTGCTACGACGAAACATCGAAACCAAGAGACCAAGCAATAAACTCGACCACAGGAAACT
AGGACCATTCAAGATTGATAAGGTAATAGGAACGGTTAATTATCGATTGAAATTACCAGAC
ACAATGAATATCCACCCAGTATTCCACATATCCTTGCTCGAACCAGCACCACCAGGAGCGCC
AAATGCGCCATTTACAGAAATTGAACCAG
TCAACCCAAACGCCATATACGATGTCGAAACAATACTAGACTGCAAATACGTCAGAAACAA
GGTCAAGTATTTGATCAAATGGTTAGACTACCCACATTCAGAAAACACATGGGAATTCAAGG
AGGATCTCAGCTGCCCTGAGAAGCTACGGGCATTCCACCTGAAGTACCCACACCTGCCAGTA
AAGCCTCAAGATCCG
CTTCGGACAACTCAGGCAAAGAAGGATCGAAGAAGTCGAAGGAAGAAGAATCAATAG
SEQ ID NO: 18-LTR for siR5
>BC1G_07532-retrotransposable element Tf2 1 protein type 1
ATGGCATCCAGAGCTACCGCCACAGGTCAATCTGCCGGAGACACCAACGACATCGAGATGA
CCGACGCCCCAAAGGAGATCACTATCAACGAAACCCTTAAGATCGCCTTACCAGACAAGTA
CCAAGGTAGTCGACAAGAGCTCGATACTTTCCTCTTACAACTTGAGATCTACTTTCGATTCAA
TGAGGACAAGTTCACTACCAAGGAATCCAAGAGTATATGGGCCGCATCATACCTTCGAGGT
GAAGCAACCAAATGGATCCAACCATATTTGCGCGACTATTTCGAGCATGACGATAAGGATC
GTATGCAACCCACCCGAACAATCTTCAATAGTTTTGAAGGATTTAAGACAGAGATTCGTAGA
ATCTTCGGAAATTCCAACGAGTTAGAGGTAGCGGAAGATAAGATCTTCAACCTCAAGCAGA
CAGGATCAGCATTGAAATATGCTACGGAATTTCGAAGATATGCTGGAACAACCAAGTGGGA
CGAAATCGCTATCATGAGTCACTACCGCAAGGGACTCAAACCAGAAGTCAGACTGGAATTA
GAAAGATCTGCCGAGAGTACAGATCTGAACGATCTAATTCAGGACTCCATCGAATCAGATG
ATCGTCTCTACAGATATCGACAAAGCCAGAGATCATACAAACCCCAAGGAAATCAGAAGCA
AGGGCGTTACCGCAAAAATGAGGGTAGACCACGTTACAATCCACAGAGATACGGAGACCCC
ATGGAACTAGACGCCACGCACTACACAAACGGGAACGATGACTCGGAAAAGAGACGAAGA
CGAGAAAACAACTTATGCTTTGAATGTGGAAAAGCAGGGCACCGAGCAGCAGACTGCCGAA
GCAAGAAGACAGGAGGAAAAAGGGGCAACTTCAAACCTAAGTTCGGCAAAGGCCAACTTA
ACGCTACCTTTACAATCCCAGAAAATCCAACTAAATCCGAAAATACTGAGACTTTCACCGTT
GAGGAATTCCAGCAATTACTAAAGGAATTACCACGAAATCAAGAGGGCATGAATGCAATAG
ACTTATGGGAGCAAGAGTATTACAGAACCCCAACACCCTCTGTGACAGAAGAAAGTCATCA
GGACGAGGCAGAAGCAGACCACGCCACGATGAGCTGGACAGCTTGCTATGATGAATTCTGC
GGAATCCATCGATCAGATAAAGAAGCAACCGGATGGTCCCCTAAGAAGAGAAAGACGAAG
AACCATCAGAATAATGTAACATGCGAGGATTTAACTCCCAATATAACTTCGCAAGAAGTTCG
CAAAGTTACCCAGCAGTTAAATGCTACGGGACAGGCAGGACAGATATACTGCAAGGTTCAG
ATAAATGGACACATACAATCAGCCATGATAGATTCAGGGGCTACAGGAAATTTTATTGCACC
GGAAGCTGTAAAGTACTTGGGAATACCACTTCAAACGAAACAACACCCCTATCGATTGCAG
GACACGCTAGAGGCGTCCGCGAGACGTAACACGCGCCAAGGAGAGTTGAACGCGAACAAC
ACCGGCGACGTAGGACACCCAGTCCAGGGTCCTCCATTAAGAGCGAAGGCCAGTACACCTC
CTCTACAAATGCAGAAGCCAACGACACGGCACGAAATCGCAATCGAGGCAAAAGAAAGGC
CTACGATACCAGAACAGTACAAGAAATATGAACATGTTTTCAAAGAACCAGGGATCCATGA
GGCTTTACCGGAACACAAGCCATGGGATCATGAGATAATATTGGAGGAAGGCAAGATGCCT
GTGCACACCCCAATTTATTCAATGTCAGCCGATGAGTTAAAAAGGCTCAGAGAATACATCGA
CGACAATTTAGCCAAGGGATGGATCAGGGAATCCGCGTCCCAAGTGGCCAGTCCAACTATG
TGGGTACCCAAGAAGGATGGACCCGATAGACTAGTTGTAGACTATAGAAAGCTTAACACAC
TCACTAAGAAGGATCGATATCCACTTCCATTAGCTACGGAATTAAGAGATCGGTTAGGCGGA
GCTACGATATTTACCAAGATGGACCTACGTAATGGTTACCACTTGATCAGAATGAAGGAAGG
CGAAGAATGGAAGACCGCTTTCAAAACAAGATACGGGCTATACGAGTACCAAGTTATGCCG
TTCGGGCTAACCAACGCACCAGCTACTTTCATGAGGCTTATGAACAATGTGTTGTCACAATA
CTTGGATACTTGCTGTATATGCTACTTGGACGACATCCTAGTATATTCAAACAACAAGGTTC
AACACATTAAGGACGTTAGCAACATCCTCGAAAGCCTATCCAAGGCAGACTTGCTGTGCAA
ACCAAGCAAATGCGAATTCCATGTCACAGAGACAGAATTCTTGGGATTCACCGTATCAAGCC
AAGGGCTCAAGATGAGCAAAGACAAGGTTAAGGCAGTGCTCGAATGGAAGCAGCCAACCA
CAATCAAGGAAGTACAATCCTTTCTAGGGTTCGTCAACTTCTACAGAAGATTTATCAAGGGT
TATTCAGGGATTACTACACCCTTGACCACGTTAACCAGAAAAGATCAAGAAAGCTTCGAATG
GACTGCCATAGCACAGGAGTCATTCGATACGCTCAAACAAGCAGTGGCAGAAGAGCCAATA
CTATTGACTTTTGACCCAGAGAAAGAAATCATAGTGGAGACGGACTCCTCGGATTTCGCTAT
AGGAGCAGTTCTGAGCCAACCGGGCCAGAATGGAAAATACCAGCCAATCGCATTCTACTCC
CGAAAACTATCACCAGCCGAATTGAATTACGAGATATATGACAAAGAATTGCTGGCGATAG
TCGATGCATTTAGAGAATGGCGAGTATATTTGGAAGGATCGAAATACACAGTACAGGTGTAT
ACAGATCATAAGAACTTGGTTTACTTCACCACAACGAAGCAGTTAAACAGACGACAGGTCA
GATGGTCGGAGACCATGGCCAACTACAATTTCAGAATTTCATATGTCAAAGGATCAGAAAA
CGCTAGAGCCGACGCTCTTAGCCGAAAACCAGAATATCAAGAAAACAAAACGTACGAGTCA
TACGCTATATTCAAGAAAGACGGCGAATCACTGGTTTACAATGCACCACAGCTTGCAGCAAC
ACACCTGTTGGAAGACAACTACCTTAGGAAACAGATCCAATCACACTACGACAAGGATGCT
ACTGCCACACGCATACGCAAGACAATAGAACCAGGATTCACTATAGAAAATGATACCATAT
ACTTTCATGGAAAAGTATACATTCCGAGTCAAATGACCAAGGAATTTGTGACGGAACAAC
ATGGGTTGCCGGCACATGGACACCAAGGAATTGCAAGGACATTTGCAAGAATACGGGAAAT
CAGTTACTTCCCACGAATGAGAACGATAGTTGAAGAAGTTGTTGGAAATTGTGACACCTGCA
TACGAAACAAGTCATCACGGCATGCTCCGTATGGTCAGCTCCAGACCCCAGACATGCCTTCT
CAGCCATGGAAGTCCATCACATGGGACTTTGTGGTCAAACTACCACTCTCAAAGGATCCTAC
TACAGGAATTGAGTACGACGCGATACTCAATATAGTAGACAGGCTAACGAAATTTGCATAT
ATGATACCATTCAAGGAAACATGGGATGCTGAACAACTAGCATATGTGTTCCTAAGGATCAT
AGTAAGCATACACGGAGTACCAGATGAGATAATCTCGGATCGAGACAAGCTCTTTACCTCG
AAATTCTGGACTACCTTATTAGCACTTATGGGTATCAAGAGAAAGCTATCGACATCTTTCCA
CCCACAAACAGATGGTCAAACAGAGAGGACCAATCAGACAATGGAAGCATATCTTAGATGC
TATCGTATAAAATCCCGATACCACAAGAAGTTAATGCCGAAT CAGCAATAG
SEQ ID NO: 19-LTR for siR5
>BC1G_09712-enzymatic polyprotein
ATGGCATCCAGAGCTACCGCCACAGGTCAGTCTACCGAAGATACCAACGACATCGAGATGA
CCGATGCCCCAAAGGAGATCACTATCAACGAAACACTTAAGATCGCCTTACCAGACAAGTA
CCAAGGTAGTCGACAAGAGCTCGATACTTTCCTCTTACAACTTGAGATCTACTTCCGATTCA
ATGAAGACAAGTTCACTACCAAGGAATCCAAGAGCATATGGGCTGCATCATACCTCCGAGG
TGAAGCAACCAAATGGATTCAACCATATTTGCGCGACTATTTCGAGCATGACGATAAGGATC
GCATGCAACCCACCCGAACAATCTTCAATAGTTTTGAAGGATTTAAGACAGAGATTCGTAGA
ATCTTCGGAAATTCCAACGAGTTAGAGGTAGCGGAAGATAAGATCTTCAACCTCAAGCAGA
CAGGATCAGCATTGAAATATGCTACGGAATTTCGAAGATATGCTGGAACAACCAAGTGGGA
CGAAATCGCTATCATGAGTCACTACTGTAAGGGACTCAAACCAGAAGTCAGACTAGAGTTA
GAAAGATCTGCCGAGAGTACAGATCTGAACGATCTAATTCAGGACTCCATCGAATCAGATG
ATCGTCTCTATAGATATCGACAAAGCCAAAGATCATACAAACCCCAAGGAAACCAAAAGCA
AGGGCGTTACCGCAAGAATGAGGGTAGACCACGTTACAATCCACAGAGATACGGAGACCCC
ATGGAACTAGACGCCACGCACTACACAAACGGGAACGATGACTCAGAAAAGAGACGAAGA
CGAGAAAACAACTTATGCTTTGAATGTGGAAAAGCAGGGCACCGAGCAGTAGACTGCCGAA
GCAAGAAGACAGGAGGAAAAAGGGGCAACTTCAAACCTAAGTTCGGCAAGGGCCAACTTA
ACGCCACCTTTGCCATCTCAGAAAACTCAACTAAAACCGAAAATACTGAGACTTTCACCGTT
GAGGAATTTCAGCAATTACTAAAGGAATTACCACGAAATAAAGAGGGCATGAATGCAATAG
ACTTATGGGAACAAGAGTATTACAGAACCCCAACACCCTCTGTGACAGAAGAAAGTCACCA
GGACGAGGCAGAAGCGGACCACGCCACGATGAGCTGGACAGCTTGCTATGATGAATTCTGC
GGAATCCATCGATCAGATAAAGAAGCAACCGGATGGTTCCCCAAGAAAAGGAAGACGAAG
AACCATCAGAATAATGTAACATGCGAGGATTTAACTCCCAATATAACTTCGCAAGAAGTTCG
CAAAGTTACCCAGCAGTTGAATGCTACGGGACAGGCAGGACAAGTGTACTGCAAGGTCCAG
ATAAATGGACACATACAATCAGCCATGATAGATTCAGGGGCTACAGGAAATTTTATTGCACC
AGAAGCTGCAAAGTACTTGGAAATACCACTTCAAACGAAACAACACCCCTATCGATTGCAG
GACACGCTAGAGGCGTCCGCGAGACGTAACACGCGCCAAGGGGAGTTGAACGCGAACAAC
ACCGGCGACGTAGGACACCCAGTCCAGGGTCCTCCATTAAGAGCGAAGGCCAGTACACCTC
CTCTACAAATGCAGAAGCCAACGACACGGCACGAAATCGCAATCGAGGCAAAAGAAAGGC
CTACGATACCAGAACAGTACAAGAAATATGAACATGTTTTCAAAGAACCAGGGATCCATGA
GGCTTTACCGGAACACAAGCCATGGGATCATGAGATAATATTGGAGGAAGGCAAGATGCCT
GTGCACACCCCAATTTATTCAATGTCAGCCGATGAGTTAAAAAGGCTCAGAGAATACATCGA
CGACAATTTAGCCAAGGGATGGATCAGGGAATCCGCGTCCCAAGTGGCCAGTCCAACTATG
TGGGTACCCAAGAAGGATGGACCCGATAGACTAGTTGTAGACTATAGAAAGCTTAACGCAC
TCACTAAGAAGGATCGATATCCACTTCCATTAGCTACGGAATTAAGAGATCGATTAGGCGGA
GCTACGATATTCACCAAGATGGACCTACGTAATGGTTACCACTTGATCAGAATGAAGGAAG
GCGAAGAATGGAAAACCGCTTTCAAAACAAGATACGGGCTATACGAGTACCAAGTTATGCC
ATTCGGGCTAACCAACGCACCAGCTACTTTCATGAGGCTTATGAACAATGTGTTGTCACAAT
ATTTGGATACTTGCTGTATATGCTACTTGGACGACATCCTAGTATATTCAAACAACAAGGTTC
AACACATTAAGGACGTTAGCAACATCCTCGAAAGTCT
ATCCAAAGCAGACTTGCTGTGCAAACCAAGCAAATGCGAATTCCATGTCACAGAAACAGAA
TTCTTGGGATTCACCGTATCAAGCCAAGGGCTCAAGATGAGCAAAGACAAGGTTAAGGCAG
TGCTCGAATGGAAGCAGCCAACCACAATCAAGGAGGTACAATCCTTTCTAGGGTTCGTCAAC
TTCTACAGAAGATTTATCAAGGGTTATTCAGGGATTACTACACCCTTGACCACGTTAACCAG
AAAAGATCAAGGAAGCTTCGAATGGACTGCCAAAGCACAGGAGTCATTCGATACACTCAAA
CAAGCAGTGGCAGAAGAACCAATACTGTTGACTTTTGACCCAGAGAAAGAAATCATAGTGG
AAACGGATTCCTCAGATTTCGCTATAGGAGCAGTTCTGAGCCAACCGGGCCAGAATGGAAA
ATACCAGCCAATCGCATTCTACTCCCGAAAACTATCACCAGCTGAGTTAAATTACGAGATAT
ATGACAAAGAATTACTGGCAATAGTCGATGCATTTAGAGAATGGCGAGTATATTTGGAAGG
ATCGAAATACACAGTACAGGTGTATACAGATCATAAGAACTTGGTTTACTTCACCACAACGA
AGCAGTTAAACAGACGACAGGTCAGATGGTCGGAGACCATGGCCAACTACAATTTCAGAAT
TTCATATGTCAAAGGATCAGAAAACGCTAGAGCCGACGCTCTTAGCCGAAAACCAGAATAT
CAAGAAAACAAAACGTACGAGTCATACGCTATATTCAAGAAAGACGGCGAATCACTGGTCT
ACAATGCACCACAGCTTGCAGCAACACACCTGTTGGAAGACAACCACCTCAGGAAACAGAT
CCAATCACACTACAACAAGGATGCTACTGCCACACGCATACGCAAGACAATAGAACCAGGA
TTCACTATAGAAGATGATACCATATACTTTCATGGAAAAGTATACATTCCGAGTCAAATGA
CCAAGGAATTTGTGACGGAACAACACGGATTGCCGGCACATGGACACCAAGGAATTGCAAG
GACATTTGCAAGAATACGGGAAATCAGTTACTTCCCACGAATGAGAACGATAGTTGAAGAA
GTTGTTGGAAATTGTGACACCTGCATACGAAACAAGTCATCACGACATGCTCCGTATGGTCA
GCTCCAGACCCCAGACATGCCTTCTCAGCCATGGAAGTCCATCACATGGGACTTTGTGGTCA
AACTACCACTCTCAAAGGATCCTACTACAGGAATTGAGTACGACGCGATACTCAATATAGTA
GACAGGCTAACGAAATTTGCATATATGATACCATTCAAGGAAACATGGGATGCTGAGCAAC
TAGCATATGTGTTCCTAAGGGTCATAGTAAGCATACACGGAGTACCAGATGAGATAATCTCG
GATCGAGACAAGCTCTTTACCTCGAAATTCTGGACTACCTTATTAGCACTTATGGGTATCAA
GAGAAAGCTATCGACATCTTTCCACCCACAAACAGATGGTCAAACAGAGAGGACCAATCAG
ACAATGGAAGCATATCTTAGATGCTATCGTATAAAATCCCGATACCACAAGAAGTTAATGCC
GAAT CAGCAATAG
SEQ ID NO: 20-LTR for siR5
>BC1G_15972-enzymatic polyprotein
ATGGCATCCAGAGCTACCGCCACAGGTCAATCTGCCGGAGACACCAACGACATCGAGATGA
CCGACGCTCCAAAGGAGATCACTATCAACGAAACCCTTAAGATCGCCTTACCAGACAAGTA
CCAAGGTAGTCGACAAGAGCTCGATACTTTCCTCTTACAACTTGAGATCTACTTCCGATTCA
ATGAGGACAAGTTCACTACCAAGGAATCCAAGAGCATATGGGCCGCGTCATACCTTCGAGG
TGAAGCAACCAAATGGATTCAACCATATTTGCGCGACTATTTCGAGCATGACGATAAGGATC
GCATGCAACCCACCCGAACAATCTTCAATAGTTTTGAAGGATTTAAGACAGAGGTTCGTAGA
ATCTTCGGAAATTCCAACGAGTTAGAGGTAGCGGAAGATAAGATCTTCAACCTCAAGCAGA
CAGGATCAGCATTGAAATATGCTACGGAATTTCGAAGATATGCTGGAACAACCAAGTGGGA
CGAAATCGCTATCATGAGTCACTACCGCAAGGGACTCAAACCAGAAGTCAGACTAGAATTA
GAAAGATCTGCCGAGAGTACAGATCTAAACGATCTAATTCAGGACTCCATCGAATCAGATG
ATCGTCTCTACAGATATCGACAAAGCCAAAGATCATACAAACCCCAAGGAAATCAGAAGCA
AGGGCGTTACCGCAAGAATGAGGGTAGACCACGTTACAATCCACAGAGATACGGAGACCCC
ATGGAACTAGACGCTACGCACTACACAAACGGGAACGATGACTCAGAAAAGAGACGAAGA
CGAGATAACAACTTATGCTTTGAATGTGGAAAAGCAGGGCACCGAGCAGCAGACTGCCGAA
GCAAGAAGACAGGAGGAAAAAGGGGCAACTTCAAACCTAAGTTCGGCAAGGGCCAACTTA
ATGCCACCTTTGCAATCTCAGAAAACTCAACTAAACCCGAAAATACTGAGACTTTCACCGTT
GAGGAATTCCAGCAATTATTAGAGGAATTACCACGAAACCAAGAGGGCATGAATGCAATAG
ACTTATGGGAACAAGAGTATTACAGAACTCCAACACCCTCTGTGACAGAAGAAAGTCACCA
GGACGAGGCAGAAGCGGACCACGCCACGATAAGCTGGACAGCTTGCTATGACGAATTCTGC
GGAATCCATCGATCAGATAAAGAAGCAACCGGATGGTTCCCCAAGAAGAGAAAGACGAAG
AACCGACAGAATAATGTAACATGCAAGGATTTAACTCCAAATGTAACTTCGCGAAAAGTTC
GCAAAGTTACACAGCAATTGAATGCTACGGGACAGGCAGGACAAATATACTGCACGGTTCA
GATAAATGGACACATACAATCAGCCATGATAGATTCAGGGGCTACAGGGAATTTTATTGCAC
CAGAAGCTGCAAAGTACTTGGAAATACCACTTCAAACGAAACAACACCCCTACCGATTGCA
GTTAGTTGACGGACAGCTAGCAGGGTCTGACGGAAAGATTTCGCAGGAGACAATCCCAGTA
CGAATGGGCATAACCCAACATACAGAGGTTATACAGCTTGACGTTGTGCCATTGGGCCAACA
ACAGATCATCTTAGGAATGCCATGGTTAAAGGCACATAATCCGAAAATAGATTGGGCACAA
GGAATTGTGACATTTGATCAGTGCAAAAGCGGTCACAGGGACACGCTAGAGGCGTTCGCGA
GACGTAACACGCGCCAAGGAGAGTTGAACGCGAACAACACCGGCGACGTAGGACACCCAGT
CCAGGGTCCTCCATTAAGAGCGAAGGCCAGTACACCTCCTCTACAAATGCAGAAGCCAACG
ACACGGCACGAAATCGCAATCGAGGCAAAAGAAAAGCCTACGATACCAGAACAGTACAAG
AATTATGAACATGTTTTCAAAGAACCAGGGATCCATGAGGCTTTACCGGAACACAAGCCATG
GGATCATGAGATAATATTGGAGGAAGGCAAGATGCCTGTGCACACCCCAATTTATTCAATGT
CAGCCGATGAGTTAAAGAGGCTCAGAGAGTACATCGACGACAATTTAGCCAAGGGATGGAT
CAGGGAATCCGCGTCCCAAGTGGCCAGTCCAACTATGTGGGTACCCAAGAAGGATGGACCC
GATAGACTAGTTGTAGACTATAGAAAGCTTAACGCACTCACTAAGAAGGATCGATATCCACT
TCCATTAGCTACGGAATTAAGAGATCGATTAGGCGGAGCTACGATATTTACCAAGATGGACC
TACGTAATGGTTACCACTTGATCAGAATGAAGGAAGG
CGAAGAATGGAAAACCGCTTTCAAAACAAGATACGGGCTATACGAGTACCAAGTTATGCCA
TTCGGGCTAACCAACGCACCAGCTACTTTCATGAGGCTTATGAACAATGTGTTGTCACAATA
TTTGGATACTTGCTGTATATGCTACTTGGACGACATCCTAGTATATTCAAACAACAAGGTTCA
ACACATTAAGGACGTTAGCAACATCCTCGAAAGCCTATCCAAGGCAGACTTGCTGTGCAAAC
CAAGCAAATGCGAATTCCATGTCACAGAGACAGAATTCTTGGGATTCACCGTATCAAGCCAA
GGGCTCAAGATGAGCAAAGGCAAGGTTAAGGCAGTGCTCGAATGGAAGCAGCCGACCACA
ATCAAGGAAGTACAATCCTTTCTAGGGTTCGTCAACTTCTACAGAAGATTTATCAAAGGTTA
TTCAGGGATTACTACACCCTTGACCACGTTAACCAGAAAAGATCAAGGAAGCTTCGAATGG
ACTGCCAAAGCACAGGAGTCATTCGATACGCTCAAACAAGCAGTGGCAGAAGAGCCAATAC
TATTGACTTTTGACCCAGAGAAAGAAATCATAGTGGAGACGGACTCCTCGGATTTCGCTATA
GGAGCAGTTCTGAGCCAACCGGGTCAGAATGGAAAATACCAGCCAATCGCATTCTACTCCC
GAAAACTATCACCAGCTGAGTTGAATTACGAGATATATGACAAAGAATTACTGGCGATAGT
CGATGCATTTAGAGAATGGCGAGTATATTTGGAAGGATCGAAATACACAGTACAGGTGTAC
ACAGATCATAAGAACTTGGTTTACTTCACCACAACGAAGCAGTTAAACAGACGACAGGTCA
GATGGTCGGAGACCATGGCCAACTACAACTTTAGAATTTCATATGTCAAAGGATCAGAAAAT
GCTAGAGCCGACGCTCTTAACCGAAAACCAGAATATCAAGAAAACAAAGCGTACGAGTCAT
ACGCTATATTCAAGAAAGACAGCGAATCACTGGTTTACAATACACCACAGCTTGCAACAAC
ACACCTGTTGGAAGACAACCACCTCAGGAAACAGATCCAATCACACTACGACAAGGATACT
ACTGCCACACGCATACGCAAAACAATAGAACCAGGATTCACTATAGAAAATGATACCATAT
ACTTTCATAGAAAAGTATACATTCCGAGTCAAATGACCAAGGAATTTGTGACGGAACAAC
ACGGGTTGCCGGCACATGGACACCAAGGAATTGCAAGGACATTTGCAAGAATACGGGAAAT
CAGTTACTTCCCACGAATGAGAACGATAGTTGAAGAAGTTGTTGGAAATTGTGACACCTGCA
TACGAAACAAGTCATCACGACATGCTCCGTATGGTCAGCTCCAGACCCCAGACATGCCTTCT
CAGCCATGGAAGTCCATCACATGGGACTTTGTGGTCAAACTACCACTCTCAAAGGATCCTAC
TACAGGAATTGAGTACGACGCGATACTCAATATAGTAGACAGGCTAACGAAATTTGCATAT
ATGATACCATTCAAGGAAACATGGGATGCTGAGCAACTAGCATATGTGTTCCTAAGGATCAT
AGTAAGCATACACGGAGTACCAGATGAGATAATCTCGGATCGAGACAAGCTCTTTACCTCG
AAATTCTGGACTACCTTATTAGCACTTATGGGTATCAAGAGAAAGCTATCGACATCTTTCCA
CCCACAAACAGATGGTCAAACAGAGAGGACCAATCAGACAATGGAAGCATATCTTAGATGC
TATGTAAATTATCGACAAGACAATTGGGTAGAGCTATTACCCATGGCACAGTTCGCATACAA
TACATCAGAAACGGAAACCACGAAAATCACACCAGCACGAGCTAATTTTGGGTTTAATCCA
CAAGCGTATAAAATCCCGATACCACAAGAAGTTAATGCCGAATCAGCGATAGTACAAGTCG
AACAGTTGAAAGATCTCCAAGAGCAACTGGCTCTTGATCTAAGATTCATATCTTCCAGAACA
GCAGCGTACTACAATACGAAACGTAGTATGGAACCTACGCTTAAAGAGGGGGATAAAGTTT
ATTTGCTACGACGAAACATCGAAACCAAGAGACCAAGCAATAAACTCGACCACAGGAAACT
AGGACCATTCAAGATTGATAAGGTAATAGGAACGGTTAATTATCAATTGAAATTACCAGAC
ACAATGAATATCCACCCAGTATTCCACATATCCTTGCTCGAACCAGCACCACCAGGAGCGCC
AAATGCGCCATTTACAGAAATTGAACCAG
TCAACCCAAACGCCATATACGATGTCGAAACAATACTAGACTGCAAATACGTCAGAAACAA
GGTCAAGTATTTGATCAAATGGTTAGACTACCCACATTCAGAAAACACATGGGAACTCAAG
GAAGATCTCAGCTGCCCTGAGAAACTACGGGCATTCCACCTGAAGTACCCACATCTGCCAAC
AAAGCCTCAAGCTCCG
CATCAGACAACAAAGGCAACGAGGGGTCGAAGAAACCAAAAGAAGAACCACTAG
SEQ ID NO: 21-LTR for siR5
>BC1G_13999 retrotransposable element Tf2 1 protein type 1
ATGTGGGTACCCAAGAAGGATGGACCCGATAGACTAGTTGTAGACTATAGAAAGCTTAACG
CACTCACTAAGAAGGATCGATATCCACTTCCATTAGCTACGGAATTAAGAGATCGATTAGGC
GGAGCTACGATATTCACCAAGATGGACCTACTATATTCAAACAACAAGGTTCAACACATTAA
GGACGTTAGCAACATCCTCGAAAGCCTATCCAAGGCAGACTTGCTGTGCAAACCAAGCAAA
TGCGAATTCCATGTCACAGAGACAGAATTCTTGGGATTCACCGTATCAAGCCAAGGGCTCAA
GATGAGCAAAGGCAAGGTTAAGGCAGTGCTCGAATGGAAGCAGCCGACCACAATCAAGGA
AGTACAATCCTTTCTAGGGTTCGTCAACTTCTACAGAAGATTTATCAAAGGTTATTCAGGGA
TTACTACACCCTTGACCACGTTAACCAGAAAAGATCAAGGAAGCTTCGAATGGACTGCCAA
AGCACAGGAGTCATTCGATACGCTCAAACAAGCAGTGGCAGAAGAGCCAATACTATTGACT
TTTGACCCAGAGAAAGAAATCATAGTGGAGACGGACTCCTCGGATTTCGCTATAGGAGCAG
TTCTGAGCCAACCGGGTCAGAATGGAAAATACCAGCCAATCGCATTCTACTCCCGAAAACTA
TCACCAGCTGAGTTGAATTACGAGATATATGACAAAGAATTACTGGCGATAGTCGATGCATT
TAGAGAATGGCGAGTATATTTGGAAGGATCGAAATACACAGTACAGGTGTACACAGATCAT
AAGAACTTGGTTTACTTCACCACAACGAAGCAGTTAAACAGACGACAGGTCAGATGGTCGG
AGACCATGGCCAACTACAACTTTAGAATTTCATATGTCAAAGGATCAGAAAATGCTAGAGCC
GACGCTCTTAGCCGAAAACCAGAATATCAAGAAAACAAAACGTACGAGTCATACGCTATAT
TCAAGAAAGACGGCGAATCACTGGTTTACAATGCACCACAGCTTGCAGCAACACACCTGTTG
GAAGACAACCACCTCAGGAAACAGATCCAATCACACTACGACAAGGATGCTACTGCCACAC
GCATACGCAAGACAATAGAACCAGGATTCACTATAGAAAATGATACCATATACTTTCATGG
AAAAGTATACATTCCGAGTCAAATGACCAAGGAATTTGTGACGGAACAACACGGGTTGCC
GGCACATGGACACCAAGGAATTGCAAGGACATTTGCAAGAATACGGGAAATCAGTTACTTC
CCACGAATGAGAACGATAGTTGAAGAAGTTGTTGGAAATTGTGACACCTGCATACGAAACA
AGTCATCACGACATGCTCCGTATGGTCAGCTCCAGACCCCAGACATGCCTTCTCAGCCATGG
AAGTCCATCACATGGGACTTTGTGGTCAAACTACCACTCTCAAAGGATCCTACTACAGGAAT
TGAGTACGACGCGATACTCAATATAGTAGACAGGCTAACGAAATTTGCATATATGATACCAT
TCAAGGAAACATGGGATGCTGAGCAACTAGCATATGTGTTCCTAAGGATCATAGTAAGCAT
ACACGGAGTACCAGATGAGATAATCTCGGATCGAGACAAGCTCTTTACCTCGAAATTCTGGA
CTACCTTATTAGCACTTATGGGTATCAAGAGAAAGCTATCGACATCTTTCCACCCACAAACA
GATGGTCAAACAGAGAGGACCAATCAGACAATGGAAGCATATCTTAGATGCTATGTAAATT
ATCGACAAGACAATTGGGTAGAGCTATTACCCATGGCACAGTTCGCATACAATACATCAGA
AACGGAAACCACGAAAATCACACCAGCACGAGCTAATTTTGGGTTTAATCCACAAGCGTAT
AAAATCCCGATACCACAAGAAGTTAATGCCGAATCAGCGATATATGGAACCTACGCTTAA
SEQ ID NO: 22-LTR for siR5
>BC1G_04888.1 retrotransposable element Tf2 1 protein type 1
(Transcript: BCIT_04888)
ATGGCCAACTACAATTTTAGAATTTCATATGTCAAAGGATCAGAAAACGCTAGAGCCGACGC
TCTTAGCCGAAAACCAGAATATCAAGAAAACAAAACGTACGAGTCATACGCTATATTCAAG
AAAGACGGCGAATCACTGGTCTACAATGCACCACAGCTTGCAGCAACACACCTGTTGGAAG
ACAACCACCTCAGGAAACAGATCCAATCACACTACAACAAGGATGCTACTGCCACACGCAT
ACGCAAGACAATAGAACCAGGATTCACTATAGAAGATGATACCATATACTTTCATGGAAAA
GTATACATTCCGAGTCAAATGACCAAGGAATTTGTGACGGAACAACATGGGTTGCCGGCA
CACGGACATCAAGGGATTGCAAGAACATTTGCAAGAATCCGGGAAATCAGTTACTTCCCAC
GAATGAGAACGATAGTTGAAGAAGTTGTTGGAAATTGTGACACCTGCATACGAAACAAGTC
ATCACGACATGCGCCGTATGGTCAGCTCCAGACCCCAGACATGCCTTCTCAGCCATGGAAGT
CCATCACATGGGACTTTGTGGTCAAACTACCACTCTCAAAGGATCCTACTACAGGAATTGAG
TACGACGCGATACTCAATATAGTAGACAGGCTAACGAAATTCGCATATATGATACCATTCAA
GGAAACATGGGATGCTGAGCAACTAGCATATGTGTTCCTAAGGATCATAGTAAGCATACAC
GGAGTACCAGATGAGATAATCTCGGATCGAGACAAGCTCTTTACCTCGAAATTCTGGACTAC
CTTATTAGCACTTATGGGTATCAAGAGAAAGCTATCGACATCTTTCCACCCACAAACAGATG
GTCAAACAGAGAGGACCAATCAGACAATGGAAGCATATCTTAGATGCTATCGTATAAAATC
CCGATACCACAAGAAGTTAATGCCGAATCAGCGATAG
SEQ ID NO: 23-LTR for siR5
>BC1G_16375.1 hypothetical protein similar to truncated Pol
(Transcript: BCIT_16375)
ATCCAATCACACTACAACAAGGATGCTACTGCCACACGCATACGCAAGACAATAGAACCAGG
ATTCACTATAGAAGATGATACCATATACTTTCATGGAAAAGTATACATTCCGAGTCAAATGAC
CAAGGAATTTGTGACGGAACAACATGGGTTGCCGGCACACGGACATCAAGGGATTGCAAGA
ACATTTGCAAGAATCCGGGAAATCAGTTACTTCCCACGAATGAGAACGATAGTTGAAGAAGTT
GTTGGAAATTGTGACACCTGCATACGAAACAAGTCATCACGACATGCGCCGTATGGTCAGCTC
CAGACCCCAGACATGCCTTCTCAGCCATGGAAGTCCATCACATGGGACTTTGTGGTCAAACTA
CCACTCTCAAAGGATCCTACTACAGGAATTGACATACACGGAGTACCAGATGAGATAATCTCG
GATCGAGACAAGCTCTTTACCTCGAAATTCTGGACTACCTTATTAGCACTTATGGGTATCAAGA
GAAAGCTATCGACATCTTTCCACCCACAAACAGATGGTCAAACAGAGAGGACCAATCAGACA
ATGGAAGCATATCTTAGATGCTATGTAAATTATCGACAAGACAATTGGGTAGAGCTATTACCCAT
GGCACAGTTCGCATACAATACATCGGAAACGGAAACCACGAAAATCACCCCAGCACGAGCTA
ATTTTGGGTTTAATCCACAAGCGTATAAAATCCCGATACCACAAGAAGTTAATGCCGAATCAGC
AATAGTACAAGTCGAACAGCTGAAAGATCTCCAAGAGCAACTGGCTCTTGATCTAAGATTCAT
ATCTTCCAGAACAGCAGCGTACTACAATACGAAACGTAGTATGGAACCTACGCTTAAAGAGGG
GGATAAAGTTTATTTGCTACAACGAAACATCGAAACCAAGAGACCAAGCAATAAACTCGACC
ACAGGAAACTAGGACCATTCAAGATTGATAAGGTAATAGGAACG
SEQ ID NO: 24-LTR for siR5
>BC1G_06254.1 retrotransposable element Tf2 1 protein type 1
(Transcript: BCIT_06254)
ATGGCATCCAGAGCCACCGCCACAGGTCAGTCTACCGGAGATACCAACGACATCGAGATGAC
CGATGCCCCAAAGGAGATCACTATCAACGAAACACTTAAGATCGCCTTACCAGACAAGTACC
AAGGTAGTCGACAAGAGCTCGATACTTTCCTCTTACAACTTGAGATCTACTTCCGATTCAATGA
AGACAAGTTCACTACCAAGGAATCCAAGAGCATATGGGCTGCATCATACCTCCGAGGTGAAG
CAACCAAATGGATTCAACCATATTTGCGCGACTATTTCGAGCATGACGATAAGGATCGCATGCA
ACCCACCCGAACAATCTTCAATAGTTTTGAAGGATTTAAGACAGAGATTCGTAGAATCTTCGG
AAATTCCAACGAGTTAGAGGTAGCGGAAGATAAGATCTTCAACCTCAAGCAGACAGGATCAG
CATTGAAATATGCTACGGAATTTCGAAGATATGCTGGAACAACCAAGTGGGACGAAATCGCTA
TCATGAGTCACTACCGTAAGGGACTCAAACCAGAAGTCAGACTAGAGTTAGAAAGATCTGCC
GAGAGTACAGATCTGAACGATCTAATTCAGGACTCCATCGAATCAGATGATCGTCTCTATAGAT
ATCGACAAAGCCAAAGATCATACAAACCCCAAGGAAACCAAAAGCAAGGGCGTTACCGCAA
GAATGAGGGTAGACCACGTTACAATCCACAGAGATACGGAGACCCCATGGAACTAGACGCCA
CGCACTACACAAACGGGAACGATGACTCAGAAAAGAGACGAAGACGAGAAAACAACTTATG
CTTTGAATGTGGAAAAGCAGGGCACCGAGCAGTAGACTGCCGAAGCAAGAAGACAGGAGGA
AAAAGGGGCAACTTCAAACCTAAGTTCGGCAAGGGCCAACTTAACGCCACCTTTGCCATCTC
AGAAAACTCAACTAAAACCGAAAATACTGAGACTTTCACCGTTGAGGAATTTCAGCAATTACT
AAAGGAATTACCACGAAATAAAGAGGGCATGAATGCAATAGACTTATGGGAACAAGAGTATTA
CAGAACCCCAACACCCTCTGTGACAGAAGAAAGTCACCAGGACGAGGCAGAAGCGGACCAC
GCCACGATGAGCTGGACAGCTTGCTATGATGAATTCTGCGGAATCCATCGATCAGATAAAGAA
GCAACCGGATGGTTCCCCAAGAAAAGGAAGACGAAGAACCATCAGAATAATGTAACATGCGA
GGATTTAACTCCCAATATAACTTCGCAAGAAGTTCGCAAAGTTACCCAGCAGTTGAATGCTAC
GGGACAGGCAGGACAAGTGTACTGCAAGGTCCAGATAAATGGACACATACAATCAGCCATGA
TAGATTCAGGGGCTACAGGAAATTTTATTGCACCAGAAGCTGCAAAGTACTTGGAAATACCAC
TTCAAACGAAACAACATCCCTACCGATTGCAGGACACGCTAGAGGCGTCCGCGAGACGTAAC
ACGCGCCAAGGGGAGTTGAACGCGAACAACACCGGCGACGTAGGACACCCAGTCCAGGGTC
CTCCATTAAGAGCGAAGGCCAGTACACCTCCTCTACAAATGCAGAAGCCAACGACACGGCAC
GAAATCGCAATCGAGGCAAAAGAAAGGCCTACGATACCAGAACAGTACAAGAAATATGAACA
TGTTTTCAAAGAACCAGGGATCCATGAGGCTTTACCGGAACACAAGCCATGGGATCATGAGAT
AATATTGGAGGAAGGCAAGATGCCTGTGCACACCCCAATTTATTCAATGTCAGCCGATGAGTT
AAAAAGGCTCAGAGAATACATCGACGACAATTTAGCCAAGGGATGGATCAGGGAATCCGCGT
CCCAAGTGGCCAGTCCAACTATGTGGGTACCCAAGAAGGATGGACCCGATAGACTAGTTGTA
GACTATAGAAAGCTTAACGCACTCACTAAGAAGGATCGATATCCACTTCCATTAGCTACGGAAT
TAAGAGATCGATTAGGCGGAGCTACGATATTCACCAAGATGGACCTACGTAATGGTTACCACTT
GATCAGAATGAAGGAAGGCGAAGAATGGAAAACCGCTTTCAAAACAAGATACGGGCTATACG
AGTACCAAGTTATGCCATTCGGGCTAACCAACGCACCAGCTACTTTCATGAGGCTTATGAACA
ATGTGTTGTCACAATATTTGGATACTTGCTATCAAGGAAGCTTCGAATGGACTGCCAAAGCAC
AGGAGTCATTCGATACGCTCAAGCAAGCAGTGGCAGAAGAACCAATACTGTT
GACTTTTGACCCAGAGAAAGAAATCATAGTGGAGACGGACTCCTCGGATTTCGCTATAGGAGC
AGTTCTGAGCCAACCGGGCCAGAATGGAAAATACCAGCCAATCGCATTCTACTCCCGAAAACT
ATCACCAGCTGAGTTAAATTACGAGATATATGACAAAGAATTACTGGCAATAGTCGATGCATTT
AGAGAATGGCGAGCATATTTGGAAGGATCGAAATACACAGTACAGGTATATACAGATCATAAG
AACTTGGTTTACTTCACCACAACGAAGCAGTTAAACAGACGACAGGTCAGATGGTCGGAGAC
CATGGCCAACTACAACTTTAGAATTTCATATGTCAAAGGATCAGAAAACGCTAGAGCCGACGC
TCTTAGCCGAAAACCAGAATATCAAGAAAACAAAACGTACGAGTCATACGCTATATTCAAGAA
AGACGGCGAATCACTGGTCTACAATGCACCACAGCTTGCAGCAACACACCTGTTGGAAGACA
ACCACCTCAGGAAACAGATCCAATCACACTACAACAAGGATGCTACTGCCACACGCATACGC
AAGACAATAGAACCAGGATTCACTATAGAAGATGATACCATATACTTTCATGGAAAAGTATAC
ATTCCGAGTCAAATGACCAAGGAATTTGTGACGGAACAACATGGGTTGCCGGCACACGGAC
ATCAAGGGATTGCAAGAACATTTGCAAGAATCCGGGAAATCAGTTACTTCCCACGAATGAGA
ACGATAGTTGAAGAAGTTGTTGGAAATTGTGACACCTGCATACGAAACAAGTCATCACGACAT
GCGCCGTATGGTCAGCTCCAGACCCCAGACATGCCTTCTCAGCCATGGAAGTCCATCACATGG
GACTTTGTGGTCAAACTACCACTCTCAAAGGATCCTACTACAGGAATTGAGTACGACGCGATA
CTCAATATAGTAGACAGGCTAACGAAATTTGCATATATGATACCATTCAAGGAAACATGGGATG
CTGAGCAACTAGCATATGTGTTCCTAAGGGTCATAGTAAGCATACACGGAGTACCAGATGAGA
TAATCTCGGATCGAGACAAGCTCTTTACCTCGAAATTCTGGACTACCTTATTAGCACTTATGGG
TATCAAGAGAAAGCTATCGACATCTTTCCACCCACAAACAGATGGTCAAACAGAGAGGACCA
ATCAGAC
AATGGAAGCATATCTTAGATGCTATCGTATAAAATCCCGATACCACAAGAAGTTAATGCCGAAT
CAGCAATAG
SEQ ID NO: 25-LTR for siR5
>BC1G_08449.1 retrotransposable element Tf2 1 protein type 1
(Transcript: BC1T_08449)
ATGGCATCCAGAGATACCGCCACAGGTCAATCTGCCGGAGACACCAACGACATCGAGATGAC
CGATGCCCCAAAGGAGATCACTATCAACGAAACCCTTAAGATCGCCTTACCAGACAAGTACCA
AGGTAGTCGACAAGAGCTCGATACTTTCCTCTTACAACTTGAGATCTACTTCCGATTCAATGAG
GACAAGTTCACTACCAAGGAATCCAAGAGTATATGGGCCGCATCATACCTCCGAGGTGAAGCA
ACCAAATGGATTCAACCATATTTGCGCGACTATTTCGAACATGACGATAAGAATCGCATGCAAC
CCACCCGAACAATCTTCAATAGTTTTGAAGGATTTAAGACAGAGATTCGTAGAATCTTCGGAA
ATTCCAACGAGTTAGAGGTAGCGGAAGATAAGATCTTCAACCTCAAGCAGACAGGATCAGCA
TTGAAATATGCTACGGAATTTCGAAGATATGCTGGAACAACCAAGTGGGACGAAATCGCTATC
ATGAGTCACTACCGCAAGGGACTCAAACCAGAAGTCAGACTGGAATTAGAAAGATCTGCCGA
GAGTACAGATCTGAACGATCTAATTCAGGACTCCATCGAATCAGATGATCGTCTCTACAGATAT
CGACAAAGCCAGAGATCATACAAACCCCAAGGAAATCAGAAGCAAGGGCGTTACCGCAAGA
ATGAGGGTAGACCACGTTACAATCCACAGAGGTACGGAGACCCAATGGAACTAGACGCTACG
CACTACACAAACGGGAACGATGACTCAGAAAAGAGACGAAGACGAGAAAACAACTTATGCT
TTGAATGTGGAAAAGCAGGGCACCGAGCAGCAGAGTGCCGAAGCAAGAAGACAGGAGGAA
AAAGGGGCAACTTCAAACCTAAGTTCGGCAAGGGCCAACTTAACGCCACCTTTGCAATCCCA
GAAAACCCAACTAAATCCGAAAATACTGAGACTTTCACCATTGAAGAATTCCAGCAATTACTA
GAGGAATTACCACGAAATCAAGAGGGCATGAATGCAATAGACTTATGGGAACAAGAGTATTAC
AGAACCCCAACACCCTCTGTAACAGAAGAAAGTCACCAGGACGAGGCAGAAGCAGACCACG
CCACAATGAGCTGGACAGCCTGCTATGATGAATTCTGCGGAATTCATCGATCAGATAAAGAAG
CAACCGGATGGTTCCCCAAGAAAAGGAAGACGAAGAACCATCAGAATAATGTAACATGCGAG
GATTTAACTCCCAATACAACTTCGCAAGAAGTTCGCAAAGTTACCCAGCAGTTGAATGCTACG
GGACAGGCAGGACAGATATACTGCAAAGTTCAGATAAATGGACACATACAATCAGCCATGATA
GATTCAGGGGCTACAGGAAATTTTATTGCACCAGAAGCTGCAAAGTACTTGGAAATACCACTT
CAGACGAAACAACACCCCTACCGATTGCAGGACACGCTAGAGGCGTCCGCGAGACGTAACA
CGCGCCAAGGAGAGTTGAACGCGAACGACACCGGCGACGTAGGACACCCAGTCCAGGGTCC
TCCATTAAGAGCGAAGGCCAGTACACCTCCTCTACAAATGCAGAAGCCAACGACACGGCACG
AAATCGCAATCGAGGCAAAAGAAAAGCCTACGATACCAGAACAGTACAAGAATTATGAACAT
GTTTTCAAAGAACCAGGGATCCATGAGGCTTTACCGGAACACAAGCCATGGGATCATGAGATA
ATATTGGAGGAAGGCAAGATGCCTGTGCACACCCCAATTTATTCAATGTCAGCCGATGAGTTA
AAAAGGCTCAGAGAATACATCGACGACAATTTAGCCAAGGGATGGATCAGGGAATCCGCGTC
CCAAGTGGCCAGTCCAACTATGTGGGTACCCAAGAAGGATGGACCCGATAGACTAGTTGTAG
ACTATAGAAAGCTTAACGCACTCACTAAGAAGGATCGATATCCACTTCCATTAGCTACGGAATT
AAGAGATCGATTAGGCGGAGCTACGATATTTACCAAGATGGACCTACGTAATGGTTACCACTT
GATCAGAATGAAGGAAGGCGAAGAATGGAAAACCGCTTTCAAAACAAGATACGGGCTATACG
AGTACCAAGTTATGCCATTCGGGCTAACCAACGCACCAGCTACTTTCATGAGGCTTATGAACA
ATGTGTTGTCACAATATTTGGATACTTGCTGTATATGCTACTTGGACGACATCCTAGTATATTCA
AACAACAAGGTTCAACACATTAAGGACGTTAGCAACATCCTCGAAAGCCT
ATCCAAGGCAGACTTGCTGTGCAAACCAAGCAAATGCGAATTCCATGTCACAGAGACAGAAT
TCTTGGGATTCACCGTATCAAGCCAAGGGCTCAAGATGAGCAAAGGCAAGGTTAAGGCAGTG
CTCGAATGGAAGCAGCCGACCACAATCAAGGAAGTACAATCCTTTCTAGGGTTCGTCAACTTC
TACAGAAGATTTATCAAAGGTTATTCAGGGATTACTACACCCTTGACCACGTTAACCAGAAAA
GATCAAGGAAGCTTCGAATGGACTGCCAAAGCACAGGAGTCATTCGATACGCTCAAACAAGC
AGTGGCAGAAGAGCCAATACTATTGACTTTTGACCCAGAGAAAGAAATCATAGTGGAGACGG
ACTCCTCGGATTTCGCTATAGGAGCAGTTCTGAGCCAACCGGGTCAGAATGGAAAATACCAGC
CAATCGCATTCTACTCCCGAAAACTATCACCAGCCGAATTAAATTATGAAATATACGACAAAGA
ATTACTGGCAATAGTCGATGCATTTAGAGAATGGCGAGTATATTTGGAAGGATCGAAATACACA
GTACAGGTGTACACAGATCATAAGAACTTGGTTTACTTCACCACAACGAAGCAGTTAAACAG
ACGACAGGTCAGATGGTCGGAGACCATGGCCAACTACAATTTTAGAATTTCATATGTCAAAGG
ATCAGAAAACGCTAGAGCCGACGCTCTTAGCCGAAAACCAGAATATCAAGAAAACAAAACGT
ACGAGTCATACGCTATATTCAAGAAAGACGGCGAATCACTGGTTTACAATGCACCACAGCTTG
CAGCAACACACCTGTTGGAAGACAACCACCTCAGGAAACAGATCCAATCACACTACGACAA
GGATGCTACTGCCACACGCATACGCAAGACAATAGAACCAGGATTCACTATAGAAAATGATAC
CATATACTTTCATGGAAAAGTATACATTCCGAGTCAAATGACCAAGGAATTTGTGACGGAAC
AACATGGGTTGCCGGCACATGGACATCAAGGAATTGCAAGGACATTTGCAAGAATACGGGGA
ATCAGTTACTTCCCACGAATGAGAACGATAGTTGAAGAAGTTGTTGGAAATTGTGACACCTGC
ATACGAAACAAGTCATCACGACATGCTCCGTATGGTCAGCTCCAGACCCCAGACATGCCTTCT
CAGCCATGGAAGTCCATCACATGGGACTTTGTGATCAAACTACCACTCTCAAAGGATCCTACT
ACAGGAATTGAGTACGACGCGATACTCAATATAGTAGACAGGCTAACGAAATTTGCATATATGA
TACCATTCAAGGAAACATGGGATGCTGAGCAACTAGCATATGTGTTCCTAAGGGTCATAGTAA
GCATACACGGAGTACCAGATGAGATAATCTCGGATCGAGACAAGCTCTTTACCTCGAAATTCT
GGACTACCTTATTAGCACTTATGGGTATCAAGAGAAAGCTATCGACATCTTTCCACCCACAAAC
AGATGGTCAAACAGAGAGGACCAATCAGACAATGGAAGCATATCTTAGATGCTATCGTATAAA
ATCCCGATACCACAAGAAGTTAATGCCGAAT CAGCGATAG
SEQ ID NO: 26-LTR for siR5
>BC1G_16170.1 hypothetical protein similar to integrase
(Transcript: BCIT_16170)
ATGACCAAGGAATTTGTGACGGAACAACATGGGTTGCCGGCACACGGACATCAAGGGATTG
CAAGAACATTTGCAAGAATCCGGGAAATCAGTTACTTCCCACGAATGAGAACGATAGTTGA
AGAAGTTGTTGGAAATTGTACACACCTGCATACGAAACAAGTCATCACGACATGCGCCGTAT
GGTCAGCTCCAGACCCCAGACATGCCTTCTCAGCCATGGAAGTCCATCACATGGGACTTTGT
GGTCAAACTACCACTCTCAAAGGATCCTACTACAGGAATTGA
SEQ ID NO: 27-Botrytis LTR genomic DNA sequence
>B. cinerea (B05.10) Botrytis cinerea supercontig 1.56 [DNA]
215700-227000 +
CAAAGGGGGCATTACGCTTCCAACTGCCGAAACCCTGTTGTATGTCAACACTGTAAAGGA
AGTCACGGATCCAGAGAGTGCCCAGGAACTATGTCACAGCCTTCCCGACAGGGAAACGCT
TAGACCCAGCTGTTATTCTGAGCGTCCCACTGACGCTGGGTCCCCAAATAGAAGGACGTA
CTACCTTTACCATTATAGCAATGTTCCAACCAAAAGATAAGCCGATAGCGCTTCGATGCC
TTATCGACTCAGGAGCACAAGCCAACATCATCCAACAATCCAAGTGTATCGAATGGGACT
GGCTGCCTATTAAGAAAGGAACAGCTTTAGTATCTGCGAACGGTACCACGATGCCGTCGT
ATGGTAACCATCAGTTCCCCGTCGAAGTAAAAGATCAAAAGGGAGAGAAGAGAACCTTCA
CCCACGAGTTTACTGCTGCTGTACTAGACTTACCCAAAATCGATGCTATATTTGGATTAC
CCTGGCTACAAGCGGTAAACCCAGATATCGACTGGAAATCGACGTCTCTTCACTATCGCC
CCTCTCTTAGCGACCTCGAAATGATTTCTGCAAGCGAACTCTATAGCGAAGTGAAAAAGG
GCGTCCATGTATATGTTATACTACCAGAGATCCAGCCCCATTACCGTAGAGACAACGGGT
ACCGCCGGGTACTCACGCTCTCCACACTAAATATCCCCGAAGAATACCAAGAATACCAAC
AAGCCTTTTCCGAGGAAGAAAGCAGTACTCTACCAGAACACCACTCGATGGAGCATCGCA
TTGATCTCGAAGCCGATTCGAAACCTCCTTGGGGGCCAATCTATTCTTTATCTGAAGAGG
AATCAATAGTATTAAGGGAATACTTAGTAGAATATCAAAAAAAGGGATGGATAAGGAGGT
CCATTAGTTCGGCAGGAGCGCCAATCATGTTTGTTCCCAAGAAGGGGGGAGGCTATCGGC
TTTGTGTCGACTACCGGGGTCTAAATAGGATAACCAAAAAGGATCGAACCCCGCTACCCC
TAATCAGCGAGTCCTTAGACCGACTTCGACAAGGTGTCGTCTTCACTAAATTGGACCTGC
GAGATGCCTACCACCGTATTCGTATCAGGGAAGGCGACGAATGGAAGACGGCGTTCCGCA
CGCGGTACGGGCAATTCGAATACTTAGTTATGCCATTCGGCCTGACCAATGCTCCAGCAA
CGTTCCAAACATACATCAATCAAGCACTGTCAGGCTTGACAGACACCATATGCGTAGTGT
ACCTAGATGATATCCTGATTTACTCTGAGGATAGAGAAAGCCACACGCGGGATGTCCGCA
GGGTCCTCGAACGCCTTATAGAATACAAGCTGTTCGCAAAACTGAAAAAATGTGTCTTTT
ACACCCATGAGGTTGAATTCCTAGGATTCGTCGTCTCGGGAGCGGGAGTGACGATGGAAT
CCAGCCGCATTCAAACTATTATAGAATGGCCAACACCTACAAACCTTAGGGAGCTACAGG
TGTTCCTGGGCTTCGCGAACTTCTATCGACGGTTTATCAGGACCTATTCGACGGTAGCCC
ACGGGATGACCGCCCTTATGAAGGGAACAAAGAAAGGTAAAATGGTAGGGGAGTTTATAT
GGACAAAGGAGGCCCAAGATGCATTTGAGGCACTAAAGAAAGCATTCACCACGGCACCGA
TACTCAAGCACTTCGAACCATCGCTCCGCATCATGGTCGAAACCGACTCGTCGGTGTTTG
CTCTAGGATGCATCCTATCGCAACTATTCGAAGGAGGGACTGCAGAAGCACCGATACGAC
GGTGGCACCCCGTCGCGTTCTATTCGAGAAAGCTGAACCCTGCAGAACAACGATACTTCA
CTCACGATCAGGAATTATTAGCAATATACACTGCATTCATGCAATGGCGCCATTACTTGA
TAGGTAGTCGGCACACAATCGTGGTGAAATCGGACCATAACAGCTTACAACATTTTATGG
TGAAAAAGACCCTCAATGGCAGACAAGCTAGATGGGCGGAAGTACTAGCAGCCTACGACT
TCGAAATAGTGTACAGGGCAGGGAAACTGAATCCAGCCGACGGGCCATCGCGCCGCCCCG
ACTACGCTACCGACACGGAGGGTATCAATGATATGCTACCCACACTCCAGAATAAATTAA
AAAGTACCGCAGTTATCGCGAGTTTATTTTACGAATCCACCGTGAAAACGGAACCCCTGC
GTATTGCTATTAGTCGCTTGCAAAGGGAAGGGTATAGCTTGCCATTACGTGGACAGTTAG
TTTCACTGGTAAAAACTGGTTGCAAACAGTCGATACCACGTCGGATTGCCAGTGTTTTCG
CATCCGACGAAACGGCATTCGAACCTATATCGGAGTCGATGGGAAAAGCTTTATTGCGGC
TTCAGAAAGAAGACGATTTTATAAAGAATAAAGAGTACCTAAGACAAAGATTACGTTCCG
CCGGAGACGCCTCACCACGGCAGGTGGGCGCCGACGAGCTCCTTAGACACAAGGGGAGCG
CGTACGTACCGCCAGACAGCGCTCTCAGAGCAGAAATCTTAGAAACGCATCACGATGACC
CTATTGGAGGTCATTGGGGTGTCGCTAAAACATTGGAAATACTGAAGTCTAAATATTATT
GGCCTTCAATGAGAAAAGACGTCAAACAACATGTCAAAACATGTGCGGTATGCCAGCGAA
CCGCTATCAAAAGACATAAGCCACACGGCGAGTTACAGACCCTCCCTATTCCAAAAGGAC
CCTGGAAAGAGATAACTATGGATTTTATTACAGATTTACCTCCTTCGAAACACGGAAAAC
ACGTATACGATTCTATTCTAGTAGTAGTCGACAGGTTCACGAAGCTAGCCCGATATATCG
CCGTCAACAAGACGATATCGTCTCCTGAATTAGCTGACACTATGGTCAGCACAGTATTTA
AAGACTTTGGTGTGCCAGAGGGCATAGTCTCCGATAGGGGACCGCAATTCGTCAGTAAAT
TTTGGAGTAGCCTAATGTTTTACTTGCGAATCCGTCGTAAGCTGTCGACGGCGTTCCACC
CGCAGACCGACGGTCAAACCGAACGACAAAACCAAAATTTGATTCACTATATAAGTTGCT
ACACCAACTATAGGCAAGACGACTGGGCATCGCTATTGCCCCTTGCTGAATTCACATATA
ACGCGACATGGCACAGTACAACCAATACAAGCCCATTCCAGGCTATGTATGGGTTCCAAC
CCACATTCCATTATATCGGCGAGGACGCCGATTTAGAGGGAAGGGCGCCGGCAGCACGCG
AGCGCATCGACGCTTTAGAGAAAGAAAGAGAAAAGCTGAAAGAATTCTGGAAATCGGCAA
CCAATCAGAAAAACAAGAACACTACGAAGGGGTCACCACAGCGATATAGCATCGGGGACA
AGGTGATGCTAAGCACAAAGAACATTAAACAACTGCGACCTAAGAAAAAATTCTCCGATC
GATTTATAGGCCCCTTTGTCGTGACGGGTATAAAAACCAGCGGGCAAGCATACGAACTTA
GATTACCGCCCACCTACAAGATCCACAATGTATTCCACGTCTCTTTACTCGAACCATGGC
ACGAACGACAGGGTACCGCCGACCCGCCGCCGCCAGAAGAAATTGACGATCACATAGAGC
ATGAGGTGGAAAGGATTTTAGCACATAGAAAAAGAGGCAGAGGTGTGCAATACCTGGTGC
GATGGAAGGGCTACCAACCGGCGGAAGACACGTGGGAAGCACCCTACAACCTAGAAAATG
CGAAAGCAGCGATGGGAGAATATCATAAAGAAGAAGCATTACCAATACAGAAAAAGAAGA
GAACAAGAAAAAAGCTTAAAAATACTTGATACAAAAAGACCTCACGAGACCCACACCCAG
AACGCATGCATCACACCAAGCTACCCAAAAAGGACTATCCAACAAAGAAACCAGAAAGGA
CAACTCCTCCGAACCCACAGCATACCGACAACCAAACCCAAGTGACCCATCACGCAAAGA
GAACTCTGGAAGGTCGAAATCAGTTCCCTAATTCATCTGCCTGATCCAGGAGGTCAGCTG
CAATATCTCGATCGCCAAGAAGGACAGAACCTACATCGCTGGCATATCCCCCGGGACTCG
CGAGCCCGATCTGATCAACCTCACCCCCCCCACTGATCTCATCTTGATCACCGACTCCTT
CCTCCTCGTTCTCGCGAATCTCGCTGACAGGGCGAGCTCCTGTCGGAAAACCAGCGGCAA
CCCGTTGCCTCTTGGAGACCCGCTCCACATCAGAATTCTCAGAGCGAAGCCTTTGGGGCG
AATCTGCTTGACTATCAGACTCACCAATATAGACTTGTTGAACAGTGCTGGGAGCCCTCT
TTCGGGATTTAAGAGAGCCTACGGGATGAGAGCGGGGTGTCGGTGTGTTGCGAGAGGCAA
GCTGCGACGACTTAGAAGCGGAAAGGGGAATGGCGGATCTAGTCGCGAGCTTATCGGACC
GAGAACGACGAGGCGTAGCAGGAGGAGCGTTCTTTCTACCCAAGCTTCCAAACAATAAAG
CCGGTACACTCTCATCCAAGGGGTGACGCGGGGCACCGGTGGTCTGATGAGGTTGATCCG
ACACATCATCCTCTTCAGGAGCAGACTCTTCGGATTCATTGGGATCCACGATTACACTCC
TCTTCAACTTCGCAGAACCTTTCCGGAGGCCTTGCTGAGCCGTCGTGAAGACAGCGGGGC
CCGTCCGGCTCGATGAAGCAGAAGCATGACGGCGACCTAGGGAACCAGCTACCATATTCA
GGCCCTGGTTACCACCTGGGGGCCCCGGAGGAGCTTGAACACCCTGCAGTGCGTCGGCGA
CAGCGCGCACAGCGCGAGGGTGTATAACCGGAGCCATGAACTCCCCTTCTTCGTCCGAGT
TTAAGGCGGCGATTAATGGTGCAGTAGGAGCTGCGGCGGCGACGTCCAAGGCAGGCAGGT
TATTCTAAGAACCATCAAATCAGCTATCAACAGCACCAAGGGGCAAGAGCAGGGCAACCG
ACAACGAATCGATACGAATCCACAATGCGCTCCATAAGATCGCCAATTCTACTCAATTTG
GACACCAGAAGAAGAGTCAATTCCTCTGCGGACTGCGGTTTGTGAGTCCCCCCTTGTTTA
ACCTCTTTTCGAAGGAAAGCCTCCACTGCCTTGACGTATCGATTACACCGGCGAATCACC
ACCTCGGAGGCGTCCTCATCCTCTTGGCTATTATCCGGAGTAAGGCACCAAAAGGCATGA
GTCTGATATAAGAGGTTCACGTCGGGAACAAAGCGAGGAGGTATCGGGAGGCAGACTTTC
TTTTGTTTGCGACAATAACTACATTTAGCAGCAGGACCTTTATCAAAATGGCAAAATTCG
TCGTTAGAAGAGACGGCAATTCGCTTAGAACATCGAAGGCAAGTAGGGATAACTAACGCG
GAAGGGGCCGCTGCGGACCGATCAGCGATAGCAGCCGGAGCGATGGGTTGCAGTGCAGCC
ATCTTCGTATGAGTAAATAAGGGGGAATAATCTGATTGTGGGAGATATATCAGAGGCAAG
AAGACCCCCCTTATAGAACTATCGGTGATCTGCCGGTAAGGCGGTGAGGCGCGTAAGAAT
GCCGCCGTTTGCTTGTTTATTGTTTGTAATGCCTAAACAAGATTGGAATTGCTTTTGGAA
TGCGGCGCAGGGTCGGGCATGCAGCGACGCGACGACGCGACCCACATTCCGAGTAAACAA
TACGGAAGGAAGCAAACACTTCTCGGGACGCGAAGTGTAAAGAGAGGGGCTCTGTTACGG
GACAAAACGTGACCGGCTCAATTAGGCACGTGACAGTGGACCTCTCGGGTCTACTGCGTG
CCGAATGGGGCCCGCACACGTATAAATTGTATAATTTGCATAGTTATAGAAAAGCAATGA
AAAGTCTTGGTGCCACAATATACTAGTTGATTCATTTGTTACGGAGGTACCCGCACCGCA
ACATGGATTATAAGATAAACCTAAGGCCTTGGTGTTGGAACCTACGAAAACAGCACTGTA
GGGACAGTTGAATTAAAGGGTAACTAAAGATAGCAGTAACCGAATCAATAAGCAATGATT
AAAAGATAGGTACCTATCTTTTGTTGGCACCTACCCTACAGTAGGCACAGGAGGGATAGC
GGTTATAGGTTATCTAGTAAGCACAGGTTAGATAAGCAGTAGTATCATGTAGGTCACGGG
GCAAGTGTCACGTGATGGATAGACAGGATAGGCAGGCTATCCAGGCTATCCGTGGATAGA
CAGGATAGACAGTCTACCCAAGCTATCCAGACGAGAACGAAGGTCTATATAAGGGAATGG
GTTTCATTACAATGTAGAGCTTCGTGCTCAAGAACAATCATTAGTTTCATTACTATAGTT
ACGAGAATTGCAACCAGTTACAACCTTATTGAATTCCTACTTGAAGTCTAGTCTAAACCA
CCTCGAGAGATCTCTAGACACTTCCACGTGACCCTAGAGGCAGCTCCCGTAACACTTTGA
GCACCCTTTCTGCTTCAAGTACCGATTCGATAACCAACCGCTAATATGGCATCCAGAGCT
ACCGCCACAGGTCAGTCTACCGAAGATACCAACGACATCGAGATGACCGATGCCCCAAAG
GAGATCACTATCAACGAAACACTTAAGATCGCCTTACCAGACAAGTACCAAGGTAGTCGA
CAAGAGCTCGATACTTTCCTCTTACAACTTGAGATCTACTTCCGATTCAATGAAGACAAG
TTCACTACCAAGGAATCCAAGAGCATATGGGCTGCATCATACCTCCGAGGTGAAGCAACC
AAATGGATTCAACCATATTTGCGCGACTATTTCGAGCATGACGATAAGGATCGCATGCAA
CCCACCCGAACAATCTTCAATAGTTTTGAAGGATTTAAGACAGAGATTCGTAGAATCTTC
GGAAATTCCAACGAGTTAGAGGTAGCGGAAGATAAGATCTTCAACCTCAAGCAGACAGGA
TCAGCATTGAAATATGCTACGGAATTTCGAAGATATGCTGGAACAACCAAGTGGGACGAA
ATCGCTATCATGAGTCACTACTGTAAGGGACTCAAACCAGAAGTCAGACTAGAGTTAGAA
AGATCTGCCGAGAGTACAGATCTGAACGATCTAATTCAGGACTCCATCGAATCAGATGAT
CGTCTCTATAGATATCGACAAAGCCAAAGATCATACAAACCCCAAGGAAACCAAAAGCAA
GGGCGTTACCGCAAGAATGAGGGTAGACCACGTTACAATCCACAGAGATACGGAGACCCC
ATGGAACTAGACGCCACGCACTACACAAACGGGAACGATGACTCAGAAAAGAGACGAAGA
CGAGAAAACAACTTATGCTTTGAATGTGGAAAAGCAGGGCACCGAGCAGTAGACTGCCGA
AGCAAGAAGACAGGAGGAAAAAGGGGCAACTTCAAACCTAAGTTCGGCAAGGGCCAACTT
AACGCCACCTTTGCCATCTCAGAAAACTCAACTAAAACCGAAAATACTGAGACTTTCACC
GTTGAGGAATTTCAGCAATTACTAAAGGAATTACCACGAAATAAAGAGGGCATGAATGCA
ATAGACTTATGGGAACAAGAGTATTACAGAACCCCAACACCCTCTGTGACAGAAGAAAGT
CACCAGGACGAGGCAGAAGCGGACCACGCCACGATGAGCTGGACAGCTTGCTATGATGAA
TTCTGCGGAATCCATCGATCAGATAAAGAAGCAACCGGATGGTTCCCCAAGAAAAGGAAG
ACGAAGAACCATCAGAATAATGTAACATGCGAGGATTTAACTCCCAATATAACTTCGCAA
GAAGTTCGCAAAGTTACCCAGCAGTTGAATGCTACGGGACAGGCAGGACAAGTGTACTGC
AAGGTCCAGATAAATGGACACATACAATCAGCCATGATAGATTCAGGGGCTACAGGAAAT
TTTATTGCACCAGAAGCTGCAAAGTACTTGGAAATACCACTTCAAACGAAACAACACCCC
TATCGATTGCAGTTAGTTGATGGACAGCTAGCAGGGTCTGACGGAAAGATTTCGCAGGAG
ACAATCCCAGTACGAATGGGCATAACCCAACATACAGAGGTTATACAGCTTGACGTTGTG
CCATTGGGCCAACAACAGATCATCTTAGGAATGCCATGGTTGAAGGCACATAATCCGAAA
ATAGATTGGGCACAAGGAATTGTGACATTTGATCAGTGCAAAAGCGGTCACAGGGACACG
CTAGAGGCGTCCGCGAGACGTAACACGCGCCAAGGGGAGTTGAACGCGAACAACACCGGC
GACGTAGGACACCCAGTCCAGGGTCCTCCATTAAGAGCGAAGGCCAGTACACCTCCTCTA
CAAATGCAGAAGCCAACGACACGGCACGAAATCGCAATCGAGGCAAAAGAAAGGCCTACG
ATACCAGAACAGTACAAGAAATATGAACATGTTTTCAAAGAACCAGGGATCCATGAGGCT
TTACCGGAACACAAGCCATGGGATCATGAGATAATATTGGAGGAAGGCAAGATGCCTGTG
CACACCCCAATTTATTCAATGTCAGCCGATGAGTTAAAAAGGCTCAGAGAATACATCGAC
GACAATTTAGCCAAGGGATGGATCAGGGAATCCGCGTCCCAAGTGGCCAGTCCAACTATG
TGGGTACCCAAGAAGGATGGACCCGATAGACTAGTTGTAGACTATAGAAAGCTTAACGCA
CTCACTAAGAAGGATCGATATCCACTTCCATTAGCTACGGAATTAAGAGATCGATTAGGC
GGAGCTACGATATTCACCAAGATGGACCTACGTAATGGTTACCACTTGATCAGAATGAAG
GAAGGCGAAGAATGGAAAACCGCTTTCAAAACAAGATACGGGCTATACGAGTACCAAGTT
ATGCCATTCGGGCTAACCAACGCACCAGCTACTTTCATGAGGCTTATGAACAATGTGTTG
TCACAATATTTGGATACTTGCTGTATATGCTACTTGGACGACATCCTAGTATATTCAAAC
AACAAGGTTCAACACATTAAGGACGTTAGCAACATCCTCGAAAGTCTATCCAAAGCAGAC
TTGCTGTGCAAACCAAGCAAATGCGAATTCCATGTCACAGAAACAGAATTCTTGGGATTC
ACCGTATCAAGCCAAGGGCTCAAGATGAGCAAAGACAAGGTTAAGGCAGTGCTCGAATGG
AAGCAGCCAACCACAATCAAGGAGGTACAATCCTTTCTAGGGTTCGTCAACTTCTACAGA
AGATTTATCAAGGGTTATTCAGGGATTACTACACCCTTGACCACGTTAACCAGAAAAGAT
CAAGGAAGCTTCGAATGGACTGCCAAAGCACAGGAGTCATTCGATACACTCAAACAAGCA
GTGGCAGAAGAACCAATACTGTTGACTTTTGACCCAGAGAAAGAAATCATAGTGGAAACG
GATTCCTCAGATTTCGCTATAGGAGCAGTTCTGAGCCAACCGGGCCAGAATGGAAAATAC
CAGCCAATCGCATTCTACTCCCGAAAACTATCACCAGCTGAGTTAAATTACGAGATATAT
GACAAAGAATTACTGGCAATAGTCGATGCATTTAGAGAATGGCGAGTATATTTGGAAGGA
TCGAAATACACAGTACAGGTGTATACAGATCATAAGAACTTGGTTTACTTCACCACAACG
AAGCAGTTAAACAGACGACAGGTCAGATGGTCGGAGACCATGGCCAACTACAATTTCAGA
ATTTCATATGTCAAAGGATCAGAAAACGCTAGAGCCGACGCTCTTAGCCGAAAACCAGAA
TATCAAGAAAACAAAACGTACGAGTCATACGCTATATTCAAGAAAGACGGCGAATCACTG
GTCTACAATGCACCACAGCTTGCAGCAACACACCTGTTGGAAGACAACCACCTCAGGAAA
CAGATCCAATCACACTACAACAAGGATGCTACTGCCACACGCATACGCAAGACAATAGAA
CCAGGATTCACTATAGAAGATGATACCATATACTTTCATGGAAAAGTATACATTCCGAGT
CAAATGACCAAGGAATTTGTGACGGAACAACACGGATTGCCGGCACATGGACACCAAGGA
ATTGCAAGGACATTTGCAAGAATACGGGAAATCAGTTACTTCCCACGAATGAGAACGATA
GTTGAAGAAGTTGTTGGAAATTGTGACACCTGCATACGAAACAAGTCATCACGACATGCT
CCGTATGGTCAGCTCCAGACCCCAGACATGCCTTCTCAGCCATGGAAGTCCATCACATGG
GACTTTGTGGTCAAACTACCACTCTCAAAGGATCCTACTACAGGAATTGAGTACGACGCG
ATACTCAATATAGTAGACAGGCTAACGAAATTTGCATATATGATACCATTCAAGGAAACA
TGGGATGCTGAGCAACTAGCATATGTGTTCCTAAGGGTCATAGTAAGCATACACGGAGTA
CCAGATGAGATAATCTCGGATCGAGACAAGCTCTTTACCTCGAAATTCTGGACTACCTTA
TTAGCACTTATGGGTATCAAGAGAAAGCTATCGACATCTTTCCACCCACAAACAGATGGT
CAAACAGAGAGGACCAATCAGACAATGGAAGCATATCTTAGATGCTATGTAAATTATCGA
CAAGACAATTGGGTAGAGCTATTACCCATGGCACAGTTCGCATACAATACATCGGAAACG
GAAACCACGAAAATCACCCCAGCACGAGCTAATTTTGGGTTTAATCCACAAGCGTATAAA
ATCCCGATACCACAAGAAGTTAATGCCGAATCAGCAATAGTACAAGTCGAACAGCTGAAA
GATCTCCAAGAGCAACTGGCTCTTGATCTAAGATTCATATCTTCCAGAACAGCAGCGTAC
TACAATACGAAACGTAGTATGGAACCTACGCTTAAAGAGGGGGATAAAGTTTATTTGCTA
CAACGAAACATCGAAACCAAGAGACCAAGCAATAAACTCGACCACAGGAAAATAGGACCA
TTCAAGATTGATAAGGTAATAGGAACGGTTAATTATCGATTGAAATTACCAGACACAATG
AATATCCACCCAGTATTCCACATATCCTTGCTCGAACCAGCACCACCAGGAGCGCCAAAT
GCGCCATTTACAGAAATCGAACCAGTCAACCCAAACGCCATATACGACGTTGAAACAATA
CTAGATTGTAAATATGTCAGGGGCAAAATCAAGTATTTGATCAAATGGTTAGACTACCCA
CATTCGGAAAACACATGGGAA
SEQ ID NO: 28-Botrytis DCL1 promoter sequence
>B. cinerea (B05.10) Botrytis cinerea supercontig 1.69 [DNA] 45790-46725-
GAAGAGGTTGTTGGCAATATTTTGAAGAAAGCTGAGGCTGATTTGAATGGAGATTAAAAGG
GGAATGAAGCTGCGGGGCCACCGATAGCACAAAAACTACTGAAGATTTGAAGCACGTTAAA
ATTACACTCAGGAATAAACGGATGGCAAGCTTTTCGATCGCCCAAACACGGATCTACGACTA
CGAGTTACGCACGACATGATTTAGCCTTTTGTGTGCAATGATGATTAGATAGCATTGCATTTC
TCGAAATTGACGGCACGACTTTTACGGGCAGATAATATCAAAGATTCCTAGTGAGCAAGCG
GTGATGATACGATGTCATTCCAAAAGTTTTTTCCTCGCGAATTTTATTTCATTTCGAAGGCAT
CTTTGCTTAGCAGCATATTCACCTTTGATGTCCTCTGTAGGGGATGGAGTCTCTAATCTCGCG
GTCACAATGAGACGTGATGCGCTGCGAAGTGGTGACAATTTCCCTTTACTTAGAATAGATCA
TGCACACATGCATGATGCATAGCTAGCTAGTTTTTTATTCAATGATAGTTTAATGACAAACA
CGTATCTAGATATCCTCATTCATGTATCTGTGGGAGGTTGACTTAAGTTATGGCTGACTTGAT
AGTTTCATTATATATGTATATGTGATATCTAAGTAAAGATTAAAGTGAAATCGAAATGCAAC
GCCGAAATTCTATTAATTCCATGAAATGATGTGATATGGCATGACATGATATCCAAACTCCG
ATTTGAAATGCTCCAGCTTCGCTTTCTAAAATTGGTAAAAGGGACATTATTTCGTCTGGTTGT
GGGTTTTCATTTCTGTGCTCCTACTAGGTGTGAATGATAGAGTATGCTGTGGTGTGGTGTGAT
CTCGGAATTTGGAAATTTGAGGGCTGTATATCACCTCATTTCGTGTGTCCGAATTTCTACAGA
CT
SEQ ID NO: 29-Botrytis DCL2 promoter sequence
>B. cinerea (B05.10) Botrytis cinerea supercontig 1.78 [DNA] 26792-27461-
AGAGCATTTGTAGGGGAAGGAGGAAAAATTGAGGAGGAGGATAAGATGAATTTTGATAAAT
TTATTTCCTAACATCAGGTCACAATCTATGAATTACATTTGATAGTATTACGTATGCCGGTCT
GTACACAACACAACCATATAGTAAGGTATCAATCAAATGCGATGGATAGTCATTTCAATTTC
TTAGTGAATAATTACAACGAACCAGTAAAATAGCAATAACTCTGAAAAGCTTCCGGACTGCC
AAAAGGTCTCCAGGACGAGATTATTACGAAGAACCCAAGAATTCGCCTAGGAACCAAGATA
AACAAATCATCGACGTGTTGCACTTCCATCTATGCGACAATTATGCCAAGCGAGCCGCCAGT
TCTTGGGGGTGGAGCGCTAGGAATAGGGGGCCGGATTGCCATATCCTTATCTAGATCTAGAT
GGTATCGATATGATAAATCAATGCAATGGAGAGTTAAAAAGTTATATGCCATATGATTGATA
ATTATTGACAATGCAGGCTATCGCGGGACAATGGTAAATGGTTGTAAAATATGGAGTCTATT
TCCTTAGCTAGCGATAAGATGGGTGGTTTAAACACATCCCGCCTTCTCTTTATCATTCTCCTT
CTCGTATTCATATATCATAATTGCAAAGTAAGGTTGTATTTTGGACTGTG
SEQ ID NO: 30-Verticillium DCL1 promoter sequence
>V. dahliae VdLs.17 supercont1.1 of Verticillium dahliae (VdLs.17) [DNA]
1574620-1574964-
AAGCTGTCAATTGATGCGGAGGGTGAGTGAACGTCTCGTCGGCGGGGCCCCTTGAGGCGAG
CGCCCGTTGGGGGGTGTTGTGGCACTAGGTTCTCTAGGCCGGCGGTGACTTTCATTACTATAT
TAGAAGCAAATACGGCGCCTTCATCACAATAATAAATATCGATCTCGAGTCGATTCCAGACC
CGTTATAAACCTATGTCTGTGCAACCAGTTGGGTGCTAATTTCTTGCATTATCATCATGGATG
TTGTCTATTTGAGTCTCAGGTCCAGCTGGTGCTTATAGGTCATCTCCAGTATGCGACTACCTC
TCTCCCTCTTTGCCATTCCTAACTGATTCTAAC
SEQ ID NO: 31-Verticillium DCL2 promoter sequence
>V. dahliae VdLs.17 supercont1.15 of Verticillium dahliae (VdLs.17) [DNA]
194566-195565 +
CTTCATCTTCCAACCGCCATTACCTCCCCCATACGCGTCCTGCCAAAGAATCATAACTGGCTA
AAACATAAGACGGGACTGGTCATCCGCTGAACCATTCCGAGCTATGTGTCCTGATTGACCCA
TCTCGGCTTATTCGCTCTCAAATACGACTGCAATCGCGTGTGGCTTGGAAACCGTGGAATAC
CATCCTCATATTGTCAGCACCTGTAGCGATACAGCACAATGCTTGACGATTCGGAATCATTTT
CCGCTTCTTTGCGGAGCAGCGGATGTCCAATTGACGATGACTTGACTCCAGAACCAACGTCC
GAATCACGCGACTCAACCTCCCTACCGTATGGCCTTCAGGACGACATCGGCCCCCTTGCTGC
CACCCCGAGCCAGTCGAGTAACGTCACAATCAATGCACGGGCATACCAGTTGGAGATGCTG
GCGGAAAGTAGGAAGAGGAATATCATTCTAGCTGTGCGAACCTTCCCTCTTGCGCCAGTCAA
CCTCGATTGACACCTCCATAGATGGACACAGGCAGTGGCAAGACCCAAGTGTACGTTCCCTG
CAAGCCGAACCTGATTATTGATACTGATTTTCCCAGTGCCGTCCTCCGAATTCGAGCAGAGC
TAGAAGAAGGGGCTTCAGACAAGGTTTGACAAACTCCACTTGGTAGCTTCGCAATCACTTAC
AGGGTTTTAGCTTGTATGGTTCGTGGCTCACAATGTTGAGCTTTGCGCTCAGCAGCATTCTGT
ACTGCAGTCTCAGATTCCTGCAGTTCAGACCAAGCTGCTTCTTGGCAGCGATAATGTTGATTC
ATGGTCCAACCAAGAGACTTGGAACGCTGTGCTTCTCAACGTCAAAATTGTGGTGTCAACCC
CTCAAGTTCTCTGCGATGCCTTGAGCCACGGCTTTGTCCAGATGGGTTCATTATCCTTGCTTG
TCTTTGATGAAGGTATTCAATCAGCGCAGTTTATCAAGTGTTCTTGCCCTAACAACGGTGTAG
CGC
SEQ ID NO: 32-Botrytis cinerea, Bc_DCTN, BC1G_10508
GCAGGGGTCGGATCAACATGTCTATAAACAAACATATGTACCGGCGTTGATCTCTCCTGCAG
ACTGCATTTGCACTTGCTTCCCTCTTCCTCCTCCCGTTTCCTGGTCTTCTTCTACAAGCTGCAG
GCGAGAGAGATAACTTCTACGCACCTTCCATATCCCTCACCTCTTCTCTCCCCACAAGTTCGT
TCATAATCCTTTCGTCCTGTTGTTTTGTCTAGCATTACCTTGCAATTCTTAACAACGGCCGATC
GTGGACATCAATCAATAAAAAGGACGACAAATCATCTTATAATTATTATCCCAAACTTTCAT
TGCACAAATTTGAATTGGATACTCATTTGGCTTTATTCGGAGCGATAAACGTAGAAATTAAT
CGTATAGGGGCTTTTATCAGACAATCAAGAACGGTGATTGGCTCACAGCGGTGAATTGTGAG
GGGTGGTAATACAGAAAACAAATAGTATAGGGAGTATTTTTGGGTGGATTGTTACCAATGTC
TACCACAAGAATCTCAACACCGAAAAGGTCCCCCAAAAAATCGACTTTTGTCAAAACTGGA
ATCTTGACCACCAAATCAACGCCCAATCTCAACGCCTCCTATAATTTGGCATTACTACAAGC
TTCAGGAGCTACACCCGTTCCTGCATATCCTTCCAATAACGGTCAAAGTTTTGCCCTAAATAA
TCCTAGGTCGCAACCGTCTCGACAAGTCTCACTCGCTTCCCTTACCTCGAATTCACTTGCGAC
AATCCCGGATGCAAGCAAGAGATACCCTCTTTCTACAGTCTTTGATGAGGATATGCCAACAG
TAGGCAACATGCCGCCATACACACCTGCTCGAGTTGGCGGTGGACCGGAAGAACTAGAGGT
TGGTGATATAGTCGATGTGCCAGGTAACATGTATGGTATCGTCAAATTTGTTGGCAGTGTGC
AAGGCAAAAAGGGTGTATTTGCTGGGGTAGAATTAAGTGAAACGTTTGCTTCGAAAGGGAA
AAACAATGGCGATGTCGAAGGAATTCAATACTTTGACACAACCATCGATGGTGCTGGGATTT
TTCTTCCAGTCAACAGGGCGAAGAGACGTAGCACCCCTTCGTCGCATGATGAGTCATTTCCC
CTTTCACCGGCGTCTCCATCGATGGGCAATAGGGCTGGGAGATTAGGATCTGAATTAAATGG
TCAGCCAACACCTTTGTTACCAAAATTCGGTCAATCTGTTGGTCCAGGCAGAGCGGCAAACC
CATATGTCCAAAAAACACGTCCATCCATGGCTACACCTACCACCTCAAGACCGGAATCACCA
GTTCGAAGAGCAGCCAATGCCAACCCATCATTAAATACACCTGCACAAAGAGTCCCATCTCG
ATATGCAAGCCCTGCGCAGGCAAACTTTGGACAGAGCGTTAGAGGAACACAAGATTCTAGA
GATCCAAGTAAGAAAGTTGGCTACACCCCCCGAAATGGCATGAAAACACCAATACCTCCAC
GAAGTGTTTCTGCACTTGGAACGGGGAATAGACCTGCACCAATGAACTCGATGAATTTCAGT
GATGAAGAGACACCTCCTGCAGAGATTGCACGTACGGCAACAAACGGAAGCGTAGGCTCAG
TCTCTTCTTTCAACGCGAAATTACGTCCAGCATCAAGATCCGCATCGCGTACAACTTCCAGG
GCTACCGACGACGAATTTGAGCGATTGAGAAGTTTGTTAGAAGATCGCGATAGGGAAATAA
AAGAACAGGCTTCTATTATAGAAGACATGGAGAAAACTCTCAGTGAAGCACAATCGTTGAT
GGAGAACAATAACGAGAACGCAAGTGGTAGACATAGTCAGGGAAGTGTGGATGACAAGGA
CGCAACACAGTTGAGAGCAATAATACGTGAAAAGAACGACAAAATCGCCATGCTGACTGCC
GAGTTTGATCAGCATCGAGCTGATTTCAGAAGCACGATAGACACGCTCGAAATGGCCGGTG
CGGAAACCGAGCGAGTGTACGACGAGCGCATGCGTGTTCTCGTAATGGAGCTCGATACAAT
GCACGAGAATAGTCATGATGTAAAGCACGTTGCTGTACAACTGAAACAGCTAGAAGAGCTC
GTTCAGGAGCTCGAGGAAGGTCTTGAAGATGCACGACGTGGTGAAGCCGAAGCTCGGGGAG
AAGTTGAGTTCTTGCGTGGAGAGGTTGAAAGAACTCGATCTGAACTCCGCCGCGAGCGAGA
GAAGACTGCCGAAGCTCTTAGCAACGCAAATTCTCCTACGAGCGCAAGTGCGGAAACACAT
TCCAAAGAGATTGCTCAGAGAGATGACGAGATTCGTGGATTGAAAGCCATCATCCACTCGCT
CAGCAGAGATGCCATACCTGATGGGAATTTCTCGGATCATGAGGCAACACCAAATATTCTAC
GACCTGGACTAAACCGAAGTCGAACAGAAAGTGCTTCGGTTTCTGAGGAGGAGCGCCGTAC
TCGGGAAAAGCTAGAGCGAGAAGTGAGTGAGCTTCGTGCTCTCGTCGAAAGCAAAGACAAT
AAAGAAGAACAAATGGAGCGCGAGTTGGAGGGATTGCGAAGAGGAAGTGTTAGCAATCCT
ACTACGCATCGTACTAGTGCCATGAGCAGCGGAACTGTGACTCAGGATAGGAATTCTCTCCA
AGACAATAAGAGCACAGTTGTAAGCTGGCGAGAACGTGGTGCCTCAGATGCTCGCCGCTAC
AATCTGGATTCAATGCCAGAGAATGACAGCTACTCCTCTGCAGCTGAGGATTTCTGTGAATT
ATGCGAAACCTCAGGTCATGATGTTCTACATTGCCCGATGTTTGGCCCCAATGGTAACAGCA
GCAATTCTAAGGATGAGTCACCTAAACAGCAACGAACAGGAAAAGACGTTGTCATGGAGGG
ACTTAAATTATCACCCAAACCTTCTCAAGAAGAATACAAACCGGCGCCGTTAGCGCCAGCTA
AGAAGTCGCCTGATGCGTCGCCTATCAAGACTGTTCCCAACCTTATGGAACCAGGACCTGCC
CCAGGAAAGGAAAGTGGAGTAATCAACATGGATAAATGGTGCGGTGTATGTGAAAGAGATG
GACATGACAGTATTGATTGTCCTTTTGAAGATGCTTTTTAGGAGACTACTGCTTTCGATGTTT
CAGGATAAGCAGTCACAACGACGACTTTTTTCATAGATTTTCTTTGTTAATCATAGGCAAGG
CCGCATTGCATTGCAGGAGCGTAATCCGTCTGCGATATACCCTTTCGGTTCTCTGTTTGAAGT
ATGCTTTTCAAGCGATAAGTTTAGAGGGGAAGATGATGTTTTTACGAGGATTGAATGAGATG
GATGAATGCAGGCTAAATCGGGGAAGGGGGAGGGAAGACAAACATGAGTTGAACGGACGT
AATGATCATGTAGTATACTTTGTCAAATTAATGATCCAAATGCA
SEQ ID NO: 33-Sclerotinia sclerotiorum, Ss_DCTN, SS1G_04144
ATGTCGACTACAAGAATCTCAACTCCAAAAAGGTCTCCAAAAAAATCGACATTCACTAAAA
CAGGAATTCAAGTCACAAAATCAACTCCCAATCTCGGTGCCTCCTACAATTTGGCTTTATTAC
AAGCTTCAGGAGCTTCACCGGTTCTTGCACATTTTTCCAATAACGGTCAGGGTTTTGGTCTAA
ACAATCCTAGGTCGAAGCCATCTCGACAAGTCTCACTCGCATCCCTTACCTCAAATTCACTG
GCGGCAATACCGGATGCTAGTAAAAGATACCCTCTTTCAACCGTTTTTGATGAGGATATGCC
ACCAGCAGGCAACATGTATACACCTTCTCGAGTTGGTGGTGGGCCCGATGAGTTGGAGGTGG
GTGACATAGTTGATGTTCCTGGTAACATGTATGGTACTGTCAGATTTGTCGGCAGTGTGCAA
GGCAAGAAGGGGGTCTTTGCCGGAGTGGAATTGGATGAGATGTTTGCTTCCAAAGGGAAGA
ACAATGGTGATGTTGAAGGTCAATCAGTTGGCCCAGGTAGAATTCAAAAAACCCGACCATC
GATAGCCACACCAACCACATCACGACCAGAGTCTCCAGTACGAAGAGCAGCCGCTGCTAGG
ACATCAATAAATGCACCCGGGCAGAGAGTCCCATCTCGATATGGAAGTCCTGCAGCGGCGA
ACTTTGGGCAGAACATTAGAGGAGTGCAAGATGCTAGAGACCCAAGCAAGAAAGTCGGTTA
CGCCCCAACAAATGGCATGAAGACACCAGTCCCTCCACGAAGTGTTTCGGCACTTGGCACAG
GGAGTAGACCTGCAGCAATGAACCTCAGTGATGAAGATACACCTTCTGCTGGAATTACACG
GACGGCAACAAACGGGAGTGTGAGCTCAATCTCTTCCTTCAACGCAAAGTTACGACCTGCAT
CAAGATCCGCCTCGCGTGCGTCCCGAGCTACTGACGACGAGGTCGAGCGATTGAGAGGTCT
ACTGGAGGAGCGCGATCGGGAAATAAAAGCACAAGCTTCAATCATAGAAGACATGGAAAA
GACTCTTAGTGAAGCTCAGTCACTGATGGAGGACAACAATGAGAACGCGGGCGGTCATAGA
GATAGCCGGGGAAGCATGGAGGACAAAGACGCAGCACAATTGAGAGCAATAATTCGTGAA
AAGAATGAAAAAATCGCCATGCTGACTGCTGAGTTTGATCAGCATCGAGCTGATTTCAGAAG
TACAATAGACACACTTGAGATGGCTGGTGCTGAAACCGAAAGAGTCTACGATGAGCGCATG
AGTAATCTTGTAATGGAGCTCAGGACGATGCATGAGAACAGTCATGATGTGAAGCATGTTGC
TGTACAACTGAAACAGCTAGAAGAGCTTGTTCAGGAGCTTGAGGAAGGTCTTGAAGATGCG
CGGCGTGGTGAAGCCGAGGCTCGCGGTGAGGTCGAGTTCTTGCGTGGAGAGGTTGAAAGAA
CTCGATCTGAGCTTCGTCGTGAGCGGGAGAAAACTGCTGAAGCTCTCAGTAACGCAAATCCT
GCTACGGGTGTGGGTGCAGCAACACTTTCTAAAGAGATTGCACAAAGAGATGACGAGATCC
GCGGTTTGAAAGCTATCATTCACTCGCTTAGCCGAGATGCCATACCTGATGGGAATTTCTCG
GATCATGAAAAGACACCAAGTGTTACACGACCAGGGCTACATCGAAGCCGTACGGAAAGCG
CTTCAGCTTCAGAGGAGGAGCGTCTTAGCCGGGAGAAGTTGGAACGAGAAGTGAGCGAACT
TCGTGCCGTCGTAGAAAGTAAAGACAGCAAGGAAGAAGAAATGGAGCGTGAGCTAGAGGG
GCTACGAAGGGGAAGTGTCAGCAATTCTACTACGCAGCGTACTAGTGCCATTAGCAGTGGA
ACTGCAACCCAGGATAGAAACTCTGTCCGAGATTCCAAAGGCACAGTTGGAAGCTGGCGGG
ACCGCGAAGGAACATCGGATGTTCACCACCACAACTTGGAGTCAATGCCAGAGATTGACGG
TTACTCTTCAGCAGCGGAGGATTTCTGTGAATTGTGCGAGGCATCAGGTCATGATGTTCTAC
ATTGCCCCATGTTCGGTCCTAATGGTAATAGTGGCAACTCTAGAGAGGAGTCTCCTAAAGAG
CAACGAACAGGAAAAGACGTTGTCATGGAAGGACTCAAACTATCACCCAAACTAGCGCAAG
AAGAATACGAACCAGCACCTTTAGCACCAGCCAAGAAGTCGTCTGATGACTCGCCTATTAAA
ACCATCCCTAACCTCATGGACCCAGGTGCTGCTCCAGGAAAAGCAAGTGGAGTCATCAATAT
GGACAAATGGTGCGGTGTATGTGAACGAGATGGACATGACAGCATTGACTGTCCGTTTGAA
GATGCATTTTAG
SEQ ID NO: 34-Botrytis cinerea, Bc_VPS51, BC1G_10728
GACACATGCGATATGCAAAGTCTAGAACCTCGAATACTGATTCGAAAAAGACTGGCAATTC
CATAAATCTACAGTATATTTTAATCCGCAACTCATGAATGACTACATTTAATACGAATTACA
AACATTCCCTAACGCCAAAATGGCAGCTACGATTCCCCTCTCCACTACAACATGCTTGACCT
CCTCAGAAGCTTTCAAATATCCTCTTCCACAGATTCGTCAATTCCACCGCGATCTCACTACAG
AGCTTGACGAGAAAAATGCACGTCTGCGGACACTGGTCGGAGGGAGTTATAGACAATTACT
TGGAACCGCCGAGCAAATCTTACAGATGCGACAGGATATTAGTGGAGTAGAGGAAAAGTTA
GGCAAAGTAGGAGAAGGATGTGGGAGAAATGTGTTGGTTGGAATGGTTGGCGGATTGGGAA
AATTACAGGGAGAAATGAAGAATGGAAAGAAGGGCGAGGAAATGCGGGTTGTGGCTAAGA
TGAAGGTATTGGGTATGTGTGGGATTGTGGTTGGGAAGCTCTTGAGGAGACCAGGGCGAAT
GGATGGGGATGGTGGGAGAGGGAAGGAATTAGTAGTTGCTGCGAAAGTCTTAGTTTTGAGC
CGATTGTTGGCGAAGAGCTTGGAGAATACTGGAGATAAGGAATTCGTTGAAGAAGCGAAGA
AGAAGAGGTCGGCTTTGACGAAGCGATTGTTACGCGCAGTTGAAAAGACATTGGTTTCCGTC
AAGGATGCTGAAGATAGAGACGATTTGGTACAGACACTTTGTGCATACAGTCTAGCTACTAG
TTCTGGCACCAAAGACGTCTTGCGACATTTCTTAAATGTTCGTGGTGAAGCAATGGCTTTAG
CGTTTGACGATGAAGAGGAGTCGAACAAGCAGACCTCAGGTGTCCTACGCGCTTTGGAAAT
ATATACGAGAACTTTACTAGATGTACAGGCTCTAGTGCCAAGGAGGCTGAGCGAAGCGTTG
GCTGTGCTGAAGACGAAACCTTTACTGAAAGATGACAGCATTCGGGAAATGGAGGGATTGA
GGTTGGATGTATGTGAGCGGTGGTTTGGCGATGAGATTATTTACTTCACACCTTATGTCCGGC
ATGATGATTTGGAAGGGTCATTGGCGGTTGAAACACTACGAGGTTGGGCGAAGAAAGCGTC
AGAAGTGTTACTGGAAGGTTTTACGAAGACTCTTCAAGGGGGATTAGACTTTAAAGTAGTTG
TTGAACTACGAACAAAGATTCTGGAGGTGTGGGTTAGAGATGGAGGCAAAGCAAGGGGATT
CGATCCCTCTATACTTCTAAATGGCTTACGAGACGTTATAAACAAACGACTCGTAGAGTTAT
TAGAAACTAGAGTTGGCAAACTTCATCTAGTGGGGACAGAGATAGAGTCCACATTAGCAAC
ATGGCAAGAAGGAATCACCGACATACATGCAAGTCTTTGGGACGAAGATATGATGGCAACC
GAGCTCAGCAATGGTGGTAACATTTTCAAGCAAGACATACTTGCTCGCACGTTCGGACGGAA
CGATGCTGTTTCAAGAGTTGTTAACAGTTTTCACACTTGGAGACATCTCATCGAGGAAATTG
GTACTTATATTGATGAACTGAAGAAACAAAGATGGGATGATGATTTGGAAGATATGGAAGA
TGATGAAAGTCTCGAATCACGACAAAACCTTCTTAGCAAGGAAGATCCACAAATGCTACAA
GATCATCTCGATTCAAGCTTAGAAAATTCGTTCCAGGAGTTACACGCAAAGATCACTTCACT
GGTGGACCAGCAAAAAGATAGTAAACATATCGGGAAAATATCGATATATATTCTCCGAATT
CTACGAGATATCAGAGCAGAATTACCTAGTAACCCTGCACTACAAAAGTTTGGACTCTCACT
TGTCTCATCACTGCACGAAAATCTCGCAGGTATGGTCTCAGAAAACGCCATCTTAGCCCTTG
CAAAATCTCTCAAGAAGAAGAAGGTTGCGGGCAGAGCATTATGGGAGGGTACACCGGAACT
TCCTGTTCAGCCCTCCCCAGCAACATTCAAATTTTTGAGAGGTTTATCGACTGCTATGGCTGA
TGCTGGAGCCGATCTATGGAGCCCTGTTGCCGTCAAAGTGTTGAAAGCGCGTCTGGACACCC
AAGTTGAAGACCAATGGAGTAAGGCTCTAAAAGAAAAAGAGGAAGAGCCTAGCAATGGAA
TCTCTGGTTCTCCCACCAATGCTCCCGAAGCAGATGCCGAGGAAAAAGAAGGGGACGCTTCT
GCTCCTAATCCTGCTGCTGCTGTAGAAGTAGATGAAGAAAAACAAAAGGATTTACTAAAGC
AATCACTGTTCGATATATCTGTCTTGCAGCAAGCTTTAGAATCACAGTCAGACAATAAGGAG
AACAAACTTAAGAACTTAGCGGATGAGGTGGGAGGAAAACTAGATCTCGAGGCGAGGGAA
AGGAAACGTATGGTTAATGGCGCGGCGGAGTATTGGAAGAGGTGCAGTCTTTTGTTTGGACT
TTTAGCGTAGATTCCAGATGGATGAATTAGTGAGAGGCTTATAATGAATTATATTACGAATA
CTTTACTTTTGAGTATTCA
SEQ ID NO: 35-Sclerotinia sclerotiorum, Ss_VPS51, SSIG_09028
ATGGCATCTACAACCCTCTCCACAACAACATGCTTCACTTCCTCGGAAGCATTTAAACATCCT
CTCCCTCAAATCCGGCAATTCCACCGCGATCTCACCACCGAACTTGATGAGAAAAACGCACG
TCTACGTACACTTGTCGGAGGTAGTTATAGACAATTACTGGGAACCGCTGAACAAATCCTAC
AAATGCGCAAGGATATCCGTGAAGTGGAGGAAAAGTTGGGGGAAGTAGGGGAAGGATGTG
GAAGAAATGTATTAGTTGGGATGGCTTCTGGATTAGGTAAATTACAGGGAGAAATGAAGAA
TGGGAAGAAAGGGGAGGAAATAAGGGGATTGGCTAGAATGAAGGGTTTGGGTATGTGTGG
GATTGTGGTTGGGAAACTTTTGAGGAGGCAGGGAAGAGTGGATGGGGAGGGGAGAGGGAA
AAGTTTAGTGATTGCTGCGAAAGTTTTGGTTTTGAGTCGGTTGTTGGCGAAGAGTTTGGAGG
GTTGTGTGAATAGTGCGGATAGAGAATTTGTTGAGGAGGCAAAGAAGAAGAGGGTGGTTTT
GACGAAACGATTGTTACGGGCGGTTGAGAAGACATTAGTCTCGACCAAGGATGGTGAAGAT
AGAGAAGACCTGGTACAGGCTCTTTGCGCGTATAGTCTTGCTACTAGCTCTGGTGCGAAAGA
CGTTTTACGACATTTTCTAAATGTCCGAGGGGAAGCAATGGCATTAGCATTCGAAGACGAAG
AGGAATCGAACCAGGAGACATCAGGTGTTTTGCGGGCATTGGAAATATATACGAGGACTTT
ACTTGATGTACAAGCATTGGTACCGAGTAGACTTAGCCAAGCATTGGCTGCGCTGAAGACGA
AACCTTTATTGAAAGATGAAAGTATTCGAGATTTGGAGGGATTGAGATTAGATGTATGTGAG
CGGTGGTTTGGTGATGAAATTCTTTACTTTACACCTTATGTTCGACACGATGATTTGGAAGGA
TCATTAGCCGTTGAGACATTAAGAGGTTGGGCGAAGAAAGCATCAGAGGTACTACTGGAAG
GATTCACAAAGACTCTTCAAGGTGGCTTGGACTTCAAGGTAGTAGTCGAATTACGGACAAAG
ATATTGGAGGTATGGATACGGGATGGAGGAAAGGCAAGAGGGTTTGATCCGTCTATACTTC
GAGATGGACTGCGAGGTGTTGTTAACGAACGACTTGTAGAGTTATTGGAAACTCGAGTTGGC
AAACTTCATCTAGTGGGAACAGAAATAGAATCCACATTGGCTACATGGGAGAAATGGATTA
CTGATCATCATGCTAGTCTATGGGATGAAGATATGATGGCAACGGAACTCAGCAATGGAGG
TAATATGTTCAAACAAGACATTCTTGCTCGTACCTTTGGACGTAATGATGCTGTTTCAAGAGT
AGTCAACAGTTTTCAGACTTGGAGACATCTCATCAAGGAAATAGGTACTGTTATTGATGAAT
TGAAGAAACAAAGATGGGATGATGATTTAGAAGATATCGAAGATGAAGAAAGTCTTGAGTC
GCGACAAAATCTTCTTAGTAAGAAAGATCCACAAATGTTGCAAGATCATCTTGATTCAAGCT
TAGAAAAAGCTTTTCAGGAGTTACATACGAAAATCACGACACTTGTGGAGCAATACAAAGA
TAGCGAGCATATCGGAAAGATATCAATGTATATTTTACGAATTTTACGAGATATCCGAGCAG
AGCTACCGACAAATCCATCACTACAACAATTCGGTCTTTCACTGATCCCATTACTACACGAG
AGCCTTGCCAGCACAGTTTCTGAAAACCCTATCTCTTCTCTAGCAAAATCGCTCAAGAAAAA
AAAAGTTGCAGGAAGAGCATTATGGGAAGGAACACCGGAACTTCCAATTCAACCTTCACCT
GCTACATTTAAATTTCTTCGTGCTTTATCAAATGCTATGGCTGATGCTGGAGCAGATCTTTGG
AGTCCTATTGCTATTAAGACTTTGAAAGTACATCTCGATTCCCAAATTAATGAGAAATGGAG
CATAGCCTTGTCAGAGAAGATGGCTAGTAATAAAACAACTACTTCTTCCAGCAATCCACCCG
ATACTGAAAAATCCGCGGAAACAGAAGAACCAAAAAATGAAGTTCAATCCCCGTTGGATAA
AGAAGTAGAAGAAGAAAAAGAAAAAAATCTACTAAAACAATATTTATTCGATATCTTCGTC
TTACAACAAGCTTTAGCGCTACAATCTATACAATTTGGGGATAAGGAAAAGGAAAAGGAAA
AAGGGATTATGGGGATGAAAATCAAGAATTTGAGTGATGAGATTGAATTGGAATTGAAGCT
TGAGATGCAGGAGAGGAAGAGGGTGGGGAATGGTGCGAGGGAGTATTGGAAGAGGACGGG
GCTTTTGTTTGGGTTTTTGGTGTAG
SEQ ID NO: 36-(Botrytis cinerea, Bc_SAC1 BC1G_08464)
GATCCACCCACATCCTTCCTCATATGACTTCGATGATAATTACATAGACACTGCCAGTATGCC
TGGCCTCGTTCGCAAACTCCTTATCTTTGCCGCCATCGATGGGTTGATTTTGCAACCAGCAGC
GCCAAAAGGCCAACGCCCCGCCCCCGCAACGAAGATCGCATACAAAGATAAGCATATCGGG
CCAGTATTGAGTGATTTGCAGGATCTGGAGGGGTCGTCTGCGAAAAGTTTCGAGGCATTTGG
TATTGTCGGTCTCTTGACGGTTTCCAAAAGCTCCTTCCTGATATCGATTACGAAAAGAGAGC
AAGTCGCACAAATACAAGGGAAACCTATATATGTTATTACTGAAGTGGCTTTGACCCCATTA
AGTTCCAAGAACGAAGCAGAGATCTCGATTGATAGTACGAAAGCGGGGTTATTGAAGAGTA
ATATCGAGGGGCAGCATGGCTTGGACGAGAGTGATAGCGAGGATGATGTCGTTAGCGATGA
AGTGGAGGACGATACAGCAGTAGAAGCACACAAAAGAACGAGTAGCGTAGCTGAAGATGT
GATCTCGAAGAAGGGGGGATATGGAAGATTTGCTCAAAAATGGTTCTCGAAGAAAGGATGG
GCCGTGGACCAGAAGAAGAACCTGGGGATGAGCGCTGAGCCGTATTCCACAGTGGAGCAAG
CTTCCAAGGCCACCGATGTACCAGCTACGATTTCAGGAGTCACTGAAGGAAAATCTGATATC
TCAATTCCCGATAAGGGCAAGGAAATTGAGGACATTGAAACTCCTGAAAATATTAGCGACA
TTGCAGAGAGCATGCTGCCAAAATTACTACGAACATCGCAGATATTGTTTGGGGCCTCTCGG
AGTTACTACTTTTCTTACGACCATGATATCACAAGAAGTTTGGCAAATAAGAGGAATACAAA
TTCTGAATTGCCATTGCACAAGGAAGTTGATCCACTCTTCTTCTGGAATCGGCATCTTACTTT
ACCATTTATTGATGCTGGCCAGTCTTCTCTTGCCTTGCCTCTTATGCAGGGCTTTGTAGGACA
GCGTGCATTTTCAATGGATAGTAATCCACCAAACCCTGCTATAGGTTCAGACACTGGAAAGA
CTTCCGTGCAGATGAAGGATATTACAACAAGTAGTTCGGATGAGCAAATTTACACAGCACGT
GCTGGTACAGACAAGTCGTATCTATTGACGTTAATATCTAGAAGGTCAGTCAAACGTGCCGG
GCTTAGATATTTACGCCGGGGTGTGGATGAGGACGGCAATACAGCCAATGGCGTGGAAACA
GAGCAAATCTTATCGGATTCTGCTTGGGGCCCTTCGAGTAAGACATATTCGTTCGTTCAGAT
ACGTGGCAGCATTCCCATATTCTTCTCCCAGTCACCTTACTCTTTTAAACCTGTACCTCAAGT
TCACCACTCTACCGAAACAAATTATGAAGCTTTCAAGAAGCATTTTGATAATATAAGTGATC
GCTACGGGGCCATTCAAGTGGCTTCCTTGGTGGAGAAGCATGGAAACGAGGCAATAGTCGG
TGGAGAGTACGAGAAATTGATGACTCTCCTTAATGTCTCCCGAGCTAGCGAGCTTAGGAAAT
CCATTGGGTTTGAATGGTTTGATTTCCATGCTATTTGCAAAGGTATGAAATTTGAGAATGTCA
GCCTGCTCATGGAAATACTGGACAAGAAGCTTGACTCGTTTTCGCACACTGTTGAAACCGAT
GGGAAACTTGTATCGAAACAGAATGGCGTTTTAAGGACTAACTGTATGGATTGTCTGGATCG
AACAAACGTTGTTCAAAGTGCAGTGGCAAAGCGAGCACTTGAAATGCAGTTAAAGAATGAG
GGACTAGATGTCACTCTACAAATTGATCAAACTCAACAATGGTTCAATACTTTGTGGGCCGA
CAATGGTGACGCCATTTCTAAGCAATACGCTTCTACAGCAGCATTGAAGGGAGACTTTACTC
GTACTAGGAAGCGGGATTATAAGGGGGCCATCACAGATATGGGGCTTTCTATCTCCAGATTT
TATAGCGGCATTGTAAATGACTACTTCAGTCAAGCTGCCATTGATTTCCTGCTTGGAAATGTG
AGCTATCTTGTTTTTGAAGACTTCGAGGCAAACATGATGAGCGGTGATCCTGGCGTTTCGAT
GCAAAAAATGAGGCAACAAGCCATTGATGTTTCTCAGAAACTCGTTGTTGCTGACGACCGTG
AAGAATTTATTGGAGGATGGACATTTCTCACTCCGCAGGTACCCAATACGATCAAATCTAGT
CCTTTTGAGGAATCCGTCCTCCTATTGACAGATGCTGCATTGTATATGTGCAATTTTGATTGG
AATATCGAGAAAGTATCATCTTTCGTGAGAGTGGACTTGAACCAGGTGAACGGCATCAAGTT
TGGAACATACATCACGAGTACTTTGTCACAAGCCCAGGCAGATGAGAAGAGGAATGTGGGC
TTTGTAATAACTTATAAGGCTGGTTCAAACGACATTATTCGCGTGAACACGAGATCTATGGC
TACGGAATTTCCTTCTTCGAAACTCTCTCTCGAAGACAAAACATCCACGCCCGCTTCTACATC
TACCACCAACTCTGTCGTCGCCCCAATTGCCGCCGGGTTTGCAAACCTAATCTCAGGTTTACA
AAATCAAAGTATAGCGGAACCTAAAGATCTCGTGAAGGTTCTCGCATTCAAGGCTCTACCCT
CCAGATCTGCGGTATCAGATGAAGGAGTTAGTGAGGCCGAGCAAGTGAAGAGTGTCTGTGG
AGAGATTAGAAGAATGGTTGAGATTGGAAGTATAAGAGAGGCTGGAGAGGAGAGAAAGGA
TATTGTAGAGGAGGGTACTATCATTAGTTTGGCCGAGGCCAAGAAAAGCACGGGACTATTC
GATGTGCTGGGACATCAGGTGAAGAAACTGGTTTGGGCTTAATGAAAGTGTATCGATACTCG
TGCTAGTAATGCTTAGAGCAAAAGAAGCACTTCTTGAAGGATTTACGAATGGAATTGTGGAA
GTTGGCAGGGAGGTTAGCGATCGTCAAGAACGGGTATGTGGAATTCAATTCCATATTGAAGC
TGCGAAACTCATTAACTTCAATAGAAGTGGATGTGTAGATAGACCCGAGTATATGGTATTGG
CCAGATAAGTAATTTTAATGGGGA
SEQ ID NO: 37-Sclerotinia sclerotiorum, Ss_SAC1, SSIG_10257
ATGCCTGGCCTCGTTCGAAAGCTTCTTATCTTTGCCGCCATTGATGGCTTGATTCTGCAACCA
ACGGCGCAAAAAGGCCAGCGCCCCGCCCCCGCAACGAAGATCACGTATAAAGATAAGCATG
TCGGACCAGCATCTTATGATTCTCACGATTACGAGGGGCCGTCTGCCAAAGGCTTTGAAGCA
TTCGGGATTGTCGGTCTCTTGACGGTTTCTAAAAGCTCCTTCTTAATATCGATTACGAAAAGG
GAACAAGTCGCACAAATACAAGGAAAACCTATATATGTTATTACTGAAGTAGCTTTGACCCC
TCTAGCTTCCAGGATAGAAGCAGAGAACTCGATCAACAAAACAAGAGCGGGATTGTTAAAG
AGTAGTATTGAAGATCATGGATTGGACGACAGTGATAGTGAGGATGACGAAGTCAATGTTA
GTGACGAAGTGGAGGACGATACAGCAATAGAAACACATACAAGAACGAGCAGTGTGGCCG
AAGATGTAATTTCGAAGAAGGGAGGGTATGGGAGATTCGCTCAAAAATGGTTCTCGAAGAA
AGGATGGGCTGTGGACCAGAAGAGGAACCTGGGAATGAGCACTGAACCGTATGCTGCACGA
GAGCAAGATGCCAGGTCTGCCGACGTAGCAGCTACCACTTCAAAGGATGCTGAAGTGGAAC
CTGAGGTTTTGATTTCCGATGAGGTCAGGGACATTGAAAATGTTGGAAAGTCTGACAAGGTT
AAGAACGTTCAGGATATTGCTGAGAGCATGCTGCCAAAGTTACTGCGTACGACACAAATATT
GTTTGGGACCTCCCGGAGTTACTATTTTTCTTACGATCATGATATCACAAGAAGTTTGGCCAA
TAAAAGGAACACAAACTCTGAATTGCCATTGCATAAGGAGTCGATCCACTCTTCTTCTGGAA
CCGACACCTTCTGTTACCATTTATTGATGCTGGGCAAGCTTCACTTGCCTTGCCTATTATGCA
GGGCTTCGTAGGACAACGAGCATTTGTAATGGATAGCAATCCGCCAAAGCCTGTTGTAGGTT
CGGACACTGAGAAGACCTCCATGGAACTGAATGAGATCACAACAGATAGTTCGGATGAACA
AATCTCCACAGCACGTGTTAGTGCAGATAAGCCATATCTATTGACATTAGTGTCTAGAAGAT
CGGTTAAGCGTGCCGGGCTTAGATATCTTCGTCGAGGTGTGGATGAGGACGGCAATACCGCC
AATGGTGTGGAGACGGAGCAAATTTTAATCAGATTCTACTTGGGCTCCTTCAAGTAA
SEQ ID NO: 38-Bc-VPS5 1+ DCTN1 + SAC1-dsRNA (VDS)
TTCGTTCCAGGAGTTACACGCAAAGATCACTTCACTGGTGGACCAGCAAAAAGATAGTAAA
CATATCGGGAAAATATCGATATATATTCTCCGAATTCTACGAGATATCAGAGCAGAATTACC
TAGTAACCCTGCACTACAAAAGTTTGGACTCTCACTTGTCTCATCATTCGTGCTCTCGTCGAA
AGCAAAGACAATAAAGAAGAACAAATGGAGCGCGAGTTGGAGGGATTGCGAAGAGGAAGT
GTTAGCAATCCTACTACGCATCGTACTAGTGCCATGAGCAGCGGAACTGTGACTCAGGATAG
GAATTCTCTCCAAGACAATAAGAGCACAGTTGTAAGCTGGACGTTGTTCAAAGTGCAGTGGC
AAAGCGAGCACTTGAAATGCAGTTAAAGAATGAGGGACTAGATGTCACTCTACAAATTGAT
CAAACTCAACAATGGTTCAATACTTTGTGGGCCGACAATGGTGACGCCATTTCTAAGCAATA
CGCTTCTACAGCAGCATTGAAGG
SEQ ID NO: 39-BcDCL1/DCL2
TGCGGAAGAACTTGAAGGTTTGCTACACAGTCAAATATGTACTGCAGAAGATCCCAGCTTGC
TGCAGTACTCAATCAAAGGTAAACCTGAGACTCTTGCCTACTATGATCCCTTGGGCCCGAAA
TTCAATACTCCTCTTTATCTTCAAATGCTCCCGCTTCTAAAAGACAATCCTATCTTTCGGAAG
CCATTTGTATTTGGGACAGAAGCCAGTAGAACTCTAGGATCTTGGTGTGTTGACCAGATCTG
GACGGATGCCATTTGCTGCACGCCAAAAATACATCGAGCAGATCTTCGCCTTCGAGTAAAGC
TACCACTTCTATCTATTATCTACTATACCCCAGAGTCAAATATCATCGTGACGAAAACTGTGG
CGAGCCTGAGAAAGATTGTGCAAAGTCTCAACATTTTCGAAGACCCCTACGTTTTGACACTA
AAAAGGAGTGATAGCGAAAAAAGTCAACGTGAGCTGGCGAAAGTACTCAAGAGT
SEQ ID NO: 40-R. Solani PG
ATGCACTATCTTTCCTTTGCAGCTCTTGCTTTTGCGCCCATCTTGGCTATTGCGACTCCTGTTA
GCCGTTGCACGGGCACTATCGCCTCTCTGGATGACGTCGCTGCTGCCCAGAAATGCACTACT
GTCACTATCAAAGGCTTTACTGTCCCTGCCGGAAAGACGTTTGAGCTTTCTCTCCTAGACAAC
ACCGTTGTCAACATGGAAGGAGACGTAAAGTTCGGAGTTGCGAACTGGGCCGGGCCGCTAT
TTTCCGTCTCGGGAAAGGGTATCACATTCAACGGCAATGGCCACACGTTCGATGGTCAAGGC
CCGTCCTACTGGGATGGTCAGGGCGGTAATGGAGGTGTGACCAAGCCCCACCCGATGATGA
AGATCAAGATTTCGGGTACATACTCCAACGTAAAGGTCCTCAACTCGCCCGCACATACCTAC
AGCATCTCGAACCCTGCAAAGCTGGTCATGTCCAAGCTTACAATTGACAACTGTAAGTGCCC
ACATAATCCACGGGTGGCACCGATATATGTACTAACATCGTCTCTAGCTGCAGGAGATGCCC
CGAATAATCAATCCGGAGGCAAGGCCGCCGGTCACAATACTGATGGCTTTGATGTTTCCACC
ACCGACCTCACCATTGAGGACAGCACCATCCGTAACCAGGATGACTGCATTGCCATTAACAA
GGGCTCGAACATCATCTTCCAGCGCAACTCTTGCACCGGCGGTCATGGTATCTCTATCGGTTC
GATCTCGACCGGAGCGACCGTCCAAAACGTACAGATCCTGAACAACCAGATCATCAACAAC
GACCAGGCTCTCCGCATTAAGACTAAAGCGGATGCTACCAGTGCTTCTGTCTCTGGGATCAC
TTTCTCTGGCAACACTGCAACTGGCACAAAGAAATTCGGTGTGATTGTTGACCAGGGATATC
CCACTACACTCGGAGCTCCTGGAAATGGGGTCAAGATTTCGGTGAGGCTCTTGCTAGAAACA
TGCTTCAATTCGTCGACCGGCAACACCAACAACATCGCAGTCACTTCCAGCGCTCAGCGAGT
GGCCGTTAACTGTGGCACAGGATGCACAGGCACATGGGACTGGTCCAAATTGACTGTGACC
GGAGGAAAGGCCTCTGACAGCAAGTACAGGTATTCGGGCGTCAAAGGAGCGCCATGGCGCT
GTAGATCTCCAACTCGATCAAATTCCGTAATGGGGAACAAAGTACTCGGTTTGCCACCTCAC
ATTACCCCATTCCACTCGCTAATTGACGTCTTCTTATCGTCGGTCCTAATCACAAACCGGATG
CAGGCCTATCTCAGCACTTTTGCAGCTACTCCAACAGATGGTCGCGATACGTTAACTTCGCTT
GCGCAGCTATCGGTTGAGCTTACTTCGGGCACCAGTGTGAAACTTGACCGACCCGCTCACGC
TCGGTGGGCCTACACTTCACTCATCCAGGGACTTCCCGGCCGGTATACCTCACAAGACGCGT
CCCAGCCGTGGCTCATTTATTGGGCATTACAAACCCTTACATGTCTTGGGGTTCAATTGGACC
CCGCCACCAAACAGCGCACTATTGATACGATCATCGCAAATCAGCATCCTGATGGTGGTTTT
GGAGGAGGACCTGATATCCGAGATTTACGCCATGGTTTCTCCAGGCAGAAGTGCTATGAATT
TTTTATGAGGATGAAACAGCCGGATGGATCATTTGTCGTTAACAAGGACGCCGAAGTGGATG
TCAGGGGAACATATTGTCTTTTAGTTGTAGCAACTCTCCTCGACATATTAACTCCAGAATTGG
TGGAGGGAACTTCCGAGTTCTTACGCAGCTGTCAGACATATGAGGGAGGGTTCGCGTCCTCT
TCTCACCCATATTACAGCCCAGAGGATGGTAAACCTCAAGTGCTATCTGAAATTCGTCCAAC
CCTAGGAGAGGCCCACGGCGGCTATACGTCATGCGCTATTGCTAGCTGGATATTACTACAAC
CCTACCAGAAGCCGGAAGATCCCAAGGTCAATGTGAAAAAGCTGGTACGATGGGCGACTGG
AATGCAAGGTCTTCCGATAGAGGGAGGAGGGTTCCGCGGCCGGACCAACAAATTAGTTGAT
GGCTGTTATTCGTGGTGGATTGGAGGGCTCGAGCCCCTTTTGTTGGAGCTGCTCGGGCTTGGT
AATGACGAAGGAGAGACTGAGGTAGTGAGTCATGTCACAGAGGAAACAGACAACGCCCCG
ATGGCCTTGTTCGATAAGACATCACTGCAACGGTTCACCTTGGTCTCATCTCAGCTCTCATCC
GGTGGGCTCCGCGACAAACCCGGAAAGGCTGCCGATCTTTACCATACGGCATACAATCTAGC
AGGCTATTCAACGGCTCAGCATCGAGTTTATCGATCTTTAGTCACAGAAAGGAAATTGCTTG
ATGCCTGGAAGAGCTCAAGCGGTGTCATTCAAGGTTCAGAAGAAAAGATACGGAAGATAAC
TTGGGCTAGGATATGCGCATGGCAGGAAGATGAAGGTGCACATTTCTACCTCGGAGGGGAG
GGAAATCGGGTGCAGATTGGTCTACAGAATGCTACTCACCCTCTATTCAACCTGACGATATC
ACACACGCGTGCAATGATGAACTATTTCTACCAGCAAGAGGGGCTCTAG
SEQ ID NO: 41-Exemplary R. Solani PG SIGS sequence
CCCCATTCCACTCGCTAATTGACGTCTTCTTATCGTCGGTCCTAATCACAAACCGGATGCAGG
CCTATCTCAGCACTTTTGCAGCTACTCCAACAGATGGTCGCGATACGTTAACTTCGCTTGCGC
AGCTATCGGTTGAGCTTACTTCGGGCACCAG
SEQ ID NO: 42-A. niger pgxB
ATGTACCTCCTTCCCTTGACGCTCTTCCTCACCGCCGCTTTCGGCGTCTCAATCCCTAGATCTC
CCCTCATCCCCGGCGCACAAATCGTCCCCGCATCCAGCACAGCAGATCTACGAGCCATTGGT
GCTCAACATCACAAGTATCCAGACCGAGAGACAGTTACTATTCGGGCCTCGAGGAACGCCCT
CGACGATGTGTCCAGTGACTTCCTCTGGGGCTTGAAGCAGGCGAACCATGGCGGTCGGTTGT
TGTTGAAGCAGGGGGAGACCTACGTGATTGGGAAGAAGTTAGATTTGACATTCTTGGATAAT
ATTGAGGTGCAGCTTGAGGGAGAAATTCAGGTACTTTCCTTGCCCTTCTTCAATACGGAGTA
TTGAAAATATGATACTGATTTCGGTGGTCCTGCTTAGTTCACAAACAACATCACCTACTGGC
AAGCCAACAACTTTTACTACGACTTCCAGAAATCCATCACCTTCTGGCGCTGGGGTGGCCAG
GACATCAAGATCTTCGGGAGTGGTGTGTTGAACGGCAATGGACAGAAATGGTATGATGAGT
TTGCGGGGAAGCAGATCTTGGTATGTCACACCATGATACCATCCGTACCTCCCTGAAAGAAC
AGACAATGCTGATGACAGCAACGATGATAGGACTCAGATAACACGTTCTACCGTCCCATTCT
CTTCCTCACCGATAATGCAACCCGTATCTCCGTCGAGGGCATCACGCAGCTGAACTCGCCGT
GCTGGACGAACTTTTTCGTTCGGACCAATGATGTCTCGTTTGATAATGTGTATATTCATGCGT
TCTCGACCAATGCTTCAGTCAGTCCTCTATTCCTCTGGCTTTTAGTTGATTTCCATTGCATGGA
TGCTAACTGATGACAGTCCGACCCCGCCAACACCGACGGTATGGACTCTCTCGACGTCGATG
GCGTCAGCTTCACCAATATGCGCATCGATGTCGGAGATGACTGCTTCTCGCCGAAGCCGAAC
ACAACCAACATTTTCGTGCAGAACATGTGGTGCAATAACACGCACGGGGTGAGTATGGGTA
GTATTGGCCAGTACGCGGGCGAGATGGATATCATTGAGAACGTGTACATTGAGAATGTGAC
GTTGCTGAATGGACAGGTACGTCTTCTTGTTCCCCACTGACCCATATTACAAGACTGATGTG
GAATAGAACGGCGCCCGCCTCAAAGCCTGGGCCGGCCAAGACGTCGGCTACGGCCGCATCA
ATAACGTCACGTACAAGAACATCCAGATCCAGAACACGGATGCGCCGATCGTGCTGGACCA
GTGCTACTTTGATATCAACGCTACAGAG
TGTGCCAAGTACCCGTCTGCTGTGAATATCACGAATATCCTGTTCGAGAATATCTGGGGCTC
TTCCTCGGGCAAAGATGGCAAGATTGTAGCTGATCTGGTGTGTTCGCCAGATGCGGTGTGCA
CGAACATTACTTTGTCGAATGTCAACTTGACGAGCCCGAAGGGCACTGCAGAGATTGTTTGC
GATGACATTCAGGGAGGAATTGGGGTGGATTGTGTGAGTGACGAGAGTGTTACGCGGTAG
SEQ ID NO: 43-Exemplary A. niger pgxB SIGS sequence
CGACGATGTGTCCAGTGACTTCCTCTGGGGCTTGAAGCAGGCGAACCATGGCGGTCGGTTGT
TGTTGAAGCAGGGGGAGACCTACGTGATTGGGAAGAAGTTAGATTTGACATTCTTGGATAAT
ATTGAGGTGCAGCTTGAGGGAGAAATTCAGGTACTTTCCTTGCCCTTCTTCAATACGGAGTA
TTGAAAATATGATACTGATTTCGGTGGTCCTGC

What is claimed is:

1. A composition comprising an antifungal RNA and a lipid vesicle, wherein the antifungal RNA comprises a double-stranded RNA, a small RNA, or a small RNA duplex, and wherein the lipid vesicle is an artificial vesicle comprising a tertiary amine cationic lipid or a plant-derived vesicle.

2. The composition of claim 1, wherein the antifungal RNA targets a dicer-like (DCL) gene of a fungal pathogen.

3. The composition of claim 1, wherein the antifungal RNA targets the vacuolar protein sorting 51 (VPS51) gene, the dynactin (DCTN1) gene, or the suppressor of actin (SAC1) gene of a fungal pathogen, or a combination thereof.

4. The composition of claim 1, wherein the antifungal RNA targets a polygalacturonase gene or an exo-polygalacturonase gene of a fungal pathogen, or a combination thereof.

5. The composition of claim 1, wherein the antifungal RNA targets the long terminal repeat (LTR) region of a fungal pathogen, or a combination thereof.

6. The composition of claim 2, wherein the pathogen is Botrytis, Sclerotinia, or Verticillium.

7. The composition of claim 1, wherein the lipid vesicle is the plant-derived vesicle.

8. The composition of claim 7, wherein the antifungal RNA is not expressed by the plant from which the plant-derived vesicle is derived.

9. The composition of claim 7 or claim 8, wherein the plant-derived vesicle is obtained from N. benthamiana leaves, a fruit, a vegetable, or a combination thereof.

10. The composition of claim 1, wherein the lipid vesicle is the artificial vesicle comprising the tertiary amine cationic lipid.

11. The composition of claim 10, wherein the cationic lipid is N,N-dimethyl-2,3-dioleyloxy)propylamine (DODMA) or a salt thereof.

12. The composition of claim 10 or claim 11, wherein the ratio of the secondary amine in the cationic lipid to phosphate in the RNA ranges from about 1:1 to about 10:1.

13. The composition of claim 13, wherein the ratio of secondary amine in the cationic lipid to phosphate in the RNA is about 4:1.

14. The composition of claim 10, wherein the vesicle further comprises a sterol.

15. The composition of claim 14, comprising the cationic lipid and cholesterol in a molar ratio ranging from about 1:1 to about 10:1.

16. The composition of claim 10, wherein the vesicle is a micelle, a small unilamellar vesicle, a large unilamellar vesicle, or a multilamellar vesicle.

17. A method of increasing pathogen resistance in a plant or a part of a plant, the method comprising contacting the plant or the part of the plan with a composition according to claim 1.

18. The method of claim 17, wherein the double-stranded RNA, small RNA, or small RNA duplex is sprayed onto the plant or the part of the plant.

19. The method of claim 17 or claim 18, wherein the plant is a fruit- or vegetable-producing plant.

20. The method of claim 17, wherein the part of the plant is a fruit, a vegetable, or a flower.


SEQUENCE LISTING (43 sequences)

SEQ ID NO: 1 - DNA, 5529 bp, Botrytis cinerea
atgacgagagacgcagcagcagcaaaaagtctctaccattggcgaagaaaaggcgtcactccttcagccgaagaggatcttctatcgtttgatgatattgttactgccgttccacctacaatcttgtcttcgtctgtcgctccatatacttctcgagataagataccttctgcatctggcaacggagatgctatagcagatgttagcagtggttacctcaaacaggctaccgtatcttctcattctgctcaagtccgatcatcttcaaacggcaatcaaggtgatgccaaaagttctccctctctttcacctgatagtaaactggaattcatctttgggcctcctttaagggagccagagaagccattctttaataaatcttcttattcgtttcgagattcgagagggttgagcagaaatcgggcttcttcttctatggaaaattcgagaactctcgatccaaagatactcaaaccagttatcatcaataatcaccagggcgaatgcttccaagaggcttccagaacaggtatacctcaggctgatacttttgataaatcttcccttgctaagactgcggatatggatttgtcaccagtttctcaccatgcggatgtgcttgcgacgacggtcactgcacagcattctgcaatagccgcccagaacgcagctcaaagctctaagatgccaggtcctgaagcttttttacttgccgaaaaggacgaggcaggttctcccgttgttatatcactgggttctgcaaaccaaattccttctggaaacatttctttgcagcttgattcaccatctctggaaaaccattctccaaatgtgaccccaatcaacaaagtccctacaccattcgcactttctacaaggacaaccgatgacgttttcgcagaacttaggcggcctttgcatccccaagctattcagagccagattgatatcaagacttcctcttgtgttgatagttataacacgaatgatgagattctagacaacaatcaaggttccaatcaaaaagatctgcatgttgttgaaaaggataaggaagaggaagaggaagaggatatgaaccaagccatacccgatatcaaacgtatctcagcacgaaaacaaaagaacgctgccatatttgacgtttttcttaaggaagctaccaaactaccaaagacagaaaagacttcacatgcgaatgatgaagcaattcagtctactaggtggttgattgaccaagcagaaaaacagcatattatagaaagtcccagggactatcaacttgaattgtttgagaaggcaaagaaacagaacattatagctgtacttgatacaggatctggcaagacattcattgcagttctcttacttcggtggatcatagaccaagagcttgaagatagagctattggcaagcctcatcgtgtttcattcttcctgtggaagaaacgactggatacgaatatggtcattgtctgcactgcagaaattttgcgccaatgcctgcaccattcgtttgttacaatggctcaaataaatctgctaattttcgatgaagcccaccatgcaaagaaggatcatccttatgctaggattattaaagatttttatcgcaatgacacggaaaaggatatcgctctgcctaaaatatttgggatgacagcatcaccggtagatgctagagataatgtcaagaaagctgcggaagaacttgaaggtttgctacacagtcaaatatgtactgcagaagatcccagcttgctgcagtactcaatcaaaggtaaacctgagactcttgcctactatgatcccttgggcccgaaattcaatactcctctttatcttcaaatgctcccgcttctaaaagacaatcctatctttcggaagccatttgtatttgggacagaagccagtagaactctaggatcttggtgtgttgaccagatctggactttctgtcttcaagaagaagagtctaagaaactacaagcaaggacggagcaggcgcatcataagaagagagtcccggagccacttgaagtgctagagaaacgcaaggaacaacttgaacaagccaaatccattgtcgaaaatcacactttcgagccaccacactttgcatcaagattattggatgatttcacaacaaaagttcactattcgaataatttatctactaaagtcgttgctctcttgagtattctcaaagatcgtttccaacgacccaccaatgacaagtgtattgtatttgtcaaagaaagatacaccgcacgccttctagcctcacttctctccacacctgaagctgggacaccattcttgaaggctgcaccgctggttggtactacgtctgcttcagccggggaaatgcatatcacatttagatcacaaactcttactatgcacaactttcgcaatggtaaaatcaactgccttatcgcaacatcagttgctgaagaaggtcttgacattcctgactgtaacctcgttgtcagattcgatttgtacaatacagtcattcagtacattcaatctagaggtcgtgctaggcatatcaattcaaggtactaccatatggtagagagccacaacgaggaacagattcgtacaatcaaagaggttttgaagcatgagaaaatgctaaagctttttgcttctgctcttccagaagatcgaaaattgaccggaaacaacttcaatatggattacttcctcagaaaagaacgaggccacagaatttaccctgtcccgaatagtgacgcaaaacttacttacagaatgagcttaacggtcctatctgccttcgttgactcacttcctcgagccccagagtcggttcttcgagtggattatgtcgtcacaactgtcgataagcagtttatctgtgaggccattttgccagaagaagcacccatacgcggagcaattggtcggccagcaacaactaaacaagtggccaaatgctcagcagcctttgaaacttgtgtgattctgcaccagaaaggatacatcaacgactacctactttctacatttaaaagatcagcacacatgatgagaaatgcacttttggctgtggatggaaagaagcaagaagcttatgatatgcagactaaaccaactttatggtcttcgaaagggaaacaaggcatattttatatgactgtcttgtctctcaaatctccagataatcttgacagagcatctcagccattgggcttactgacaagatcacccttgcctgatttgccagaatttgttcttcatttcggagcagggcgaaactctccaacctcgtgcgtacctctcgcttcctcaattacgctcgaaaaaaacaagcttgaccaagttaatatgttcaccctatgtttattccaagatgtgttcagtaaagcatacaaatcagatccggatagtatgccatactttctggttcctatcaactgcctgaatgctattgtcgactggaaatcacaaaacccaatgtcaataatcgattgggagacagttgaatatgtccaagacttcgagaataagcaagctgataagccatgggagcacaagccatggttaggaaagcctgacgattatttcaaagacaaattcataactgatccctttgacgggtctcgaaaattgtggtccgttggaatcacaaaagaatacagaccattggatccagtcccaccaaacacggcgcccaggaagggagctagaaagaacaatagtaatatcatggagtatagttgtagtctctgggcaaaggctagagcaaaacgaacttttgatgaagaacagcctgttattgaagcaacctacatttcacttcggagaaatttgcttgatgaatttgatggaggtgagctcgagacttcaaagaagagttttattattttagaaccattgaaggtatcacctcttccaactaccgtgggtgcaatggcctatcttttacctgcaattattcatcgagttgagtcatatctcattgctcttgaagcaacagacttgttacatcttgatatccgtcctgatcttgcgctagaggctgttaccaaggattccgacaattctggagagcatggtgaggaacagacaaactttcaacgtggaatgggcaataattatgaacgattggaatttcttggggactgcttcttgaagatgggaacgtcaatatctctatacggtctaaatcctgatagtgatgaattccgctaccatgttgatcgtatgtgtctgatttgcaacaaaaatctgttcaatacggctttgaaattagagctttacaaatacattcggtcggcagccttcaaccgacgagcttggtatcccgaaggccccgaattattaagaggaaagacagccacggcaccaaatacccacaagctcggcgataagtcagttgcagatgtttgtgaagcaatgattggagctgctttactaagccaccacgaaagcaagtccatggataatgcggttcgcgccgttactgaagttgtcaatagtgacaaccacaatgctgttgtatggtctgattattacaaattgtatgagaaaccaaaatggcaaactgctacagctacagctgcacaaatagatatggcaagacaagttgaaatgaaacatccatatcatttcaaacacccacgcctgttaagatcagctttcatccatccggcatacttgttcatctatgaacaaattccttgttatcaacgtctcgaatttttgggtgattcgctactcgatatggcatgtgtcaacttcctttttcacaaccacccaacaaaagatcctcagtggctcactgagcacaagatggctatagtatccaatcagtttcttggagctctttgtgtcaaattaggcttccacaaacatctactgacactcgattctcaagttcaaaaaatgattgcagattactcctcagatatcaatgaagctctcattcaagccaaaacggacgcaaagagagtcggcaaagtagaagatgattacgctcgtgattattggattgccgtccgtcaacctcctaaatgtcttcccgatattgtagaagcattcattggtgccatttttgtcgactctgagtatgactacggtgaagttgagaagttctttgaaatgcatatcagatggtactttgaggatatgggcatctacgatacctatgctaacaagcacccaaccactttccttactaatttcttgcaaaagaacatgggatgtgaggactgggcaccagttagtaaggaagtacctggagaggatggtagaaagaatgttgtagtttgcggggtcatcatacacaataaggtggtatcaactgccactgccgaaagtatgagatatgctagggtcggagcagcgaggaatgccttgagaaaattggagggaatgagtgtccgagaattcagggatgaatacgggtgctcatgtgaaggtgatgttgttgatgaagagggcaatattgaatttgttgaacgtgaagacgggatggaggggatcggtatgggatattga

SEQ ID NO: 2 - protein, 1842 aa, Botrytis cinerea
MTRDAAAAKSLYHWRRKGVTPSAEEDLLSFDDIVTAVPPTILSSSVAPYTSRDKIPSASGNGDAIADVSSGYLKQATVSSHSAQVRSSSNGNQGDAKSSPSLSPDSKLEFIFGPPLREPEKPFFNKSSYSFRDSRGLSRNRASSSMENSRTLDPKILKPVIINNHQGECFQEASRTGIPQADTFDKSSLAKTADMDLSPVSHHADVLATTVTAQHSAIAAQNAAQSSKMPGPEAFLLAEKDEAGSPVVISLGSANQIPSGNISLQLDSPSLENHSPNVTPINKVPTPFALSTRTTDDVFAELRRPLHPQAIQSQIDIKTSSCVDSYNTNDEILDNNQGSNQKDLHVVEKDKEEEEEEDMNQAIPDIKRISARKQKNAAIFDVFLKEATKLPKTEKTSHANDEAIQSTRWLIDQAEKQHIIESPRDYQLELFEKAKKQNIIAVLDTGSGKTFIAVLLLRWIIDQELEDRAIGKPHRVSFFLWKKRLDTNMVIVCTAEILRQCLHHSFVTMAQINLLIFDEAHHAKKDHPYARIIKDFYRNDTEKDIALPKIFGMTASPVDARDNVKKAAEELEGLLHSQICTAEDPSLLQYSIKGKPETLAYYDPLGPKFNTPLYLQMLPLLKDNPIFRKPFVFGTEASRTLGSWCVDQIWTFCLQEEESKKLQARTEQAHHKKRVPEPLEVLEKRKEQLEQAKSIVENHTFEPPHFASRLLDDFTTKVHYSNNLSTKVVALLSILKDRFQRPTNDKCIVFVKERYTARLLASLLSTPEAGTPFLKAAPLVGTTSASAGEMHITFRSQTLTMHNFRNGKINCLIATSVAEEGLDIPDCNLVVRFDLYNTVIQYIQSRGRARHINSRYYHMVESHNEEQIRTIKEVLKHEKMLKLFASALPEDRKLTGNNFNMDYFLRKERGHRIYPVPNSDAKLTYRMSLTVLSAFVDSLPRAPESVLRVDYVVTTVDKQFICEAILPEEAPIRGAIGRPATTKQVAKCSAAFETCVILHQKGYINDYLLSTFKRSAHMMRNALLAVDGKKQEAYDMQTKPTLWSSKGKQGIFYMTVLSLKSPDNLDRASQPLGLLTRSPLPDLPEFVLHFGAGRNSPTSCVPLASSITLEKNKLDQVNMFTLCLFQDVFSKAYKSDPDSMPYFLVPINCLNAIVDWKSQNPMSIIDWETVEYVQDFENKQADKPWEHKPWLGKPDDYFKDKFITDPFDGSRKLWSVGITKEYRPLDPVPPNTAPRKGARKNNSNIMEYSCSLWAKARAKRTFDEEQPVIEATYISLRRNLLDEFDGGELETSKKSFIILEPLKVSPLPTTVGAMAYLLPAIIHRVESYLIALEATDLLHLDIRPDLALEAVTKDSDNSGEHGEEQTNFQRGMGNNYERLEFLGDCFLKMGTSISLYGLNPDSDEFRYHVDRMCLICNKNLFNTALKLELYKYIRSAAFNRRAWYPEGPELLRGKTATAPNTHKLGDKSVADVCEAMIGAALLSHHESKSMDNAVRAVTEVVNSDNHNAVVWSDYYKLYEKPKWQTATATAAQIDMARQVEMKHPYHFKHPRLLRSAFIHPAYLFIYEQIPCYQRLEFLGDSLLDMACVNFLFHNHPTKDPQWLTEHKMAIVSNQFLGALCVKLGFHKHLLTLDSQVQKMIADYSSDINEALIQAKTDAKRVGKVEDDYARDYWIAVRQPPKCLPDIVEAFIGAIFVDSEYDYGEVEKFFEMHIRWYFEDMGIYDTYANKHPTTFLTNFLQKNMGCEDWAPVSKEVPGEDGRKNVVVCGVIIHNKVVSTATAESMRYARVGAARNALRKLEGMSVREFRDEYGCSCEGDVVDEEGNIEFVEREDGMEGIGMGY

SEQ ID NO: 3 - DNA, 4197 bp, Botrytis cinerea
atggaatacacttcggaacctgacactgacccggatacacgcggtagccttatcgatggtcgagatgggattgaaggggatcttattgctttgacgtctggggaacgacttaatgagactgtagaggatttatgtagtgactcatcaggattgattgttgagaatgaagatgatgataacagcgcaggggagaagggagagattgtgatagtaacaccaagaacataccaactggaaatgttggaagagagtttgaaaaggaatgtcatcgttgcgatggatacaggaagtggcaagacacatgtggccgttctccgaatactagcggaacttgagcggatgaagcctggcaagataatatggtttcttgcgcctaccgttgcgctctgtgctcagcatcacgaatatctccagctgaatattccctctgttttgatcaaaatgcttattggtgctgatggtgtggatcgatggacagagcagagacagtgggatacggtcttgaaggatgtcaaggtagtcgtatcttcctatcaagttcttctagatgcccttacacacggattcgtacgcatggggcgtctgtccttgatcatttttgatgaagcacataattgtgtaaataaagcgccaggggctaaaattatgaaatctttctatcatccgtataaatcgatattcccacttccccacattctgggcctctcggccagccctgtcatgagatccagtccacaatctttaagtgatatcgaggagactttggatgccatttgctgcacgccaaaaatacatcgagcagatcttcgccttcgagtaaagctaccacttctatctattatctactataccccagagtcaaatatcatcgtgacgaaaactgtggcgagcctgagaaagattgtgcaaagtctcaacattttcgaagacccctacgttttgacactaaaaaggagtgatagcgaaaaaagtcaacgtgagctggcgaaagtactcaagagttttaagacatatagtcaaacccaattaaagtcaatcgacaaaactagcaacgagattattcttgtagagctaggcccatgggctgcagattactatatctcaacagtggtgacgagatacttgaaggcaatgtcggcaaaggacactttcattgttgaagattcaccagctgccgagaagctatatattgccaaggctctcagacaagtcgaaatctctccttcaactctctcagatacaggcaaaatttctaacaaggttgaaaagctactggggataattgcgcaacagaagcctcccttttccgctattatatttgtccaagaaagagccacggtgtctgtgctagcccatctattatcgcatcatccattgacaaaggatcgttttaagattggaaccatggttggcacatccttaaatggcaagcgtacagaccaaataggagagcttgtcgatgttaatcaacaaaaagacactttgtcaagtttcaagcgtggaaaaattgatatccttatagctacaaatgtattggaagagggaattgatgttcctgcctgtaatctagtgatctgctttagtaaaccagcaaacctcaaatctttcgtacaaagacgagggcgagcaagacagcaagattctaagctgattcttcttgatgcttcaggtgataaagcgacaaattggcatgagcttgaaagaaaaatgcgagaggagtacggaaaggaaatgcgagaattgcaacacatctacgaaattgagacagctgatgaacagtcggaagatgatagggtcttgcgaatagaaagcactggggctcaattagaccttgacagtgctttaccacatctctatcatttctgttcagtcttaacaacaaaagattttgttgacctcaggccagacttcgtctactcctccgaactgggatcggaatatgttcgagcaaaggtcatcctgcctggatcggtttctaaacccctgcgagtccatgaaagccgcggatcgtggttgagcgagaggtcggctgcaaaagatgcagcgtttgaggcgtattccgcattatacagggggggcttagtgaatgataacctactgcccctgatggtgcacgacaaagtcatcgatgagttgacttcaaagcccgtggatactcgcgcgtctcttctggaggtgaaggaaagattaaatccatggattgacattgctagagcatggaaagaggcagaacaccatgctggaattgttcgcacatcggtaatgatcttcaatgggatgaagctggaactctgtcttccaattgatccaccggcaatacccccattaaagctttattgggatgctgacaccgagttctttgttgactttacaaacgatatcgagatcggcaccagcgagaatatgttggcacaggcgttgaacgataccaatctactattatcagatcgtggtcgtaaagttcacatccagtcacgtcgaacagttgtgcaatttatcttgcttcaagattcgggctcgctcagttcagattgttttccggttgaccccaacggtaatattaaaagtacaggttttatcagagaagtcggtaaactagaatcgccctacatctttgaaaaatggttgcccaatgcaccagaagacgtcccatatctagctgtggttaaagtaagtcgccgtgcagactttttgcacaaggtacagaacgaaaaaccctcgtcattcactaaacaattctcgtctgttctacctgcctcgacatgtgtacaggatgtaatgcccgcacagttgtctcggttcggcatgatgattccttccatcacacaccacattgaggtgcaactcgttgtagaccgactatccaggaccatcctcaaggatctcgaaattagtgaccagagtcttattcagaccgccatcacacatgccagttattcgttagactcgaattatcagcgtctcgaatttctgggcgactcaattctcaaattgtgtacatcggtacaattggtggcagagcatctagattggcacgaaggatatttgtcggctatgaaggatcgtatcgtgtccaattcacggtcatcaagagcggcggctgaagtcggtttggatgagtatataatgaccaagaaattcacaggtgcaaaatggcgaccaatgtacgtggatgatctggtcgtcacagaacaaaaaacaagagaaatgtcctccaaaattctttccgacgttgtggaagcactcatcggcgcatctctccggcccgtcgagcaaatcctcgcatataccttcaccaaaaaatctctcctcgtcgaagccatgacgcacccctcttacaccagcggcacgcaatccctcgagcgactcgagttcctcggcgattccattctcgacaacatcatcgtcacagccatgtggtcgcactcgacgccgctctcccacttccacatgcatctcctgcgctctgcgctcgtcaacgccgatttcctcgcctttctctgcatggaaatgagcatcgaccaaaacgtcaccaatctgaccgaaggaaaaaaccatcgcatccacgaaacccactcgcgacgccgcgtttccctcgtcagttttctccgtcactcaagcgttcgtctctctatctatcaaaaagaagcgctttctcgccatgcagaattgcgcgatcagatcctcgaggcaatatacaccggtgatacattcccctgggctctattatcccgattggacgcgcggaaatttttctccgatatgattgagagtttgctgggcgcggtatggattgatagcggctcgatggaagtgtgcacgcagctgatcgaaagaatgggcgtcctgagatacatgcgacggattttgaaagatggcgtgcgcatcatgcatccgaaggaggaactgggcatcgtggccgattctgaaaacgtcaggtacgttttgcggcgggagaagatgggtggggatgctaccgaggtaaatgcggacgcggatgaagaggtacgcacggagtaccggtgcacagtatttgtgggcggggaggaaattgtagaggtgaggggtggagcgaggaaagaggagattcaggcaagggctgcggagcaggcggtgcggattttgaaggcgaggggtcatgagaagaggaatgggggtgcgggggaggggaaaaagagaaaatcgctggatgaatag

SEQ ID NO: 4 - protein, 1398 aa, Botrytis cinerea
MEYTSEPDTDPDTRGSLIDGRDGIEGDLIALTSGERLNETVEDLCSDSSGLIVENEDDDNSAGEKGEIVIVTPRTYQLEMLEESLKRNVIVAMDTGSGKTHVAVLRILAELERMKPGKIIWFLAPTVALCAQHHEYLQLNIPSVLIKMLIGADGVDRWTEQRQWDTVLKDVKVVVSSYQVLLDALTHGFVRMGRLSLIIFDEAHNCVNKAPGAKIMKSFYHPYKSIFPLPHILGLSASPVMRSSPQSLSDIEETLDAICCTPKIHRADLRLRVKLPLLSIIYYTPESNIIVTKTVASLRKIVQSLNIFEDPYVLTLKRSDSEKSQRELAKVLKSFKTYSQTQLKSIDKTSNEIILVELGPWAADYYISTVVTRYLKAMSAKDTFIVEDSPAAEKLYIAKALRQVEISPSTLSDTGKISNKVEKLLGIIAQQKPPFSAIIFVQERATVSVLAHLLSHHPLTKDRFKIGTMVGTSLNGKRTDQIGELVDVNQQKDTLSSFKRGKIDILIATNVLEEGIDVPACNLVICFSKPANLKSFVQRRGRARQQDSKLILLDASGDKATNWHELERKMREEYGKEMRELQHIYEIETADEQSEDDRVLRIESTGAQLDLDSALPHLYHFCSVLTTKDFVDLRPDFVYSSELGSEYVRAKVILPGSVSKPLRVHESRGSWLSERSAAKDAAFEAYSALYRGGLVNDNLLPLMVHDKVIDELTSKPVDTRASLLEVKERLNPWIDIARAWKEAEHHAGIVRTSVMIFNGMKLELCLPIDPPAIPPLKLYWDADTEFFVDFTNDIEIGTSENMLAQALNDTNLLLSDRGRKVHIQSRRTVVQFILLQDSGSLSSDCFPVDPNGNIKSTGFIREVGKLESPYIFEKWLPNAPEDVPYLAVVKVSRRADFLHKVQNEKPSSFTKQFSSVLPASTCVQDVMPAQLSRFGMMIPSITHHIEVQLVVDRLSRTILKDLEISDQSLIQTAITHASYSLDSNYQRLEFLGDSILKLCTSVQLVAEHLDWHEGYLSAMKDRIVSNSRSSRAAAEVGLDEYIMTKKFTGAKWRPMYVDDLVVTEQKTREMSSKILSDVVEALIGASLRPVEQILAYTFTKKSLLVEAMTHPSYTSGTQSLERLEFLGDSILDNIIVTAMWSHSTPLSHFHMHLLRSALVNADFLAFLCMEMSIDQNVTNLTEGKNHRIHETHSRRRVSLVSFLRHSSVRLSIYQKEALSRHAELRDQILEAIYTGDTFPWALLSRLDARKFFSDMIESLLGAVWIDSGSMEVCTQLIERMGVLRYMRRILKDGVRIMHPKEELGIVADSENVRYVLRREKMGGDATEVNADADEEVRTEYRCTVFVGGEEIVEVRGGARKEEIQARAAEQAVRILKARGHEKRNGGAGEGKKRKSLDE

SEQ ID NO: 5 - DNA, 4692 bp, Verticillium dahliae
atgacgactgacgagctctctgttggtctggacgccaccggcatctcaatcctcgcagatggaccggaaaacatatcgtccagcacatcaacatctacgactggaaaggaagatggatacctctgtatcaacagattcactcagaataccgccacgacccaggacaaccagagccgagattctgacgacgatgaggatgactgcggcagccacgatgaagctgacgaagattcagacgaaagacagtacagcatgaccccagaaaggcctcataaaattaccgagaagaagcgcgcagatcatgctgcctttcacgactggcttcagagcaactccagcgagattgctcagtcaacccctcagccggctcaaaacctcaaccacacctccacggccctgatggtacgcgagagtgagaatcgtaagatcatcgaaaatcctcgggagtatcagattgagctcttcgagcgggcgaagcgaaagaacatcattgccgtgttacccactggatcaggaaagaccttaatcgcagcccttcttctgcgacacaccctcgaacaagaaaccgcggatcgacgcgcgggcaagcccaagagaatcgcctttttcctcgtggaaaaggttgctcttgccctccaacagcacgcggttctggagtgcaatctggaatttcccattgaccgggtatgcggtgacatggtacggtcggactggatcaaggagtcatggatgaaaagatgggatgacaacatggtcatggtctgcaccgccgccatccttcagcaatgccttgccagatcattcatccgcatggatcagatcaacctgcttgtcttcgatgaagcacatcacgccaagggaaatcatccgtacgcccggatcatcaaggactactacattacggaacctgacaaagaaaggcgccccaagatcttcggcatgactgcctctccggtggatgccctcaccgacgtcaagattgctgccgctcaactcgaaggtttgttgcatagtgagattgcgacaatcgaggaggactctgtatcattcaaacaaatccagaaagaggtcgtcgaacaagactgcaagtaccctgccctcgaaccacccttcaccaccaatcttcataagaagatccaagaacaggtgcgctacaacaagaacttcgcaaaggcgctgagcaattctttagaaatgtcgagctcccttggcagctggtgtgtcgatcgcttctggcagatatttctgaccgaagaaaccctcgcgagattggcagcgcaaactgcacaagacaacatttttgccgatcgcgccgaaaaggagcgcgttgccattgaggaggtccgcaacatcatcaagcaacatcagttcctcccaatcaccaaaaccctgcaagacttgtcgtccaaagtgctgtgcctcctcggccaactggaattgcgcttcagtgcccctaccgatcacaagtgcatcatcttcgtggagaaacgaaacacagccatgattctggctcacctcctctccttgcctggtattggacctctatatctgaaaccggctgcgcttgtcgggaacccatctgacaacagccctcttgccatgtcgtacaaagagcaagtgatgacaataacaaagttcagacgtggtgaatacaactgtcttctcgccacttctgtggccgaggagggcattgacatcgcagactgcaacattgtcattcgattcgatcttttcaactcggtgattcagtacatacaatccaaaggccgcgctcggcacttgaactcggagtatatttgcatggccgagctaggcaacggcaagcatacaagggcgaagatacaagcaaattatgacctctccctcatccgccaattctgcagcacactgccagaagaccgcaagatcgtgggctgggaccccgaggcagctcttcaccatggcgagcgcgaccataagttccacatcgttccatccaccggggccaaactcacctggaccggcagcctcgtggttctgtcaaattttgcctcttctctacaggtgaacgacgaaacactaagtccttcctatatggtctctctcatcggtagcgagtacatctgcgaggtccagcttccgagcaagtctcccattttgagcgtgtcaggcacgctccaaaagaacaaagcagaggccaggtgctccgcagcgtttgagatgtgcatgaagctcatcaaaggtgggttcatcagcagtcaccttcagccgacgtttaccaggaagctcccggccatgcgaaacgcacgcctagccatcagctccaagaagcgtgaacggtacaatatgagggtcaagccagaggtatggtcacggcgtggaccggcatcctctctgttcctcacagtcctgaagcttcgtacacctggtgcattgaacagaccatcacagccactcgccctcctcacacgagaggcactgccagagcttccaggagttccgctatttttcggtaactgtggtcggtccatagcggaggtagtatctgtggcgaaacccatgcacttggatgaagtacgtctagacagcctcagagtattcaccctgcgcattttcaaagatgtcttcagcaaggtatacgattctcaagtcgcagaccttccatacttcctggcacctgctgctcatgaccacagtcatgagttctcaccgaatgaagacccagggtcactgatcgactggagccatctgctgtcgaccaaagaggttgagtacttgccttgggatgaagatcacagtcccagcttctatcaaagcaagtttgtgattgatccatacacgggatcgcgcaagctgtttctcagaggtattcggacagatctcaagccgaccgacttggttccagatggagttcccgaacccacattcaggctctggaaggacgttgagcataccataaaggaatacagcatcagcctctgggcaaagagtcgagcccggagagctggcgaatggttggacactcaacccgtggtagaagccgagttggtctcgctgcgccggaatcttctcgacgaatttgccgattccaagcatgaagggtctagggtctgttatgtgattctccagccgctacagatctcaacactccctgtcgaggtcgtcgctatggcctacaactttcccgccatcatccatcggattgaatcgaatatgatcgcccttgacgcctgccgtatgttgaaccttcgagttcgtcccgacctggctctcgaggcgatgaccaaagattcaagcaacagtgaagagcacgatcaggaaaagattgatttccaggccggcatgggcaataattatgagcgactcgagtttctcggagactgctttctcaaaatggcaaccaccatcgcactttttactcggatccctgacagcaacgagtttgagtgtcacgtcgagcgaatgcttcttatttgcaaccagaatctgttcaatgtcgcattaaagaagaacttgcaagagtacattcgatcaaagcaattcgatcgacgcagttggtacccccagggtctgaagcagaaggcgggcaaagcccaaggagcacaaaactcacactcattggccgacaagtctattgctgatgtatgcgaggccatcattggcgcctcatatttgtcgtacactgacgagggcaactttgacatggccgtacgcgctgtgacggccgtcgtgaggaacaaaaatcacgacatgaaatcatacgaggactattacaaagcatttaagatgccgatctggcaagcggcggagccaagtgctgtgcagatggaagcgtctttacagattaaagagcagatgggatatgagttcaagtctcctgccctgctgcggagtgccttcaagcacccgtcctacccccgtcagtttgagagcgtgcccaattatcagcgcctcgagttcctcggtgacgcgcttctagacatggtctgcgtagactttctcttcaggaagtttcccgacgccgatcctcaatggctcactgaacacaagatggccatggtttcgaaccacttcctcggaagtctgagtgtagagttgggcttctaccggcgtgtccttcactttaacagcatcatggccaatcaaatcaaggactacgtcgacgcacttactcatgcacgccaagaagccgaagcggtggcccagatctctggcacagtctcgcgagattactggctcaacgtgaagcacccccccaaattcctctcagacgtggtcgaggcatacatcggtgctattttcgttgattcaggatacgattatggccaggtacaggcgttcttcgagaagcatatccggcctttcttcgcagacatggcgctatatgattcctttgccagcagccaccctgtcacaacgctggcgcgtatgatgcagcaggactttggctgccaggactggcggcttcttgtaagtgaactgccgccgagctgcgaagacggcggggcagctgcgatcactgagacggaagtgatttgtgggttcatggtccacggaagaatcctgctacatgccaagtcgtcgagtggacggtacgccaaagtgggtgctgcaaagagagcggtcgagaagctcatgggtctcggcaacgacaaagaggtctttcggacggacttcggctgtgactgtgactgtgaaggtcaagcaatctag

SEQ ID NO: 6 - protein, 1563 aa, Verticillium dahliae
MTTDELSVGLDATGISILADGPENISSSTSTSTTGKEDGYLCINRFTQNTATTQDNQSRDSDDDEDDCGSHDEADEDSDERQYSMTPERPHKITEKKRADHAAFHDWLQSNSSEIAQSTPQPAQNLNHTSTALMVRESENRKIIENPREYQIELFERAKRKNIIAVLPTGSGKTLIAALLLRHTLEQETADRRAGKPKRIAFFLVEKVALALQQHAVLECNLEFPIDRVCGDMVRSDWIKESWMKRWDDNMVMVCTAAILQQCLARSFIRMDQINLLVFDEAHHAKGNHPYARIIKDYYITEPDKERRPKIFGMTASPVDALTDVKIAAAQLEGLLHSEIATIEEDSVSFKQIQKEVVEQDCKYPALEPPFTTNLHKKIQEQVRYNKNFAKALSNSLEMSSSLGSWCVDRFWQIFLTEETLARLAAQTAQDNIFADRAEKERVAIEEVRNIIKQHQFLPITKTLQDLSSKVLCLLGQLELRFSAPTDHKCIIFVEKRNTAMILAHLLSLPGIGPLYLKPAALVGNPSDNSPLAMSYKEQVMTITKFRRGEYNCLLATSVAEEGIDIADCNIVIRFDLFNSVIQYIQSKGRARHLNSEYICMAELGNGKHTRAKIQANYDLSLIRQFCSTLPEDRKIVGWDPEAALHHGERDHKFHIVPSTGAKLTWTGSLVVLSNFASSLQVNDETLSPSYMVSLIGSEYICEVQLPSKSPILSVSGTLQKNKAEARCSAAFEMCMKLIKGGFISSHLQPTFTRKLPAMRNARLAISSKKRERYNMRVKPEVWSRRGPASSLFLTVLKLRTPGALNRPSQPLALLTREALPELPGVPLFFGNCGRSIAEVVSVAKPMHLDEVRLDSLRVFTLRIFKDVFSKVYDSQVADLPYFLAPAAHDHSHEFSPNEDPGSLIDWSHLLSTKEVEYLPWDEDHSPSFYQSKFVIDPYTGSRKLFLRGIRTDLKPTDLVPDGVPEPTFRLWKDVEHTIKEYSISLWAKSRARRAGEWLDTQPVVEAELVSLRRNLLDEFADSKHEGSRVCYVILQPLQISTLPVEVVAMAYNFPAIIHRIESNMIALDACRMLNLRVRPDLALEAMTKDSSNSEEHDQEKIDFQAGMGNNYERLEFLGDCFLKMATTIALFTRIPDSNEFECHVERMLLICNQNLFNVALKKNLQEYIRSKQFDRRSWYPQGLKQKAGKAQGAQNSHSLADKSIADVCEAIIGASYLSYTDEGNFDMAVRAVTAVVRNKNHDMKSYEDYYKAFKMPIWQAAEPSAVQMEASLQIKEQMGYEFKSPALLRSAFKHPSYPRQFESVPNYQRLEFLGDALLDMVCVDFLFRKFPDADPQWLTEHKMAMVSNHFLGSLSVELGFYRRVLHFNSIMANQIKDYVDALTHARQEAEAVAQISGTVSRDYWLNVKHPPKFLSDVVEAYIGAIFVDSGYDYGQVQAFFEKHIRPFFADMALYDSFASSHPVTTLARMMQQDFGCQDWRLLVSELPPSCEDGGAAAITETEVICGFMVHGRILLHAKSSSGRYAKVGAAKRAVEKLMGLGNDKEVFRTDFGCDCDCEGQAI

SEQ ID NO: 7 - DNA, 3593 bp, Verticillium dahliae
atcactctacgggtaaaagcgctgagagaatgatcatgatgaatttctatcatccacgcaaacaatcggcactatctgttccccacgtcctgggactgaccgcaagccccataatgcgatctaggctcgaaggccttgaggcactggaacagacactggactcggtttgcgttacgcccagattgcaccgagatgacttaatgacccatgtcaaaaggcccaccgtctgttatgtccattacgaaacgacagatgctaaggatgagcccaagccggtcagcatttcaagtcttcgcgaagcatgcagaaatatggacatcaggcaagatccatacgttatctgtctaagagacaaaggcactgatcgagcacgacgtgagctcatcaaggtccttacaagccataaaacagattcgcaacagcaaatgaagtctttcttcaatcaaagcttgcgagtcctgcgagatctcgggccctgggcggccgagtactacatttggaaggttgttacagattttctggcaatcattgaagcaagagatcaccgcatgaatcaacggaataccgaagaaaagcagtatctggccaacatccttcgacaaatcagtatcagcgagccgccagtcagcatgttgagtgctcataacacgtcgaacaaagtaatggtgctcatggaatacttgtcatctaaagctaccgatggtactgtcgggatcatatttgtcaaagagcgatcaactgcggcgatgcttgcacacgtgattgagtcgcatccactgacacagaataggcactcgagcgttggggttgttgttggtgcttccactcatctggtaaggaagaaagacatgtgggatctgtctcgagcagcccacgagacagagccccttcttcagttcagatctggccacctcaatttgctcatcgccacgagtgtgcttgaagagggcatcgacgttcctgcctgcaacctcgtgatctgttttgatgagcccgagaatctcaaagcctttgtccagcggcgcggccgagcccggaagaaggattctagcctcgtggttcttctccccgggacagaccacgtgcctcaggactgggaaagcatggaagcgacaatgaggacacactacgagagagaacagcgcgaaatacaaatcatggagcagatcgaagcatccgagtctgcaaagtacgaagagtacgttgtcgagagtactaatgccagactcgacttcgagaacgccaaagcgcatctcagcaacttttgtgggcagctctctcccggggagtttatagacaagaggcccgaatacataccccgtgtggtagacaacggagtacctccatctctgagggtcacggtactgttgccaagctatgttccagctgccgtccgccatgctgagagtcgtcgaagctggaagtcggagcatcaggcctcaaaggatgccgcttttcaggcatacgtggctctttacaaagcgggactggtcaatgaacacatgcttccactcacggtaaaagatatcgtacccgcaaacgaacctcgagtagcaaccttgcaggtcaatggcctcttgaatgtctggcttggtattgcccaggcctggatcacgagcactgaaacctggttaactccagtgcacctccgagacgcgacgggattgacgcgaggaacgtatatcatgagaatgccggtagcattgccggcactgccttccacgccggtgtacttcgatcgcgaaggaccatggcttctggattttggcccacaagaacgaaaggagaatcttgaaatgcctgatcatacttcagtgctgcttgcactccactttggccatcactggtctattgctcatggtcagcagcaggttatcagcttcgcttcacaagatggcgaactgaatatcaggcaattaagtgcacggggtttcacaaccgcagatgccgaccgagaggaaatgctgtacctggtacgggacgagtcaggatgcccgtatgtgtacgaccactttctaaatggcaagccgtcacttgaacttgttcaacgacctttccggcgcatcggggactctccaggctttcaagacgcacccagtaacatcccctacttggctctcagaaagtggccgcggtacctggccctcttgcaccaacagaaggtcaacgatctactgccacaggcgacaaacaagaagccatatgctagggtttatccggcaccgtgggcgaaagtcgacacgattccattagatcatgcttactttggggcgttgatccctttcatttcacacattgtcgaggttcgactggttgcagaacagctttcctcgagcctacttcgtgacctcaatttctcagatccctctcttgtcctggcggccattagcactaagggttccttggaagccacaaactacgagcgccttgagcttttgggtgactctatcctcaagctttgcaccacggccaatgccgccgctctgcatggcttagtgtcgaactcgagattgtgtagggctgcactggatgctggccttgacaaatttgttctaactgaaaacttcacttgtcgcacgtggcgccctatctacgtcaacgacatgatggaaaagggtgctcgcgactcaggaccccgtatcatgtcgacgaagacgctcgccgatattgtggaagcactcataggggccgcatacattgacggtggcctcccaaaggcacttgggtgcatttcgatcttcctgagggagctcgattggaaaccgttgccagcttgccaggagatcctttacagtttggcgtcccctgatgtgcctttgccgccaatgcttgttccgctggaggacctgatcggctacacgatgcatctcctcaagactgcttcggtcaacggcgatcttctaggcttccttgcactcgagtgccatgccgaggaagacgaggtgatcattgatatcgatttttctccttccgatacggacttcaatcctcaaaattccgccggggtggaacagaagctcaaacagacacgccggaaaatccccctttggaagtttatgcgccactcctcaatagaggttgtgcagcagcagaccaaagctgccagcgttcatgccgatctccgaggacagatcatgcacgctctggaacatgggtcaagctacccctggtctcttctcgcccgtttacatcccgcaaagttcttctccgacatggtcgaagctgtactgggtgccgtctgggtcgattcgggcgacatgggcgcgtgcattcgtgtggcggaacgactgggcattctgcctgtgctctcccgactggcaaaggaggacgttcatgtgctgcatccgaagcaagagctgggagagatcgctggtccccggacagtcaaatatctcctcactttgcccgaggacgcagccggcctgcaaagtgcaacaagaaaatatgcctgcaaggtcatggtcggggatcgctgtgttgcagaggtggatgacggggtcgctcgagatgaggttgagacaaaggctgcagaggttgcggtacagaccttgaagaatgaacaggctgacgcgaaacaagtagcagaacactaa

SEQ ID NO: 8 - protein, 1187 aa, Verticillium dahliae
MIMMNFYHPRKQSALSVPHVLGLTASPIMRSRLEGLEALEQTLDSVCVTPRLHRDDLMTHVKRPTVCYVHYETTDAKDEPKPVSISSLREACRNMDIRQDPYVICLRDKGTDRARRELIKVLTSHKTDSQQQMKSFFNQSLRVLRDLGPWAAEYYIWKVVTDFLAIIEARDHRMNQRNTEEKQYLANILRQISISEPPVSMLSAHNTSNKVMVLMEYLSSKATDGTVGIIFVKERSTAAMLAHVIESHPLTQNRHSSVGVVVGASTHLVRKKDMWDLSRAAHETEPLLQFRSGHLNLLIATSVLEEGIDVPACNLVICFDEPENLKAFVQRRGRARKKDSSLVVLLPGTDHVPQDWESMEATMRTHYEREQREIQIMEQIEASESAKYEEYVVESTNARLDFENAKAHLSNFCGQLSPGEFIDKRPEYIPRVVDNGVPPSLRVTVLLPSYVPAAVRHAESRRSWKSEHQASKDAAFQAYVALYKAGLVNEHMLPLTVKDIVPANEPRVATLQVNGLLNVWLGIAQAWITSTETWLTPVHLRDATGLTRGTYIMRMPVALPALPSTPVYFDREGPWLLDFGPQERKENLEMPDHTSVLLALHFGHHWSIAHGQQQVISFASQDGELNIRQLSARGFTTADADREEMLYLVRDESGCPYVYDHFLNGKPSLELVQRPFRRIGDSPGFQDAPSNIPYLALRKWPRYLALLHQQKVNDLLPQATNKKPYARVYPAPWAKVDTIPLDHAYFGALIPFISHIVEVRLVAEQLSSSLLRDLNFSDPSLVLAAISTKGSLEATNYERLELLGDSILKLCTTANAAALHGLVSNSRLCRAALDAGLDKFVLTENFTCRTWRPIYVNDMMEKGARDSGPRIMSTKTLADIVEALIGAAYIDGGLPKALGCISIFLRELDWKPLPACQEILYSLASPDVPLPPMLVPLEDLIGYTMHLLKTASVNGDLLGFLALECHAEEDEVIIDIDFSPSDTDFNPQNSAGVEQKLKQTRRKIPLWKFMRHSSIEVVQQQTKAASVHADLRGQIMHALEHGSSYPWSLLARLHPAKFFSDMVEAVLGAVWVDSGDMGACIRVAERLGILPVLSRLAKEDVHVLHPKQELGEIAGPRTVKYLLTLPEDAAGLQSATRKYACKVMVGDRCVAEVDDGVARDEVETKAAEVAVQTLKNEQADAKQVAEH

SEQ ID NO: 9 - DNA, 260 bp, Artificial Sequence
  Description of Artificial Sequence Synthetic polynucleotide
tgcggaagaacttgaaggtttgctacacagtcaaatatgtactgcagaagatcccagcttgctgcagtactcaatcaaaggtaaacctgagactcttgcctactatgatcccttgggcccgaaattcaatactcctctttatcttcaaatgctcccgcttctaaaagacaatcctatctttcggaagccatttgtatttgggacagaagccagtagaactctaggatcttggtgtgttgaccagatctggactttctgtc

SEQ ID NO: 10 - DNA, 296 bp, Artificial Sequence
  Description of Artificial Sequence Synthetic polynucleotide
tctttaagtgatatcgaggagactttggatgccatttgctgcacgccaaaaatacatcgagcagatcttcgccttcgagtaaagctaccacttctatctattatctactataccccagagtcaaatatcatcgtgacgaaaactgtggcgagcctgagaaagattgtgcaaagtctcaacattttcgaagacccctacgttttgacactaaaaaggagtgatagcgaaaaaagtcaacgtgagctggcgaaagtactcaagagttttaagacatatagtcaaacccaattaaagtc

SEQ ID NO: 11 - DNA, 293 bp, Artificial Sequence
  Description of Artificial Sequence Synthetic polynucleotide
ggcaagcccaagagaatcgcctttttcctcgtggaaaaggttgctcttgccctccaacagcacgcggttctggagtgcaatctggaatttcccattgaccgggtatgcggtgacatggtacggtcggactggatcaaggagtcatggatgaaaagatgggatgacaacatggtcatggtctgcaccgccgccatccttcagcaatgccttgccagatcattcatccgcatggatcagatcaacctgcttgtcttcgatgaagcacatcacgccaagggaaatcatccgtacgc

SEQ ID NO: 12 - DNA, 319 bp, Artificial Sequence
  Description of Artificial Sequence Synthetic polynucleotide
acagacacgccggaaaatccccctttggaagtttatgcgccactcctcaatagaggttgtgcagcagcagaccaaagctgccagcgttcatgccgatctccgaggacagatcatgcacgctctggaacatgggtcaagctacccctggtctcttctcgcccgtttacatcccgcaaagttcttctccgacatggtcgaagctgtactgggtgccgtctgggtcgattcgggcgacatgggcgcgtgcattcgtgtggcggaacgactgggcattctgcctgtgctctcccgactggcaaaggaggacgttcatgtgctg

SEQ ID NO: 13 - DNA, 1021 bp, Artificial Sequence
  Description of Artificial Sequence Synthetic polynucleotide
ctcctggatcaggcagatgaattagggaactgatttcgaccttccagagttctctttgcgtgatgggtcacttgggtttggttgtcggtatgctgtgggttcggaggagttgtcctttctggtttctttgttggatagtcctttttgggtagcttggtgtgatgcatgcgttctgggtgtgggtctcgtgaggtctttttgtatcaagtatttttaagcttttttcttgttctcttctttttctgtattggtaatgcttcttctttatgatattctcccatcgctgctttcgcattttctaggttgtagggtgcttcccacgtgtcttccgccggttggtagcccttccatcgcaccaggtattgcacacctctgcctctttttctatgtgctaaaatcctttccacctcatgctctatgtgatcgtcaatttcttctggcggcggcgggtcggcggtaccctgtcgttcgtgccatggttcgagtaaagagacgtggaatacattgtggatcttgtaggtgggcggtaatctaagttcgtatgcttgcccgctggtttttatacccgtcacgacaaaggggcctataaatcgatcggagaattttttcttaggtcgcagttgtttaatgttctttgtgcttagcatcaccttgtccccgatgctatatcgctgtggtgaccccttcgtagtgttcttgtttttctgattggttgccgatttccagaattctttcagcttttctctttctttctctaaagcgtcgatgcgctcgcgtgctgccggcgcccttccctctaaatcggcgtcctcgccgatataatggaatgtgggttggaacccatacatagcctggaatgggcttgtattggttgtactgtgccatgtcgcgttatatgtgaattcagcaaggggcaatagcgatgcccagtcgtcttgcctatagttggtgtagcaacttatatagtgaatcaaattttggttttgtcgttcggtttgaccgtcggtctgcgggtggaacg

SEQ ID NO: 14 - DNA, 3927 bp, Artificial Sequence
  Description of Artificial Sequence Synthetic polynucleotide
atggcatccagagctaccgccacaggtcaatctgccggagacaccaacgacatcgagatgaccgacgccccaaaggagatcactatcaacgaaacccttaagatcgccttaccagacaagtaccaaggtagtcgacaagagctcgatactttcctcttacaacttgagatctactttcgattcaatgaggacaagttcactaccaaggaatccaagagtatatgggccgcatcataccttcgaggtgaagcaaccaaatggatccaaccatatttgcgcgactatttcgagcatgacgataaggatcgtatgcaacccacccgaacaatcttcaatagttttgaaggatttaagacagagattcgtagaatcttcggaaattccaacgagttagaggtagcggaagataagatcttcaacctcaagcagacaggatcagcattgaaatatgctacggaatttcgaagatatgctggaacaaccaagtgggacgaaatcgctatcatgagtcactaccgcaagggactcaaaccagaagtcagactggaattagaaagatctgccgagagtacagatctgaacgatctaattcaggactccatcgaatcagatgatcgtctctacagatatcgacaaagccagagatcatacaaaccccaaggaaatcagaagcaagggcgttaccgcaaaaatgagggtagaccacgttacaatccacagagatacggagaccccatggaactagacgccacgcactacacaaacgggaacgatgactcggaaaagagacgaagacgagaaaacaacttatgctttgaatgtggaaaagcagggcaccgagcagcagactgccgaagcaagaagacaggaggaaaaaggggcaacttcaaacctaagttcggcaaaggccaacttaacgctacctttacaatcccagaaaatccaactaaatccgaaaatactgagactttcaccgttgaggaattccagcaattactaaaggaattaccacgaaatcaagagggcatgaatgcaatagacttatgggagcaagagtattacagaaccccaacaccctctgtgacagaagaaagtcatcaggacgaggcagaagcagaccacgccacgatgagctggacagcttgctatgatgaattctgcggaatccatcgatcagataaagaagcaaccggatggtcccctaagaagagaaagacgaagaaccatcagaataatgtaacatgcgaggatttaactcccaatataacttcgcaagaagttcgcaaagttacccagcagttaaatgctacgggacaggcaggacagatatactgcaaggttcagataaatggacacatacaatcagccatgatagattcaggggctacaggaaattttattgcaccggaagctgagacaatcccaatacgaatgggcataacccaacatacagaggttatacagcttgacgttgtgccattgggccaacaacagatcatcttaggaatgccatggttaaaggcacataatccgaaaatagattgggcacaaggaattgtgacatttgatcagtgcaaaagcggtcacagggacacgctagaggcgttcgcgagacgtaacacgcgccaaggagagttgaacgcgaacaacaccggcgacgtaggacacccagtccagggtcctccattaagagcgaaggccagtacacctcctctacaaatgcagaagccaacgacacggcacgaaatcgcaatcgaggcaaaagaaaagcctacgataccagaacagtacaagaattatgaacatgttttcaaagaaccagggatccatgaggctttaccggaacacaagccatgggatcatgagataatattggaggaaggcaagatgcctgtgcacaccccaatttattcaatgtcagccgatgagttaaagaggctcagagagtacatcgacgacaatttagccaagggatggatcagggaatccgcgtcccaagtggccagtccaactatgtggaaggatcgatatccacttccattagctacggaattaagagatcgattaggcggagctacgatatttaccaagatggacctacgtaatggttaccacttgatcagaatgaaggaaggcgaagaatggaaaaccgcttcaaaacaagatacgggctatacgactactttcatgaggcttatgaacaatgtgttgtcacaatatttggatacttgctgtatatgctacttggacgacatcctagtatattcaaacaacaaggttcaacacattaaggacgttagcaacatcctcgaaagcctatccaaggcagacttgctgtgcaaaccaagcaaatgcgaattccatgtcacagagacagaattcttgggattcaccgtatcaagccaagggctcaagatgagcaaaggcaaggttaaggcagtgctcgaatggaagcagccgaccacaatcaaggaagtacaatcctttctagggttcgtcaacttctacagaagattcatcaagggttattcagggattactacacccttgaccacgttaaccagaaaagatcaaggaagcttcgaatggactgccaaagcacaggagtcattcgatacgctcaaacaagcagtggcagaagagccaatactgttgacttttgacccagagaaagaaatcatagtggagacggactcctcggatttcgctataggagcagttctgagccaaccgggccagaatggaaaataccagccaatcgcattctactcccgaaaactatcaccagccgaattgaattacgagatatatgacaaagaattactggcgatagtcgatgcatttagagaatggcgagtgtatttggaaggatcgaaatacacggtacaggtgtatacagatcataagaacttggtttacttcaccacaacgaagcagttaaacagacgacaggtcagatggtcggagaccatggccaactacaatttcagaatttcatatgtcaaaggatcagaaaacgctagagccgacgctcttagccgaaaaccagaatatcaagaaaacaaaacgtacgagtcatacgctatattcaagaaagacggcgaatcactggtttacaatgcaccacagcttgcagcaacacacctgttggaagacaaccacctcaggaaacagatccaatcacactacgacaaggatgctactgccacacgcatacgcaagacaatagaaccaggattcactatagaaaatgataccatatactttcatggaaaagtatacattccgagtcaaatgaccaaggaatttgtgacggaacaacacgggttgccggcacatggacaccaaggaattgcaaggacatttgcaagaatacgggaaatcagttacttcccacgaatgagaacgatagttgaagaagttgttggaaattgtgacacctgcatacgaaacaagtcatcacgacatgctccgtatggtcagctccagaccccagacatgccttctcagccatggaagtccatcacatgggactttgtggtcaaactaccactctcaaaagatcctactacaggaattgagtacgacgcgatactcaatatagtagacaggctaacgaaatttgcatatatgataccattcaaggaaacatgggatgctgagcaactagcatatgtgttcctaaggatcatagtaagcatacacggagtaccagatgagataatctcggatcgagacaagctctttacctcgaaattctggactaccttattagcacttatgggtatcaagagaaagctatcgacatctttccacccacaaacagatggtcaaacagagaggaccaatcagacaatggaagcatatcttagatgctatcgtataaaatcccgataccacaagaagttaatgccgaatcagcgatag

SEQ ID NO: 15 - DNA, 4857 bp, Artificial Sequence
  Description of Artificial Sequence Synthetic polynucleotide
atggcatccagagatatcgccacaggtcaatctgccggagacaccaacgacatcgagatgaccgatgccccaaaagagatcactatcaacgaaacacttaagatcgccttaccagacaagtaccaaggtagtcgacaagaactcgatactttcctcttacaacttgagatctacttccgattcaatgaggacaagttcactaccaaggaatccaagagtatatgggccgcatcataccttcgaggtgaagcaaccaaatggatccaaccatatttgcgcgactatttcgagcatgacgataaggatcgcatgcaacccacccgaacaatcttcaatagttttgaaggatttaagacagagattcgtagaatcttcggaaattccaacgagttagaggtagcggaagataagatcttcaacctcaagcagacaggatcagcattgaaatatgctacggaatttcgaagatatgctggaacaaccaagtgggacgaaatcgctatcatgagtcactaccgtaagggactcaaaccagaagtcagactggaattagaaagatctgccgagagtacagatctgaacgatctaattcaggactccatcgaatcagatgatcgtctctacagatatcgacaaagccagagatcatacaaaccccaaggaaatcagaagcaagggcgttaccgcaagaatgagggtagaccacgttacaacccacagagatacggagaccccatggaactagacgctacgcactacacaaacgggaacgatgactcagaaaagagacgaagacgagaaaacaacttatgctttgaatgtggaaaagcagggcaccgagcagcagaatgccgaagcaagaagacaggaggaaaaaggggcaacttcaaacctaagttcggcaagggccaacttaatgccacctttgcaatctcagaaaactcaactaaacccgaaaatactgagactttcaccgttgaggaattccagcaattattagaggaattaccacgaaaccaagagggcatgaatgcaatagacttatgggaacaagagtattacagaactccaacaccctctgtgacagaagaaagtcaccaggacgaggcagaagcggaccacgccacgataagctggacagcttgctatgacgaattctgcggaatccatcgatcagataaagaagcaaccggatggttccctaagaagagaaagacgaagaaccatcagaataatgtaacatgcgaggatttaactcccaatataacttcgcaagaagttcgcaaagttacccagcagttaaatgctacgggacaggcaggacagatatactgcaaggttcagataaatggacacatacaatcagccatgatagattcaggggctacaggaaattttattgcaccagaagcggcaaagtacttggaaataccacttcagaggaaacaatacccctatcgattgcagttagttgacggacagctagcagggtctgacggaaagatttcgcaggagacaatcccagtacgaatgagcataacccaacatacagaggttatacagcttgatgttgtgccattgggccaacaacagatcatcttaggaatgccatggttaaaggcacataatccgaaaatagattgggcacaaggagttgtgacatttgatcagtgcaaaagcggtcacagggacacgatagaggcgtccgcgagacgtaacacgcgccaaggagagttgaacgcgaacaacaccggcgacgtaggacacccagtccagggtcctccattaagagcgaaggccagtacacctcctctacaaatgcagaagccaacgacacggcacgaaatcgcaatcgaggcaaaagaaaggcctacgataccagaacagtacaagaaatatgaacatgttttcaaagaaccagggatccatgaggctttaccggaacacaagccatgggatcatgagataatattggaggaaggcaagatgcctgtgcacaccccaatttattcaatgtcagccgatgagttaaagaggctcagagagtacatcgacgacaatttagccaagggatggatcagggaatccgcgtcccaagtggccagtccaactatgtgggtacccaagaaggatggacccgatagactagttgtagactatagaaagcttaacgcactcactaagaaggatcgatatccacttccattagctacggaattaagagatcgattaggcggagctacgatattcaccaagatggacctacgtaatggttaccacttgatcagaatgaaggaaggcgaagaatggaaaaccgctttcaaaacaagatacgggctatacgagtaccaagttatgccattcgggctaaccaacgcaccagctactttcatgaggcttatgaacaatgtgttgtcacaatatttggatacttgctgtatatgctacttggacgacatcctagtatattcaaacaacaaggttcaacacattaaggacgttagcaacatcctcgaaagcctatccaaggcagacttgctgtgcaaaccaagcaaatgcgaattccatgtcacagagacagaattcttgggattcaccgtatcaagccaagggctcaagatgagcaaagacaaggttaaggcagtgctcgaatggaagcagccgaccacaatcaaggaagtacaatcctttctagggttcgtcaacttctacagaagatttatcaagggttattcagggattactacacccttgaccacgttaaccagaaaagatcaaggaagcttcgaatggactgccaaagcacaggagtcattcgatacgctcaaacaagcagtggcagaagaaccaatactgttgacttttgacccagagaaagaaatcatagtggagacggactcctcggatttcgctataggagcagttctgagccaaccgggccagaatggaaaataccagccaatcgcattctactcccgaaaactatcaccagccgaattgaattacgagatatatgacaaagaattactggcgatagtcgatgcatttagagaatggcgagtgtatttggaaggatcgaaatacacggtacaggtgtatacagatcataagaacttggtttacttcaccacaacgaagcagttaaacagacgacaggtcagatggtcggagaccatggccaactacaatttcagaatttcatatgtcaaaggatcagaaaacgctagagccgacgctcttagccgaaaaccagaatatcaagaaaacaaaacgtacgagtcatacgctatattcaagaaagacggcgaatcactggtttacaatgcaccacagcttgcagcaacacacctgttggaagacaaccacctcaggaaacagatccaatcacactacgacaaggatgctactgccacacgcatacgcaagacaatagaaccaggattcactatagaaaatgataccatatactttcatggaaaagtatacattccgagtcaaatgaccaaggaatttgtgacggaacaacacgggttgccggcacatggacaccaaggaattgcaaggacatttgcaagaatacgggaaatcagttacttcccacgaatgagaacgatagttgaagaagttgttggaaattgtgacacctgcatacgaaacaagtcatcacgacatgctccgtatggtcagctccagaccccagacatgccttctcagccatggaagtccatcacatgggactttgtggtcaaactaccactctcaaaggatcctactacaggaattgagtacgacgcgatactcaatatagtagacaggctaacgaaatttgcatatatgataccattcaaggaaacatgggatgctgagcaactagcatatgtgttcctaaggatcatagtaagcatacacggagtaccagatgagataatctcggatcgagacaagctctttacctcaaaattctggactaccttattagcacttatgggtatcaagagaaagctatcgacatctttccacccacaaacagatggtcaaacagagaggaccaatcagacaatggaagcatatcttagatgctatgtaaattatcgacaagacaattgggtagagctattacccatggcacagttcgcatacaatacatcagaaacggaaaccacgaaaatcacaccagcacgagctaattttgggtttaatccacaagcgtataaaatcccgataccacaagaagttaatgccgaatcagcgatagtacaaatcgaacagctgaaagatctccaagagcaactggctcttgatctaagattcatatcttccagaacagcagcgtactacaatacgaaacgtagtatggaacctacgcttaaagagggggataaagtttatttgctacgacgaaacatcgaaaccaagagaccaagcaataaactcgaccacaggaaactaggaccattcaagattgataaggtaataggaacggttaattatcgattgaaattaccagacacaatgaatatccacccagtattccacatatccttgctcgaaccagcaccaccaggagcgccaaatgcgccatttacagaaattgaaccagtcaacccaaacgccatatacgatgtcgaaacaatactagactgcaaatacgtcagaaacaaggtcaagtatttgatcaaatggttagactacccacattcagaaaacacatgggaactcaaggaagatctcagctgccctgagaagctacgggcattccacctgaagtacccacacctgccaataaagcctcaagatccgcttcggacaactcaggcaaagaaggatcgaagaaatcgaaggaagaagaatcaatag

SEQ ID NO: 16 - DNA, 3909 bp, Artificial Sequence
  Description of Artificial Sequence Synthetic polynucleotide
atggcatccagagctaccgccacaggtcagtctaccggagataccaacgacatcgagatgaccgatgccccaaaggagatcactatcaacgaaacacttaagatcgccttaccagacaagtaccaaggtagtcgacaagagctcgatactttcctcttacaacttgagatctacttccgattcaatgaggacaagttcactaccaaggaatccaagagcatatgggctgcatcatacctccgaggtgaagcaaccaaatggattcaaccatatttgcgcgactatttcgagcatgacgataaggatcgcatgcaacccacccgaacaatcttcaatagttttgaaggatttaagacagagattcgtagaatcttcggaaattccaacgagttagaggtagcggaagataagatcttcaacctcaagcagacaggatcagcattgaaatatgctacggaatttcgaagatatgctggaacaaccaagtgggacgaaatcgctatcatgagtcactatcgtaagggactcaaaccagaagtcagactggaattagaaagatctgccgagagtacagatctgaacgatctaattcaggactccatcgaatcagatgatcgtctctacagatatcgacaaagccagagatcatacaaaccccaaggaaatcagaagcaagggcgttaccgcaaaaatgagggtagaccacgttacaatccacagagatacggagaccccatggaactagacgccacgcactacacaaacgggaacgatgactcggaaaagagacgaagacgagaaaacaacttatgctttgaatgtggaaaagcagggcaccgagcagcagactgccgaagcaagaagacaggaggaaaaaggggcaacttcaaacctaagttcggcaaaggccaacttaacgctacctttacaatcccagaaaatccaactaaatccgaaaatactgagactttcaccgttgaggaattccagcaattactaaaggaattaccacgaaatcaagagggcatgaatgcaatagacttatgggagcaagagtattacagaaccccaacaccctctgtgacagaagaaagtcatcaggacgaggcagaagcagaccacgccacgatgagctggacagcttgctatgatgaattctgcggaatccatcgatcagataaagaagcaaccggatggttccctaagaagagaaagacgaagaaccatcagaataatgtaacatgcgaggatttaactcccaatataacttcgcaagaagttcgcaaagttacccagcagttaaatgctacgggacaggcaggacagatatactgcaaggttcagataaatggacacatacaatcagccatgatagattcaggggctacaggaaattttattgcaccggaagctgtaaagtacttgggaataccacttcaaacgaaacaacacccctatcgattgcaggacacgctagaggcgtccgcgagacgtaacacgcgccaaggagagttgaacgcgaacaacaccggcgacgtaggacacccagtccagggtcctccattaagagcgaaggccagtacacctcctctacaaatgcagaagccaacgacacggcacgaaatcgcaatcgaggcaaaagaaaggcctacgataccagaacagtacaagaaatatgaacatgttttcaaagaaccagggatccatgaggctttaccagaacacaagccatgggatcatgagataatattggaggaaggcaagatgcctgtgcacaccccaatttattcaatgtcagccgatgagttaaaaaggctcagagaatacatcgacgacaatttagccaagggatggatcagggaatccgcgtcccaagtggccagtccaactatgtgggtacccaagaaggatggacccgatagactagttgtagactatagaaagcttaacgcactcactaagaaggatcgatatccacttccattagctacggaattaagagatcgattaggcggagctacgatatttaccaagatggacctacgtaatggttaccacttgatcagaatgaaggaaggcgaagaatggaagaccgctttcaaaacaagatacgggctatacgagtaccaagttatgccgttcgggctaaccaacgcaccagctactttcatgaggcttatgaacaatgtgttgtcacaatacttggatacttgctgtatatgctacttggacgacatcctagtatattcaaacaacaaggttcaacacattaaggacgttagcaacatcctcgaaagcctatccaaggcagacttgctgtgcaaaccaagcaaatgcgaattccatgtcacagagacagacttcttgggattcaccgtatcaagccaagggctcaagatgagcaaagacaaggttaaggcagtgctcgaatggaaacagccaaccacaatcaaggaggtacaatcctttctagggttcgtcaacttctacagaagatttatcaagggttattcagggattactacacccttgaccacgttaaccagaaaagatcaaggaagcttcgaatggactgccaaagcacaggagtcattcgatacgctcaaacaagcagtggcagaagagccaatactattgacttttgacccagagaaagaaatcatagtggagacggactcctcggatttcgctataggagcagttctgagccaaccgggccagaatggaaaataccagccaatcgcattctattcccgaaaactatcaccagctgagttgaattacgagatatatgacaaagaattgctggcgatagtcgatgcatttagagaatggcgagtatatttggaaggatcgaaatacacagtacaggtgtatacagatcataagaacttggtttacttcaccacaacgaagcagttaaacagacgacaggtcagatggtcggagaccatggccaactacaatttcagaatttcatatgtcaaaggatcagaaaacgctagagccgacgctcttagccgaaaaccagaatatcaagaaaacaaaacgtacgagtcatacgctatattcaagaaagacggcgaatcactggtttacaatgcaccacagcttgcagcaacacacctgttggaagacaactaccttaggaaacagatccaatcacactacgacaaggatgctactgccacacgcatacgtaagacaatagaaccaggattcactatagaaaatgataccatatactttcatggaaaagtatacattccgagtcaaatgaccaaggaatttgtgacggaacaacacggattgccggcacatggacaccaaggaattgcaaggacatttgcaagaatacgggaaatcagttacttcccacgaatgagaacgatagttgaagaagttgttggaaattgtgacacctgcatacgaaacaagtcatcacgacatgctccgtatggtcagctccagaccccagacatgccttctcagccatggaagtccatcacatgggactttgtggtcaaactaccactctccaaggatcctactacaggaattgagtacgacgcgatactcaatatagtagacaggctaacgaaatttgcatatatgataccattcaaggaaacatgggatgctgagcaactagcatatgtgttccttaggatcatagtaagcatacacggagtaccagatgagataatctcggatcgagacaagctctttacctcgaaattctggactaccttattagcacttatgggtatcaagagaaagctatcgacatctttccacccacaaacagatggtcaaacagagaggaccaatcagacaatggaagcatatcttagatgctatcgtataaaatcccgataccacaagaagttaatgccgaatcagcgatag

SEQ ID NO: 17 - DNA, 4857 bp, Artificial Sequence
  Description of Artificial Sequence Synthetic polynucleotide
atggcatccagagctaccgccacaggtcagcctaccggagataccaacgacatcgagatgaccgatgccccaaaggagatcactatcaacgaaacccttaagatcgccttaccagacaagtaccaaggtagtcgacaagagctcgatactttcctcttacaacttgagatctactttcgattcaatgaggacaagttcactaccaaggaatccaagagtatatgggccgcatcataccttcgaggtgaagcaaccaaatggatccaaccatatttgcgcgactatttcgagcatgacgataaggatcgcatgcaacccacccgaacaatcttcaatagctttgaaggatttaagacagagattcgtagaatcttcggaaattccaacgagctagaggtagcggaagataagatcttcaacctcaagcagacaggatcagcattgaaatatgctacggaatttcgaagatacgctggaacaaccaagtgggacgaaatcgctatcatgagtcactaccgcaagggactcaaaccagaagtcagactggaattagaaagatctgccgagagtacagatctgaacgatctaattcaggactccatcgaatcagatgatcgtctctacagatatcgacaaagccagagatcatacaaaccccaaggaaatcagaagcaagggcgttaccgcaagaatgagggtagaccacgttacaatccacaaagatacggagaccccatggaactagacgctacgcactacacaaacgggaacgatgactcagaaaagagacgaagacgagaaaacaacttatgctttgaatgtggaaaagcagggcaccgagcagcagactgccgaagcaagaagacaggaggaaaaaggggcaacttcaaacctaagttcggcaaaggccaacttaacgctacctttacaatcccagaaaacccaactaaatccgaaaatactgagactttcaccgttgaggaattccagcaattactaaaggaattaccacgaaataaagagggcatgaatgcaatagacttatgggaacaagagtattacagaaccccaacaccctctgtgacagaagaaagtcaccaggacgaggcagaagcggaccacgccacgatgagctggacagcttgctatgatgaattctgcggaatccatcgatcagataaagaagcaaccggatggttccccaagaaaaggaagacgaagaaccatcagaataatgtaacatgcacggatttaacttcaaatataacttcgcgaaaagttcgcaaagttacccagcagttgaatgctacgggacaagcaggacagatatactgcaaggttcagataaatggacacatacaatcagccatgatagattcaggggctacaggaaattttattgcaccagaagctgcaaagtacttggaaataccacttcagacgaaacaatacccctatcgattgcagttagttgacggacagctagcagggtctgacggaaagatttcgcaggagacaatcccagtacgaatgggcataacccaacatacagaggttatacagcttgacgttgtgccattgggccaacaacagatcatcttaggaatgccatggttgaaggcacataatccgaaaatagattgggcacaaggaattgtgacatttgatcagtgcaaaagcggtcacagggacacgctagaggcgtccgcgagacgtaacacgcgccaaggagagttgaacgcgaacaacaccggcgacgtaggacacccagtccagggtcctccattaagagcgaaggccagtacacctcctctacaaatgcagaagccaacgacacggcacgaaatcgcaatcgaggcaaaagaaaggcctacgataccagaacagtacaagaaatatgaacatgttttcaaagaaccagggatccatgaggctttaccggaacacaagccatgggatcatgagataatattggaggaaggcaagatgcctgtgcacaccccaatttattcaatgtcagccgatgagttaaaaaggctcagagaatacatcgacgacaatttagccaagggatggatcagggaatccgcgtcccaagtggccagtccaactatgtgggtacccaagaaggatggacccgatagactagttgtagactatagaaagcttaacgcactcactaagaaggatcgatatccacttccattagctacggaattaagagatcgattaggcggagctacgatattcaccaagatggacctacgtaatggttaccacttgatcagaatgaaggaaggcgaagaatggaaaaccgctttcaaaacaagatacgggctatacgagtaccaagttatgccgttcgggctaaccaacgcaccagctactttcatgaggcttatgaacaatgtgttgtcacaatatttggatacttgctgtatatgctacttggacgacatcctagtatattcaaacaacaaggttcaacacattaaggacgttagcagcatcctcgaaagtctatccaaagcagacttgctgtgcaaaccaagcaaatgcgaattccatgtcacagaaacagaattcttgggattcaccgtatcaagccaagggctcaagatgagcaaagacaaggttaaggcagtgctcgaatggaagcagccgaccacaatcaaggaagtacaatcctttctaggatttgtcaacttctatagaagatttatcaagggttattcagggattactacacccttgaccacgttaaccagaaaagatcaaggaagcttcgaatggactgccaaagcacaggagtcattcgatacactcaaacaagcagtggcagaagaaccaatactgttgacttttgacccagagaaagaaatcatagtggaaacggattcctcagatttcgctataggagcagttctgagccaaccgggccagaatggaaaataccagccaatcgcattctactcccgaaaactatcaccagccgagttgaattacgagatatatgacaaagaattactggcgatagtcgatgcatttagagaatggcgagtatatttggaaggatcgaaatacacagtacaggtgtatacagatcataagaacttggtttacttcaccacaacgaagcagttaaacagacgacaggtcagatggtcggagaccatggccaactacaatttcagaatttcatatgtcaaaggatcagaaaacgctagagccgacgctcttagccgaaaaccagaatatcaagaaaacaaaacgtacgagtcatacgctatattcaagaaagacggcgaatcactggtctacaatgcaccacagcttgcagcaacacacctgttggaagacaaccacctcagaaaacagattcaatcacactacgacaaggatgctactgccacacgcatacgcaagacaatagaaccaggattcactatagaaaatgataccatatactttcatggaaaagtatacattccgagtcaaatgaccaaggaatttgtgacggaacaacatgggttgccggcacatggacaccaaggaattgcaaggacatttgcaagaatacgggaaatcagttacttcccacgaatgagaacgatagttgaagaagttgttggaaattgtgacacctgcatacgaaacaagtcatcacgacatgctccgtatggtcagctccagaccccagacatgccttctcagccatggaagtccatcacatgggactttgtggtcaaactaccactctcaaaggatcctactacaggaattgagtacgacgcgatactcaatatagtagacaggctaacgaaatttgcatatatgataccattcaaggaaacatgggatgctgaacaactagcatatgtgttcctaaggatcatagtaagcatacacggagtaccagatgagataatctcggatcgagacaagctctttacctcaaaattctggactaccttattagcacttatgggtatcaagagaaagctatcgacatctttccacccacaaacagatggtcaaacagagaggaccaatcagacaatggaagcatatcttagatgctatgtaaattatcgacaagacaattgggtagaactattacctatggcacaattcgcatataatacatcggaaacggaaaccacgaaaatcacaccagcacgagctaattttgggtttaatccacaagcgtataaaatcccgataccacaagaagttaatgccgaatcagcaatagtacaagtcgaacagctgaaaaatctccaagagcaactggctcttgatctaagattcatatcttccagaacagcagcgtactacaatacgaaacgtagtatggaacctacgcttaaagagggggataaagtttatttgctacgacgaaacatcgaaaccaagagaccaagcaataaactcgaccacaggaaactaggaccattcaagattgataaggtaataggaacggttaattatcgattgaaattaccagacacaatgaatatccacccagtattccacatatccttgctcgaaccagcaccaccaggagcgccaaatgcgccatttacagaaattgaaccagtcaacccaaacgccatatacgatgtcgaaacaatactagactgcaaatacgtcagaaacaaggtcaagtatttgatcaaatggttagactacccacattcagaaaacacatgggaattcaaggaggatctcagctgccctgagaagctacgggcattccacctgaagtacccacacctgccagtaaagcctcaagatccgcttcggacaactcaggcaaagaaggatcgaagaagtcgaaggaagaagaatcaatag

SEQ ID NO: 18 - DNA, 3909 bp, Artificial Sequence
  Description of Artificial Sequence Synthetic polynucleotide
atggcatccagagctaccgccacaggtcaatctgccggagacaccaacgacatcgagatgaccgacgccccaaaggagatcactatcaacgaaacccttaagatcgccttaccagacaagtaccaaggtagtcgacaagagctcgatactttcctcttacaacttgagatctactttcgattcaatgaggacaagttcactaccaaggaatccaagagtatatgggccgcatcataccttcgaggtgaagcaaccaaatggatccaaccatatttgcgcgactatttcgagcatgacgataaggatcgtatgcaacccacccgaacaatcttcaatagttttgaaggatttaagacagagattcgtagaatcttcggaaattccaacgagttagaggtagcggaagataagatcttcaacctcaagcagacaggatcagcattgaaatatgctacggaatttcgaagatatgctggaacaaccaagtgggacgaaatcgctatcatgagtcactaccgcaagggactcaaaccagaagtcagactggaattagaaagatctgccgagagtacagatctgaacgatctaattcaggactccatcgaatcagatgatcgtctctacagatatcgacaaagccagagatcatacaaaccccaaggaaatcagaagcaagggcgttaccgcaaaaatgagggtagaccacgttacaatccacagagatacggagaccccatggaactagacgccacgcactacacaaacgggaacgatgactcggaaaagagacgaagacgagaaaacaacttatgctttgaatgtggaaaagcagggcaccgagcagcagactgccgaagcaagaagacaggaggaaaaaggggcaacttcaaacctaagttcggcaaaggccaacttaacgctacctttacaatcccagaaaatccaactaaatccgaaaatactgagactttcaccgttgaggaattccagcaattactaaaggaattaccacgaaatcaagagggcatgaatgcaatagacttatgggagcaagagtattacagaaccccaacaccctctgtgacagaagaaagtcatcaggacgaggcagaagcagaccacgccacgatgagctggacagcttgctatgatgaattctgcggaatccatcgatcagataaagaagcaaccggatggtcccctaagaagagaaagacgaagaaccatcagaataatgtaacatgcgaggatttaactcccaatataacttcgcaagaagttcgcaaagttacccagcagttaaatgctacgggacaggcaggacagatatactgcaaggttcagataaatggacacatacaatcagccatgatagattcaggggctacaggaaattttattgcaccggaagctgtaaagtacttgggaataccacttcaaacgaaacaacacccctatcgattgcaggacacgctagaggcgtccgcgagacgtaacacgcgccaaggagagttgaacgcgaacaacaccggcgacgtaggacacccagtccagggtcctccattaagagcgaaggccagtacacctcctctacaaatgcagaagccaacgacacggcacgaaatcgcaatcgaggcaaaagaaaggcctacgataccagaacagtacaagaaatatgaacatgttttcaaagaaccagggatccatgaggctttaccggaacacaagccatgggatcatgagataatattggaggaaggcaagatgcctgtgcacaccccaatttattcaatgtcagccgatgagttaaaaaggctcagagaatacatcgacgacaatttagccaagggatggatcagggaatccgcgtcccaagtggccagtccaactatgtgggtacccaagaaggatggacccgatagactagttgtagactatagaaagcttaacacactcactaagaaggatcgatatccacttccattagctacggaattaagagatcggttaggcggagctacgatatttaccaagatggacctacgtaatggttaccacttgatcagaatgaaggaaggcgaagaatggaagaccgctttcaaaacaagatacgggctatacgagtaccaagttatgccgttcgggctaaccaacgcaccagctactttcatgaggcttatgaacaatgtgttgtcacaatacttggatacttgctgtatatgctacttggacgacatcctagtatattcaaacaacaaggttcaacacattaaggacgttagcaacatcctcgaaagcctatccaaggcagacttgctgtgcaaaccaagcaaatgcgaattccatgtcacagagacagaattcttgggattcaccgtatcaagccaagggctcaagatgagcaaagacaaggttaaggcagtgctcgaatggaagcagccaaccacaatcaaggaagtacaatcctttctagggttcgtcaacttctacagaagatttatcaagggttattcagggattactacacccttgaccacgttaaccagaaaagatcaagaaagcttcgaatggactgccatagcacaggagtcattcgatacgctcaaacaagcagtggcagaagagccaatactattgacttttgacccagagaaagaaatcatagtggagacggactcctcggatttcgctataggagcagttctgagccaaccgggccagaatggaaaataccagccaatcgcattctactcccgaaaactatcaccagccgaattgaattacgagatatatgacaaagaattgctggcgatagtcgatgcatttagagaatggcgagtatatttggaaggatcgaaatacacagtacaggtgtatacagatcataagaacttggtttacttcaccacaacgaagcagttaaacagacgacaggtcagatggtcggagaccatggccaactacaatttcagaatttcatatgtcaaaggatcagaaaacgctagagccgacgctcttagccgaaaaccagaatatcaagaaaacaaaacgtacgagtcatacgctatattcaagaaagacggcgaatcactggtttacaatgcaccacagcttgcagcaacacacctgttggaagacaactaccttaggaaacagatccaatcacactacgacaaggatgctactgccacacgcatacgcaagacaatagaaccaggattcactatagaaaatgataccatatactttcatggaaaagtatacattccgagtcaaatgaccaaggaatttgtgacggaacaacatgggttgccggcacatggacaccaaggaattgcaaggacatttgcaagaatacgggaaatcagttacttcccacgaatgagaacgatagttgaagaagttgttggaaattgtgacacctgcatacgaaacaagtcatcacggcatgctccgtatggtcagctccagaccccagacatgccttctcagccatggaagtccatcacatgggactttgtggtcaaactaccactctcaaaggatcctactacaggaattgagtacgacgcgatactcaatatagtagacaggctaacgaaatttgcatatatgataccattcaaggaaacatgggatgctgaacaactagcatatgtgttcctaaggatcatagtaagcatacacggagtaccagatgagataatctcggatcgagacaagctctttacctcgaaattctggactaccttattagcacttatgggtatcaagagaaagctatcgacatctttccacccacaaacagatggtcaaacagagaggaccaatcagacaatggaagcatatcttagatgctatcgtataaaatcccgataccacaagaagttaatgccgaatcagcaatag

SEQ ID NO: 19 - DNA, 3909 bp, Artificial Sequence
  Description of Artificial Sequence Synthetic polynucleotide
atggcatccagagctaccgccacaggtcagtctaccgaagataccaacgacatcgagatgaccgatgccccaaaggagatcactatcaacgaaacacttaagatcgccttaccagacaagtaccaaggtagtcgacaagagctcgatactttcctcttacaacttgagatctacttccgattcaatgaagacaagttcactaccaaggaatccaagagcatatgggctgcatcatacctccgaggtgaagcaaccaaatggattcaaccatatttgcgcgactatttcgagcatgacgataaggatcgcatgcaacccacccgaacaatcttcaatagttttgaaggatttaagacagagattcgtagaatcttcggaaattccaacgagttagaggtagcggaagataagatcttcaacctcaagcagacaggatcagcattgaaatatgctacggaatttcgaagatatgctggaacaaccaagtgggacgaaatcgctatcatgagtcactactgtaagggactcaaaccagaagtcagactagagttagaaagatctgccgagagtacagatctgaacgatctaattcaggactccatcgaatcagatgatcgtctctatagatatcgacaaagccaaagatcatacaaaccccaaggaaaccaaaagcaagggcgttaccgcaagaatgagggtagaccacgttacaatccacagagatacggagaccccatggaactagacgccacgcactacacaaacgggaacgatgactcagaaaagagacgaagacgagaaaacaacttatgctttgaatgtggaaaagcagggcaccgagcagtagactgccgaagcaagaagacaggaggaaaaaggggcaacttcaaacctaagttcggcaagggccaacttaacgccacctttgccatctcagaaaactcaactaaaaccgaaaatactgagactttcaccgttgaggaatttcagcaattactaaaggaattaccacgaaataaagagggcatgaatgcaatagacttatgggaacaagagtattacagaaccccaacaccctctgtgacagaagaaagtcaccaggacgaggcagaagcggaccacgccacgatgagctggacagcttgctatgatgaattctgcggaatccatcgatcagataaagaagcaaccggatggttccccaagaaaaggaagacgaagaaccatcagaataatgtaacatgcgaggatttaactcccaatataacttcgcaagaagttcgcaaagttacccagcagttgaatgctacgggacaggcaggacaagtgtactgcaaggtccagataaatggacacatacaatcagccatgatagattcaggggctacaggaaattttattgcaccagaagctgcaaagtacttggaaataccacttcaaacgaaacaacacccctatcgattgcaggacacgctagaggcgtccgcgagacgtaacacgcgccaaggggagttgaacgcgaacaacaccggcgacgtaggacacccagtccagggtcctccattaagagcgaaggccagtacacctcctctacaaatgcagaagccaacgacacggcacgaaatcgcaatcgaggcaaaagaaaggcctacgataccagaacagtacaagaaatatgaacatgttttcaaagaaccagggatccatgaggctttaccggaacacaagccatgggatcatgagataatattggaggaaggcaagatgcctgtgcacaccccaatttattcaatgtcagccgatgagttaaaaaggctcagagaatacatcgacgacaatttagccaagggatggatcagggaatccgcgtcccaagtggccagtccaactatgtgggtacccaagaaggatggacccgatagactagttgtagactatagaaagcttaacgcactcactaagaaggatcgatatccacttccattagctacggaattaagagatcgattaggcggagctacgatattcaccaagatggacctacgtaatggttaccacttgatcagaatgaaggaaggcgaagaatggaaaaccgctttcaaaacaagatacgggctatacgagtaccaagttatgccattcgggctaaccaacgcaccagctactttcatgaggcttatgaacaatgtgttgtcacaatatttggatacttgctgtatatgctacttggacgacatcctagtatattcaaacaacaaggttcaacacattaaggacgttagcaacatcctcgaaagtctatccaaagcagacttgctgtgcaaaccaagcaaatgcgaattccatgtcacagaaacagaattcttgggattcaccgtatcaagccaagggctcaagatgagcaaagacaaggttaaggcagtgctcgaatggaagcagccaaccacaatcaaggaggtacaatcctttctagggttcgtcaacttctacagaagatttatcaagggttattcagggattactacacccttgaccacgttaaccagaaaagatcaaggaagcttcgaatggactgccaaagcacaggagtcattcgatacactcaaacaagcagtggcagaagaaccaatactgttgacttttgacccagagaaagaaatcatagtggaaacggattcctcagatttcgctataggagcagttctgagccaaccgggccagaatggaaaataccagccaatcgcattctactcccgaaaactatcaccagctgagttaaattacgagatatatgacaaagaattactggcaatagtcgatgcatttagagaatggcgagtatatttggaaggatcgaaatacacagtacaggtgtatacagatcataagaacttggtttacttcaccacaacgaagcagttaaacagacgacaggtcagatggtcggagaccatggccaactacaatttcagaatttcatatgtcaaaggatcagaaaacgctagagccgacgctcttagccgaaaaccagaatatcaagaaaacaaaacgtacgagtcatacgctatattcaagaaagacggcgaatcactggtctacaatgcaccacagcttgcagcaacacacctgttggaagacaaccacctcaggaaacagatccaatcacactacaacaaggatgctactgccacacgcatacgcaagacaatagaaccaggattcactatagaagatgataccatatactttcatggaaaagtatacattccgagtcaaatgaccaaggaatttgtgacggaacaacacggattgccggcacatggacaccaaggaattgcaaggacatttgcaagaatacgggaaatcagttacttcccacgaatgagaacgatagttgaagaagttgttggaaattgtgacacctgcatacgaaacaagtcatcacgacatgctccgtatggtcagctccagaccccagacatgccttctcagccatggaagtccatcacatgggactttgtggtcaaactaccactctcaaaggatcctactacaggaattgagtacgacgcgatactcaatatagtagacaggctaacgaaatttgcatatatgataccattcaaggaaacatgggatgctgagcaactagcatatgtgttcctaagggtcatagtaagcatacacggagtaccagatgagataatctcggatcgagacaagctctttacctcgaaattctggactaccttattagcacttatgggtatcaagagaaagctatcgacatctttccacccacaaacagatggtcaaacagagaggaccaatcagacaatggaagcatatcttagatgctatcgtataaaatcccgataccacaagaagttaatgccgaatcagcaatag

SEQ ID NO: 20 - DNA, 4854 bp, Artificial Sequence
  Description of Artificial Sequence Synthetic polynucleotide
atggcatccagagctaccgccacaggtcaatctgccggagacaccaacgacatcgagatgaccgacgctccaaaggagatcactatcaacgaaacccttaagatcgccttaccagacaagtaccaaggtagtcgacaagagctcgatactttcctcttacaacttgagatctacttccgattcaatgaggacaagttcactaccaaggaatccaagagcatatgggccgcgtcataccttcgaggtgaagcaaccaaatggattcaaccatatttgcgcgactatttcgagcatgacgataaggatcgcatgcaacccacccgaacaatcttcaatagttttgaaggatttaagacagaggttcgtagaatcttcggaaattccaacgagttagaggtagcggaagataagatcttcaacctcaagcagacaggatcagcattgaaatatgctacggaatttcgaagatatgctggaacaaccaagtgggacgaaatcgctatcatgagtcactaccgcaagggactcaaaccagaagtcagactagaattagaaagatctgccgagagtacagatctaaacgatctaattcaggactccatcgaatcagatgatcgtctctacagatatcgacaaagccaaagatcatacaaaccccaaggaaatcagaagcaagggcgttaccgcaagaatgagggtagaccacgttacaatccacagagatacggagaccccatggaactagacgctacgcactacacaaacgggaacgatgactcagaaaagagacgaagacgagataacaacttatgctttgaatgtggaaaagcagggcaccgagcagcagactgccgaagcaagaagacaggaggaaaaaggggcaacttcaaacctaagttcggcaagggccaacttaatgccacctttgcaatctcagaaaactcaactaaacccgaaaatactgagactttcaccgttgaggaattccagcaattattagaggaattaccacgaaaccaagagggcatgaatgcaatagacttatgggaacaagagtattacagaactccaacaccctctgtgacagaagaaagtcaccaggacgaggcagaagcggaccacgccacgataagctggacagcttgctatgacgaattctgcggaatccatcgatcagataaagaagcaaccggatggttccccaagaagagaaagacgaagaaccgacagaataatgtaacatgcaaggatttaactccaaatgtaacttcgcgaaaagttcgcaaagttacacagcaattgaatgctacgggacaggcaggacaaatatactgcacggttcagataaatggacacatacaatcagccatgatagattcaggggctacagggaattttattgcaccagaagctgcaaagtacttggaaataccacttcaaacgaaacaacacccctaccgattgcagttagttgacggacagctagcagggtctgacggaaagatttcgcaggagacaatcccagtacgaatgggcataacccaacatacagaggttatacagcttgacgttgtgccattgggccaacaacagatcatcttaggaatgccatggttaaaggcacataatccgaaaatagattgggcacaaggaattgtgacatttgatcagtgcaaaagcggtcacagggacacgctagaggcgttcgcgagacgtaacacgcgccaaggagagttgaacgcgaacaacaccggcgacgtaggacacccagtccagggtcctccattaagagcgaaggccagtacacctcctctacaaatgcagaagccaacgacacggcacgaaatcgcaatcgaggcaaaagaaaagcctacgataccagaacagtacaagaattatgaacatgttttcaaagaaccagggatccatgaggctttaccggaacacaagccatgggatcatgagataatattggaggaaggcaagatgcctgtgcacaccccaatttattcaatgtcagccgatgagttaaagaggctcagagagtacatcgacgacaatttagccaagggatggatcagggaatccgcgtcccaagtggccagtccaactatgtgggtacccaagaaggatggacccgatagactagttgtagactatagaaagcttaacgcactcactaagaaggatcgatatccacttccattagctacggaattaagagatcgattaggcggagctacgatatttaccaagatggacctacgtaatggttaccacttgatcagaatgaaggaaggcgaagaatggaaaaccgctttcaaaacaagatacgggctatacgagtaccaagttatgccattcgggctaaccaacgcaccagctactttcatgaggcttatgaacaatgtgttgtcacaatatttggatacttgctgtatatgctacttggacgacatcctagtatattcaaacaacaaggttcaacacattaaggacgttagcaacatcctcgaaagcctatccaaggcagacttgctgtgcaaaccaagcaaatgcgaattccatgtcacagagacagaattcttgggattcaccgtatcaagccaagggctcaagatgagcaaaggcaaggttaaggcagtgctcgaatggaagcagccgaccacaatcaaggaagtacaatcctttctagggttcgtcaacttctacagaagatttatcaaaggttattcagggattactacacccttgaccacgttaaccagaaaagatcaaggaagcttcgaatggactgccaaagcacaggagtcattcgatacgctcaaacaagcagtggcagaagagccaatactattgacttttgacccagagaaagaaatcatagtggagacggactcctcggatttcgctataggagcagttctgagccaaccgggtcagaatggaaaataccagccaatcgcattctactcccgaaaactatcaccagctgagttgaattacgagatatatgacaaagaattactggcgatagtcgatgcatttagagaatggcgagtatatttggaaggatcgaaatacacagtacaggtgtacacagatcataagaacttggtttacttcaccacaacgaagcagttaaacagacgacaggtcagatggtcggagaccatggccaactacaactttagaatttcatatgtcaaaggatcagaaaatgctagagccgacgctcttaaccgaaaaccagaatatcaagaaaacaaagcgtacgagtcatacgctatattcaagaaagacagcgaatcactggtttacaatacaccacagcttgcaacaacacacctgttggaagacaaccacctcaggaaacagatccaatcacactacgacaaggatactactgccacacgcatacgcaaaacaatagaaccaggattcactatagaaaatgataccatatactttcatagaaaagtatacattccgagtcaaatgaccaaggaatttgtgacggaacaacacgggttgccggcacatggacaccaaggaattgcaaggacatttgcaagaatacgggaaatcagttacttcccacgaatgagaacgatagttgaagaagttgttggaaattgtgacacctgcatacgaaacaagtcatcacgacatgctccgtatggtcagctccagaccccagacatgccttctcagccatggaagtccatcacatgggactttgtggtcaaactaccactctcaaaggatcctactacaggaattgagtacgacgcgatactcaatatagtagacaggctaacgaaatttgcatatatgataccattcaaggaaacatgggatgctgagcaactagcatatgtgttcctaaggatcatagtaagcatacacggagtaccagatgagataatctcggatcgagacaagctctttacctcgaaattctggactaccttattagcacttatgggtatcaagagaaagctatcgacatctttccacccacaaacagatggtcaaacagagaggaccaatcagacaatggaagcatatcttagatgctatgtaaattatcgacaagacaattgggtagagctattacccatggcacagttcgcatacaatacatcagaaacggaaaccacgaaaatcacaccagcacgagctaattttgggtttaatccacaagcgtataaaatcccgataccacaagaagttaatgccgaatcagcgatagtacaagtcgaacagttgaaagatctccaagagcaactggctcttgatctaagattcatatcttccagaacagcagcgtactacaatacgaaacgtagtatggaacctacgcttaaagagggggataaagtttatttgctacgacgaaacatcgaaaccaagagaccaagcaataaactcgaccacaggaaactaggaccattcaagattgataaggtaataggaacggttaattatcaattgaaattaccagacacaatgaatatccacccagtattccacatatccttgctcgaaccagcaccaccaggagcgccaaatgcgccatttacagaaattgaaccagtcaacccaaacgccatatacgatgtcgaaacaatactagactgcaaatacgtcagaaacaaggtcaagtatttgatcaaatggttagactacccacattcagaaaacacatgggaactcaaggaagatctcagctgccctgagaaactacgggcattccacctgaagtacccacatctgccaacaaagcctcaagctccgcatcagacaacaaaggcaacgaggggtcgaagaaaccaaaagaagaaccactag

SEQ ID NO: 21 - DNA, 1962 bp, Artificial Sequence
  Description of Artificial Sequence Synthetic polynucleotide
atgtgggtacccaagaaggatggacccgatagactagttgtagactatagaaagcttaacgcactcactaagaaggatcgatatccacttccattagctacggaattaagagatcgattaggcggagctacgatattcaccaagatggacctactatattcaaacaacaaggttcaacacattaaggacgttagcaacatcctcgaaagcctatccaaggcagacttgctgtgcaaaccaagcaaatgcgaattccatgtcacagagacagaattcttgggattcaccgtatcaagccaagggctcaagatgagcaaaggcaaggttaaggcagtgctcgaatggaagcagccgaccacaatcaaggaagtacaatcctttctagggttcgtcaacttctacagaagatttatcaaaggttattcagggattactacacccttgaccacgttaaccagaaaagatcaaggaagcttcgaatggactgccaaagcacaggagtcattcgatacgctcaaacaagcagtggcagaagagccaatactattgacttttgacccagagaaagaaatcatagtggagacggactcctcggatttcgctataggagcagttctgagccaaccgggtcagaatggaaaataccagccaatcgcattctactcccgaaaactatcaccagctgagttgaattacgagatatatgacaaagaattactggcgatagtcgatgcatttagagaatggcgagtatatttggaaggatcgaaatacacagtacaggtgtacacagatcataagaacttggtttacttcaccacaacgaagcagttaaacagacgacaggtcagatggtcggagaccatggccaactacaactttagaatttcatatgtcaaaggatcagaaaatgctagagccgacgctcttagccgaaaaccagaatatcaagaaaacaaaacgtacgagtcatacgctatattcaagaaagacggcgaatcactggtttacaatgcaccacagcttgcagcaacacacctgttggaagacaaccacctcaggaaacagatccaatcacactacgacaaggatgctactgccacacgcatacgcaagacaatagaaccaggattcactatagaaaatgataccatatactttcatggaaaagtatacattccgagtcaaatgaccaaggaatttgtgacggaacaacacgggttgccggcacatggacaccaaggaattgcaaggacatttgcaagaatacgggaaatcagttacttcccacgaatgagaacgatagttgaagaagttgttggaaattgtgacacctgcatacgaaacaagtcatcacgacatgctccgtatggtcagctccagaccccagacatgccttctcagccatggaagtccatcacatgggactttgtggtcaaactaccactctcaaaggatcctactacaggaattgagtacgacgcgatactcaatatagtagacaggctaacgaaatttgcatatatgataccattcaaggaaacatgggatgctgagcaactagcatatgtgttcctaaggatcatagtaagcatacacggagtaccagatgagataatctcggatcgagacaagctctttacctcgaaattctggactaccttattagcacttatgggtatcaagagaaagctatcgacatctttccacccacaaacagatggtcaaacagagaggaccaatcagacaatggaagcatatcttagatgctatgtaaattatcgacaagacaattgggtagagctattacccatggcacagttcgcatacaatacatcagaaacggaaaccacgaaaatcacaccagcacgagctaattttgggtttaatccacaagcgtataaaatcccgataccacaagaagttaatgccgaatcagcgatatatggaacctacgcttaa

SEQ ID NO: 22 - DNA, 957 bp, Artificial Sequence
  Description of Artificial Sequence Synthetic polynucleotide
atggccaactacaattttagaatttcatatgtcaaaggatcagaaaacgctagagccgacgctcttagccgaaaaccagaatatcaagaaaacaaaacgtacgagtcatacgctatattcaagaaagacggcgaatcactggtctacaatgcaccacagcttgcagcaacacacctgttggaagacaaccacctcaggaaacagatccaatcacactacaacaaggatgctactgccacacgcatacgcaagacaatagaaccaggattcactatagaagatgataccatatactttcatggaaaagtatacattccgagtcaaatgaccaaggaatttgtgacggaacaacatgggttgccggcacacggacatcaagggattgcaagaacatttgcaagaatccgggaaatcagttacttcccacgaatgagaacgatagttgaagaagttgttggaaattgtgacacctgcatacgaaacaagtcatcacgacatgcgccgtatggtcagctccagaccccagacatgccttctcagccatggaagtccatcacatgggactttgtggtcaaactaccactctcaaaggatcctactacaggaattgagtacgacgcgatactcaatatagtagacaggctaacgaaattcgcatatatgataccattcaaggaaacatgggatgctgagcaactagcatatgtgttcctaaggatcatagtaagcatacacggagtaccagatgagataatctcggatcgagacaagctctttacctcgaaattctggactaccttattagcacttatgggtatcaagagaaagctatcgacatctttccacccacaaacagatggtcaaacagagaggaccaatcagacaatggaagcatatcttagatgctatcgtataaaatcccgataccacaagaagttaatgccgaatcagcgatag

SEQ ID NO: 23 - DNA, 987 bp, Artificial Sequence
  Description of Artificial Sequence Synthetic polynucleotide
atccaatcacactacaacaaggatgctactgccacacgcatacgcaagacaatagaaccaggattcactatagaagatgataccatatactttcatggaaaagtatacattccgagtcaaatgaccaaggaatttgtgacggaacaacatgggttgccggcacacggacatcaagggattgcaagaacatttgcaagaatccgggaaatcagttacttcccacgaatgagaacgatagttgaagaagttgttggaaattgtgacacctgcatacgaaacaagtcatcacgacatgcgccgtatggtcagctccagaccccagacatgccttctcagccatggaagtccatcacatgggactttgtggtcaaactaccactctcaaaggatcctactacaggaattgacatacacggagtaccagatgagataatctcggatcgagacaagctctttacctcgaaattctggactaccttattagcacttatgggtatcaagagaaagctatcgacatctttccacccacaaacagatggtcaaacagagaggaccaatcagacaatggaagcatatcttagatgctatgtaaattatcgacaagacaattgggtagagctattacccatggcacagttcgcatacaatacatcggaaacggaaaccacgaaaatcaccccagcacgagctaattttgggtttaatccacaagcgtataaaatcccgataccacaagaagttaatgccgaatcagcaatagtacaagtcgaacagctgaaagatctccaagagcaactggctcttgatctaagattcatatcttccagaacagcagcgtactacaatacgaaacgtagtatggaacctacgcttaaagagggggataaagtttatttgctacaacgaaacatcgaaaccaagagaccaagcaataaactcgaccacaggaaactaggaccattcaagattgataaggtaataggaacg

SEQ ID NO: 24 - DNA, 3573 bp, Artificial Sequence
  Description of Artificial Sequence Synthetic polynucleotide
atggcatccagagccaccgccacaggtcagtctaccggagataccaacgacatcgagatgaccgatgccccaaaggagatcactatcaacgaaacacttaagatcgccttaccagacaagtaccaaggtagtcgacaagagctcgatactttcctcttacaacttgagatctacttccgattcaatgaagacaagttcactaccaaggaatccaagagcatatgggctgcatcatacctccgaggtgaagcaaccaaatggattcaaccatatttgcgcgactatttcgagcatgacgataaggatcgcatgcaacccacccgaacaatcttcaatagttttgaaggatttaagacagagattcgtagaatcttcggaaattccaacgagttagaggtagcggaagataagatcttcaacctcaagcagacaggatcagcattgaaatatgctacggaatttcgaagatatgctggaacaaccaagtgggacgaaatcgctatcatgagtcactaccgtaagggactcaaaccagaagtcagactagagttagaaagatctgccgagagtacagatctgaacgatctaattcaggactccatcgaatcagatgatcgtctctatagatatcgacaaagccaaagatcatacaaaccccaaggaaaccaaaagcaagggcgttaccgcaagaatgagggtagaccacgttacaatccacagagatacggagaccccatggaactagacgccacgcactacacaaacgggaacgatgactcagaaaagagacgaagacgagaaaacaacttatgctttgaatgtggaaaagcagggcaccgagcagtagactgccgaagcaagaagacaggaggaaaaaggggcaacttcaaacctaagttcggcaagggccaacttaacgccacctttgccatctcagaaaactcaactaaaaccgaaaatactgagactttcaccgttgaggaatttcagcaattactaaaggaattaccacgaaataaagagggcatgaatgcaatagacttatgggaacaagagtattacagaaccccaacaccctctgtgacagaagaaagtcaccaggacgaggcagaagcggaccacgccacgatgagctggacagcttgctatgatgaattctgcggaatccatcgatcagataaagaagcaaccggatggttccccaagaaaaggaagacgaagaaccatcagaataatgtaacatgcgaggatttaactcccaatataacttcgcaagaagttcgcaaagttacccagcagttgaatgctacgggacaggcaggacaagtgtactgcaaggtccagataaatggacacatacaatcagccatgatagattcaggggctacaggaaattttattgcaccagaagctgcaaagtacttggaaataccacttcaaacgaaacaacatccctaccgattgcaggacacgctagaggcgtccgcgagacgtaacacgcgccaaggggagttgaacgcgaacaacaccggcgacgtaggacacccagtccagggtcctccattaagagcgaaggccagtacacctcctctacaaatgcagaagccaacgacacggcacgaaatcgcaatcgaggcaaaagaaaggcctacgataccagaacagtacaagaaatatgaacatgttttcaaagaaccagggatccatgaggctttaccggaacacaagccatgggatcatgagataatattggaggaaggcaagatgcctgtgcacaccccaatttattcaatgtcagccgatgagttaaaaaggctcagagaatacatcgacgacaatttagccaagggatggatcagggaatccgcgtcccaagtggccagtccaactatgtgggtacccaagaaggatggacccgatagactagttgtagactatagaaagcttaacgcactcactaagaaggatcgatatccacttccattagctacggaattaagagatcgattaggcggagctacgatattcaccaagatggacctacgtaatggttaccacttgatcagaatgaaggaaggcgaagaatggaaaaccgctttcaaaacaagatacgggctatacgagtaccaagttatgccattcgggctaaccaacgcaccagctactttcatgaggcttatgaacaatgtgttgtcacaatatttggatacttgctatcaaggaagcttcgaatggactgccaaagcacaggagtcattcgatacgctcaagcaagcagtggcagaagaaccaatactgttgacttttgacccagagaaagaaatcatagtggagacggactcctcggatttcgctataggagcagttctgagccaaccgggccagaatggaaaataccagccaatcgcattctactcccgaaaactatcaccagctgagttaaattacgagatatatgacaaagaattactggcaatagtcgatgcatttagagaatggcgagcatatttggaaggatcgaaatacacagtacaggtatatacagatcataagaacttggtttacttcaccacaacgaagcagttaaacagacgacaggtcagatggtcggagaccatggccaactacaactttagaatttcatatgtcaaaggatcagaaaacgctagagccgacgctcttagccgaaaaccagaatatcaagaaaacaaaacgtacgagtcatacgctatattcaagaaagacggcgaatcactggtctacaatgcaccacagcttgcagcaacacacctgttggaagacaaccacctcaggaaacagatccaatcacactacaacaaggatgctactgccacacgcatacgcaagacaatagaaccaggattcactatagaagatgataccatatactttcatggaaaagtatacattccgagtcaaatgaccaaggaatttgtgacggaacaacatgggttgccggcacacggacatcaagggattgcaagaacatttgcaagaatccgggaaatcagttacttcccacgaatgagaacgatagttgaagaagttgttggaaattgtgacacctgcatacgaaacaagtcatcacgacatgcgccgtatggtcagctccagaccccagacatgccttctcagccatggaagtccatcacatgggactttgtggtcaaactaccactctcaaaggatcctactacaggaattgagtacgacgcgatactcaatatagtagacaggctaacgaaatttgcatatatgataccattcaaggaaacatgggatgctgagcaactagcatatgtgttcctaagggtcatagtaagcatacacggagtaccagatgagataatctcggatcgagacaagctctttacctcgaaattctggactaccttattagcacttatgggtatcaagagaaagctatcgacatctttccacccacaaacagatggtcaaacagagaggaccaatcagacaatggaagcatatcttagatgctatcgtataaaatcccgataccacaagaagttaatgccgaatcagcaatag

SEQ ID NO: 25 - DNA, 3909 bp, Artificial Sequence
  Description of Artificial Sequence Synthetic polynucleotide
atggcatccagagataccgccacaggtcaatctgccggagacaccaacgacatcgagatgaccgatgccccaaaggagatcactatcaacgaaacccttaagatcgccttaccagacaagtaccaaggtagtcgacaagagctcgatactttcctcttacaacttgagatctacttccgattcaatgaggacaagttcactaccaaggaatccaagagtatatgggccgcatcatacctccgaggtgaagcaaccaaatggattcaaccatatttgcgcgactatttcgaacatgacgataagaatcgcatgcaacccacccgaacaatcttcaatagttttgaaggatttaagacagagattcgtagaatcttcggaaattccaacgagttagaggtagcggaagataagatcttcaacctcaagcagacaggatcagcattgaaatatgctacggaatttcgaagatatgctggaacaaccaagtgggacgaaatcgctatcatgagtcactaccgcaagggactcaaaccagaagtcagactggaattagaaagatctgccgagagtacagatctgaacgatctaattcaggactccatcgaatcagatgatcgtctctacagatatcgacaaagccagagatcatacaaaccccaaggaaatcagaagcaagggcgttaccgcaagaatgagggtagaccacgttacaatccacagaggtacggagacccaatggaactagacgctacgcactacacaaacgggaacgatgactcagaaaagagacgaagacgagaaaacaacttatgctttgaatgtggaaaagcagggcaccgagcagcagagtgccgaagcaagaagacaggaggaaaaaggggcaacttcaaacctaagttcggcaagggccaacttaacgccacctttgcaatcccagaaaacccaactaaatccgaaaatactgagactttcaccattgaagaattccagcaattactagaggaattaccacgaaatcaagagggcatgaatgcaatagacttatgggaacaagagtattacagaaccccaacaccctctgtaacagaagaaagtcaccaggacgaggcagaagcagaccacgccacaatgagctggacagcctgctatgatgaattctgcggaattcatcgatcagataaagaagcaaccggatggttccccaagaaaaggaagacgaagaaccatcagaataatgtaacatgcgaggatttaactcccaatacaacttcgcaagaagttcgcaaagttacccagcagttgaatgctacgggacaggcaggacagatatactgcaaagttcagataaatggacacatacaatcagccatgatagattcaggggctacaggaaattttattgcaccagaagctgcaaagtacttggaaataccacttcagacgaaacaacacccctaccgattgcaggacacgctagaggcgtccgcgagacgtaacacgcgccaaggagagttgaacgcgaacgacaccggcgacgtaggacacccagtccagggtcctccattaagagcgaaggccagtacacctcctctacaaatgcagaagccaacgacacggcacgaaatcgcaatcgaggcaaaagaaaagcctacgataccagaacagtacaagaattatgaacatgttttcaaagaaccagggatccatgaggctttaccggaacacaagccatgggatcatgagataatattggaggaaggcaagatgcctgtgcacaccccaatttattcaatgtcagccgatgagttaaaaaggctcagagaatacatcgacgacaatttagccaagggatggatcagggaatccgcgtcccaagtggccagtccaactatgtgggtacccaagaaggatggacccgatagactagttgtagactatagaaagcttaacgcactcactaagaaggatcgatatccacttccattagctacggaattaagagatcgattaggcggagctacgatatttaccaagatggacctacgtaatggttaccacttgatcagaatgaaggaaggcgaagaatggaaaaccgctttcaaaacaagatacgggctatacgagtaccaagttatgccattcgggctaaccaacgcaccagctactttcatgaggcttatgaacaatgtgttgtcacaatatttggatacttgctgtatatgctacttggacgacatcctagtatattcaaacaacaaggttcaacacattaaggacgttagcaacatcctcgaaagcctatccaaggcagacttgctgtgcaaaccaagcaaatgcgaattccatgtcacagagacagaattcttgggattcaccgtatcaagccaagggctcaagatgagcaaaggcaaggttaaggcagtgctcgaatggaagcagccgaccacaatcaaggaagtacaatcctttctagggttcgtcaacttctacagaagatttatcaaaggttattcagggattactacacccttgaccacgttaaccagaaaagatcaaggaagcttcgaatggactgccaaagcacaggagtcattcgatacgctcaaacaagcagtggcagaagagccaatactattgacttttgacccagagaaagaaatcatagtggagacggactcctcggatttcgctataggagcagttctgagccaaccgggtcagaatggaaaataccagccaatcgcattctactcccgaaaactatcaccagccgaattaaattatgaaatatacgacaaagaattactggcaatagtcgatgcatttagagaatggcgagtatatttggaaggatcgaaatacacagtacaggtgtacacagatcataagaacttggtttacttcaccacaacgaagcagttaaacagacgacaggtcagatggtcggagaccatggccaactacaattttagaatttcatatgtcaaaggatcagaaaacgctagagccgacgctcttagccgaaaaccagaatatcaagaaaacaaaacgtacgagtcatacgctatattcaagaaagacggcgaatcactggtttacaatgcaccacagcttgcagcaacacacctgttggaagacaaccacctcaggaaacagatccaatcacactacgacaaggatgctactgccacacgcatacgcaagacaatagaaccaggattcactatagaaaatgataccatatactttcatggaaaagtatacattccgagtcaaatgaccaaggaatttgtgacggaacaacatgggttgccggcacatggacatcaaggaattgcaaggacatttgcaagaatacggggaatcagttacttcccacgaatgagaacgatagttgaagaagttgttggaaattgtgacacctgcatacgaaacaagtcatcacgacatgctccgtatggtcagctccagaccccagacatgccttctcagccatggaagtccatcacatgggactttgtgatcaaactaccactctcaaaggatcctactacaggaattgagtacgacgcgatactcaatatagtagacaggctaacgaaatttgcatatatgataccattcaaggaaacatgggatgctgagcaactagcatatgtgttcctaagggtcatagtaagcatacacggagtaccagatgagataatctcggatcgagacaagctctttacctcgaaattctggactaccttattagcacttatgggtatcaagagaaagctatcgacatctttccacccacaaacagatggtcaaacagagaggaccaatcagacaatggaagcatatcttagatgctatcgtataaaatcccgataccacaagaagttaatgccgaatcagcgatag

SEQ ID NO: 26 - DNA, 288 bp, Artificial Sequence
  Description of Artificial Sequence Synthetic polynucleotide
atgaccaaggaatttgtgacggaacaacatgggttgccggcacacggacatcaagggattgcaagaacatttgcaagaatccgggaaatcagttacttcccacgaatgagaacgatagttgaagaagttgttggaaattgtacacacctgcatacgaaacaagtcatcacgacatgcgccgtatggtcagctccagaccccagacatgccttctcagccatggaagtccatcacatgggactttgtggtcaaactaccactctcaaaggatcctactacaggaattga

SEQ ID NO: 27 - DNA, 11301 bp, Botrytis cinerea
caaagggggcattacgcttccaactgccgaaaccctgttgtatgtcaacactgtaaaggaagtcacggatccagagagtgcccaggaactatgtcacagccttcccgacagggaaacgcttagacccagctgttattctgagcgtcccactgacgctgggtccccaaatagaaggacgtactacctttaccattatagcaatgttccaaccaaaagataagccgatagcgcttcgatgccttatcgactcaggagcacaagccaacatcatccaacaatccaagtgtatcgaatgggactggctgcctattaagaaaggaacagctttagtatctgcgaacggtaccacgatgccgtcgtatggtaaccatcagttccccgtcgaagtaaaagatcaaaagggagagaagagaaccttcacccacgagtttactgctgctgtactagacttacccaaaatcgatgctatatttggattaccctggctacaagcggtaaacccagatatcgactggaaatcgacgtctcttcactatcgcccctctcttagcgacctcgaaatgatttctgcaagcgaactctatagcgaagtgaaaaagggcgtccatgtatatgttatactaccagagatccagccccattaccgtagagacaacgggtaccgccgggtactcacgctctccacactaaatatccccgaagaataccaagaataccaacaagccttttccgaggaagaaagcagtactctaccagaacaccactcgatggagcatcgcattgatctcgaagccgattcgaaacctccttgggggccaatctattctttatctgaagaggaatcaatagtattaagggaatacttagtagaatatcaaaaaaagggatggataaggaggtccattagttcggcaggagcgccaatcatgtttgttcccaagaaggggggaggctatcggctttgtgtcgactaccggggtctaaataggataaccaaaaaggatcgaaccccgctacccctaatcagcgagtccttagaccgacttcgacaaggtgtcgtcttcactaaattggacctgcgagatgcctaccaccgtattcgtatcagggaaggcgacgaatggaagacggcgttccgcacgcggtacgggcaattcgaatacttagttatgccattcggcctgaccaatgctccagcaacgttccaaacatacatcaatcaagcactgtcaggcttgacagacaccatatgcgtagtgtacctagatgatatcctgatttactctgaggatagagaaagccacacgcgggatgtccgcagggtcctcgaacgccttatagaatacaagctgttcgcaaaactgaaaaaatgtgtcttttacacccatgaggttgaattcctaggattcgtcgtctcgggagcgggagtgacgatggaatccagccgcattcaaactattatagaatggccaacacctacaaaccttagggagctacaggtgttcctgggcttcgcgaacttctatcgacggtttatcaggacctattcgacggtagcccacgggatgaccgcccttatgaagggaacaaagaaaggtaaaatggtaggggagtttatatggacaaaggaggcccaagatgcatttgaggcactaaagaaagcattcaccacggcaccgatactcaagcacttcgaaccatcgctccgcatcatggtcgaaaccgactcgtcggtgtttgctctaggatgcatcctatcgcaactattcgaaggagggactgcagaagcaccgatacgacggtggcaccccgtcgcgttctattcgagaaagctgaaccctgcagaacaacgatacttcactcacgatcaggaattattagcaatatacactgcattcatgcaatggcgccattacttgataggtagtcggcacacaatcgtggtgaaatcggaccataacagcttacaacattttatggtgaaaaagaccctcaatggcagacaagctagatgggcggaagtactagcagcctacgacttcgaaatagtgtacagggcagggaaactgaatccagccgacgggccatcgcgccgccccgactacgctaccgacacggagggtatcaatgatatgctacccacactccagaataaattaaaaagtaccgcagttatcgcgagtttattttacgaatccaccgtgaaaacggaacccctgcgtattgctattagtcgcttgcaaagggaagggtatagcttgccattacgtggacagttagtttcactggtaaaaactggttgcaaacagtcgataccacgtcggattgccagtgttttcgcatccgacgaaacggcattcgaacctatatcggagtcgatgggaaaagctttattgcggcttcagaaagaagacgattttataaagaataaagagtacctaagacaaagattacgttccgccggagacgcctcaccacggcaggtgggcgccgacgagctccttagacacaaggggagcgcgtacgtaccgccagacagcgctctcagagcagaaatcttagaaacgcatcacgatgaccctattggaggtcattggggtgtcgctaaaacattggaaatactgaagtctaaatattattggccttcaatgagaaaagacgtcaaacaacatgtcaaaacatgtgcggtatgccagcgaaccgctatcaaaagacataagccacacggcgagttacagaccctccctattccaaaaggaccctggaaagagataactatggattttattacagatttacctccttcgaaacacggaaaacacgtatacgattctattctagtagtagtcgacaggttcacgaagctagcccgatatatcgccgtcaacaagacgatatcgtctcctgaattagctgacactatggtcagcacagtatttaaagactttggtgtgccagagggcatagtctccgataggggaccgcaattcgtcagtaaattttggagtagcctaatgttttacttgcgaatccgtcgtaagctgtcgacggcgttccacccgcagaccgacggtcaaaccgaacgacaaaaccaaaatttgattcactatataagttgctacaccaactataggcaagacgactgggcatcgctattgccccttgctgaattcacatataacgcgacatggcacagtacaaccaatacaagcccattccaggctatgtatgggttccaacccacattccattatatcggcgaggacgccgatttagagggaagggcgccggcagcacgcgagcgcatcgacgctttagagaaagaaagagaaaagctgaaagaattctggaaatcggcaaccaatcagaaaaacaagaacactacgaaggggtcaccacagcgatatagcatcggggacaaggtgatgctaagcacaaagaacattaaacaactgcgacctaagaaaaaattctccgatcgatttataggcccctttgtcgtgacgggtataaaaaccagcgggcaagcatacgaacttagattaccgcccacctacaagatccacaatgtattccacgtctctttactcgaaccatggcacgaacgacagggtaccgccgacccgccgccgccagaagaaattgacgatcacatagagcatgaggtggaaaggattttagcacatagaaaaagaggcagaggtgtgcaatacctggtgcgatggaagggctaccaaccggcggaagacacgtgggaagcaccctacaacctagaaaatgcgaaagcagcgatgggagaatatcataaagaagaagcattaccaatacagaaaaagaagagaacaagaaaaaagcttaaaaatacttgatacaaaaagacctcacgagacccacacccagaacgcatgcatcacaccaagctacccaaaaaggactatccaacaaagaaaccagaaaggacaactcctccgaacccacagcataccgacaaccaaacccaagtgacccatcacgcaaagagaactctggaaggtcgaaatcagttccctaattcatctgcctgatccaggaggtcagctgcaatatctcgatcgccaagaaggacagaacctacatcgctggcatatcccccgggactcgcgagcccgatctgatcaacctcaccccccccactgatctcatcttgatcaccgactccttcctcctcgttctcgcgaatctcgctgacagggcgagctcctgtcggaaaaccagcggcaacccgttgcctcttggagacccgctccacatcagaattctcagagcgaagcctttggggcgaatctgcttgactatcagactcaccaatatagacttgttgaacagtgctgggagccctctttcgggatttaagagagcctacgggatgagagcggggtgtcggtgtgttgcgagaggcaagctgcgacgacttagaagcggaaaggggaatggcggatctagtcgcgagcttatcggaccgagaacgacgaggcgtagcaggaggagcgttctttctacccaagcttccaaacaataaagccggtacactctcatccaaggggtgacgcggggcaccggtggtctgatgaggttgatccgacacatcatcctcttcaggagcagactcttcggattcattgggatccacgattacactcctcttcaacttcgcagaacctttccggaggccttgctgagccgtcgtgaagacagcggggcccgtccggctcgatgaagcagaagcatgacggcgacctagggaaccagctaccatattcaggccctggttaccacctgggggccccggaggagcttgaacaccctgcagtgcgtcggcgacagcgcgcacagcgcgagggtgtataaccggagccatgaactccccttcttcgtccgagtttaaggcggcgattaatggtgcagtaggagctgcggcggcgacgtccaaggcaggcaggttattctaagaaccatcaaatcagctatcaacagcaccaaggggcaagagcagggcaaccgacaacgaatcgatacgaatccacaatgcgctccataagatcgccaattctactcaatttggacaccagaagaagagtcaattcctctgcggactgcggtttgtgagtccccccttgtttaacctcttttcgaaggaaagcctccactgccttgacgtatcgattacaccggcgaatcaccacctcggaggcgtcctcatcctcttggctattatccggagtaaggcaccaaaaggcatgagtctgatataagaggttcacgtcgggaacaaagcgaggaggtatcgggaggcagactttcttttgtttgcgacaataactacatttagcagcaggacctttatcaaaatggcaaaattcgtcgttagaagagacggcaattcgcttagaacatcgaaggcaagtagggataactaacgcggaaggggccgctgcggaccgatcagcgatagcagccggagcgatgggttgcagtgcagccatcttcgtatgagtaaataagggggaataatctgattgtgggagatatatcagaggcaagaagaccccccttatagaactatcggtgatctgccggtaaggcggtgaggcgcgtaagaatgccgccgtttgcttgtttattgtttgtaatgcctaaacaagattggaattgcttttggaatgcggcgcagggtcgggcatgcagcgacgcgacgacgcgacccacattccgagtaaacaatacggaaggaagcaaacacttctcgggacgcgaagtgtaaagagaggggctctgttacgggacaaaacgtgaccggctcaattaggcacgtgacagtggacctctcgggtctactgcgtgccgaatggggcccgcacacgtataaattgtataatttgcatagttatagaaaagcaatgaaaagtcttggtgccacaatatactagttgattcatttgttacggaggtacccgcaccgcaacatggattataagataaacctaaggccttggtgttggaacctacgaaaacagcactgtagggacagttgaattaaagggtaactaaagatagcagtaaccgaatcaataagcaatgattaaaagataggtacctatcttttgttggcacctaccctacagtaggcacaggagggatagcggttataggttatctagtaagcacaggttagataagcagtagtatcatgtaggtcacggggcaagtgtcacgtgatggatagacaggataggcaggctatccaggctatccgtggatagacaggatagacagtctacccaagctatccagacgagaacgaaggtctatataagggaatgggtttcattacaatgtagagcttcgtgctcaagaacaatcattagtttcattactatagttacgagaattgcaaccagttacaaccttattgaattcctacttgaagtctagtctaaaccacctcgagagatctctagacacttccacgtgaccctagaggcagctcccgtaacactttgagcaccctttctgcttcaagtaccgattcgataaccaaccgctaatatggcatccagagctaccgccacaggtcagtctaccgaagataccaacgacatcgagatgaccgatgccccaaaggagatcactatcaacgaaacacttaagatcgccttaccagacaagtaccaaggtagtcgacaagagctcgatactttcctcttacaacttgagatctacttccgattcaatgaagacaagttcactaccaaggaatccaagagcatatgggctgcatcatacctccgaggtgaagcaaccaaatggattcaaccatatttgcgcgactatttcgagcatgacgataaggatcgcatgcaacccacccgaacaatcttcaatagttttgaaggatttaagacagagattcgtagaatcttcggaaattccaacgagttagaggtagcggaagataagatcttcaacctcaagcagacaggatcagcattgaaatatgctacggaatttcgaagatatgctggaacaaccaagtgggacgaaatcgctatcatgagtcactactgtaagggactcaaaccagaagtcagactagagttagaaagatctgccgagagtacagatctgaacgatctaattcaggactccatcgaatcagatgatcgtctctatagatatcgacaaagccaaagatcatacaaaccccaaggaaaccaaaagcaagggcgttaccgcaagaatgagggtagaccacgttacaatccacagagatacggagaccccatggaactagacgccacgcactacacaaacgggaacgatgactcagaaaagagacgaagacgagaaaacaacttatgctttgaatgtggaaaagcagggcaccgagcagtagactgccgaagcaagaagacaggaggaaaaaggggcaacttcaaacctaagttcggcaagggccaacttaacgccacctttgccatctcagaaaactcaactaaaaccgaaaatactgagactttcaccgttgaggaatttcagcaattactaaaggaattaccacgaaataaagagggcatgaatgcaatagacttatgggaacaagagtattacagaaccccaacaccctctgtgacagaagaaagtcaccaggacgaggcagaagcggaccacgccacgatgagctggacagcttgctatgatgaattctgcggaatccatcgatcagataaagaagcaaccggatggttccccaagaaaaggaagacgaagaaccatcagaataatgtaacatgcgaggatttaactcccaatataacttcgcaagaagttcgcaaagttacccagcagttgaatgctacgggacaggcaggacaagtgtactgcaaggtccagataaatggacacatacaatcagccatgatagattcaggggctacaggaaattttattgcaccagaagctgcaaagtacttggaaataccacttcaaacgaaacaacacccctatcgattgcagttagttgatggacagctagcagggtctgacggaaagatttcgcaggagacaatcccagtacgaatgggcataacccaacatacagaggttatacagcttgacgttgtgccattgggccaacaacagatcatcttaggaatgccatggttgaaggcacataatccgaaaatagattgggcacaaggaattgtgacatttgatcagtgcaaaagcggtcacagggacacgctagaggcgtccgcgagacgtaacacgcgccaaggggagttgaacgcgaacaacaccggcgacgtaggacacccagtccagggtcctccattaagagcgaaggccagtacacctcctctacaaatgcagaagccaacgacacggcacgaaatcgcaatcgaggcaaaagaaaggcctacgataccagaacagtacaagaaatatgaacatgttttcaaagaaccagggatccatgaggctttaccggaacacaagccatgggatcatgagataatattggaggaaggcaagatgcctgtgcacaccccaatttattcaatgtcagccgatgagttaaaaaggctcagagaatacatcgacgacaatttagccaagggatggatcagggaatccgcgtcccaagtggccagtccaactatgtgggtacccaagaaggatggacccgatagactagttgtagactatagaaagcttaacgcactcactaagaaggatcgatatccacttccattagctacggaattaagagatcgattaggcggagctacgatattcaccaagatggacctacgtaatggttaccacttgatcagaatgaaggaaggcgaagaatggaaaaccgctttcaaaacaagatacgggctatacgagtaccaagttatgccattcgggctaaccaacgcaccagctactttcatgaggcttatgaacaatgtgttgtcacaatatttggatacttgctgtatatgctacttggacgacatcctagtatattcaaacaacaaggttcaacacattaaggacgttagcaacatcctcgaaagtctatccaaagcagacttgctgtgcaaaccaagcaaatgcgaattccatgtcacagaaacagaattcttgggattcaccgtatcaagccaagggctcaagatgagcaaagacaaggttaaggcagtgctcgaatggaagcagccaaccacaatcaaggaggtacaatcctttctagggttcgtcaacttctacagaagatttatcaagggttattcagggattactacacccttgaccacgttaaccagaaaagatcaaggaagcttcgaatggactgccaaagcacaggagtcattcgatacactcaaacaagcagtggcagaagaaccaatactgttgacttttgacccagagaaagaaatcatagtggaaacggattcctcagatttcgctataggagcagttctgagccaaccgggccagaatggaaaataccagccaatcgcattctactcccgaaaactatcaccagctgagttaaattacgagatatatgacaaagaattactggcaatagtcgatgcatttagagaatggcgagtatatttggaaggatcgaaatacacagtacaggtgtatacagatcataagaacttggtttacttcaccacaacgaagcagttaaacagacgacaggtcagatggtcggagaccatggccaactacaatttcagaatttcatatgtcaaaggatcagaaaacgctagagccgacgctcttagccgaaaaccagaatatcaagaaaacaaaacgtacgagtcatacgctatattcaagaaagacggcgaatcactggtctacaatgcaccacagcttgcagcaacacacctgttggaagacaaccacctcaggaaacagatccaatcacactacaacaaggatgctactgccacacgcatacgcaagacaatagaaccaggattcactatagaagatgataccatatactttcatggaaaagtatacattccgagtcaaatgaccaaggaatttgtgacggaacaacacggattgccggcacatggacaccaaggaattgcaaggacatttgcaagaatacgggaaatcagttacttcccacgaatgagaacgatagttgaagaagttgttggaaattgtgacacctgcatacgaaacaagtcatcacgacatgctccgtatggtcagctccagaccccagacatgccttctcagccatggaagtccatcacatgggactttgtggtcaaactaccactctcaaaggatcctactacaggaattgagtacgacgcgatactcaatatagtagacaggctaacgaaatttgcatatatgataccattcaaggaaacatgggatgctgagcaactagcatatgtgttcctaagggtcatagtaagcatacacggagtaccagatgagataatctcggatcgagacaagctctttacctcgaaattctggactaccttattagcacttatgggtatcaagagaaagctatcgacatctttccacccacaaacagatggtcaaacagagaggaccaatcagacaatggaagcatatcttagatgctatgtaaattatcgacaagacaattgggtagagctattacccatggcacagttcgcatacaatacatcggaaacggaaaccacgaaaatcaccccagcacgagctaattttgggtttaatccacaagcgtataaaatcccgataccacaagaagttaatgccgaatcagcaatagtacaagtcgaacagctgaaagatctccaagagcaactggctcttgatctaagattcatatcttccagaacagcagcgtactacaatacgaaacgtagtatggaacctacgcttaaagagggggataaagtttatttgctacaacgaaacatcgaaaccaagagaccaagcaataaactcgaccacaggaaaataggaccattcaagattgataaggtaataggaacggttaattatcgattgaaattaccagacacaatgaatatccacccagtattccacatatccttgctcgaaccagcaccaccaggagcgccaaatgcgccatttacagaaatcgaaccagtcaacccaaacgccatatacgacgttgaaacaatactagattgtaaatatgtcaggggcaaaatcaagtatttgatcaaatggttagactacccacattcggaaaacacatgggaa

SEQ ID NO: 28 - DNA, 936 bp, Botrytis cinerea
gaagaggttgttggcaatattttgaagaaagctgaggctgatttgaatggagattaaaaggggaatgaagctgcggggccaccgatagcacaaaaactactgaagatttgaagcacgttaaaattacactcaggaataaacggatggcaagcttttcgatcgcccaaacacggatctacgactacgagttacgcacgacatgatttagccttttgtgtgcaatgatgattagatagcattgcatttctcgaaattgacggcacgacttttacgggcagataatatcaaagattcctagtgagcaagcggtgatgatacgatgtcattccaaaagttttttcctcgcgaattttatttcatttcgaaggcatctttgcttagcagcatattcacctttgatgtcctctgtaggggatggagtctctaatctcgcggtcacaatgagacgtgatgcgctgcgaagtggtgacaatttccctttacttagaatagatcatgcacacatgcatgatgcatagctagctagttttttattcaatgatagtttaatgacaaacacgtatctagatatcctcattcatgtatctgtgggaggttgacttaagttatggctgacttgatagtttcattatatatgtatatgtgatatctaagtaaagattaaagtgaaatcgaaatgcaacgccgaaattctattaattccatgaaatgatgtgatatggcatgacatgatatccaaactccgatttgaaatgctccagcttcgctttctaaaattggtaaaagggacattatttcgtctggttgtgggttttcatttctgtgctcctactaggtgtgaatgatagagtatgctgtggtgtggtgtgatctcggaatttggaaatttgagggctgtatatcacctcatttcgtgtgtccgaatttctacagact

SEQ ID NO: 29 - DNA, 670 bp, Botrytis cinerea
agagcatttgtaggggaaggaggaaaaattgaggaggaggataagatgaattttgataaatttatttcctaacatcaggtcacaatctatgaattacatttgatagtattacgtatgccggtctgtacacaacacaaccatatagtaaggtatcaatcaaatgcgatggatagtcatttcaatttcttagtgaataattacaacgaaccagtaaaatagcaataactctgaaaagcttccggactgccaaaaggtctccaggacgagattattacgaagaacccaagaattcgcctaggaaccaagataaacaaatcatcgacgtgttgcacttccatctatgcgacaattatgccaagcgagccgccagttcttgggggtggagcgctaggaatagggggccggattgccatatccttatctagatctagatggtatcgatatgataaatcaatgcaatggagagttaaaaagttatatgccatatgattgataattattgacaatgcaggctatcgcgggacaatggtaaatggttgtaaaatatggagtctatttccttagctagcgataagatgggtggtttaaacacatcccgccttctctttatcattctccttctcgtattcatatatcataattgcaaagtaaggttgtattttggactgtg

SEQ ID NO: 30 - DNA, 345 bp, Verticillium dahliae
aagctgtcaattgatgcggagggtgagtgaacgtctcgtcggcggggccccttgaggcgagcgcccgttggggggtgttgtggcactaggttctctaggccggcggtgactttcattactatattagaagcaaatacggcgccttcatcacaataataaatatcgatctcgagtcgattccagacccgttataaacctatgtctgtgcaaccagttgggtgctaatttcttgcattatcatcatggatgttgtctatttgagtctcaggtccagctggtgcttataggtcatctccagtatgcgactacctctctccctctttgccattcctaactgattctaac

SEQ ID NO: 31 - DNA, 1000 bp, Verticillium dahliae
cttcatcttccaaccgccattacctcccccatacgcgtcctgccaaagaatcataactggctaaaacataagacgggactggtcatccgctgaaccattccgagctatgtgtcctgattgacccatctcggcttattcgctctcaaatacgactgcaatcgcgtgtggcttggaaaccgtggaataccatcctcatattgtcagcacctgtagcgatacagcacaatgcttgacgattcggaatcattttccgcttctttgcggagcagcggatgtccaattgacgatgacttgactccagaaccaacgtccgaatcacgcgactcaacctccctaccgtatggccttcaggacgacatcggcccccttgctgccaccccgagccagtcgagtaacgtcacaatcaatgcacgggcataccagttggagatgctggcggaaagtaggaagaggaatatcattctagctgtgcgaaccttccctcttgcgccagtcaacctcgattgacacctccatagatggacacaggcagtggcaagacccaagtgtacgttccctgcaagccgaacctgattattgatactgattttcccagtgccgtcctccgaattcgagcagagctagaagaaggggcttcagacaaggtttgacaaactccacttggtagcttcgcaatcacttacagggttttagcttgtatggttcgtggctcacaatgttgagctttgcgctcagcagcattctgtactgcagtctcagattcctgcagttcagaccaagctgcttcttggcagcgataatgttgattcatggtccaaccaagagacttggaacgctgtgcttctcaacgtcaaaattgtggtgtcaacccctcaagttctctgcgatgccttgagccacggctttgtccagatgggttcattatccttgcttgtctttgatgaaggtattcaatcagcgcagtttatcaagtgttcttgccctaacaacggtgtagcgc

SEQ ID NO: 32 - DNA, 3437 bp, Botrytis cinerea
gcaggggtcggatcaacatgtctataaacaaacatatgtaccggcgttgatctctcctgcagactgcatttgcacttgcttccctcttcctcctcccgtttcctggtcttcttctacaagctgcaggcgagagagataacttctacgcaccttccatatccctcacctcttctctccccacaagttcgttcataatcctttcgtcctgttgttttgtctagcattaccttgcaattcttaacaacggccgatcgtggacatcaatcaataaaaaggacgacaaatcatcttataattattatcccaaactttcattgcacaaatttgaattggatactcatttggctttattcggagcgataaacgtagaaattaatcgtataggggcttttatcagacaatcaagaacggtgattggctcacagcggtgaattgtgaggggtggtaatacagaaaacaaatagtatagggagtatttttgggtggattgttaccaatgtctaccacaagaatctcaacaccgaaaaggtcccccaaaaaatcgacttttgtcaaaactggaatcttgaccaccaaatcaacgcccaatctcaacgcctcctataatttggcattactacaagcttcaggagctacacccgttcctgcatatccttccaataacggtcaaagttttgccctaaataatcctaggtcgcaaccgtctcgacaagtctcactcgcttcccttacctcgaattcacttgcgacaatcccggatgcaagcaagagataccctctttctacagtctttgatgaggatatgccaacagtaggcaacatgccgccatacacacctgctcgagttggcggtggaccggaagaactagaggttggtgatatagtcgatgtgccaggtaacatgtatggtatcgtcaaatttgttggcagtgtgcaaggcaaaaagggtgtatttgctggggtagaattaagtgaaacgtttgcttcgaaagggaaaaacaatggcgatgtcgaaggaattcaatactttgacacaaccatcgatggtgctgggatttttcttccagtcaacagggcgaagagacgtagcaccccttcgtcgcatgatgagtcatttcccctttcaccggcgtctccatcgatgggcaatagggctgggagattaggatctgaattaaatggtcagccaacacctttgttaccaaaattcggtcaatctgttggtccaggcagagcggcaaacccatatgtccaaaaaacacgtccatccatggctacacctaccacctcaagaccggaatcaccagttcgaagagcagccaatgccaacccatcattaaatacacctgcacaaagagtcccatctcgatatgcaagccctgcgcaggcaaactttggacagagcgttagaggaacacaagattctagagatccaagtaagaaagttggctacaccccccgaaatggcatgaaaacaccaatacctccacgaagtgtttctgcacttggaacggggaatagacctgcaccaatgaactcgatgaatttcagtgatgaagagacacctcctgcagagattgcacgtacggcaacaaacggaagcgtaggctcagtctcttctttcaacgcgaaattacgtccagcatcaagatccgcatcgcgtacaacttccagggctaccgacgacgaatttgagcgattgagaagtttgttagaagatcgcgatagggaaataaaagaacaggcttctattatagaagacatggagaaaactctcagtgaagcacaatcgttgatggagaacaataacgagaacgcaagtggtagacatagtcagggaagtgtggatgacaaggacgcaacacagttgagagcaataatacgtgaaaagaacgacaaaatcgccatgctgactgccgagtttgatcagcatcgagctgatttcagaagcacgatagacacgctcgaaatggccggtgcggaaaccgagcgagtgtacgacgagcgcatgcgtgttctcgtaatggagctcgatacaatgcacgagaatagtcatgatgtaaagcacgttgctgtacaactgaaacagctagaagagctcgttcaggagctcgaggaaggtcttgaagatgcacgacgtggtgaagccgaagctcggggagaagttgagttcttgcgtggagaggttgaaagaactcgatctgaactccgccgcgagcgagagaagactgccgaagctcttagcaacgcaaattctcctacgagcgcaagtgcggaaacacattccaaagagattgctcagagagatgacgagattcgtggattgaaagccatcatccactcgctcagcagagatgccatacctgatgggaatttctcggatcatgaggcaacaccaaatattctacgacctggactaaaccgaagtcgaacagaaagtgcttcggtttctgaggaggagcgccgtactcgggaaaagctagagcgagaagtgagtgagcttcgtgctctcgtcgaaagcaaagacaataaagaagaacaaatggagcgcgagttggagggattgcgaagaggaagtgttagcaatcctactacgcatcgtactagtgccatgagcagcggaactgtgactcaggataggaattctctccaagacaataagagcacagttgtaagctggcgagaacgtggtgcctcagatgctcgccgctacaatctggattcaatgccagagaatgacagctactcctctgcagctgaggatttctgtgaattatgcgaaacctcaggtcatgatgttctacattgcccgatgtttggccccaatggtaacagcagcaattctaaggatgagtcacctaaacagcaacgaacaggaaaagacgttgtcatggagggacttaaattatcacccaaaccttctcaagaagaatacaaaccggcgccgttagcgccagctaagaagtcgcctgatgcgtcgcctatcaagactgttcccaaccttatggaaccaggacctgccccaggaaaggaaagtggagtaatcaacatggataaatggtgcggtgtatgtgaaagagatggacatgacagtattgattgtccttttgaagatgctttttaggagactactgctttcgatgtttcaggataagcagtcacaacgacgacttttttcatagattttctttgttaatcataggcaaggccgcattgcattgcaggagcgtaatccgtctgcgatataccctttcggttctctgtttgaagtatgcttttcaagcgataagtttagaggggaagatgatgtttttacgaggattgaatgagatggatgaatgcaggctaaatcggggaagggggagggaagacaaacatgagttgaacggacgtaatgatcatgtagtatactttgtcaaattaatgatccaaatgca

SEQ ID NO: 33 - DNA, 2406 bp, Sclerotinia sclerotiorum
atgtcgactacaagaatctcaactccaaaaaggtctccaaaaaaatcgacattcactaaaacaggaattcaagtcacaaaatcaactcccaatctcggtgcctcctacaatttggctttattacaagcttcaggagcttcaccggttcttgcacatttttccaataacggtcagggttttggtctaaacaatcctaggtcgaagccatctcgacaagtctcactcgcatcccttacctcaaattcactggcggcaataccggatgctagtaaaagataccctctttcaaccgtttttgatgaggatatgccaccagcaggcaacatgtatacaccttctcgagttggtggtgggcccgatgagttggaggtgggtgacatagttgatgttcctggtaacatgtatggtactgtcagatttgtcggcagtgtgcaaggcaagaagggggtctttgccggagtggaattggatgagatgtttgcttccaaagggaagaacaatggtgatgttgaaggtcaatcagttggcccaggtagaattcaaaaaacccgaccatcgatagccacaccaaccacatcacgaccagagtctccagtacgaagagcagccgctgctaggacatcaataaatgcacccgggcagagagtcccatctcgatatggaagtcctgcagcggcgaactttgggcagaacattagaggagtgcaagatgctagagacccaagcaagaaagtcggttacgccccaacaaatggcatgaagacaccagtccctccacgaagtgtttcggcacttggcacagggagtagacctgcagcaatgaacctcagtgatgaagatacaccttctgctggaattacacggacggcaacaaacgggagtgtgagctcaatctcttccttcaacgcaaagttacgacctgcatcaagatccgcctcgcgtgcgtcccgagctactgacgacgaggtcgagcgattgagaggtctactggaggagcgcgatcgggaaataaaagcacaagcttcaatcatagaagacatggaaaagactcttagtgaagctcagtcactgatggaggacaacaatgagaacgcgggcggtcatagagatagccggggaagcatggaggacaaagacgcagcacaattgagagcaataattcgtgaaaagaatgaaaaaatcgccatgctgactgctgagtttgatcagcatcgagctgatttcagaagtacaatagacacacttgagatggctggtgctgaaaccgaaagagtctacgatgagcgcatgagtaatcttgtaatggagctcaggacgatgcatgagaacagtcatgatgtgaagcatgttgctgtacaactgaaacagctagaagagcttgttcaggagcttgaggaaggtcttgaagatgcgcggcgtggtgaagccgaggctcgcggtgaggtcgagttcttgcgtggagaggttgaaagaactcgatctgagcttcgtcgtgagcgggagaaaactgctgaagctctcagtaacgcaaatcctgctacgggtgtgggtgcagcaacactttctaaagagattgcacaaagagatgacgagatccgcggtttgaaagctatcattcactcgcttagccgagatgccatacctgatgggaatttctcggatcatgaaaagacaccaagtgttacacgaccagggctacatcgaagccgtacggaaagcgcttcagcttcagaggaggagcgtcttagccgggagaagttggaacgagaagtgagcgaacttcgtgccgtcgtagaaagtaaagacagcaaggaagaagaaatggagcgtgagctagaggggctacgaaggggaagtgtcagcaattctactacgcagcgtactagtgccattagcagtggaactgcaacccaggatagaaactctgtccgagattccaaaggcacagttggaagctggcgggaccgcgaaggaacatcggatgttcaccaccacaacttggagtcaatgccagagattgacggttactcttcagcagcggaggatttctgtgaattgtgcgaggcatcaggtcatgatgttctacattgccccatgttcggtcctaatggtaatagtggcaactctagagaggagtctcctaaagagcaacgaacaggaaaagacgttgtcatggaaggactcaaactatcacccaaactagcgcaagaagaatacgaaccagcacctttagcaccagccaagaagtcgtctgatgactcgcctattaaaaccatccctaacctcatggacccaggtgctgctccaggaaaagcaagtggagtcatcaatatggacaaatggtgcggtgtatgtgaacgagatggacatgacagcattgactgtccgtttgaagatgcattttag

SEQ ID NO: 34 - DNA, 2601 bp, Botrytis cinerea
gacacatgcgatatgcaaagtctagaacctcgaatactgattcgaaaaagactggcaattccataaatctacagtatattttaatccgcaactcatgaatgactacatttaatacgaattacaaacattccctaacgccaaaatggcagctacgattcccctctccactacaacatgcttgacctcctcagaagctttcaaatatcctcttccacagattcgtcaattccaccgcgatctcactacagagcttgacgagaaaaatgcacgtctgcggacactggtcggagggagttatagacaattacttggaaccgccgagcaaatcttacagatgcgacaggatattagtggagtagaggaaaagttaggcaaagtaggagaaggatgtgggagaaatgtgttggttggaatggttggcggattgggaaaattacagggagaaatgaagaatggaaagaagggcgaggaaatgcgggttgtggctaagatgaaggtattgggtatgtgtgggattgtggttgggaagctcttgaggagaccagggcgaatggatggggatggtgggagagggaaggaattagtagttgctgcgaaagtcttagttttgagccgattgttggcgaagagcttggagaatactggagataaggaattcgttgaagaagcgaagaagaagaggtcggctttgacgaagcgattgttacgcgcagttgaaaagacattggtttccgtcaaggatgctgaagatagagacgatttggtacagacactttgtgcatacagtctagctactagttctggcaccaaagacgtcttgcgacatttcttaaatgttcgtggtgaagcaatggctttagcgtttgacgatgaagaggagtcgaacaagcagacctcaggtgtcctacgcgctttggaaatatatacgagaactttactagatgtacaggctctagtgccaaggaggctgagcgaagcgttggctgtgctgaagacgaaacctttactgaaagatgacagcattcgggaaatggagggattgaggttggatgtatgtgagcggtggtttggcgatgagattatttacttcacaccttatgtccggcatgatgatttggaagggtcattggcggttgaaacactacgaggttgggcgaagaaagcgtcagaagtgttactggaaggttttacgaagactcttcaagggggattagactttaaagtagttgttgaactacgaacaaagattctggaggtgtgggttagagatggaggcaaagcaaggggattcgatccctctatacttctaaatggcttacgagacgttataaacaaacgactcgtagagttattagaaactagagttggcaaacttcatctagtggggacagagatagagtccacattagcaacatggcaagaaggaatcaccgacatacatgcaagtctttgggacgaagatatgatggcaaccgagctcagcaatggtggtaacattttcaagcaagacatacttgctcgcacgttcggacggaacgatgctgtttcaagagttgttaacagttttcacacttggagacatctcatcgaggaaattggtacttatattgatgaactgaagaaacaaagatgggatgatgatttggaagatatggaagatgatgaaagtctcgaatcacgacaaaaccttcttagcaaggaagatccacaaatgctacaagatcatctcgattcaagcttagaaaattcgttccaggagttacacgcaaagatcacttcactggtggaccagcaaaaagatagtaaacatatcgggaaaatatcgatatatattctccgaattctacgagatatcagagcagaattacctagtaaccctgcactacaaaagtttggactctcacttgtctcatcactgcacgaaaatctcgcaggtatggtctcagaaaacgccatcttagcccttgcaaaatctctcaagaagaagaaggttgcgggcagagcattatgggagggtacaccggaacttcctgttcagccctccccagcaacattcaaatttttgagaggtttatcgactgctatggctgatgctggagccgatctatggagccctgttgccgtcaaagtgttgaaagcgcgtctggacacccaagttgaagaccaatggagtaaggctctaaaagaaaaagaggaagagcctagcaatggaatctctggttctcccaccaatgctcccgaagcagatgccgaggaaaaagaaggggacgcttctgctcctaatcctgctgctgctgtagaagtagatgaagaaaaacaaaaggatttactaaagcaatcactgttcgatatatctgtcttgcagcaagctttagaatcacagtcagacaataaggagaacaaacttaagaacttagcggatgaggtgggaggaaaactagatctcgaggcgagggaaaggaaacgtatggttaatggcgcggcggagtattggaagaggtgcagtcttttgtttggacttttagcgtagattccagatggatgaattagtgagaggcttataatgaattatattacgaatactttacttttgagtattca

SEQ ID NO: 35 - DNA, 2424 bp, Sclerotinia sclerotiorum
atggcatctacaaccctctccacaacaacatgcttcacttcctcggaagcatttaaacatcctctccctcaaatccggcaattccaccgcgatctcaccaccgaacttgatgagaaaaacgcacgtctacgtacacttgtcggaggtagttatagacaattactgggaaccgctgaacaaatcctacaaatgcgcaaggatatccgtgaagtggaggaaaagttgggggaagtaggggaaggatgtggaagaaatgtattagttgggatggcttctggattaggtaaattacagggagaaatgaagaatgggaagaaaggggaggaaataaggggattggctagaatgaagggtttgggtatgtgtgggattgtggttgggaaacttttgaggaggcagggaagagtggatggggaggggagagggaaaagtttagtgattgctgcgaaagttttggttttgagtcggttgttggcgaagagtttggagggttgtgtgaatagtgcggatagagaatttgttgaggaggcaaagaagaagagggtggttttgacgaaacgattgttacgggcggttgagaagacattagtctcgaccaaggatggtgaagatagagaagacctggtacaggctctttgcgcgtatagtcttgctactagctctggtgcgaaagacgttttacgacattttctaaatgtccgaggggaagcaatggcattagcattcgaagacgaagaggaatcgaaccaggagacatcaggtgttttgcgggcattggaaatatatacgaggactttacttgatgtacaagcattggtaccgagtagacttagccaagcattggctgcgctgaagacgaaacctttattgaaagatgaaagtattcgagatttggagggattgagattagatgtatgtgagcggtggtttggtgatgaaattctttactttacaccttatgttcgacacgatgatttggaaggatcattagccgttgagacattaagaggttgggcgaagaaagcatcagaggtactactggaaggattcacaaagactcttcaaggtggcttggacttcaaggtagtagtcgaattacggacaaagatattggaggtatggatacgggatggaggaaaggcaagagggtttgatccgtctatacttcgagatggactgcgaggtgttgttaacgaacgacttgtagagttattggaaactcgagttggcaaacttcatctagtgggaacagaaatagaatccacattggctacatgggagaaatggattactgatcatcatgctagtctatgggatgaagatatgatggcaacggaactcagcaatggaggtaatatgttcaaacaagacattcttgctcgtacctttggacgtaatgatgctgtttcaagagtagtcaacagttttcagacttggagacatctcatcaaggaaataggtactgttattgatgaattgaagaaacaaagatgggatgatgatttagaagatatcgaagatgaagaaagtcttgagtcgcgacaaaatcttcttagtaagaaagatccacaaatgttgcaagatcatcttgattcaagcttagaaaaagcttttcaggagttacatacgaaaatcacgacacttgtggagcaatacaaagatagcgagcatatcggaaagatatcaatgtatattttacgaattttacgagatatccgagcagagctaccgacaaatccatcactacaacaattcggtctttcactgatcccattactacacgagagccttgccagcacagtttctgaaaaccctatctcttctctagcaaaatcgctcaagaaaaaaaaagttgcaggaagagcattatgggaaggaacaccggaacttccaattcaaccttcacctgctacatttaaatttcttcgtgctttatcaaatgctatggctgatgctggagcagatctttggagtcctattgctattaagactttgaaagtacatctcgattcccaaattaatgagaaatggagcatagccttgtcagagaagatggctagtaataaaacaactacttcttccagcaatccacccgatactgaaaaatccgcggaaacagaagaaccaaaaaatgaagttcaatccccgttggataaagaagtagaagaagaaaaagaaaaaaatctactaaaacaatatttattcgatatcttcgtcttacaacaagctttagcgctacaatctatacaatttggggataaggaaaaggaaaaggaaaaagggattatggggatgaaaatcaagaatttgagtgatgagattgaattggaattgaagcttgagatgcaggagaggaagagggtggggaatggtgcgagggagtattggaagaggacggggcttttgtttgggtttttggtgtag

SEQ ID NO: 36 - DNA, 3180 bp, Botrytis cinerea
gatccacccacatccttcctcatatgacttcgatgataattacatagacactgccagtatgcctggcctcgttcgcaaactccttatctttgccgccatcgatgggttgattttgcaaccagcagcgccaaaaggccaacgccccgcccccgcaacgaagatcgcatacaaagataagcatatcgggccagtattgagtgatttgcaggatctggaggggtcgtctgcgaaaagtttcgaggcatttggtattgtcggtctcttgacggtttccaaaagctccttcctgatatcgattacgaaaagagagcaagtcgcacaaatacaagggaaacctatatatgttattactgaagtggctttgaccccattaagttccaagaacgaagcagagatctcgattgatagtacgaaagcggggttattgaagagtaatatcgaggggcagcatggcttggacgagagtgatagcgaggatgatgtcgttagcgatgaagtggaggacgatacagcagtagaagcacacaaaagaacgagtagcgtagctgaagatgtgatctcgaagaaggggggatatggaagatttgctcaaaaatggttctcgaagaaaggatgggccgtggaccagaagaagaacctggggatgagcgctgagccgtattccacagtggagcaagcttccaaggccaccgatgtaccagctacgatttcaggagtcactgaaggaaaatctgatatctcaattcccgataagggcaaggaaattgaggacattgaaactcctgaaaatattagcgacattgcagagagcatgctgccaaaattactacgaacatcgcagatattgtttggggcctctcggagttactacttttcttacgaccatgatatcacaagaagtttggcaaataagaggaatacaaattctgaattgccattgcacaaggaagttgatccactcttcttctggaatcggcatcttactttaccatttattgatgctggccagtcttctcttgccttgcctcttatgcagggctttgtaggacagcgtgcattttcaatggatagtaatccaccaaaccctgctataggttcagacactggaaagacttccgtgcagatgaaggatattacaacaagtagttcggatgagcaaatttacacagcacgtgctggtacagacaagtcgtatctattgacgttaatatctagaaggtcagtcaaacgtgccgggcttagatatttacgccggggtgtggatgaggacggcaatacagccaatggcgtggaaacagagcaaatcttatcggattctgcttggggcccttcgagtaagacatattcgttcgttcagatacgtggcagcattcccatattcttctcccagtcaccttactcttttaaacctgtacctcaagttcaccactctaccgaaacaaattatgaagctttcaagaagcattttgataatataagtgatcgctacggggccattcaagtggcttccttggtggagaagcatggaaacgaggcaatagtcggtggagagtacgagaaattgatgactctccttaatgtctcccgagctagcgagcttaggaaatccattgggtttgaatggtttgatttccatgctatttgcaaaggtatgaaatttgagaatgtcagcctgctcatggaaatactggacaagaagcttgactcgttttcgcacactgttgaaaccgatgggaaacttgtatcgaaacagaatggcgttttaaggactaactgtatggattgtctggatcgaacaaacgttgttcaaagtgcagtggcaaagcgagcacttgaaatgcagttaaagaatgagggactagatgtcactctacaaattgatcaaactcaacaatggttcaatactttgtgggccgacaatggtgacgccatttctaagcaatacgcttctacagcagcattgaagggagactttactcgtactaggaagcgggattataagggggccatcacagatatggggctttctatctccagattttatagcggcattgtaaatgactacttcagtcaagctgccattgatttcctgcttggaaatgtgagctatcttgtttttgaagacttcgaggcaaacatgatgagcggtgatcctggcgtttcgatgcaaaaaatgaggcaacaagccattgatgtttctcagaaactcgttgttgctgacgaccgtgaagaatttattggaggatggacatttctcactccgcaggtacccaatacgatcaaatctagtccttttgaggaatccgtcctcctattgacagatgctgcattgtatatgtgcaattttgattggaatatcgagaaagtatcatctttcgtgagagtggacttgaaccaggtgaacggcatcaagtttggaacatacatcacgagtactttgtcacaagcccaggcagatgagaagaggaatgtgggctttgtaataacttataaggctggttcaaacgacattattcgcgtgaacacgagatctatggctacggaatttccttcttcgaaactctctctcgaagacaaaacatccacgcccgcttctacatctaccaccaactctgtcgtcgccccaattgccgccgggtttgcaaacctaatctcaggtttacaaaatcaaagtatagcggaacctaaagatctcgtgaaggttctcgcattcaaggctctaccctccagatctgcggtatcagatgaaggagttagtgaggccgagcaagtgaagagtgtctgtggagagattagaagaatggttgagattggaagtataagagaggctggagaggagagaaaggatattgtagaggagggtactatcattagtttggccgaggccaagaaaagcacgggactattcgatgtgctgggacatcaggtgaagaaactggtttgggcttaatgaaagtgtatcgatactcgtgctagtaatgcttagagcaaaagaagcacttcttgaaggatttacgaatggaattgtggaagttggcagggaggttagcgatcgtcaagaacgggtatgtggaattcaattccatattgaagctgcgaaactcattaacttcaatagaagtggatgtgtagatagacccgagtatatggtattggccagataagtaattttaatgggga

SEQ ID NO: 37 - DNA, 1292 bp, Sclerotinia sclerotiorum
atgcctggcctcgttcgaaagcttcttatctttgccgccattgatggcttgattctgcaaccaacggcgcaaaaaggccagcgccccgcccccgcaacgaagatcacgtataaagataagcatgtcggaccagcatcttatgattctcacgattacgaggggccgtctgccaaaggctttgaagcattcgggattgtcggtctcttgacggtttctaaaagctccttcttaatatcgattacgaaaagggaacaagtcgcacaaatacaaggaaaacctatatatgttattactgaagtagctttgacccctctagcttccaggatagaagcagagaactcgatcaacaaaacaagagcgggattgttaaagagtagtattgaagatcatggattggacgacagtgatagtgaggatgacgaagtcaatgttagtgacgaagtggaggacgatacagcaatagaaacacatacaagaacgagcagtgtggccgaagatgtaatttcgaagaagggagggtatgggagattcgctcaaaaatggttctcgaagaaaggatgggctgtggaccagaagaggaacctgggaatgagcactgaaccgtatgctgcacgagagcaagatgccaggtctgccgacgtagcagctaccacttcaaaggatgctgaagtggaacctgaggttttgatttccgatgaggtcagggacattgaaaatgttggaaagtctgacaaggttaagaacgttcaggatattgctgagagcatgctgccaaagttactgcgtacgacacaaatattgtttgggacctcccggagttactatttttcttacgatcatgatatcacaagaagtttggccaataaaaggaacacaaactctgaattgccattgcataaggagtcgatccactcttcttctggaaccgacaccttctgttaccatttattgatgctgggcaagcttcacttgccttgcctattatgcagggcttcgtaggacaacgagcatttgtaatggatagcaatccgccaaagcctgttgtaggttcggacactgagaagacctccatggaactgaatgagatcacaacagatagttcggatgaacaaatctccacagcacgtgttagtgcagataagccatatctattgacattagtgtctagaagatcggttaagcgtgccgggcttagatatcttcgtcgaggtgtggatgaggacggcaataccgccaatggtgtggagacggagcaaattttaatcagattctacttgggctccttcaagtaa

SEQ ID NO: 38 - DNA, 516 bp, Artificial Sequence
  Description of Artificial Sequence Synthetic polynucleotide
ttcgttccaggagttacacgcaaagatcacttcactggtggaccagcaaaaagatagtaaacatatcgggaaaatatcgatatatattctccgaattctacgagatatcagagcagaattacctagtaaccctgcactacaaaagtttggactctcacttgtctcatcattcgtgctctcgtcgaaagcaaagacaataaagaagaacaaatggagcgcgagttggagggattgcgaagaggaagtgttagcaatcctactacgcatcgtactagtgccatgagcagcggaactgtgactcaggataggaattctctccaagacaataagagcacagttgtaagctggacgttgttcaaagtgcagtggcaaagcgagcacttgaaatgcagttaaagaatgagggactagatgtcactctacaaattgatcaaactcaacaatggttcaatactttgtgggccgacaatggtgacgccatttctaagcaatacgcttctacagcagcattgaagg

SEQ ID NO: 39 - DNA, 490 bp, Artificial Sequence
  Description of Artificial Sequence Synthetic polynucleotide
tgcggaagaacttgaaggtttgctacacagtcaaatatgtactgcagaagatcccagcttgctgcagtactcaatcaaaggtaaacctgagactcttgcctactatgatcccttgggcccgaaattcaatactcctctttatcttcaaatgctcccgcttctaaaagacaatcctatctttcggaagccatttgtatttgggacagaagccagtagaactctaggatcttggtgtgttgaccagatctggacggatgccatttgctgcacgccaaaaatacatcgagcagatcttcgccttcgagtaaagctaccacttctatctattatctactataccccagagtcaaatatcatcgtgacgaaaactgtggcgagcctgagaaagattgtgcaaagtctcaacattttcgaagacccctacgttttgacactaaaaaggagtgatagcgaaaaaagtcaacgtgagctggcgaaagtactcaagagt

SEQ ID NO: 40 - DNA, 2652 bp, Rhizoctonia solani
atgcactatctttcctttgcagctcttgcttttgcgcccatcttggctattgcgactcctgttagccgttgcacgggcactatcgcctctctggatgacgtcgctgctgcccagaaatgcactactgtcactatcaaaggctttactgtccctgccggaaagacgtttgagctttctctcctagacaacaccgttgtcaacatggaaggagacgtaaagttcggagttgcgaactgggccgggccgctattttccgtctcgggaaagggtatcacattcaacggcaatggccacacgttcgatggtcaaggcccgtcctactgggatggtcagggcggtaatggaggtgtgaccaagccccacccgatgatgaagatcaagatttcgggtacatactccaacgtaaaggtcctcaactcgcccgcacatacctacagcatctcgaaccctgcaaagctggtcatgtccaagcttacaattgacaactgtaagtgcccacataatccacgggtggcaccgatatatgtactaacatcgtctctagctgcaggagatgccccgaataatcaatccggaggcaaggccgccggtcacaatactgatggctttgatgtttccaccaccgacctcaccattgaggacagcaccatccgtaaccaggatgactgcattgccattaacaagggctcgaacatcatcttccagcgcaactcttgcaccggcggtcatggtatctctatcggttcgatctcgaccggagcgaccgtccaaaacgtacagatcctgaacaaccagatcatcaacaacgaccaggctctccgcattaagactaaagcggatgctaccagtgcttctgtctctgggatcactttctctggcaacactgcaactggcacaaagaaattcggtgtgattgttgaccagggatatcccactacactcggagctcctggaaatggggtcaagatttcggtgaggctcttgctagaaacatgcttcaattcgtcgaccggcaacaccaacaacatcgcagtcacttccagcgctcagcgagtggccgttaactgtggcacaggatgcacaggcacatgggactggtccaaattgactgtgaccggaggaaaggcctctgacagcaagtacaggtattcgggcgtcaaaggagcgccatggcgctgtagatctccaactcgatcaaattccgtaatggggaacaaagtactcggtttgccacctcacattaccccattccactcgctaattgacgtcttcttatcgtcggtcctaatcacaaaccggatgcaggcctatctcagcacttttgcagctactccaacagatggtcgcgatacgttaacttcgcttgcgcagctatcggttgagcttacttcgggcaccagtgtgaaacttgaccgacccgctcacgctcggtgggcctacacttcactcatccagggacttcccggccggtatacctcacaagacgcgtcccagccgtggctcatttattgggcattacaaacccttacatgtcttggggttcaattggaccccgccaccaaacagcgcactattgatacgatcatcgcaaatcagcatcctgatggtggttttggaggaggacctgatatccgagatttacgccatggtttctccaggcagaagtgctatgaattttttatgaggatgaaacagccggatggatcatttgtcgttaacaaggacgccgaagtggatgtcaggggaacatattgtcttttagttgtagcaactctcctcgacatattaactccagaattggtggagggaacttccgagttcttacgcagctgtcagacatatgagggagggttcgcgtcctcttctcacccatattacagcccagaggatggtaaacctcaagtgctatctgaaattcgtccaaccctaggagaggcccacggcggctatacgtcatgcgctattgctagctggatattactacaaccctaccagaagccggaagatcccaaggtcaatgtgaaaaagctggtacgatgggcgactggaatgcaaggtcttccgatagagggaggagggttccgcggccggaccaacaaattagttgatggctgttattcgtggtggattggagggctcgagccccttttgttggagctgctcgggcttggtaatgacgaaggagagactgaggtagtgagtcatgtcacagaggaaacagacaacgccccgatggccttgttcgataagacatcactgcaacggttcaccttggtctcatctcagctctcatccggtgggctccgcgacaaacccggaaaggctgccgatctttaccatacggcatacaatctagcaggctattcaacggctcagcatcgagtttatcgatctttagtcacagaaaggaaattgcttgatgcctggaagagctcaagcggtgtcattcaaggttcagaagaaaagatacggaagataacttgggctaggatatgcgcatggcaggaagatgaaggtgcacatttctacctcggaggggagggaaatcgggtgcagattggtctacagaatgctactcaccctctattcaacctgacgatatcacacacgcgtgcaatgatgaactatttctaccagcaagaggggctctag

SEQ ID NO: 41 - DNA, 157 bp, Rhizoctonia solani
ccccattccactcgctaattgacgtcttcttatcgtcggtcctaatcacaaaccggatgcaggcctatctcagcacttttgcagctactccaacagatggtcgcgatacgttaacttcgcttgcgcagctatcggttgagcttacttcgggcaccag

SEQ ID NO: 42 - DNA, 1576 bp, Aspergillus niger
atgtacctccttcccttgacgctcttcctcaccgccgctttcggcgtctcaatccctagatctcccctcatccccggcgcacaaatcgtccccgcatccagcacagcagatctacgagccattggtgctcaacatcacaagtatccagaccgagagacagttactattcgggcctcgaggaacgccctcgacgatgtgtccagtgacttcctctggggcttgaagcaggcgaaccatggcggtcggttgttgttgaagcagggggagacctacgtgattgggaagaagttagatttgacattcttggataatattgaggtgcagcttgagggagaaattcaggtactttccttgcccttcttcaatacggagtattgaaaatatgatactgatttcggtggtcctgcttagttcacaaacaacatcacctactggcaagccaacaacttttactacgacttccagaaatccatcaccttctggcgctggggtggccaggacatcaagatcttcgggagtggtgtgttgaacggcaatggacagaaatggtatgatgagtttgcggggaagcagatcttggtatgtcacaccatgataccatccgtacctccctgaaagaacagacaatgctgatgacagcaacgatgataggactcagataacacgttctaccgtcccattctcttcctcaccgataatgcaacccgtatctccgtcgagggcatcacgcagctgaactcgccgtgctggacgaactttttcgttcggaccaatgatgtctcgtttgataatgtgtatattcatgcgttctcgaccaatgcttcagtcagtcctctattcctctggcttttagttgatttccattgcatggatgctaactgatgacagtccgaccccgccaacaccgacggtatggactctctcgacgtcgatggcgtcagcttcaccaatatgcgcatcgatgtcggagatgactgcttctcgccgaagccgaacacaaccaacattttcgtgcagaacatgtggtgcaataacacgcacggggtgagtatgggtagtattggccagtacgcgggcgagatggatatcattgagaacgtgtacattgagaatgtgacgttgctgaatggacaggtacgtcttcttgttccccactgacccatattacaagactgatgtggaatagaacggcgcccgcctcaaagcctgggccggccaagacgtcggctacggccgcatcaataacgtcacgtacaagaacatccagatccagaacacggatgcgccgatcgtgctggaccagtgctactttgatatcaacgctacagagtgtgccaagtacccgtctgctgtgaatatcacgaatatcctgttcgagaatatctggggctcttcctcgggcaaagatggcaagattgtagctgatctggtgtgttcgccagatgcggtgtgcacgaacattactttgtcgaatgtcaacttgacgagcccgaagggcactgcagagattgtttgcgatgacattcagggaggaattggggtggattgtgtgagtgacgagagtgttacgcggtag

SEQ ID NO: 43 - DNA, 219 bp, Aspergillus niger
cgacgatgtgtccagtgacttcctctggggcttgaagcaggcgaaccatggcggtcggttgttgttgaagcagggggagacctacgtgattgggaagaagttagatttgacattcttggataatattgaggtgcagcttgagggagaaattcaggtactttccttgcccttcttcaatacggagtattgaaaatatgatactgatttcggtggtcctgc